(19) United States
(12) Patent Application Publication
(10) Pub. No.: US 2024/0206324 A1
(43) Pub. Date: Jun. 20, 2024

(54) HETEROCYCLIC COMPOUND, ORGANIC LIGHT-EMITTING DEVICE COMPRISING SAME, MANUFACTURING METHOD THEREFOR, AND COMPOSITION FOR ORGANIC LAYER

(71) Applicant: LT MATERIALS CO., LTD., Yongin-si, Gyeonggi-do (KR)

(72) Inventors: Min Ji PARK, Yongin-si (KR); GWANG IL DONG, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/280,046

(22) PCT Filed: Mar. 4, 2022
(86) PCT No.: PCT/KR2022/003066

§ 371 Date: Sep. 1, 2023

(30) Foreign Application Priority Data
Mar. 5, 2021 (KR) 10-2021-0029686

(51) Int. Cl. H10K 85/60; C07C 211/61; C07D 209/80; C07D 209/86; C07D 307/91; C07D 487/04; C07D 491/048; C07D 495/04; C09K 11/06

(52) U.S. Cl. CPC H10K 85/633; C07C 211/61; C07D 209/80; C07D 209/86; C07D 307/91; C07D 487/04; C07D 491/048; C07D 495/04; C09K 11/06; H10K 85/615; H10K 85/626; H10K 85/636; H10K 85/657; H10K 85/6572; H10K 50/11

(57) ABSTRACT

The present specification relates to a heterocyclic compound represented by Chemical Formula 1 and an organic light emitting device comprising the same.

### TECHNICAL FIELD

This application claims priority to and the benefits of Korean Patent Application No. 10-2021-0029686, filed with the Korean Intellectual Property Office on Mar. 5, 2021, the entire contents of which are incorporated herein by reference.

The present disclosure relates to a heterocyclic compound and an organic light emitting device comprising the same.

### BACKGROUND ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, light emitting assistance, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

An object of the present disclosure is to provide a heterocyclic compound and an organic light emitting device comprising the same.

### Technical Solution

In order to achieve the above object, the present disclosure provides a heterocyclic compound represented by the following Chemical Formula 1.

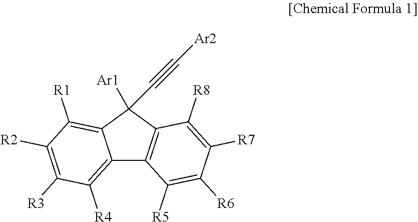

In Chemical Formula 1,
Ar1 and Ar2 are the same as or different from each other, and each independently a substituted or unsubstituted C6 to C60 aryl group; or a substituted or unsubstituted C2 to C60 heteroaryl group,
R1 to R8 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R1 to R8 is a group represented by the following Chemical Formula 2,

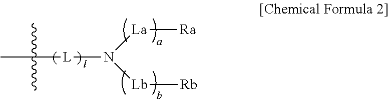

in Chemical Formula 2,
Ra and Pb are the same as or different from each other, and each independently a substituted or unsubstituted C6 to C60 aryl group; or a substituted or unsubstituted C2 to C60 heteroaryl group, or Ra and Rb bond to each other to form a substituted or unsubstituted C6 to C60 aromatic hydrocarbon ring or a substituted or unsubstituted C2 to C60 heteroring,
L, La and Lb are the same as or different from each other, and each independently a direct bond; a substituted or unsubstituted C6 to C60 arylene group; or a substituted or unsubstituted C2 to C60 heteroarylene group, and
l, a and b are the same as or different from each other, and each independently an integer of 0 to 5, and when l is 2 or greater, each L is the same as or different from each other, when a is 2 or greater, each La is the same as or different from each other, and when b is 2 or greater, each Lb is the same as or different from each other.

In addition, one embodiment of the present disclosure provides an organic light emitting device comprising:
a first electrode; a second electrode provided to face the first electrode; and one or more organic material layers provided between the first electrode and the second electrode, wherein one or more layers of the organic material layers include the heterocyclic compound represented by Chemical Formula 1.

In addition, one embodiment of the present disclosure provides an organic light emitting device, wherein the organic material layer includes a hole transfer layer, and the hole transfer layer includes the heterocyclic compound.

In addition, one embodiment of the present disclosure provides an organic light emitting device, wherein the organic material layer includes an electron blocking layer, and the electron blocking layer includes the heterocyclic compound.

In addition, one embodiment of the present disclosure provides an organic light emitting device, wherein the organic material layer includes a light emitting auxiliary layer, and the light emitting auxiliary layer includes the heterocyclic compound.

In addition, one embodiment of the present disclosure provides a method for manufacturing an organic light emitting device, the method comprising the steps of:
preparing a substrate; forming a first electrode on the substrate; forming one or more organic material layers on the first electrode; and forming a second electrode on the one or more organic material layers, wherein the forming of one or more organic material layers includes forming the one or more organic material layers using a composition for an organic material layer of an organic light emitting device including the heterocyclic compound represented by Chemical Formula 1.

### Advantageous Effects

The heterocyclic compound according to one embodiment of the present application can be used as a material of an organic material layer of an organic light emitting device. The heterocyclic compound can be used as a material of a hole injection layer, a hole transfer layer, an electron blocking layer, a light emitting auxiliary layer, a light emitting layer, an electron transfer layer, a hole blocking layer, an electron injection layer, a charge generation layer or the like in an organic light emitting device. Particularly, the heterocyclic compound represented by Chemical Formula 1 can be used as a material of a hole transfer layer, or a material of an electron blocking layer or light emitting auxiliary layer of an organic light emitting device. Specifically, the heterocyclic compound represented by Chemical Formula 1 can be used as a hole transfer layer material, or a material of an electron blocking layer or light emitting auxiliary layer either alone or as a combination with other compounds.

The heterocyclic compound represented by Chemical Formula 1 introduces an alkynylene group to a fluorene moiety separated from a group represented by Chemical Formula 2 in Chemical Formula 1, and, by the electron-abundant alkyne, acts as a screen to prevent LUMO from expanding to Ar2, and as a result, the heterocyclic compound represented by Chemical Formula 1 has a shallower LUMO. Accordingly, effects of suppressing electrons introduced from a light emitting layer and further facilitating hole transfer are obtained by relatively strengthening hole transfer properties of the compound. In addition, by being present as one or more layers between each layer of an organic light emitting device, for example, being present between a light emitting layer and a hole transfer layer, the heterocyclic compound represented by Chemical Formula 1 separates an interface of the each layer while mitigating the LUMO difference between the each layer, and an effect of improving color purity of the light emitting layer is obtained by relieving a charge trap. In addition, the heterocyclic compound represented by Chemical Formula 1 blocks migration of excitons by having a proper triplet energy level (T₁) value, and is effective in well preserving triplet excitons in the light emitting layer.

### DESCRIPTION OF DRAWINGS

FIG. 1 to FIG. 3 are diagrams each schematically illustrating a lamination structure of an organic light emitting device according to one embodiment of the present disclosure.

### MODE FOR DISCLOSURE

Hereinafter, the present application will be described in detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O) RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent linking two or more substituents selected from among the substituents illustrated above or being unsubstituted. R, R′ and R″ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to and more specifically from 1 to 20. Specific examples thereof may include a methyl group, an ethyl group, an n-propyl group, an isopropyl group, an n-butyl group, an isobutyl group, a tert-butyl group, a sec-butyl group, a 1-methyl-butyl group, a 1-ethyl-butyl group, an n-pentyl group, an isopentyl group, a neopentyl group, a tert-pentyl group, an n-hexyl group, a 1-methylpentyl group, a 2-methylpentyl group, a 4-methyl-2-pentyl group, a 3,3-dimethylbutyl group, a 2-ethylbutyl group, an n-heptyl group, a 1-methylhexyl group, a cyclopentylmethyl group, a cyclohexylmethyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be used. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

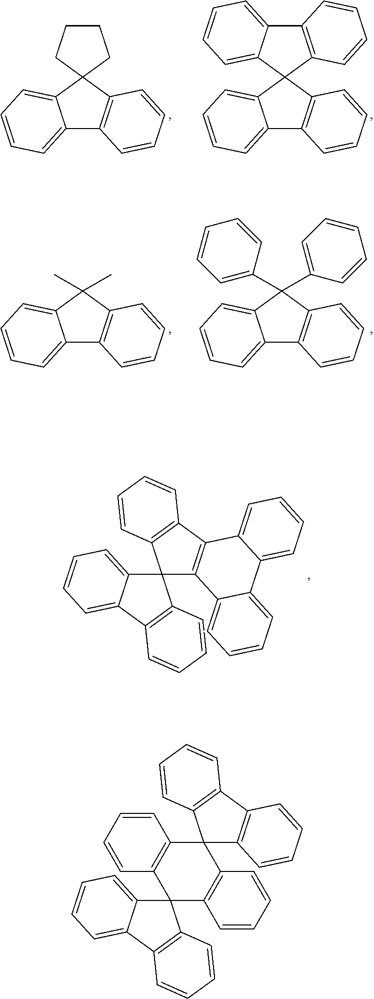

and the like may be included, however, the structure is not limited thereto.

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be mixed in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

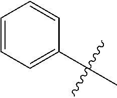 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

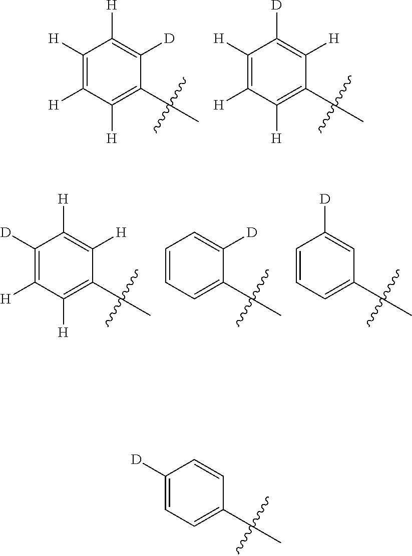 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the deuterium content may be from 0% to 100% and more preferably from 30% to 100, in the heterocyclic compound represented by Chemical Formula 1.

In the present disclosure, the C6 to C60 aromatic hydrocarbon ring means a compound including an aromatic ring formed with C6 to C60 carbons and hydrogens. Examples thereof may include benzene, biphenyl, triphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

One embodiment of the present disclosure provides a heterocyclic compound represented by the following Chemical Formula 1.

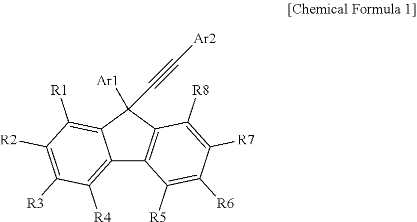

In Chemical Formula 1,
Ar1 and Ar2 are the same as or different from each other, and each independently a substituted or unsubstituted C6 to C60 aryl group; or a substituted or unsubstituted C2 to C60 heteroaryl group,
R1 to R8 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R1 to R8 is a group represented by the following Chemical Formula 2,

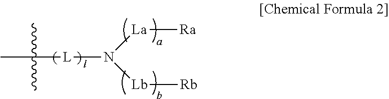

in Chemical Formula 2,
Ra and Rb are the same as or different from each other, and each independently a substituted or unsubstituted C6 to C60 aryl group; or a substituted or unsubstituted C2 to C60 heteroaryl group, or Ra and Rb bond to each other to form a substituted or unsubstituted C6 to C60 aromatic hydrocarbon ring or a substituted or unsubstituted C2 to C60 heteroring,
L, La and Lb are the same as or different from each other, and each independently a direct bond; a substituted or unsubstituted C6 to C60 arylene group; or a substituted or unsubstituted C2 to C60 heteroarylene group, and
l, a and b are the same as or different from each other, and each independently an integer of 0 to 5, and when l is 2 or greater, each L is the same as or different from each other, when a is 2 or greater, each La is the same as or different from each other, and when b is 2 or greater, each Lb is the same as or different from each other.

As the aliphatic or aromatic hydrocarbon ring or heteroring that adjacent groups may form, the structures exemplified as the cycloalkyl group, the cycloheteroalkyl group, the aryl group and the heteroaryl group described above may be used except for those that are not a monovalent group.

In one embodiment of the present disclosure, Ar and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group, biphenyl group, terphenyl group, naphthyl group, fluorenyl group or phenanthrenyl group; or a substituted or unsubstituted dibenzofuranyl group.

In one embodiment of the present disclosure, R1 to R8 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or the group represented by Chemical Formula 2.

In another embodiment of the present disclosure, R1 to R8 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or the group represented by Chemical Formula 2.

In another embodiment of the present disclosure, R1 to R8 are the same as or different from each other, and may be each independently hydrogen; deuterium; a substituted or unsubstituted C6 to C20 aryl group; a substituted or unsubstituted C2 to C20 heteroaryl group; —P(═O)R101R102; —SiR101R102R103; or the group represented by Chemical Formula 2.

In another embodiment of the present disclosure, R1 to R8 are the same as or different from each other, and may be each independently hydrogen; deuterium; or the group represented by Chemical Formula 2.

In one embodiment of the present disclosure, one of R1 to R8 is the group represented by Chemical Formula 2, and the rest may be hydrogen or deuterium.

In one embodiment of the present disclosure, L, La and Lb in Chemical Formula 2 are the same as or different from each other, and may be each independently a direct bond; a substituted or unsubstituted C6 to C30 arylene group; or a substituted or unsubstituted C2 to C30 heteroarylene group.

In another embodiment of the present disclosure, L, La and Lb in Chemical Formula 2 are the same as or different from each other, and may be each independently a direct bond; a substituted or unsubstituted C6 to C20 arylene group; or a substituted or unsubstituted C2 to C20 heteroarylene group.

In another embodiment of the present disclosure, L, La and Lb in Chemical Formula 2 are the same as or different from each other, and may be each independently a direct bond; or a substituted or unsubstituted phenylene group, biphenylene group or naphthylene group.

Specific examples of L are shown below, however, L is not limited to these examples.

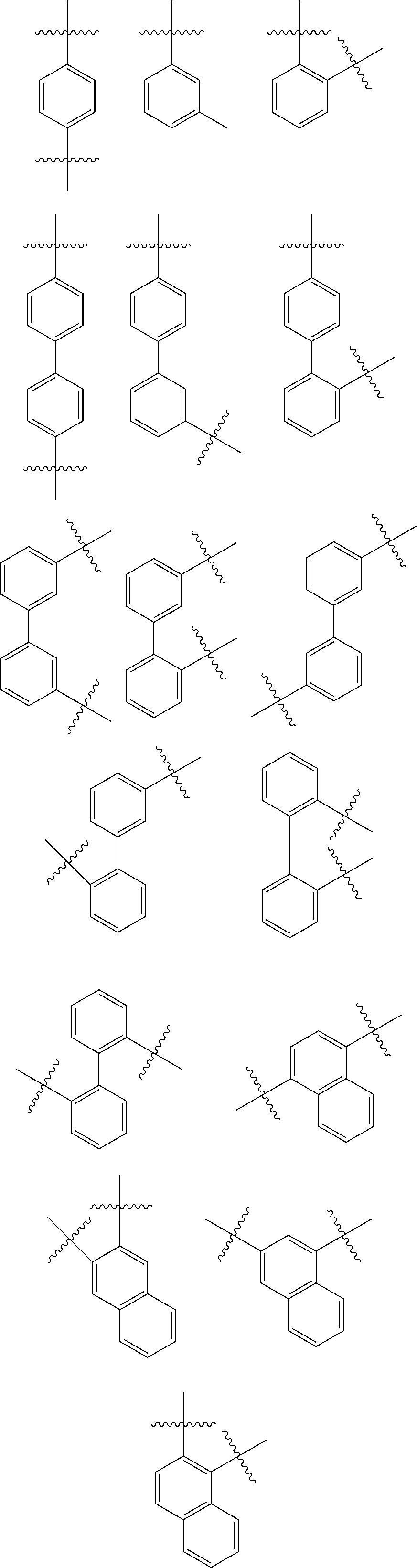

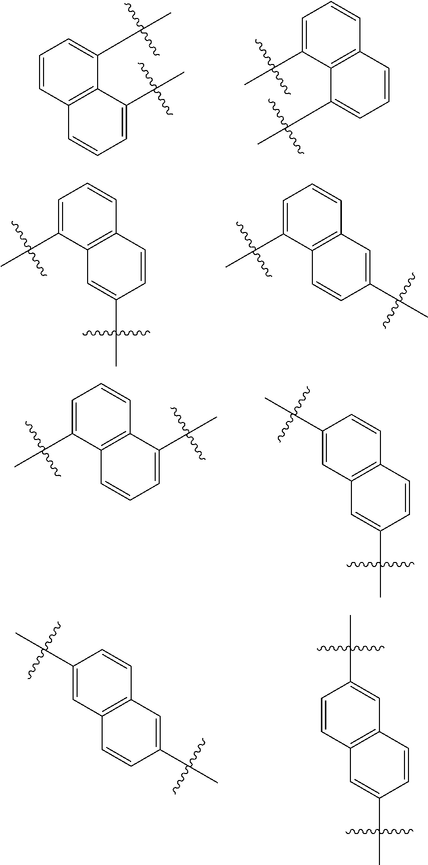

In one embodiment of the present disclosure, l, a and b in Chemical Formula 2 are the same as or different from each other, and may be each independently an integer of 0 to 3, and when l is 2 or greater, each L is the same as or different from each other, when a is 2 or greater, each La is the same as or different from each other, and when b is 2 or greater, Lbs may be the same as or different from each other.

In another embodiment of the present disclosure, l, a and b in Chemical Formula 2 are the same as or different from each other, and may be each independently an integer of 0 to 2, and when l is 2, each L is the same as or different from each other, when a is 2, each La is the same as or different from each other, and when b is 2, Lbs may be the same as or different from each other.

In one embodiment of the present disclosure, Ra and Rb in Chemical Formula 2 are the same as or different from each other, and each independently a substituted or unsubstituted C6 to C30 aryl group; or a substituted or unsubstituted C2 to C30 heteroaryl group, or Ra and Rb may bond to each other to form a substituted or unsubstituted C6 to C30 aromatic hydrocarbon ring or a substituted or unsubstituted C2 to C30 heteroring.

In another embodiment of the present disclosure, Ra and Rb in Chemical Formula 2 are the same as or different from each other, and each independently a substituted or unsubstituted C6 to C20 aryl group; or a substituted or unsubstituted C2 to C20 heteroaryl group, or Ra and Rb may bond to each other to form a substituted or unsubstituted C6 to C20 aromatic hydrocarbon ring or a substituted or unsubstituted C2 to C20 heteroring.

In another embodiment of the present disclosure, Ra and Rb in Chemical Formula 2 are the same as or different from each other, and each independently a substituted or unsubstituted phenyl group, biphenyl group, terphenyl group, naphthyl group, fluorenyl group or phenanthrenyl group; or a substituted or unsubstituted dibenzofuranyl group or carbazolyl group, or Ra and Rb may bond to each other to form a substituted or unsubstituted C6 to C20 aromatic hydrocarbon ring or a substituted or unsubstituted C2 to C20 heteroring.

In another embodiment of the present disclosure, Ra and Pb in Chemical Formula 2 are the same as or different from each other, and each independently a substituted or unsubstituted phenyl group, methylphenyl group, diphenylaminophenyl group, biphenyl group, methylbiphenyl group, terphenyl group, naphthyl group, dimethylfluorenyl group or phenanthrenyl group; or a substituted or unsubstituted dibenzofuranyl group or carbazolyl group, or Ra and Rb may bond to each other to form a substituted or unsubstituted C6 to C20 aromatic hydrocarbon ring or a substituted or unsubstituted C2 to C20 heteroring.

In one embodiment of the present disclosure, Chemical Formula 2 may be a group represented by any one of the following Chemical Formula 2-1 and Chemical Formula 2-2.

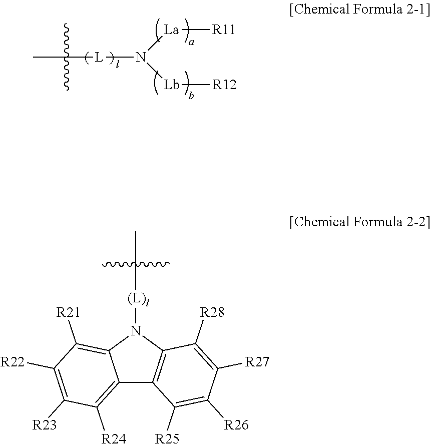

In Chemical Formula 2-1 and Chemical Formula 2-2,
R11 and R12 are the same as or different from each other, and each independently a substituted or unsubstituted C6 to C60 aryl group; or a substituted or unsubstituted C2 to C60 heteroaryl group,
R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
L, La, Lb, l, a and b have the same definitions as in Chemical Formula 2.

In one embodiment of the present disclosure, R11 and R12 in Chemical Formula 2-1 are the same as or different from each other, and may be each independently a substituted or unsubstituted C6 to C30 aryl group; or a substituted or unsubstituted C2 to C30 heteroaryl group.

In another embodiment of the present disclosure, R11 and R12 in Chemical Formula 2-1 are the same as or different from each other, and may be each independently a substituted or unsubstituted C6 to C20 aryl group; or a substituted or unsubstituted C2 to C20 heteroaryl group.

In another embodiment of the present disclosure, R11 and R12 in Chemical Formula 2-1 are the same as or different from each other, and may be each independently a substituted or unsubstituted phenyl group, biphenyl group, terphenyl group, naphthyl group, fluorenyl group or phenanthrenyl group; or a substituted or unsubstituted dibenzofuranyl group or carbazolyl group.

In another embodiment of the present disclosure, R11 and R12 in Chemical Formula 2-1 are the same as or different from each other, and may be each independently a phenyl group, a methylphenyl group, a diphenylaminophenyl group, a biphenyl group, a methylbiphenyl group, a terphenyl group, a naphthyl group, a dimethylfluorenyl group, a phenanthrenyl group; or a substituted or unsubstituted dibenzofuranyl group or carbazolyl group.

In one embodiment of the present disclosure, R21 to R28 in Chemical Formula 2-2 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; and —NR201R202, or two or more groups adjacent to each other may bond to each other to form a substituted or unsubstituted C6 to C30 aromatic hydrocarbon ring or a substituted or unsubstituted C2 to C30 heteroring.

In another embodiment of the present disclosure, R21 to R28 in Chemical Formula 2-2 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; and —NR201R202, or two or more groups adjacent to each other may bond to each other to form a substituted or unsubstituted C6 to C20 aromatic hydrocarbon ring or a substituted or unsubstituted C2 to C20 heteroring.

In another embodiment of the present disclosure, Chemical Formula 2-2 may be a group represented by any one of the following Chemical Formula 2-2-a to Chemical Formula 2-2-d.

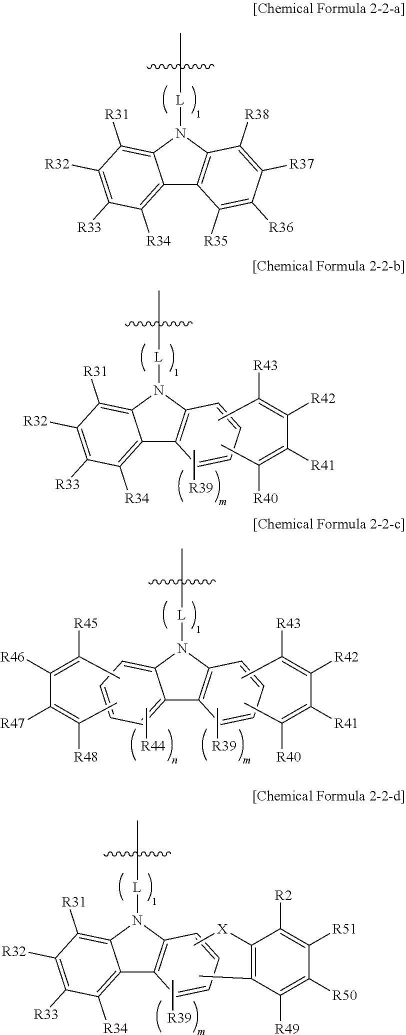

In Chemical Formula 2-2-a to Chemical Formula 2-2-d,
R31 to R52 are the same as or different from each other, and each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; or —NR201R202, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
X is O, S or NR53,
R53 is a substituted or unsubstituted C6 to C60 aryl group; or a substituted or unsubstituted C2 to C60 heteroaryl group,
m and n are the same as or different from each other, and each independently an integer of 0 to 2, and when m is 2, each R39 is the same as or different from each other, and when n is 2, each R44 is the same as or different from each other, and
L and 1 have the same definitions as in Chemical Formula 2.

In one embodiment of the present disclosure, R31 to R52 in Chemical Formula 2-2-a to Chemical Formula 2-2-d are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; or —NR201R202.

In another embodiment of the present disclosure, R31 to R52 in Chemical Formula 2-2-a to Chemical Formula 2-2-d are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; or —NR201R202.

In another embodiment of the present disclosure, R31 to R52 in Chemical Formula 2-2-a to Chemical Formula 2-2-d are the same as or different from each other, and may be each independently hydrogen; deuterium; or a substituted or unsubstituted C6 to C20 aryl group.

In another embodiment of the present disclosure, R31 to R52 in Chemical Formula 2-2-a to Chemical Formula 2-2-d are the same as or different from each other, and may be each independently hydrogen; deuterium; or a substituted or unsubstituted phenyl group.

In one embodiment of the present disclosure, X in Chemical Formula 2-2-a to Chemical Formula 2-2-d may be O or S.

In another embodiment of the present disclosure, X in Chemical Formula 2-2-a to Chemical Formula 2-2-d may be O or NR53.

In another embodiment of the present disclosure, X in Chemical Formula 2-2-a to Chemical Formula 2-2-d may be S or NR53.

In another embodiment of the present disclosure, X in Chemical Formula 2-2-a to Chemical Formula 2-2-d may be O.

In another embodiment of the present disclosure, X in Chemical Formula 2-2-a to Chemical Formula 2-2-d may be S.

In another embodiment of the present disclosure, X in Chemical Formula 2-2-a to Chemical Formula 2-2-d may be NR53.

In one embodiment of the present disclosure, R53 may be a substituted or unsubstituted C6 to C30 aryl group; or a substituted or unsubstituted C2 to C30 heteroaryl group.

In another embodiment of the present disclosure, R53 may be a substituted or unsubstituted C6 to C20 aryl group; or a substituted or unsubstituted C2 to C20 heteroaryl group.

In another embodiment of the present disclosure, R53 may be a substituted or unsubstituted phenyl group.

In one embodiment of the present disclosure, Chemical Formula 2-2-b may be represented by any one of the following chemical formulae.

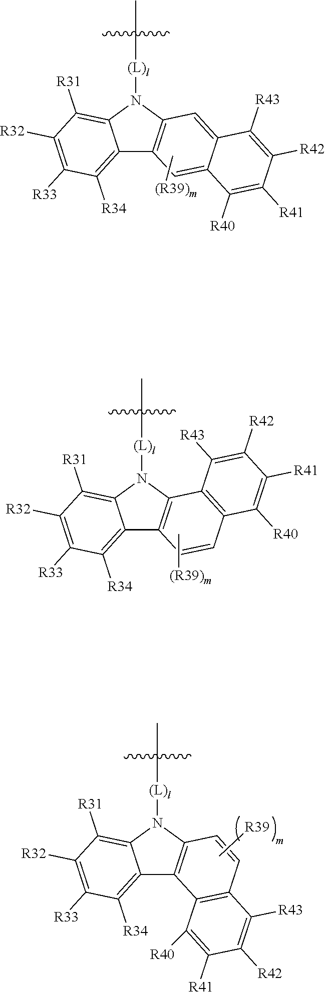

R31 to R34, R39, R40 to R43, L, l and m have the same definitions as in Chemical Formula 2-2-b.

In one embodiment of the present disclosure, Chemical Formula 2-2-c may be represented by any one of the following chemical formulae.

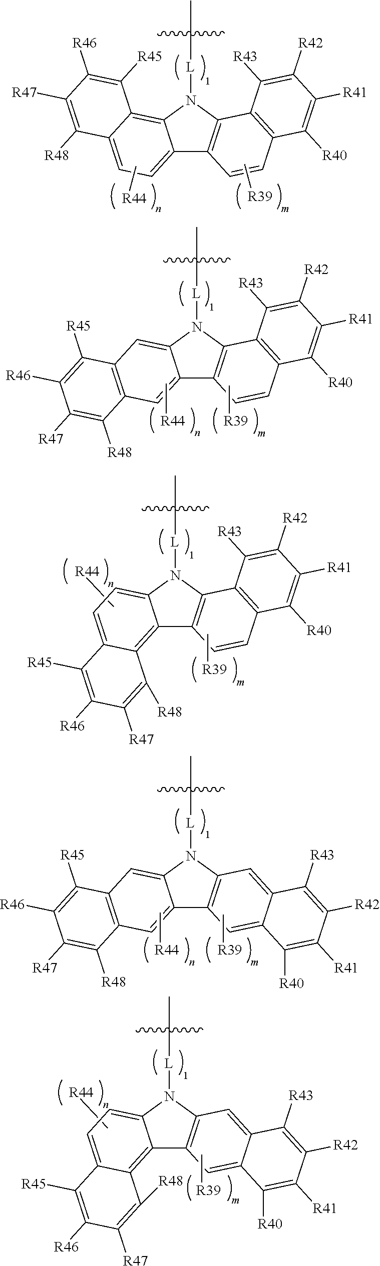

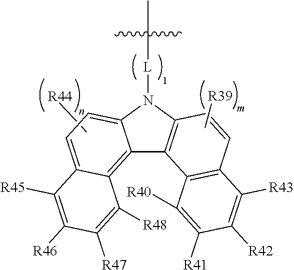

R39 to R48, L, l and m have the same definitions as in Chemical Formula 2-2-c.

In one embodiment of the present disclosure, Chemical Formula 2-2-d may be represented by any one of the following chemical formulae.

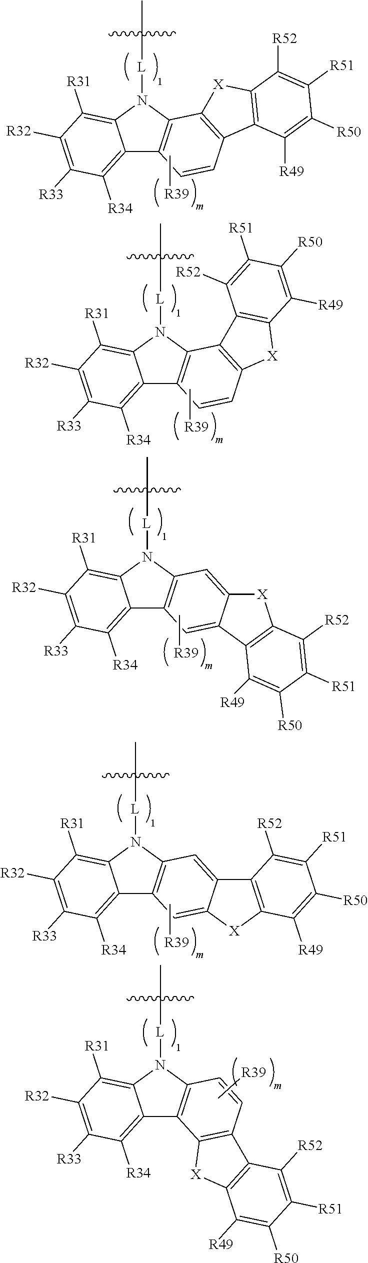

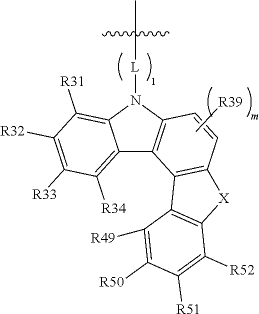

R31 to R34, R39, R49 to R52, X, L, l and m have the same definitions as in Chemical Formula 2-2-d.

Specific examples of Chemical Formula 2-2-d are shown below, however, Chemical Formula 2-2-d are not limited to these examples.

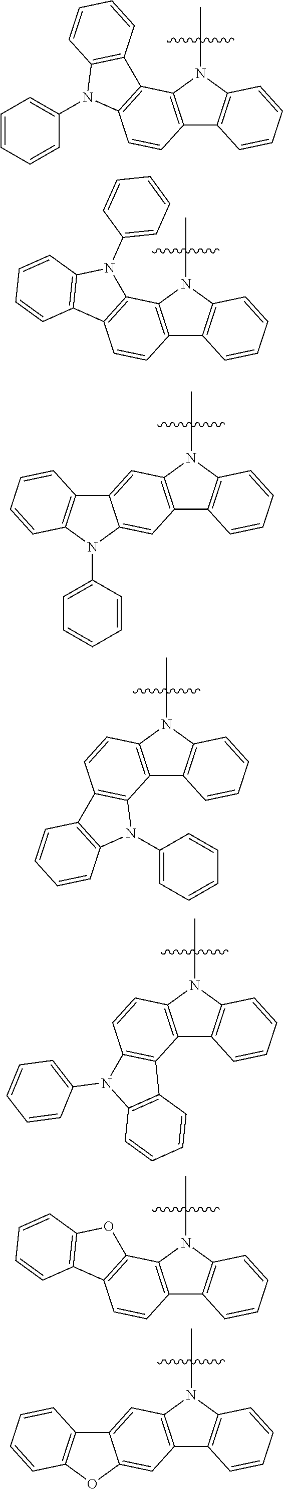

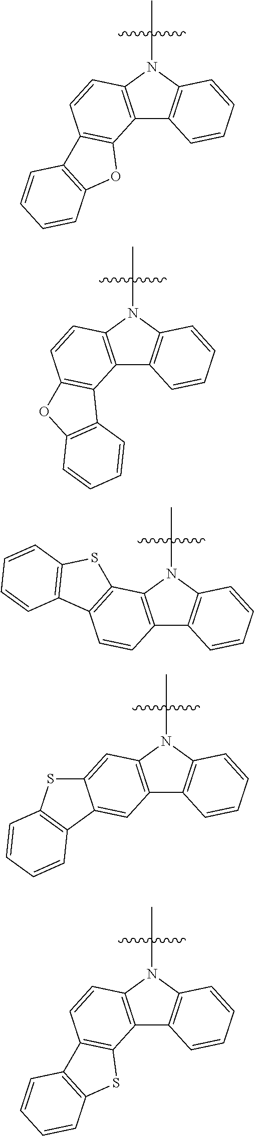 [SMILES: *n1c2ccccc2c2c3oc4ccccc4c3ccc21 ; *n1c2ccccc2c2c3c(ccc21)oc1ccccc13 ; *n1c2ccccc2c2ccc3c4ccccc4sc3c21 ; *n1c2ccccc2c2cc3c(cc21)sc1ccccc13 ; *n1c2ccccc2c2c3sc4ccccc4c3ccc21]

In one embodiment of the present disclosure, the heterocyclic compound represented by Chemical Formula 1 may be one or more types selected from among the following compounds.

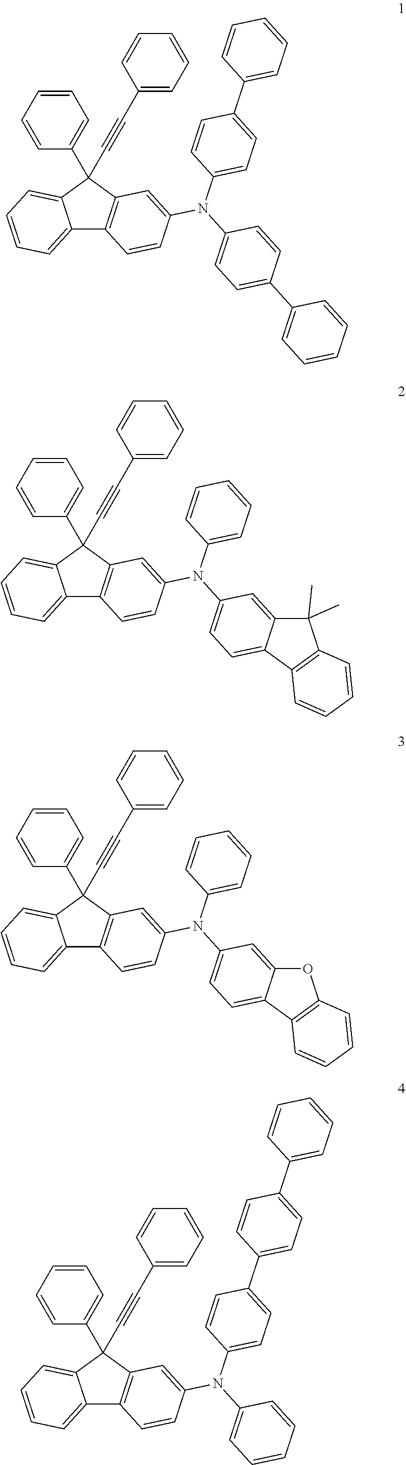

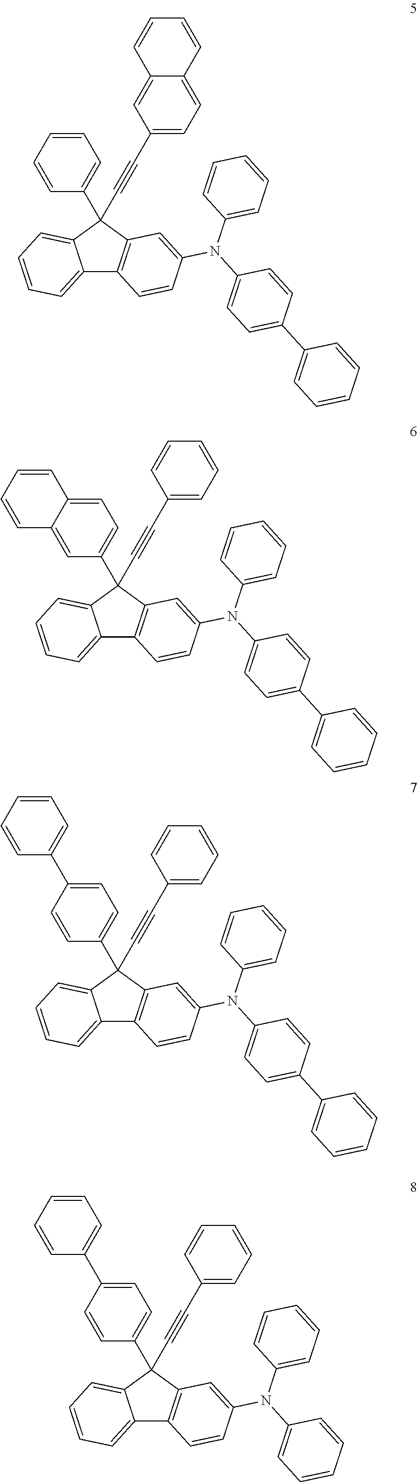 [SMILES: C(#CC1(c2ccccc2)c2ccccc2-c2ccc(N(c3ccccc3)c3ccc(-c4ccccc4)cc3)cc21)c1ccc2ccccc2c1 ; C(#CC1(c2ccc3ccccc3c2)c2ccccc2-c2ccc(N(c3ccccc3)c3ccc(-c4ccccc4)cc3)cc21)c1ccccc1 ; C(#CC1(c2ccc(-c3ccccc3)cc2)c2ccccc2-c2ccc(N(c3ccccc3)c3ccc(-c4ccccc4)cc3)cc21)c1ccccc1 ; C(#CC1(c2ccc(-c3ccccc3)cc2)c2ccccc2-c2ccc(N(c3ccccc3)c3ccccc3)cc21)c1ccccc1]

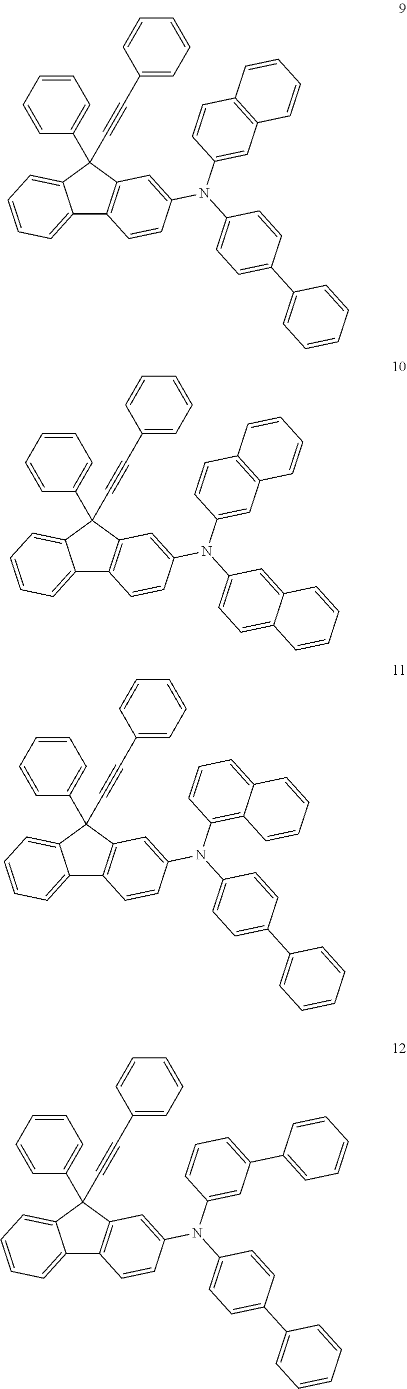 [SMILES: C(#CC1(c2ccccc2)c2ccccc2-c2ccc(N(c3ccc(-c4ccccc4)cc3)c3ccc4ccccc4c3)cc21)c1ccccc1 ; C(#CC1(c2ccccc2)c2ccccc2-c2ccc(N(c3ccc4ccccc4c3)c3ccc4ccccc4c3)cc21)c1ccccc1 ; C(#CC1(c2ccccc2)c2ccccc2-c2ccc(N(c3ccc(-c4ccccc4)cc3)c3cccc4ccccc34)cc21)c1ccccc1 ; C(#CC1(c2ccccc2)c2ccccc2-c2ccc(N(c3ccc(-c4ccccc4)cc3)c3cccc(-c4ccccc4)c3)cc21)c1ccccc1]

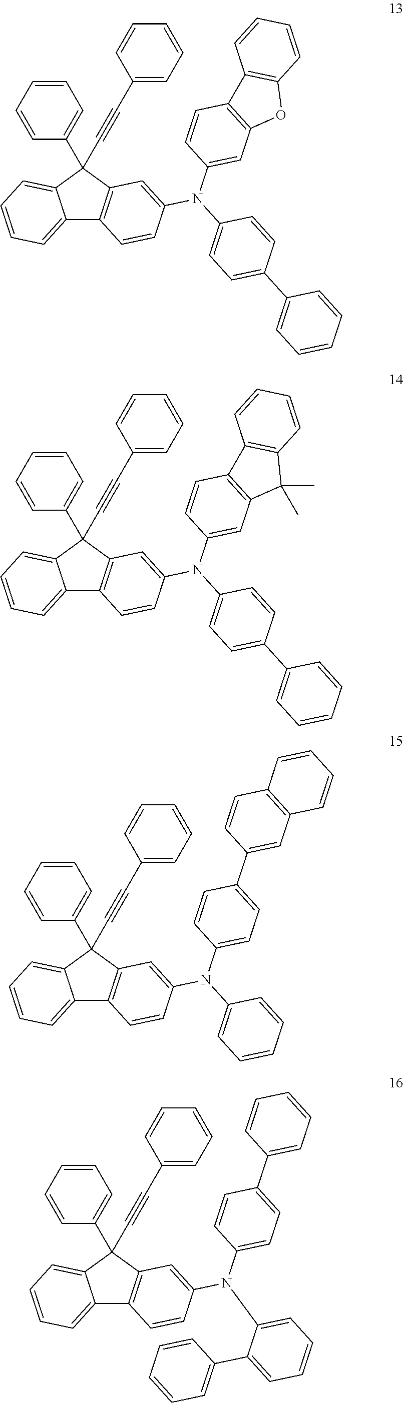

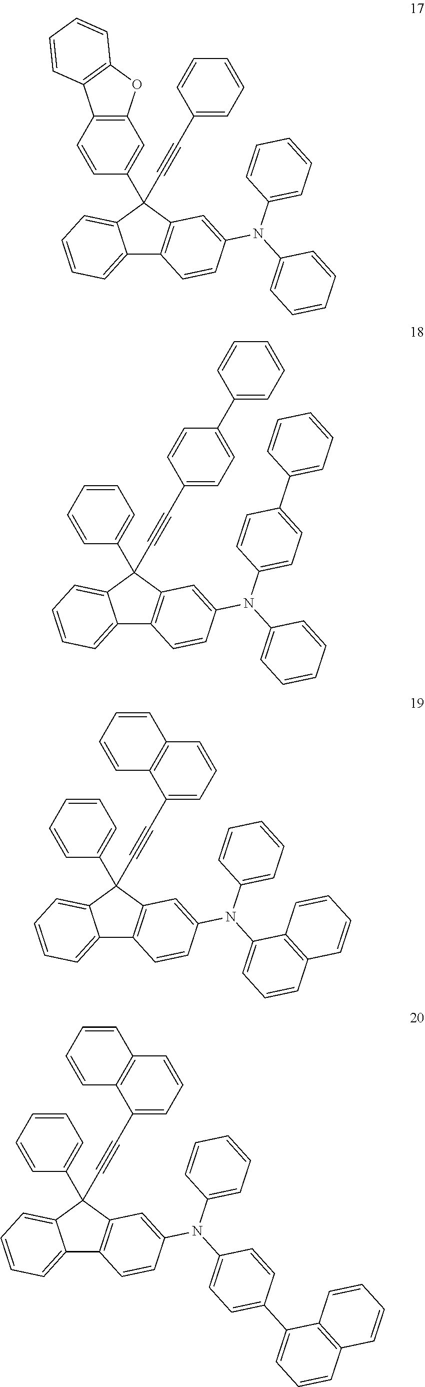

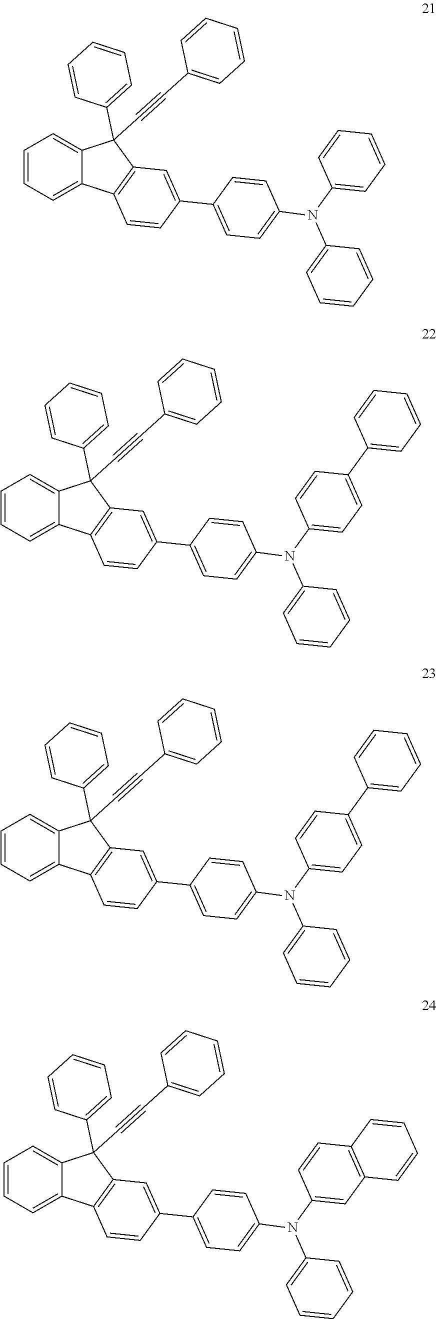 [SMILES: C(#CC1(c2ccccc2)c2ccccc2-c2ccc(-c3ccc(N(c4ccccc4)c4ccccc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccccc4)c4ccc5ccccc5c4)cc3)cc21)c1ccccc1]

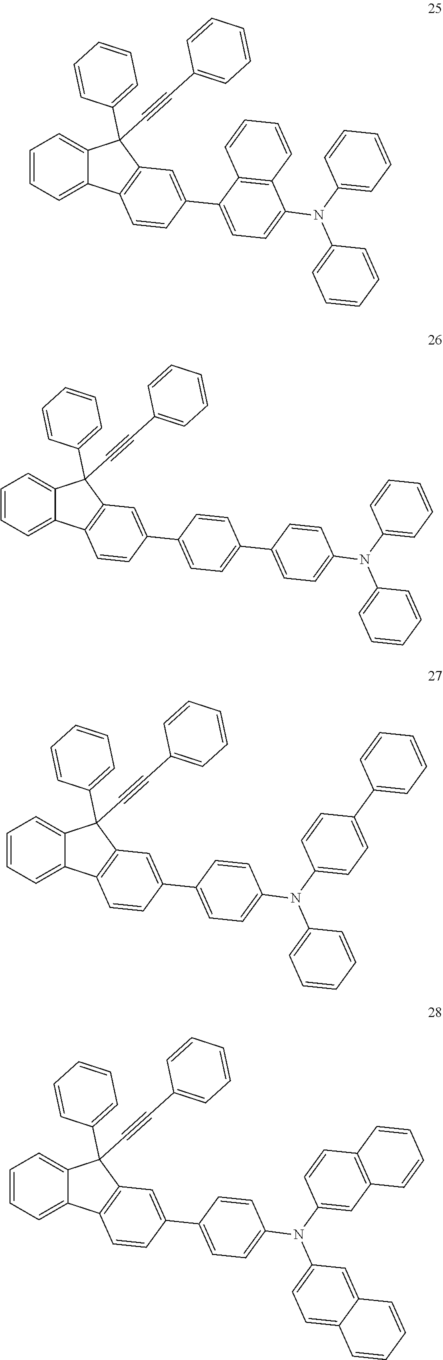 [SMILES: C(#CC1(c2ccccc2)c2ccccc2-c2ccc(-c3ccc(N(c4ccccc4)c4ccccc4)c4ccccc34)cc21)c1ccccc1 ; C(#CC1(c2ccccc2)c2ccccc2-c2ccc(-c3ccc(-c4ccc(N(c5ccccc5)c5ccccc5)cc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccc5ccccc5c4)c4ccc5ccccc5c4)cc3)cc21)c1ccccc1]

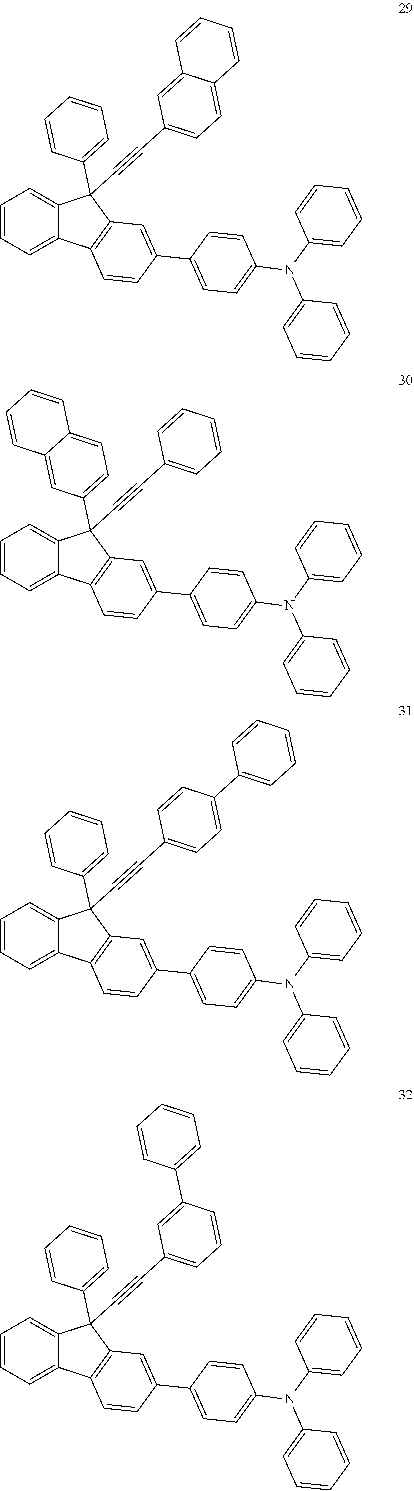

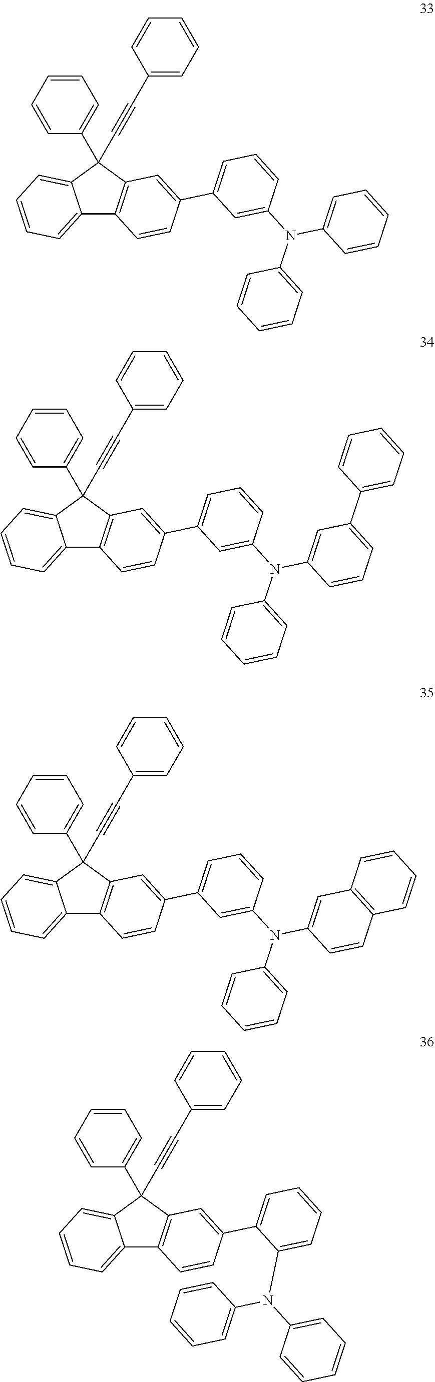

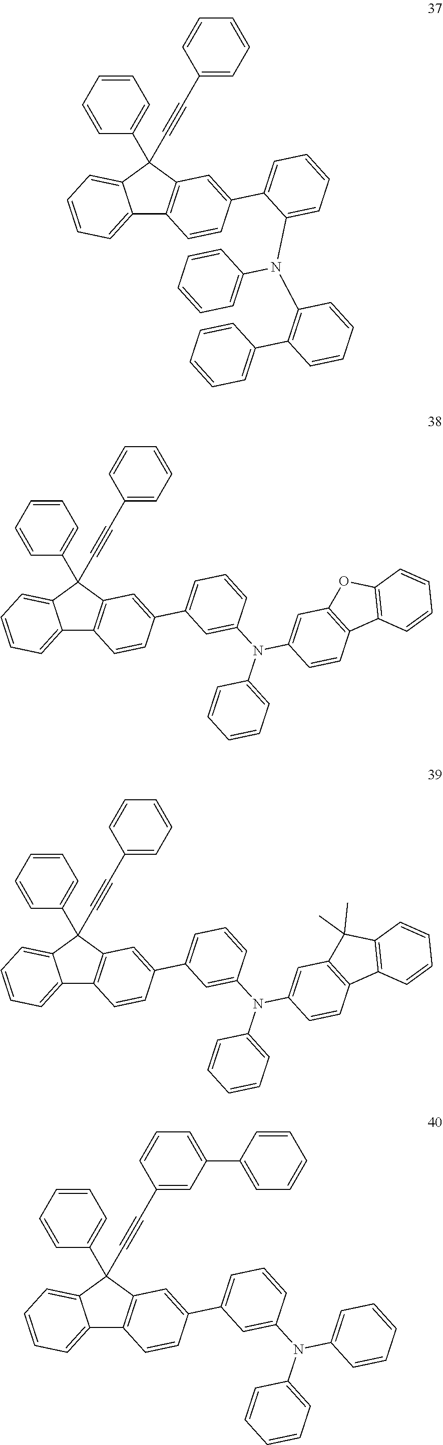 [SMILES: C(#CC1(c2ccccc2)c2ccccc2-c2ccc(-c3ccccc3N(c3ccccc3)c3ccccc3-c3ccccc3)cc21)c1ccccc1 ; C(#CC1(c2ccccc2)c2ccccc2-c2ccc(-c3cccc(N(c4ccccc4)c4ccc5c(c4)oc4ccccc45)c3)cc21)c1ccccc1 ; CC1(C)c2ccccc2-c2ccc(N(c3ccccc3)c3cccc(-c4ccc5c(c4)C(C#Cc4ccccc4)(c4ccccc4)c4ccccc4-5)c3)cc21 ; C(#CC1(c2ccccc2)c2ccccc2-c2ccc(-c3cccc(N(c4ccccc4)c4ccccc4)c3)cc21)c1cccc(-c2ccccc2)c1]

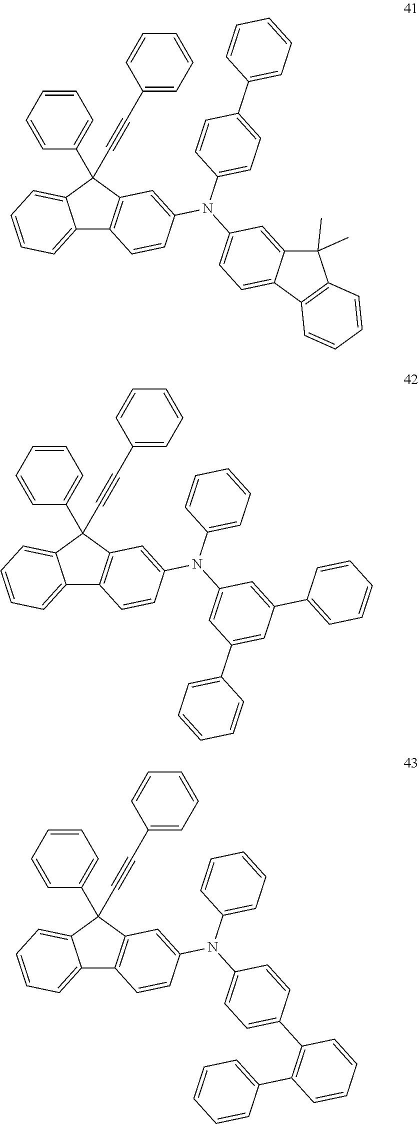

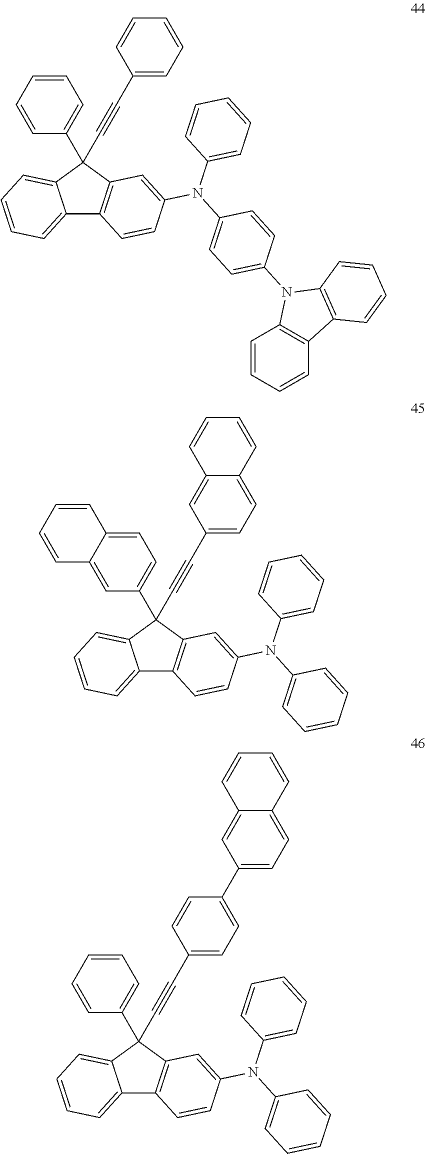

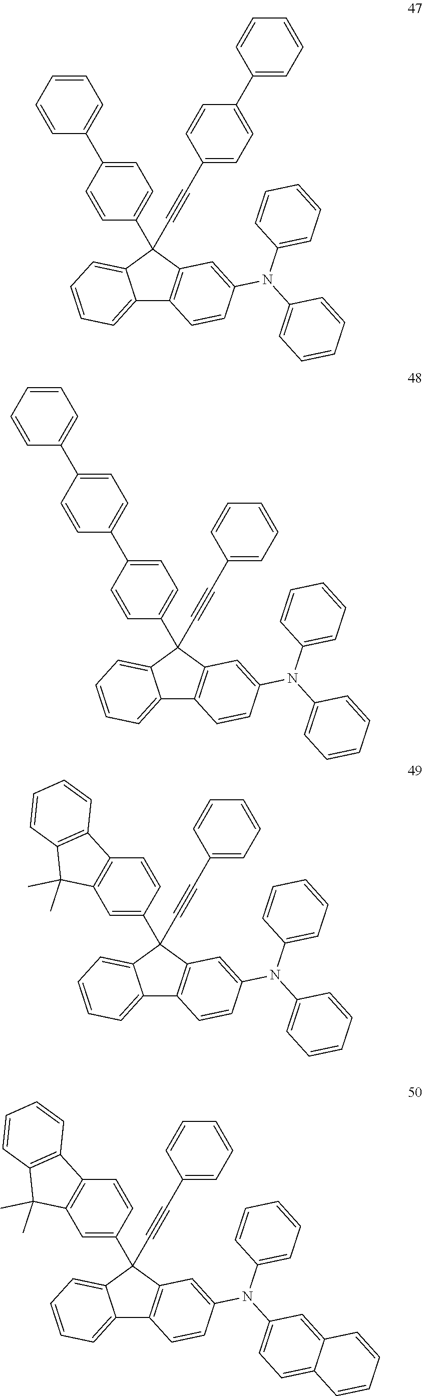 [SMILES: C(#CC1(c2ccc(-c3ccccc3)cc2)c2ccccc2-c2ccc(N(c3ccccc3)c3ccccc3)cc21)c1ccc(-c2ccccc2)cc1 ; C(#CC1(c2ccc(-c3ccc(-c4ccccc4)cc3)cc2)c2ccccc2-c2ccc(N(c3ccccc3)c3ccccc3)cc21)c1ccccc1 ; CC1(C)c2ccccc2-c2ccc(C3(C#Cc4ccccc4)c4ccccc4-c4ccc(N(c5ccccc5)c5ccccc5)cc43)cc21 ; CC1(C)c2ccccc2-c2ccc(C3(C#Cc4ccccc4)c4ccccc4-c4ccc(N(c5ccccc5)c5ccc6ccccc6c5)cc43)cc21]

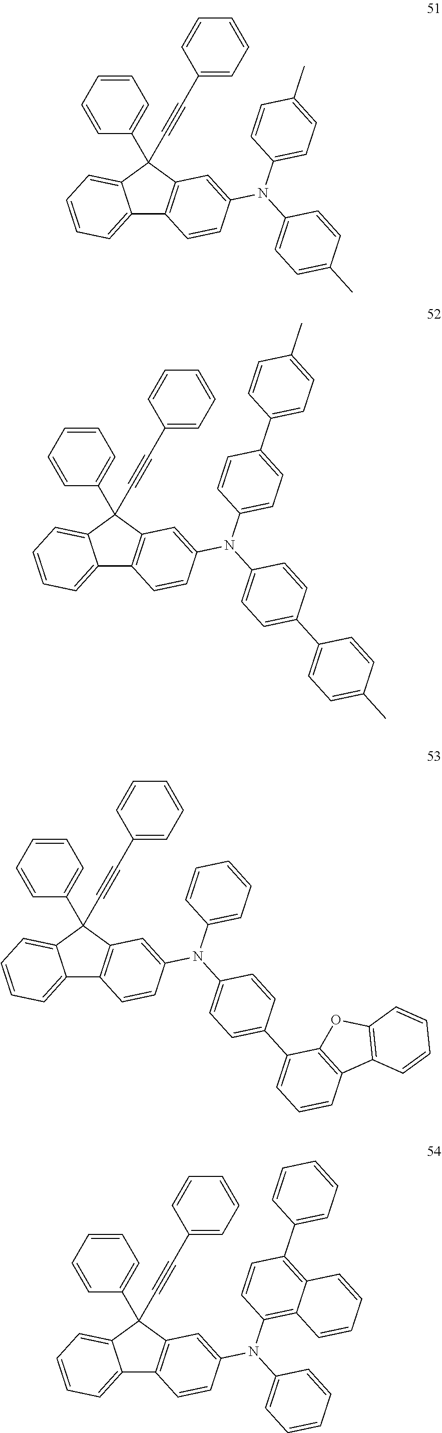 [SMILES: Cc1ccc(N(c2ccc(C)cc2)c2ccc3c(c2)C(C#Cc2ccccc2)(c2ccccc2)c2ccccc2-3)cc1 ; Cc1ccc(-c2ccc(N(c3ccc(-c4ccc(C)cc4)cc3)c3ccc4c(c3)C(C#Cc3ccccc3)(c3ccccc3)c3ccccc3-4)cc2)cc1 ; C(#CC1(c2ccccc2)c2ccccc2-c2ccc(N(c3ccccc3)c3ccc(-c4cccc5c4oc4ccccc45)cc3)cc21)c1ccccc1 ; C(#CC1(c2ccccc2)c2ccccc2-c2ccc(N(c3ccccc3)c3ccc(-c4ccccc4)c4ccccc34)cc21)c1ccccc1]

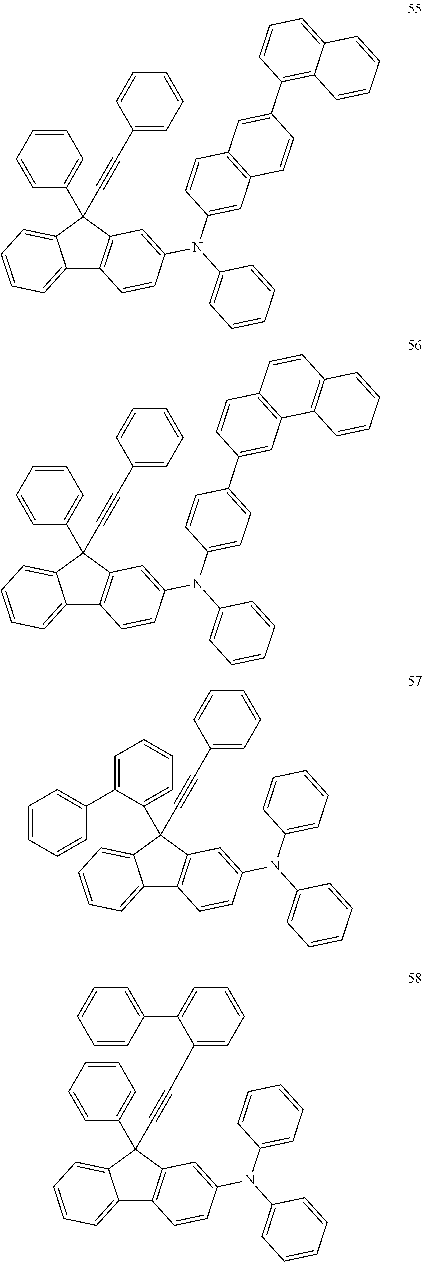

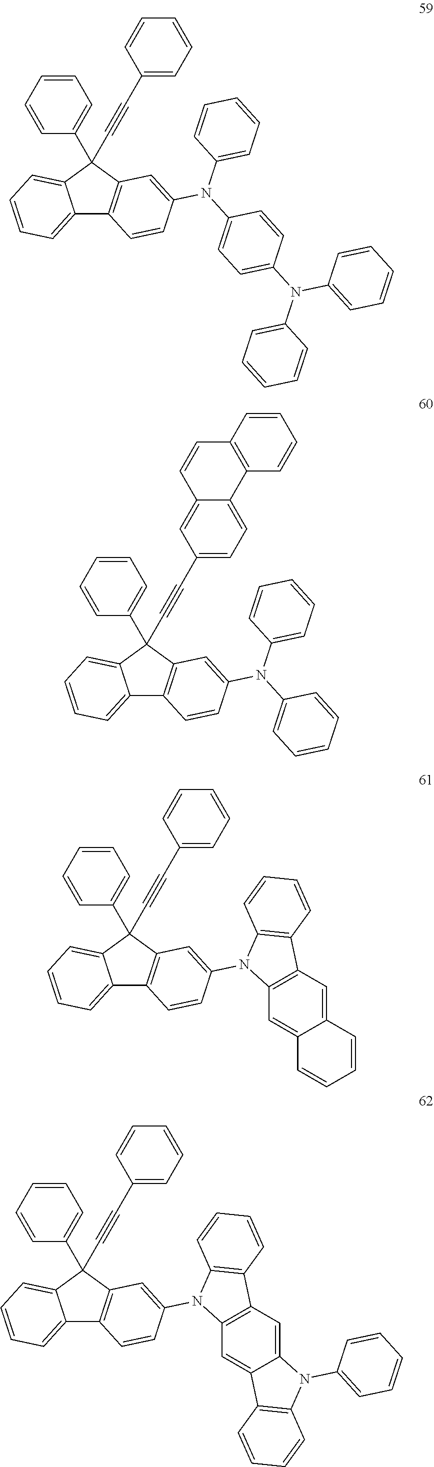

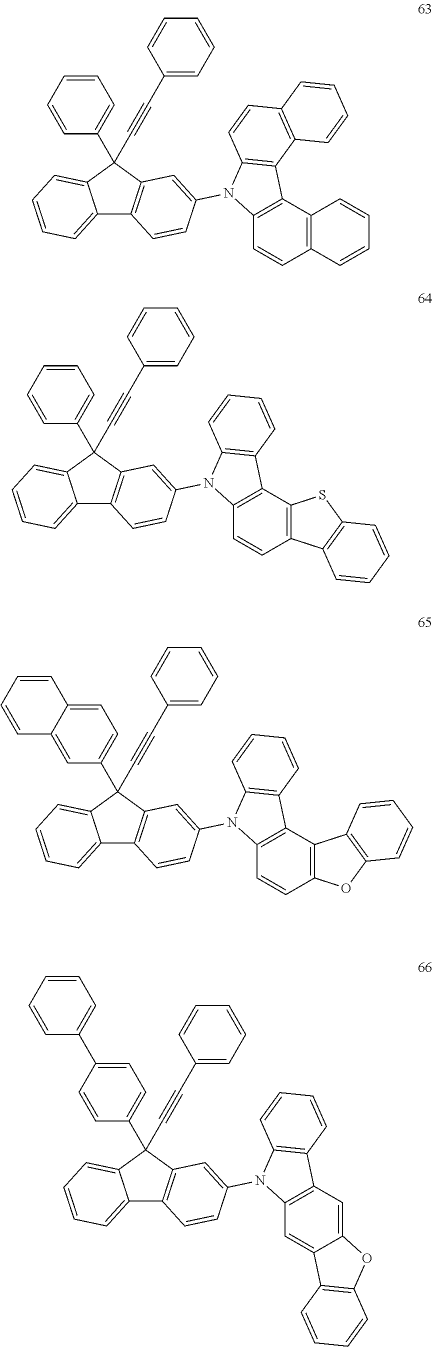 [SMILES: C(#CC1(c2ccccc2)c2ccccc2-c2ccc(-n3c4ccc5ccccc5c4c4c5ccccc5ccc43)cc21)c1ccccc1 ; C(#CC1(c2ccccc2)c2ccccc2-c2ccc(-n3c4ccccc4c4c5sc6ccccc6c5ccc43)cc21)c1ccccc1 ; C(#CC1(c2ccc3ccccc3c2)c2ccccc2-c2ccc(-n3c4ccccc4c4c5c(ccc43)oc3ccccc35)cc21)c1ccccc1 ; C(#CC1(c2ccc(-c3ccccc3)cc2)c2ccccc2-c2ccc(-n3c4ccccc4c4cc5oc6ccccc6c5cc43)cc21)c1ccccc1]

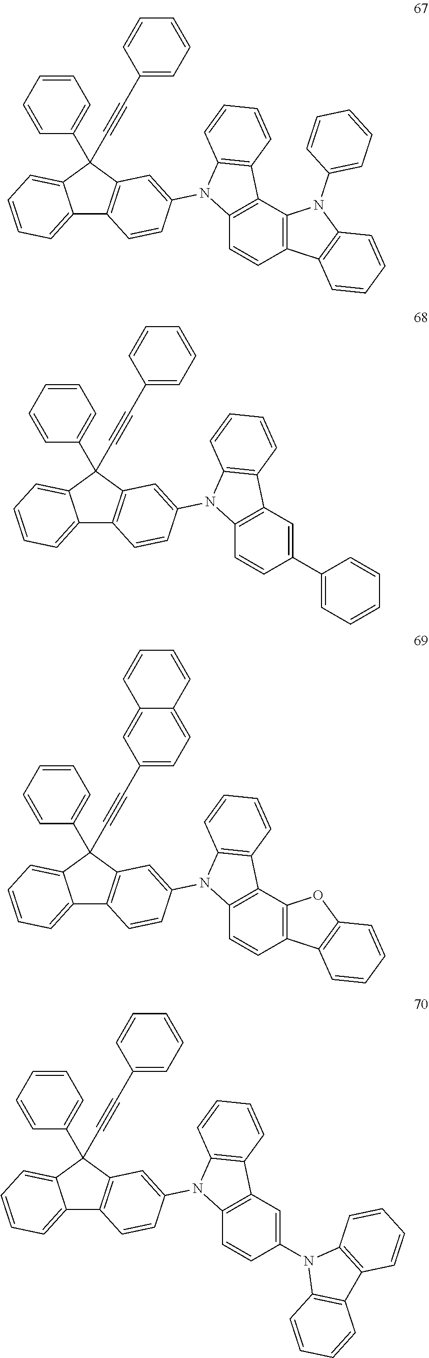 [SMILES: C(#CC1(c2ccccc2)c2ccccc2-c2ccc(-n3c4ccccc4c4c3ccc3c5ccccc5n(-c5ccccc5)c34)cc21)c1ccccc1 ; C(#CC1(c2ccccc2)c2ccccc2-c2ccc(-n3c4ccccc4c4cc(-c5ccccc5)ccc43)cc21)c1ccccc1 ; C(#CC1(c2ccccc2)c2ccccc2-c2ccc(-n3c4ccccc4c4c5oc6ccccc6c5ccc43)cc21)c1ccc2ccccc2c1 ; C(#CC1(c2ccccc2)c2ccccc2-c2ccc(-n3c4ccccc4c4cc(-n5c6ccccc6c6ccccc65)ccc43)cc21)c1ccccc1]

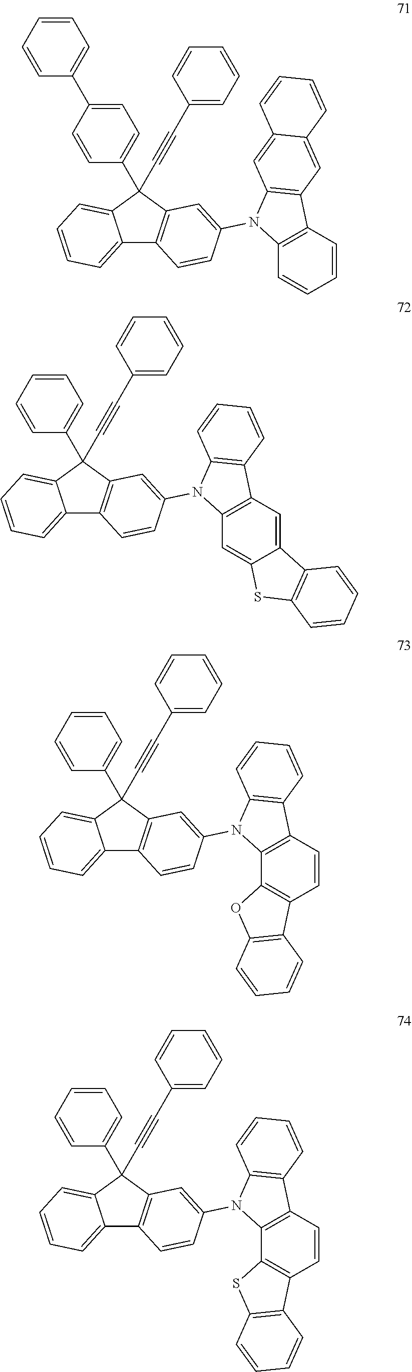 [SMILES: C(#CC1(c2ccc(-c3ccccc3)cc2)c2ccccc2-c2ccc(-n3c4ccccc4c4cc5ccccc5cc43)cc21)c1ccccc1 ; C(#CC1(c2ccccc2)c2ccccc2-c2ccc(-n3c4ccccc4c4cc5c(cc43)sc3ccccc35)cc21)c1ccccc1 ; C(#CC1(c2ccccc2)c2ccccc2-c2ccc(-n3c4ccccc4c4ccc5c6ccccc6oc5c43)cc21)c1ccccc1 ; C(#CC1(c2ccccc2)c2ccccc2-c2ccc(-n3c4ccccc4c4ccc5c6ccccc6sc5c43)cc21)c1ccccc1]

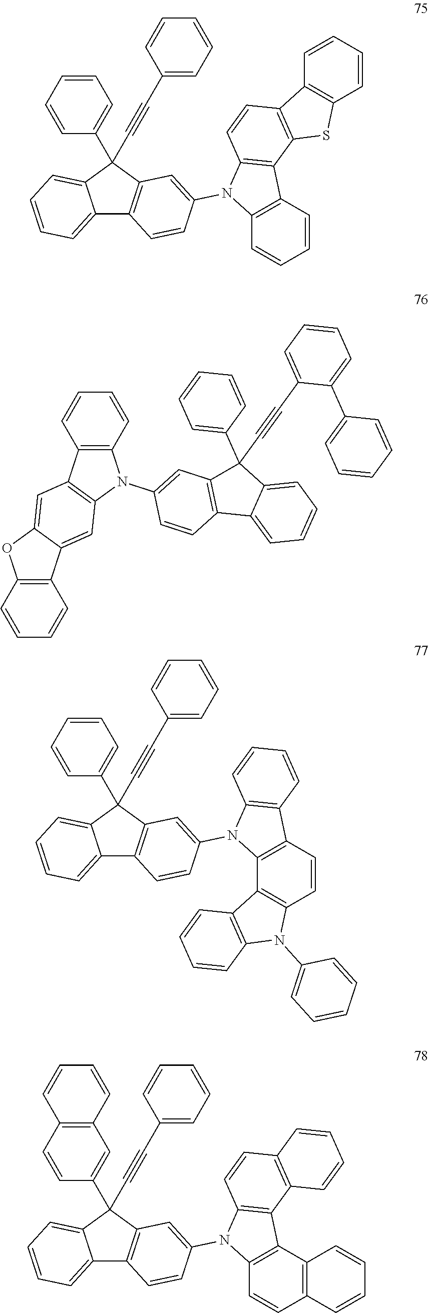 [SMILES: C(#CC1(c2ccccc2)c2ccccc2-c2ccc(-n3c4ccccc4c4c5sc6ccccc6c5ccc43)cc21)c1ccccc1 ; C(#CC1(c2ccccc2)c2ccccc2-c2ccc(-n3c4ccccc4c4cc5oc6ccccc6c5cc43)cc21)c1ccccc1-c1ccccc1 ; C(#CC1(c2ccccc2)c2ccccc2-c2ccc(-n3c4ccccc4c4ccc5c(c6ccccc6n5-c5ccccc5)c43)cc21)c1ccccc1 ; C(#CC1(c2ccc3ccccc3c2)c2ccccc2-c2ccc(-n3c4ccc5ccccc5c4c4c5ccccc5ccc43)cc21)c1ccccc1]

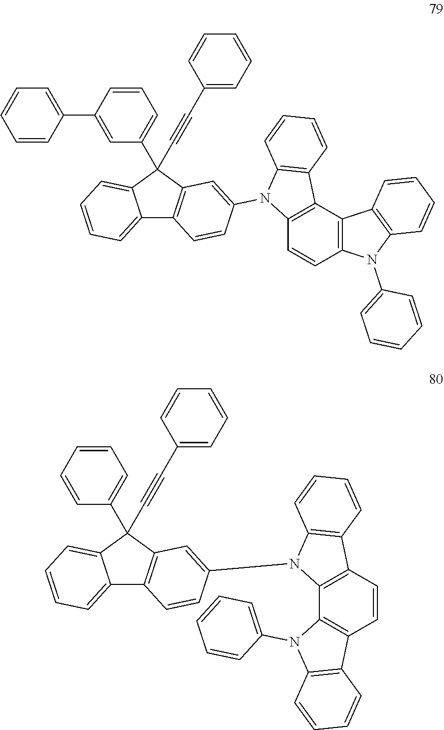 [SMILES: C(#CC1(c2cccc(-c3ccccc3)c2)c2ccccc2-c2ccc(-n3c4ccccc4c4c5c6ccccc6n(-c6ccccc6)c5ccc43)cc21)c1ccccc1 ; C(#CC1(c2ccccc2)c2ccccc2-c2ccc(-n3c4ccccc4c4ccc5c6ccccc6n(-c6ccccc6)c5c43)cc21)c1ccccc1]

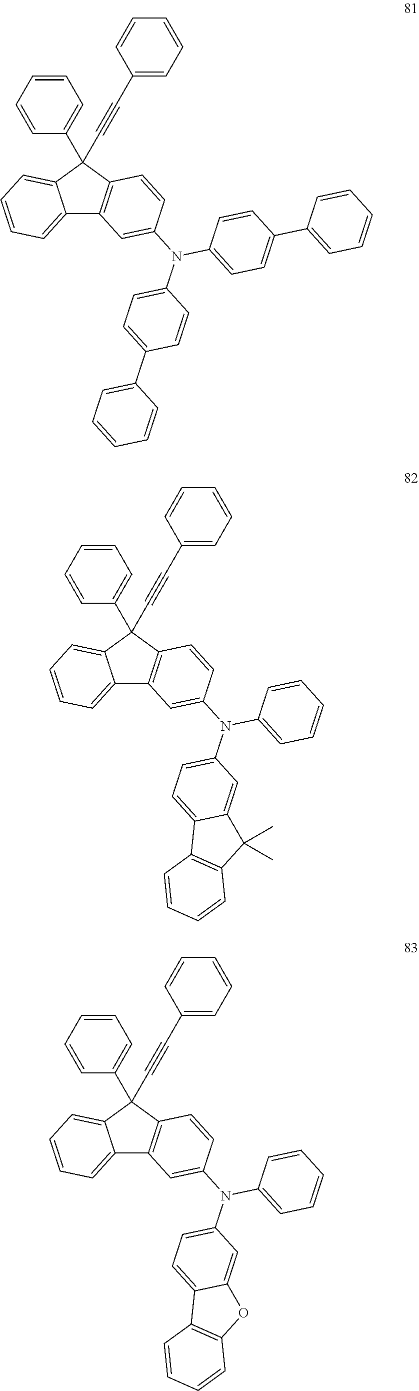

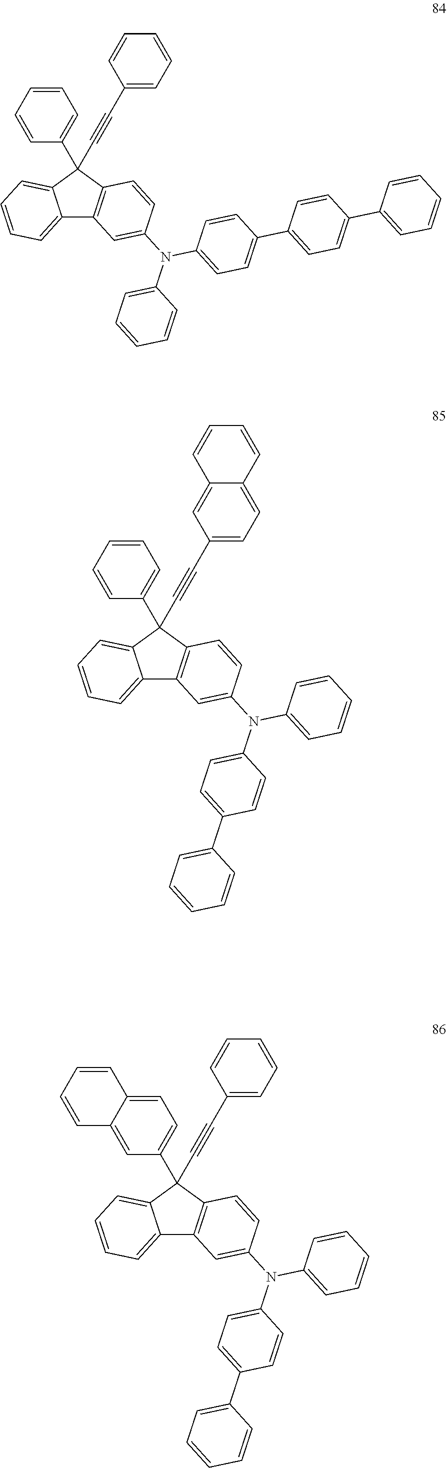 [SMILES: C(#CC1(c2ccccc2)c2ccccc2-c2cc(N(c3ccccc3)c3ccc(-c4ccc(-c5ccccc5)cc4)cc3)ccc21)c1ccccc1 ; C(#CC1(c2ccccc2)c2ccccc2-c2cc(N(c3ccccc3)c3ccc(-c4ccccc4)cc3)ccc21)c1ccc2ccccc2c1 ; C(#CC1(c2ccc3ccccc3c2)c2ccccc2-c2cc(N(c3ccccc3)c3ccc(-c4ccccc4)cc3)ccc21)c1ccccc1]

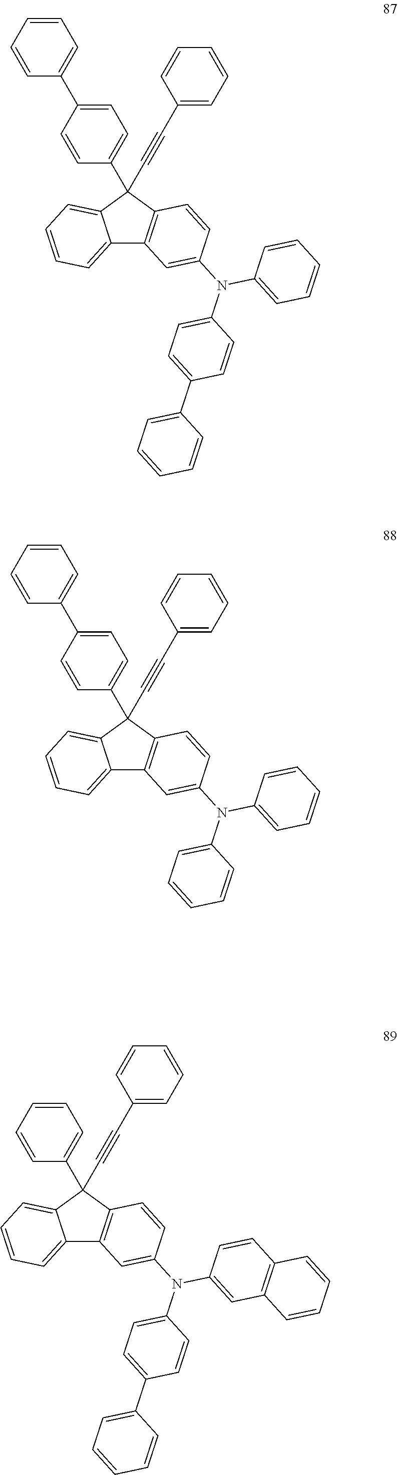 [SMILES: C(#CC1(c2ccc(-c3ccccc3)cc2)c2ccccc2-c2cc(N(c3ccccc3)c3ccc(-c4ccccc4)cc3)ccc21)c1ccccc1 ; C(#CC1(c2ccc(-c3ccccc3)cc2)c2ccccc2-c2cc(N(c3ccccc3)c3ccccc3)ccc21)c1ccccc1 ; C(#CC1(c2ccccc2)c2ccccc2-c2cc(N(c3ccc(-c4ccccc4)cc3)c3ccc4ccccc4c3)ccc21)c1ccccc1]

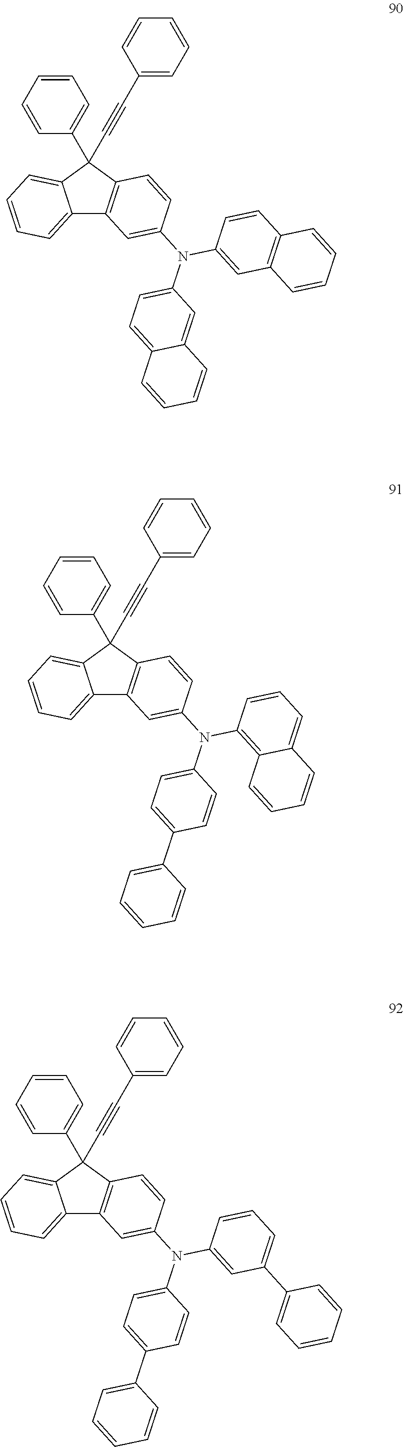 [SMILES: C(#CC1(c2ccccc2)c2ccccc2-c2cc(N(c3ccc4ccccc4c3)c3ccc4ccccc4c3)ccc21)c1ccccc1 ; C(#CC1(c2ccccc2)c2ccccc2-c2cc(N(c3ccc(-c4ccccc4)cc3)c3cccc4ccccc34)ccc21)c1ccccc1 ; C(#CC1(c2ccccc2)c2ccccc2-c2cc(N(c3ccc(-c4ccccc4)cc3)c3cccc(-c4ccccc4)c3)ccc21)c1ccccc1]

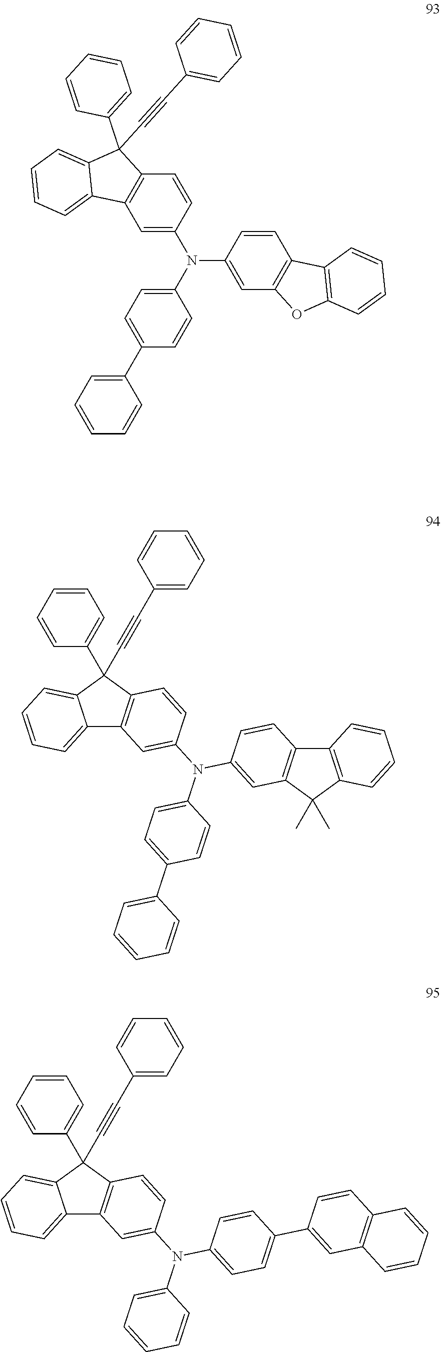 [SMILES: C(#CC1(c2ccccc2)c2ccccc2-c2cc(N(c3ccc(-c4ccccc4)cc3)c3ccc4c(c3)oc3ccccc34)ccc21)c1ccccc1 ; CC1(C)c2ccccc2-c2ccc(N(c3ccc(-c4ccccc4)cc3)c3ccc4c(c3)-c3ccccc3C4(C#Cc3ccccc3)c3ccccc3)cc21 ; C(#CC1(c2ccccc2)c2ccccc2-c2cc(N(c3ccccc3)c3ccc(-c4ccc5ccccc5c4)cc3)ccc21)c1ccccc1]

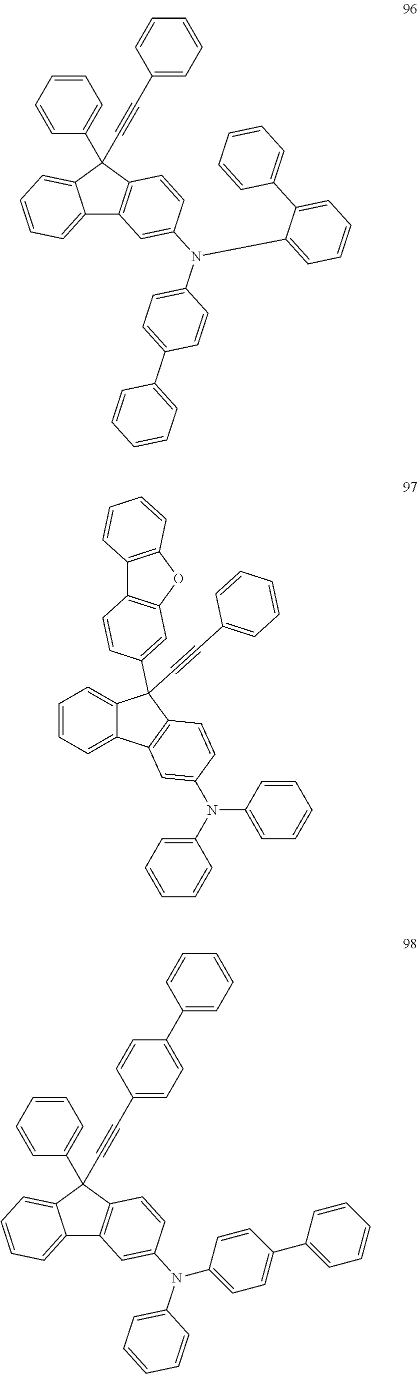 [SMILES: C(#CC1(c2ccccc2)c2ccccc2-c2cc(N(c3ccc(-c4ccccc4)cc3)c3ccccc3-c3ccccc3)ccc21)c1ccccc1 ; C(#CC1(c2ccc3c(c2)oc2ccccc23)c2ccccc2-c2cc(N(c3ccccc3)c3ccccc3)ccc21)c1ccccc1 ; C(#CC1(c2ccccc2)c2ccccc2-c2cc(N(c3ccccc3)c3ccc(-c4ccccc4)cc3)ccc21)c1ccc(-c2ccccc2)cc1]

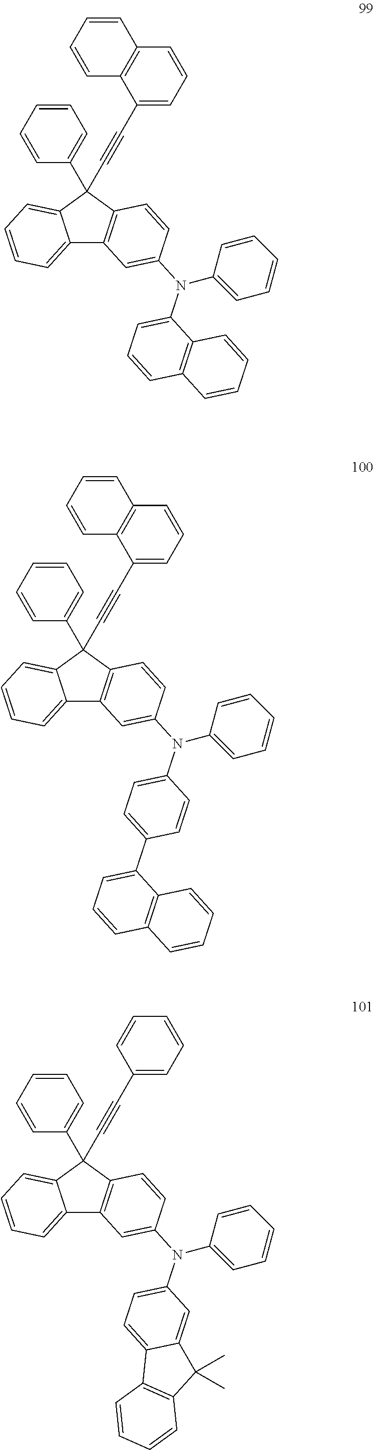 [SMILES: C(#CC1(c2ccccc2)c2ccccc2-c2cc(N(c3ccccc3)c3cccc4ccccc34)ccc21)c1cccc2ccccc12 ; C(#CC1(c2ccccc2)c2ccccc2-c2cc(N(c3ccccc3)c3ccc(-c4cccc5ccccc45)cc3)ccc21)c1cccc2ccccc12 ; CC1(C)c2ccccc2-c2ccc(N(c3ccccc3)c3ccc4c(c3)-c3ccccc3C4(C#Cc3ccccc3)c3ccccc3)cc21]

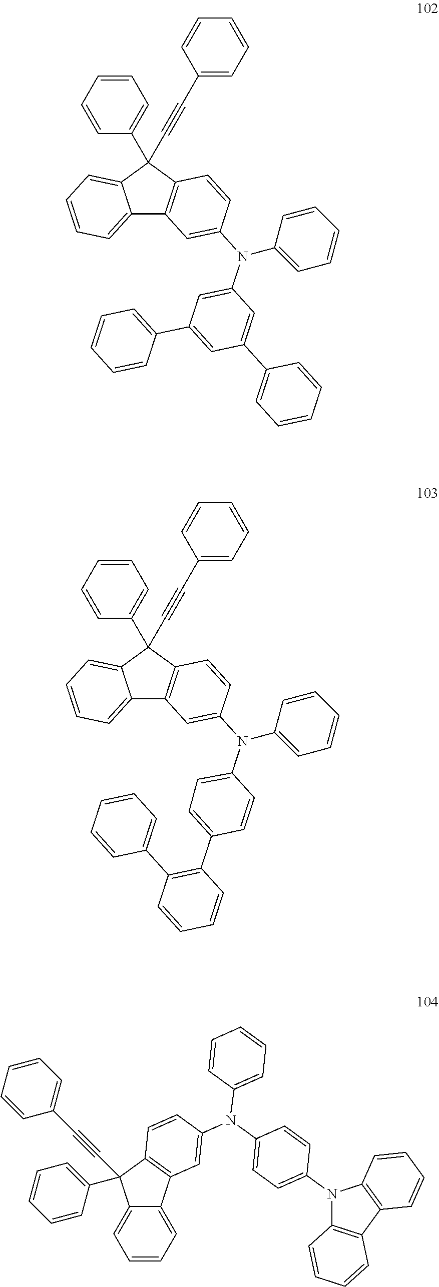 [SMILES: C(#CC1(c2ccccc2)c2ccccc2-c2cc(N(c3ccccc3)c3cc(-c4ccccc4)cc(-c4ccccc4)c3)ccc21)c1ccccc1 ; C(#CC1(c2ccccc2)c2ccccc2-c2cc(N(c3ccccc3)c3ccc(-c4ccccc4-c4ccccc4)cc3)ccc21)c1ccccc1 ; C(#CC1(c2ccccc2)c2ccccc2-c2cc(N(c3ccccc3)c3ccc(-n4c5ccccc5c5ccccc54)cc3)ccc21)c1ccccc1]

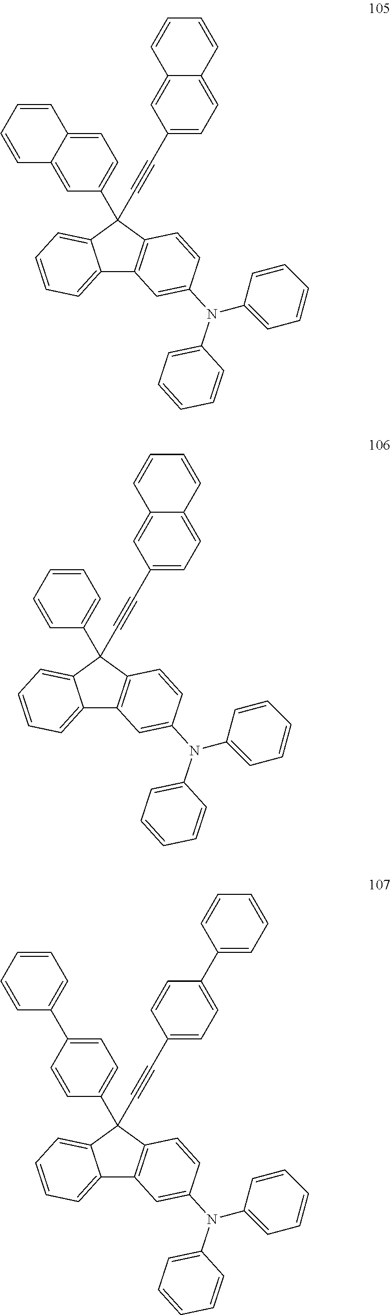

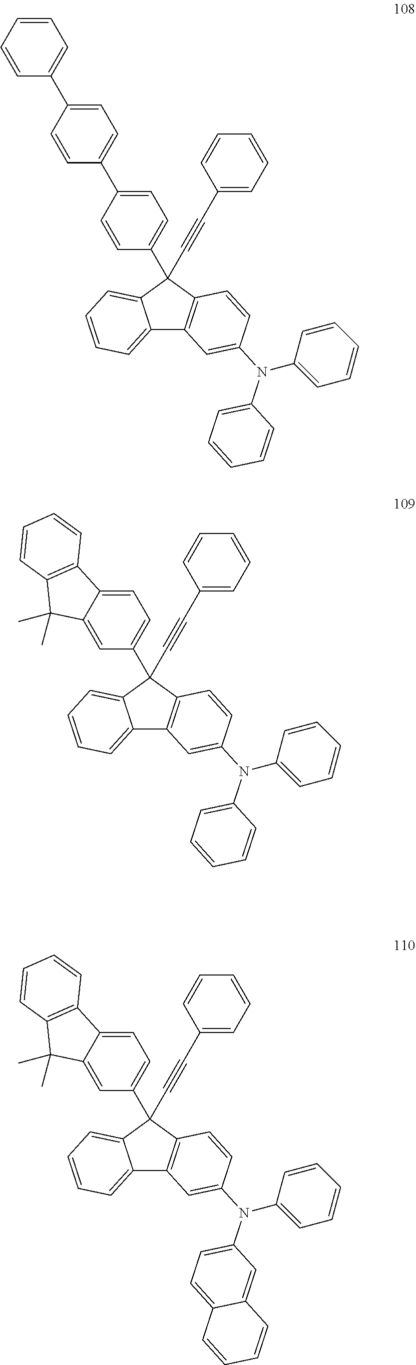 [SMILES: C(#CC1(c2ccc(-c3ccc(-c4ccccc4)cc3)cc2)c2ccccc2-c2cc(N(c3ccccc3)c3ccccc3)ccc21)c1ccccc1 ; CC1(C)c2ccccc2-c2ccc(C3(C#Cc4ccccc4)c4ccccc4-c4cc(N(c5ccccc5)c5ccccc5)ccc43)cc21 ; CC1(C)c2ccccc2-c2ccc(C3(C#Cc4ccccc4)c4ccccc4-c4cc(N(c5ccccc5)c5ccc6ccccc6c5)ccc43)cc21]

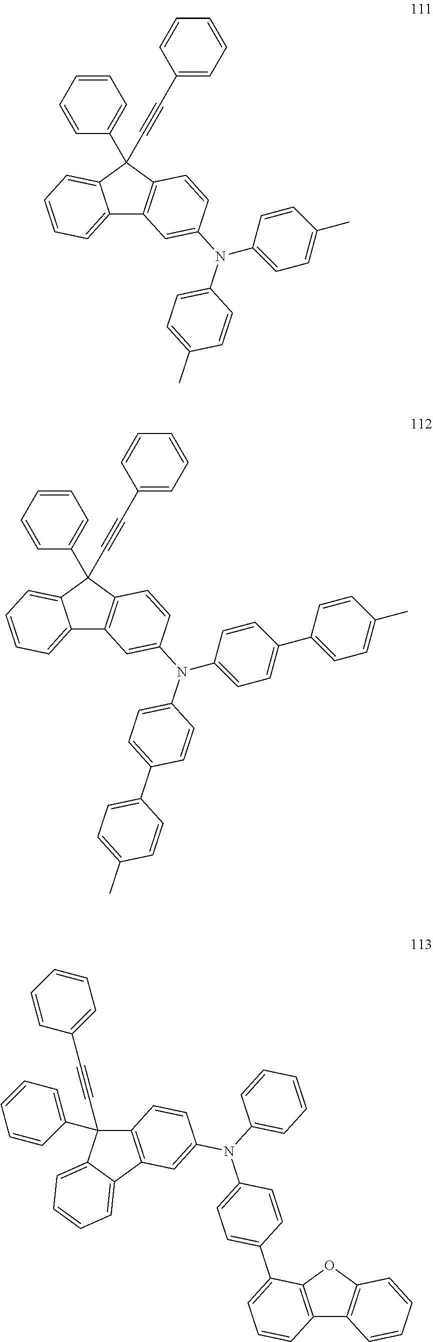 [SMILES: Cc1ccc(N(c2ccc(C)cc2)c2ccc3c(c2)-c2ccccc2C3(C#Cc2ccccc2)c2ccccc2)cc1 ; Cc1ccc(-c2ccc(N(c3ccc(-c4ccc(C)cc4)cc3)c3ccc4c(c3)-c3ccccc3C4(C#Cc3ccccc3)c3ccccc3)cc2)cc1 ; C(#CC1(c2ccccc2)c2ccccc2-c2cc(N(c3ccccc3)c3ccc(-c4cccc5c4oc4ccccc45)cc3)ccc21)c1ccccc1]

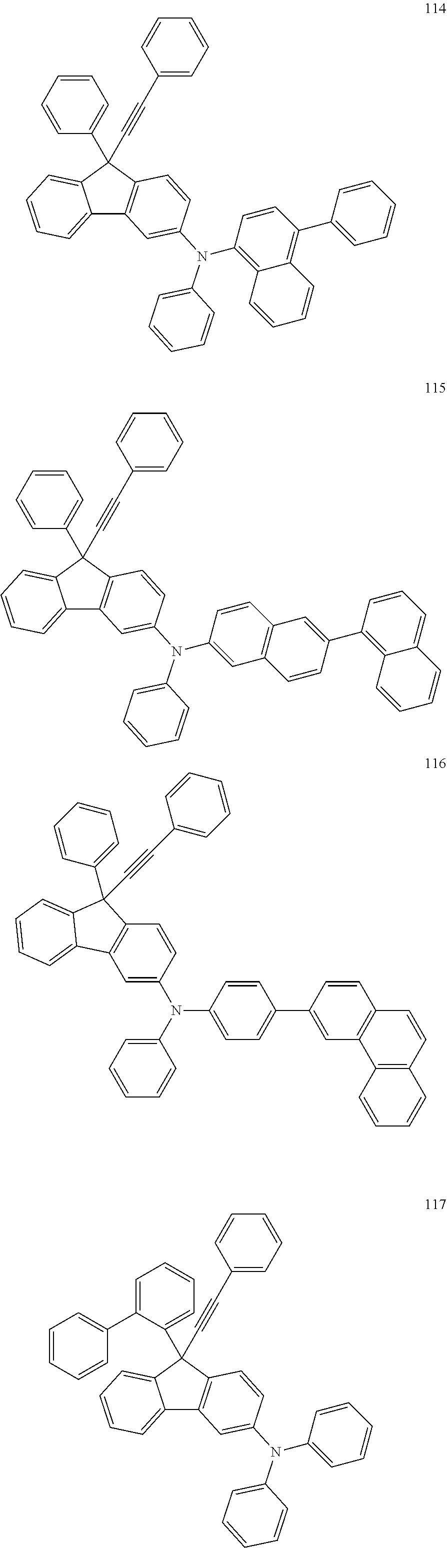

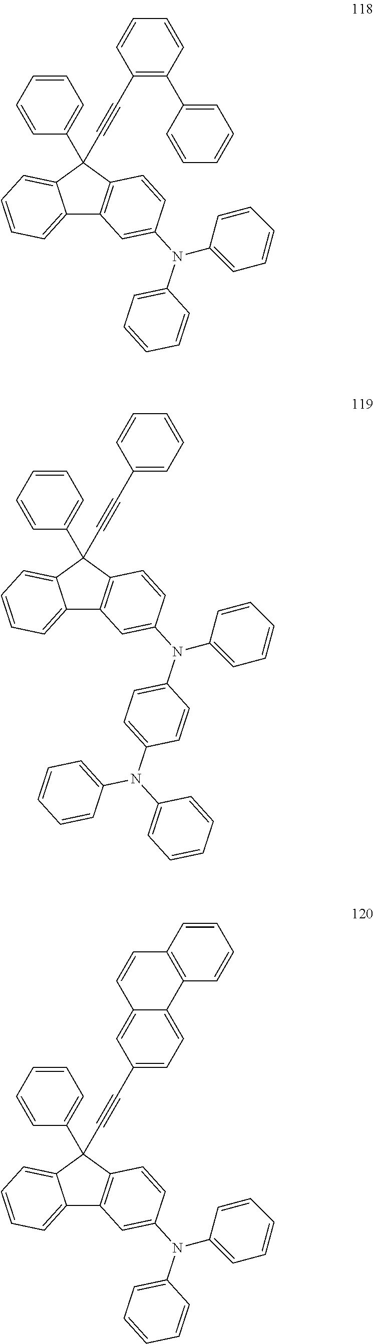 [SMILES: C(#CC1(c2ccccc2)c2ccccc2-c2cc(N(c3ccccc3)c3ccccc3)ccc21)c1ccccc1-c1ccccc1 ; C(#CC1(c2ccccc2)c2ccccc2-c2cc(N(c3ccccc3)c3ccc(N(c4ccccc4)c4ccccc4)cc3)ccc21)c1ccccc1 ; C(#CC1(c2ccccc2)c2ccccc2-c2cc(N(c3ccccc3)c3ccccc3)ccc21)c1ccc2c(ccc3ccccc32)c1]

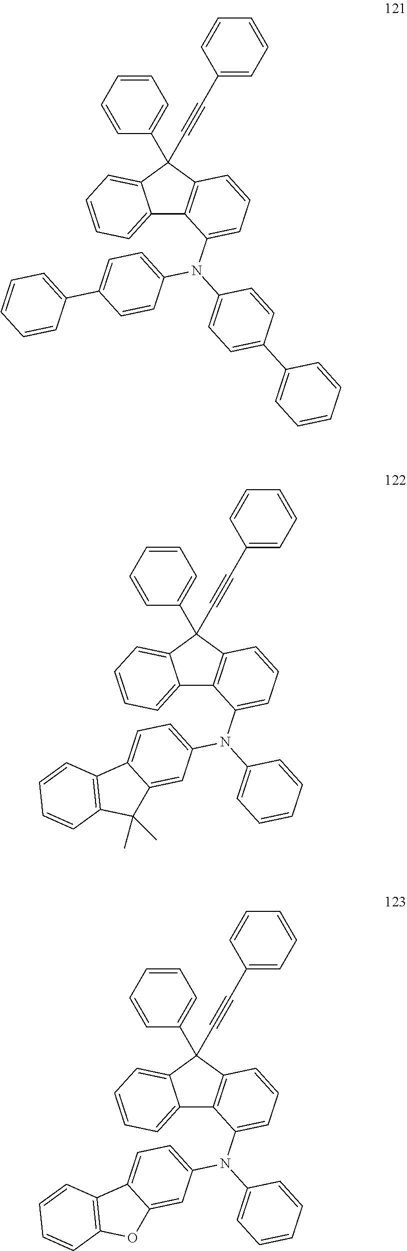 [SMILES: C(#CC1(c2ccccc2)c2ccccc2-c2c(N(c3ccc(-c4ccccc4)cc3)c3ccc(-c4ccccc4)cc3)cccc21)c1ccccc1 ; CC1(C)c2ccccc2-c2ccc(N(c3ccccc3)c3cccc4c3-c3ccccc3C4(C#Cc3ccccc3)c3ccccc3)cc21 ; C(#CC1(c2ccccc2)c2ccccc2-c2c(N(c3ccccc3)c3ccc4c(c3)oc3ccccc34)cccc21)c1ccccc1]

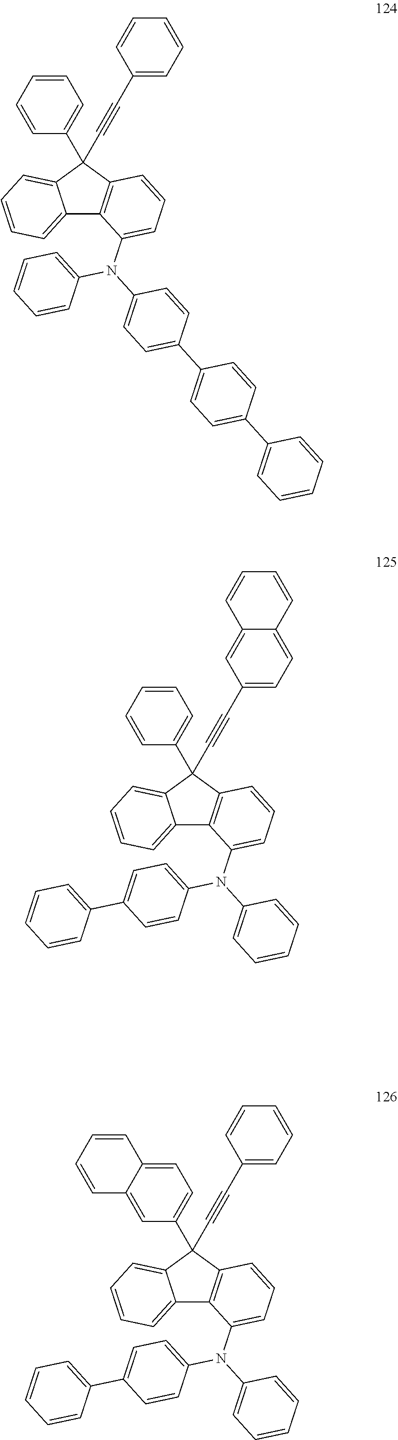 [SMILES: C(#CC1(c2ccccc2)c2ccccc2-c2c(N(c3ccccc3)c3ccc(-c4ccc(-c5ccccc5)cc4)cc3)cccc21)c1ccccc1 ; C(#CC1(c2ccccc2)c2ccccc2-c2c(N(c3ccccc3)c3ccc(-c4ccccc4)cc3)cccc21)c1ccc2ccccc2c1 ; C(#CC1(c2ccc3ccccc3c2)c2ccccc2-c2c(N(c3ccccc3)c3ccc(-c4ccccc4)cc3)cccc21)c1ccccc1]

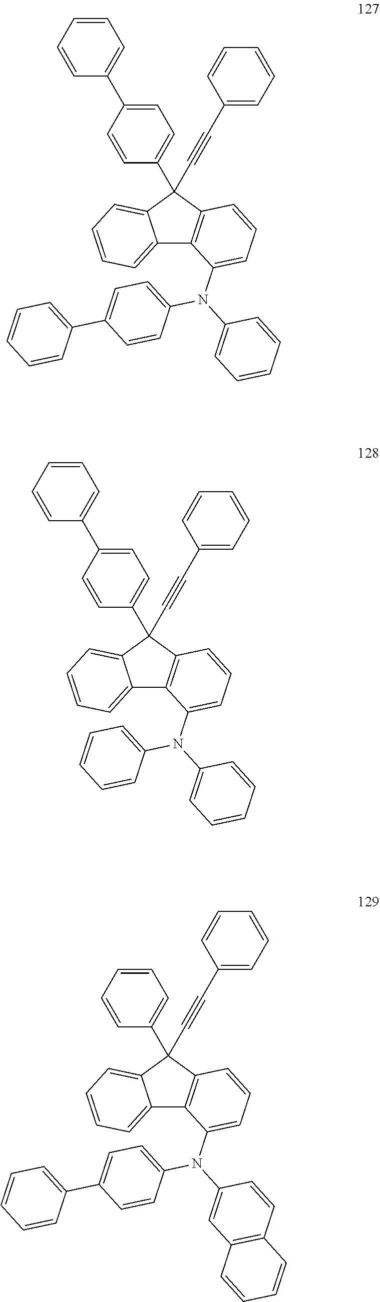 [SMILES: C(#CC1(c2ccc(-c3ccccc3)cc2)c2ccccc2-c2c(N(c3ccccc3)c3ccc(-c4ccccc4)cc3)cccc21)c1ccccc1 ; C(#CC1(c2ccc(-c3ccccc3)cc2)c2ccccc2-c2c(N(c3ccccc3)c3ccccc3)cccc21)c1ccccc1 ; C(#CC1(c2ccccc2)c2ccccc2-c2c(N(c3ccc(-c4ccccc4)cc3)c3ccc4ccccc4c3)cccc21)c1ccccc1]

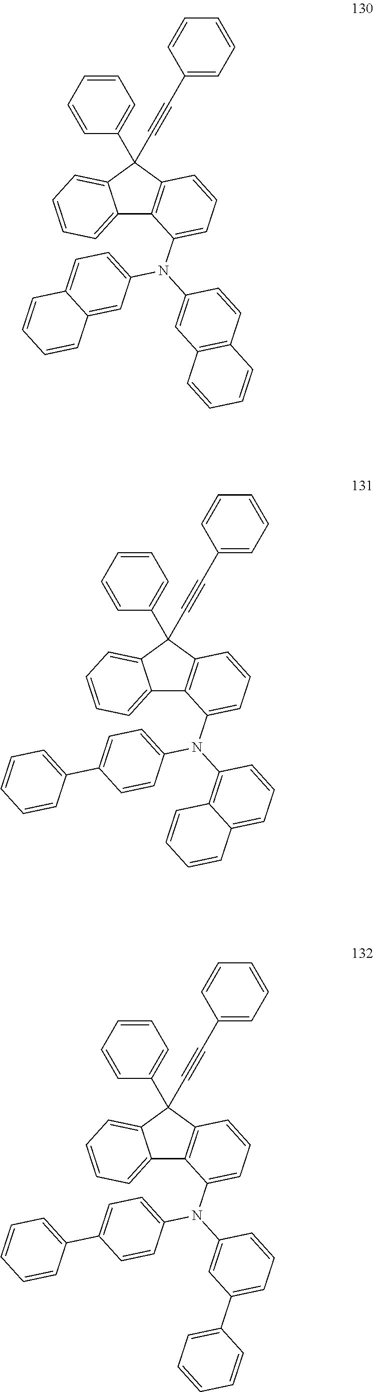 [SMILES: C(#CC1(c2ccccc2)c2ccccc2-c2c(N(c3ccc4ccccc4c3)c3ccc4ccccc4c3)cccc21)c1ccccc1 ; C(#CC1(c2ccccc2)c2ccccc2-c2c(N(c3ccc(-c4ccccc4)cc3)c3cccc4ccccc34)cccc21)c1ccccc1 ; C(#CC1(c2ccccc2)c2ccccc2-c2c(N(c3ccc(-c4ccccc4)cc3)c3cccc(-c4ccccc4)c3)cccc21)c1ccccc1]

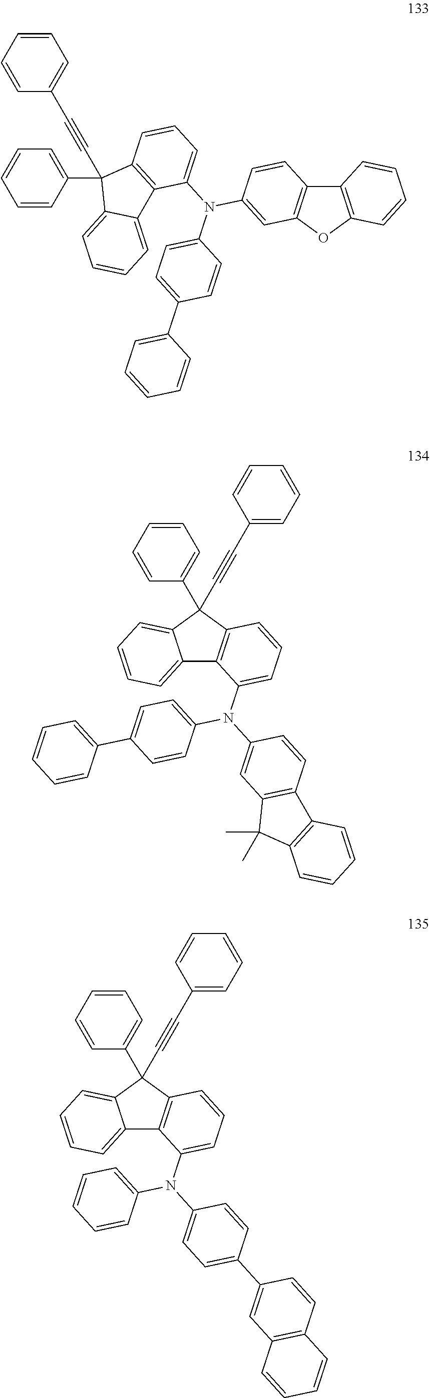 [SMILES: C(#CC1(c2ccccc2)c2ccccc2-c2c(N(c3ccc(-c4ccccc4)cc3)c3ccc4c(c3)oc3ccccc34)cccc21)c1ccccc1 ; CC1(C)c2ccccc2-c2ccc(N(c3ccc(-c4ccccc4)cc3)c3cccc4c3-c3ccccc3C4(C#Cc3ccccc3)c3ccccc3)cc21 ; C(#CC1(c2ccccc2)c2ccccc2-c2c(N(c3ccccc3)c3ccc(-c4ccc5ccccc5c4)cc3)cccc21)c1ccccc1]

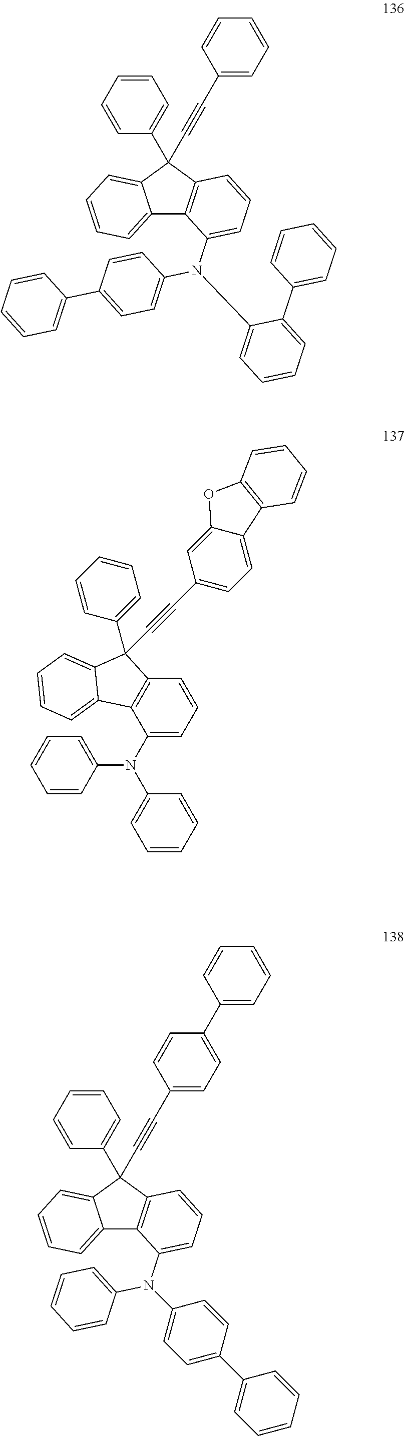 [SMILES: C(#CC1(c2ccccc2)c2ccccc2-c2c(N(c3ccc(-c4ccccc4)cc3)c3ccccc3-c3ccccc3)cccc21)c1ccccc1 ; C(#CC1(c2ccccc2)c2ccccc2-c2c(N(c3ccccc3)c3ccccc3)cccc21)c1ccc2c(c1)oc1ccccc12 ; C(#CC1(c2ccccc2)c2ccccc2-c2c(N(c3ccccc3)c3ccc(-c4ccccc4)cc3)cccc21)c1ccc(-c2ccccc2)cc1]

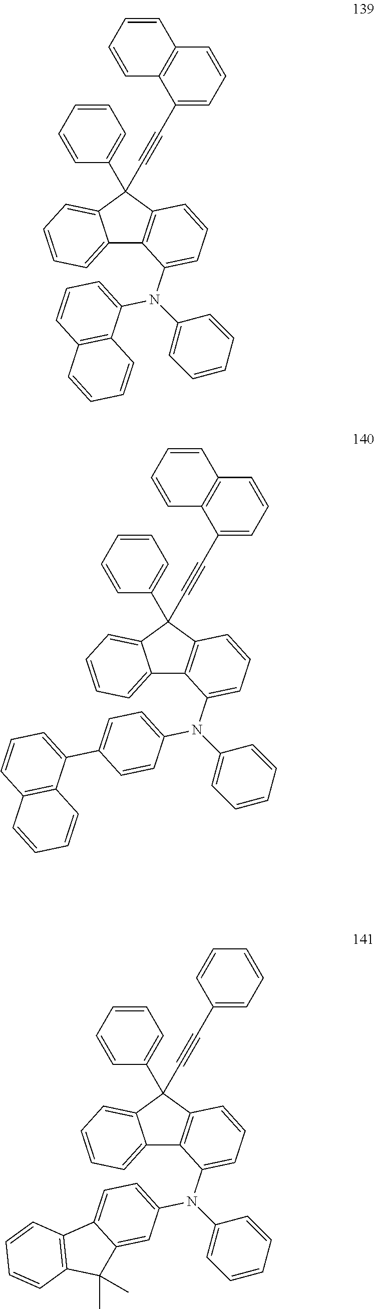 [SMILES: C(#CC1(c2ccccc2)c2ccccc2-c2c(N(c3ccccc3)c3cccc4ccccc34)cccc21)c1cccc2ccccc12 ; C(#CC1(c2ccccc2)c2ccccc2-c2c(N(c3ccccc3)c3ccc(-c4cccc5ccccc45)cc3)cccc21)c1cccc2ccccc12 ; CC1(C)c2ccccc2-c2ccc(N(c3ccccc3)c3cccc4c3-c3ccccc3C4(C#Cc3ccccc3)c3ccccc3)cc21]

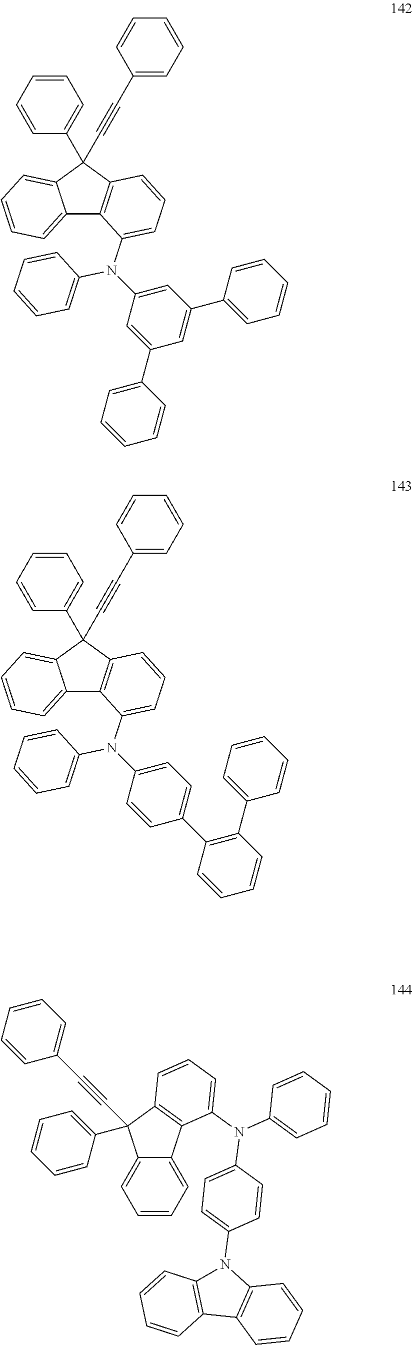 [SMILES: C(#CC1(c2ccccc2)c2ccccc2-c2c(N(c3ccccc3)c3cc(-c4ccccc4)cc(-c4ccccc4)c3)cccc21)c1ccccc1 ; C(#CC1(c2ccccc2)c2ccccc2-c2c(N(c3ccccc3)c3ccc(-c4ccccc4-c4ccccc4)cc3)cccc21)c1ccccc1 ; C(#CC1(c2ccccc2)c2ccccc2-c2c(N(c3ccccc3)c3ccc(-n4c5ccccc5c5ccccc54)cc3)cccc21)c1ccccc1]

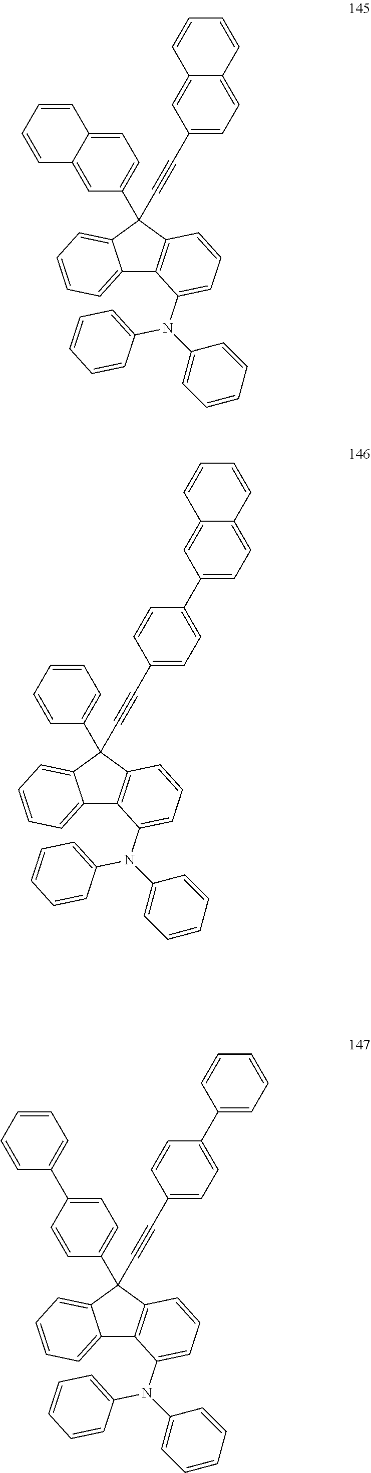 [SMILES: C(#CC1(c2ccc3ccccc3c2)c2ccccc2-c2c(N(c3ccccc3)c3ccccc3)cccc21)c1ccc2ccccc2c1 ; C(#CC1(c2ccccc2)c2ccccc2-c2c(N(c3ccccc3)c3ccccc3)cccc21)c1ccc(-c2ccc3ccccc3c2)cc1 ; C(#CC1(c2ccc(-c3ccccc3)cc2)c2ccccc2-c2c(N(c3ccccc3)c3ccccc3)cccc21)c1ccc(-c2ccccc2)cc1]

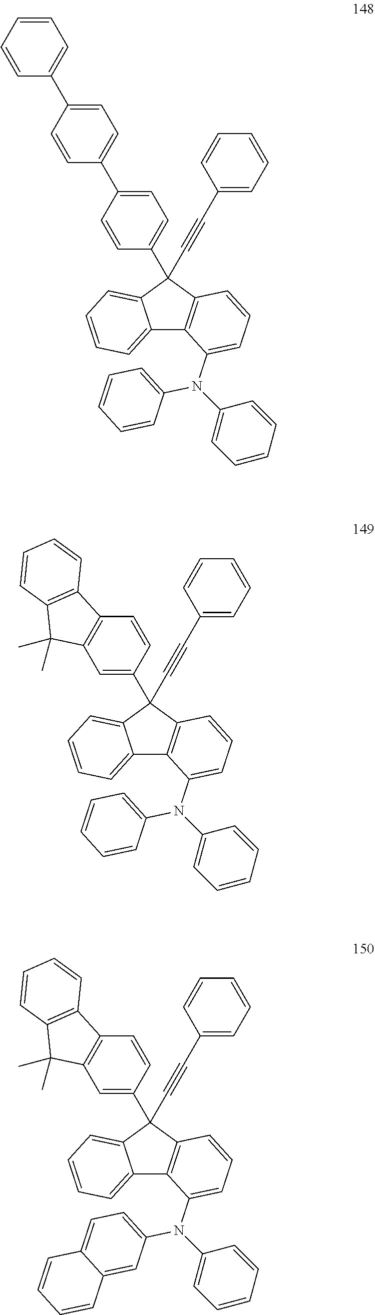 [SMILES: C(#CC1(c2ccc(-c3ccc(-c4ccccc4)cc3)cc2)c2ccccc2-c2c(N(c3ccccc3)c3ccccc3)cccc21)c1ccccc1 ; CC1(C)c2ccccc2-c2ccc(C3(C#Cc4ccccc4)c4ccccc4-c4c(N(c5ccccc5)c5ccccc5)cccc43)cc21 ; CC1(C)c2ccccc2-c2ccc(C3(C#Cc4ccccc4)c4ccccc4-c4c(N(c5ccccc5)c5ccc6ccccc6c5)cccc43)cc21]

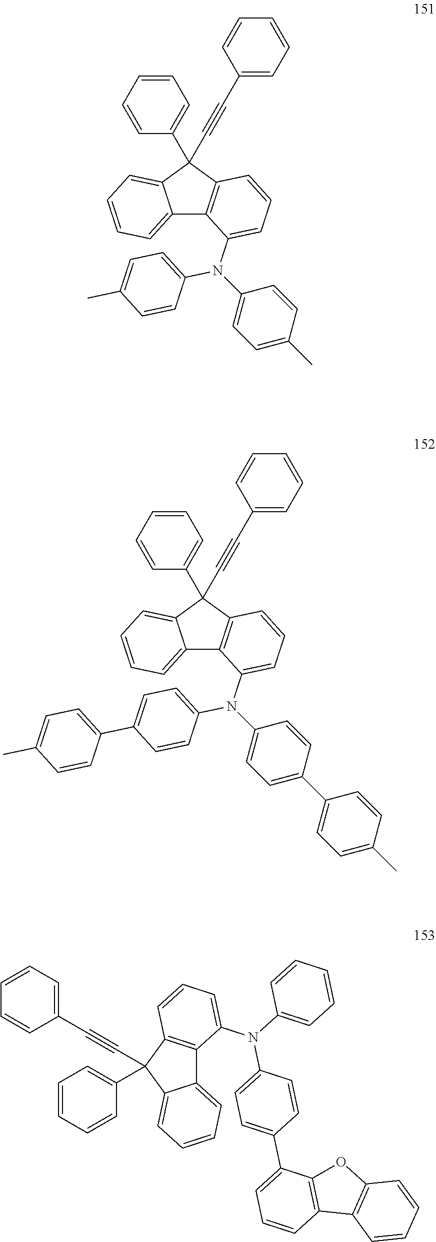

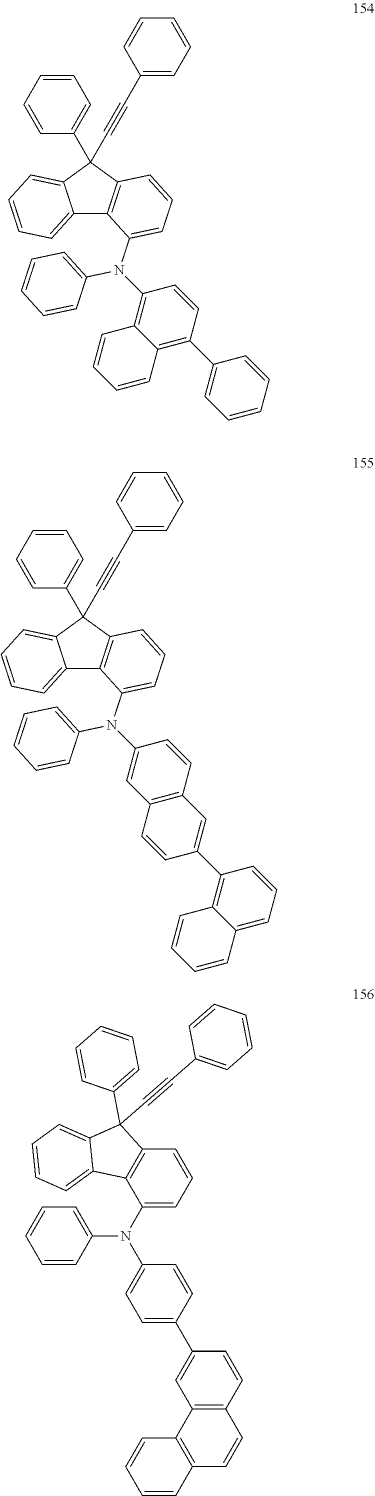

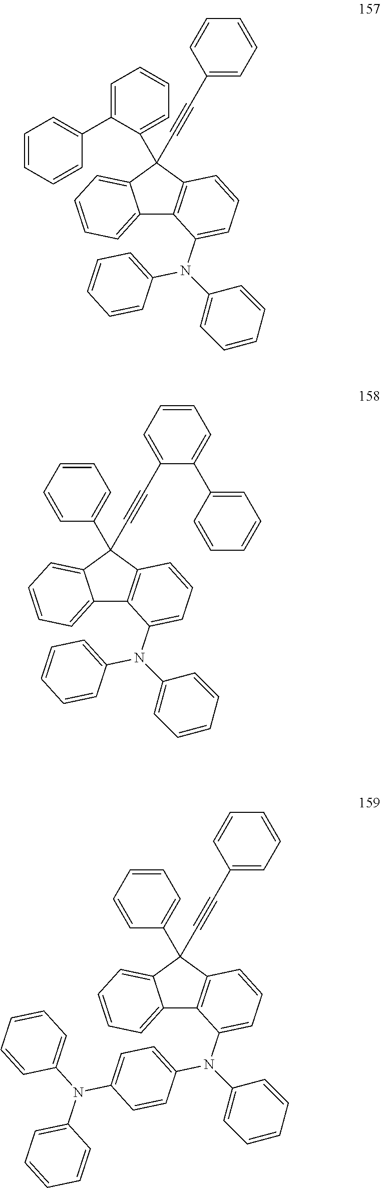 [SMILES: C(#CC1(c2ccccc2-c2ccccc2)c2ccccc2-c2c(N(c3ccccc3)c3ccccc3)cccc21)c1ccccc1 ; C(#CC1(c2ccccc2)c2ccccc2-c2c(N(c3ccccc3)c3ccccc3)cccc21)c1ccccc1-c1ccccc1 ; C(#CC1(c2ccccc2)c2ccccc2-c2c(N(c3ccccc3)c3ccc(N(c4ccccc4)c4ccccc4)cc3)cccc21)c1ccccc1]

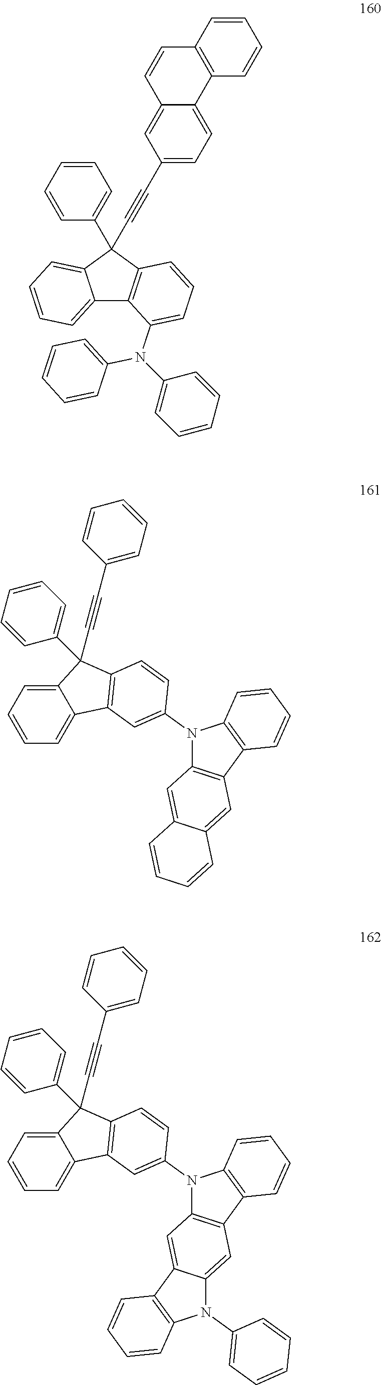 [SMILES: C(#CC1(c2ccccc2)c2ccccc2-c2c(N(c3ccccc3)c3ccccc3)cccc21)c1ccc2c(ccc3ccccc32)c1 ; C(#CC1(c2ccccc2)c2ccccc2-c2cc(-n3c4ccccc4c4cc5ccccc5cc43)ccc21)c1ccccc1 ; C(#CC1(c2ccccc2)c2ccccc2-c2cc(-n3c4ccccc4c4cc5c(cc43)c3ccccc3n5-c3ccccc3)ccc21)c1ccccc1]

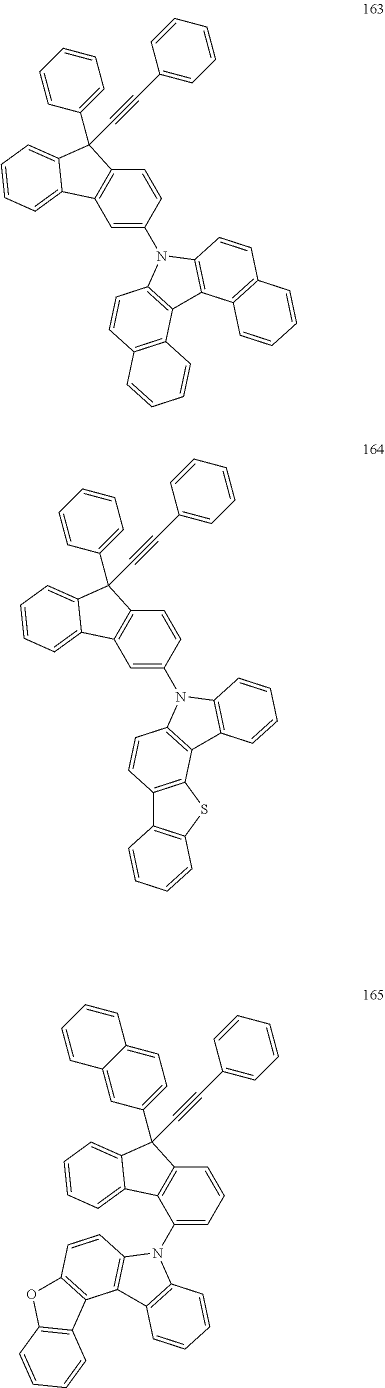 [SMILES: C(#CC1(c2ccccc2)c2ccccc2-c2cc(-n3c4ccc5ccccc5c4c4c5ccccc5ccc43)ccc21)c1ccccc1 ; C(#CC1(c2ccccc2)c2ccccc2-c2cc(-n3c4ccccc4c4c5sc6ccccc6c5ccc43)ccc21)c1ccccc1 ; C(#CC1(c2ccc3ccccc3c2)c2ccccc2-c2c(-n3c4ccccc4c4c5c(ccc43)oc3ccccc35)cccc21)c1ccccc1]

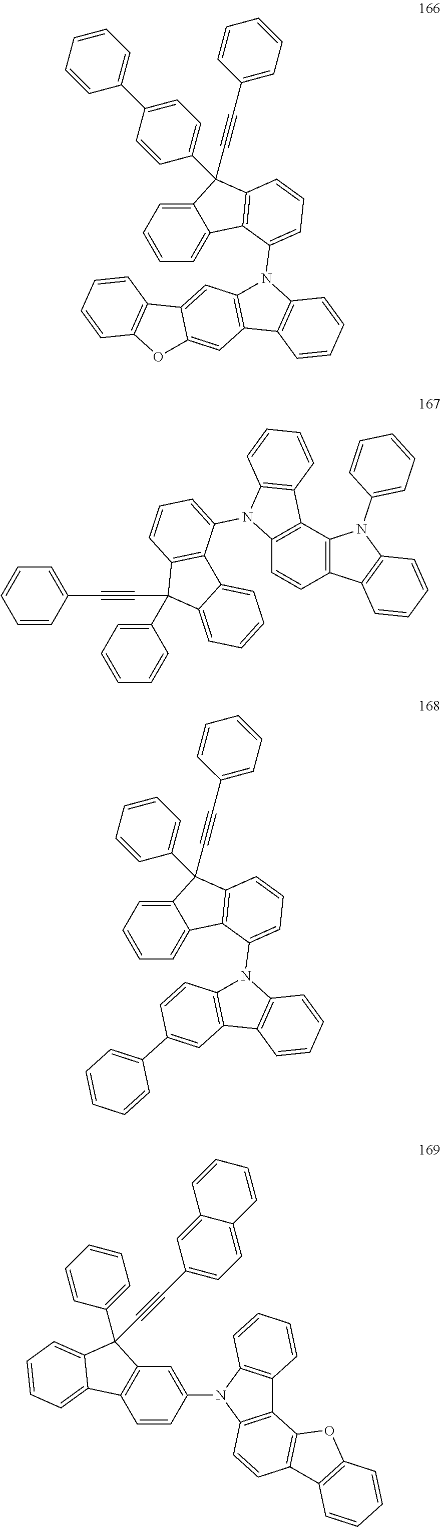 [SMILES: C(#CC1(c2ccc(-c3ccccc3)cc2)c2ccccc2-c2c(-n3c4ccccc4c4cc5oc6ccccc6c5cc43)cccc21)c1ccccc1 ; C(#CC1(c2ccccc2)c2ccccc2-c2c(-n3c4ccccc4c4c3ccc3c5ccccc5n(-c5ccccc5)c34)cccc21)c1ccccc1 ; C(#CC1(c2ccccc2)c2ccccc2-c2c(-n3c4ccccc4c4cc(-c5ccccc5)ccc43)cccc21)c1ccccc1 ; C(#CC1(c2ccccc2)c2ccccc2-c2ccc(-n3c4ccccc4c4c5oc6ccccc6c5ccc43)cc21)c1ccc2ccccc2c1]

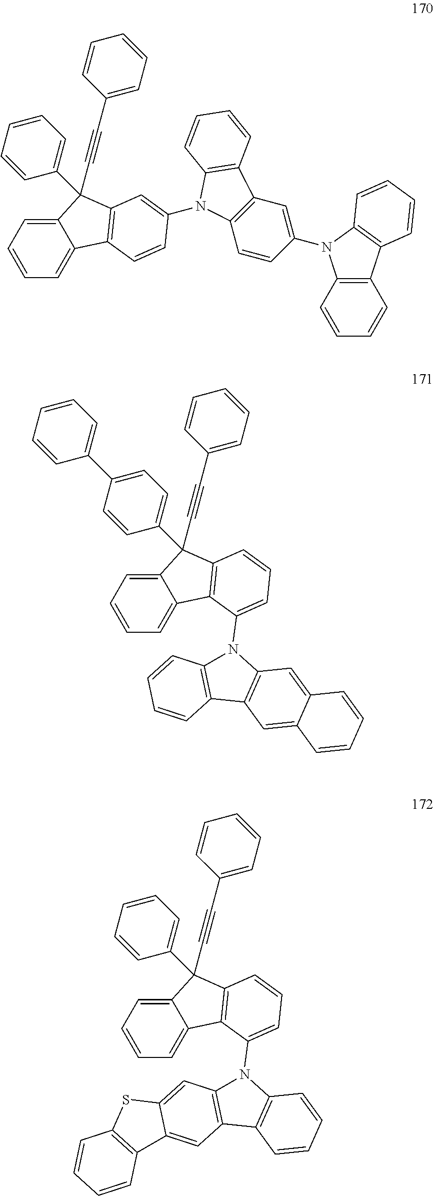 [SMILES: C(#CC1(c2ccccc2)c2ccccc2-c2ccc(-n3c4ccccc4c4cc(-n5c6ccccc6c6ccccc65)ccc43)cc21)c1ccccc1 ; C(#CC1(c2ccc(-c3ccccc3)cc2)c2ccccc2-c2c(-n3c4ccccc4c4cc5ccccc5cc43)cccc21)c1ccccc1 ; C(#CC1(c2ccccc2)c2ccccc2-c2c(-n3c4ccccc4c4cc5c(cc43)sc3ccccc35)cccc21)c1ccccc1]

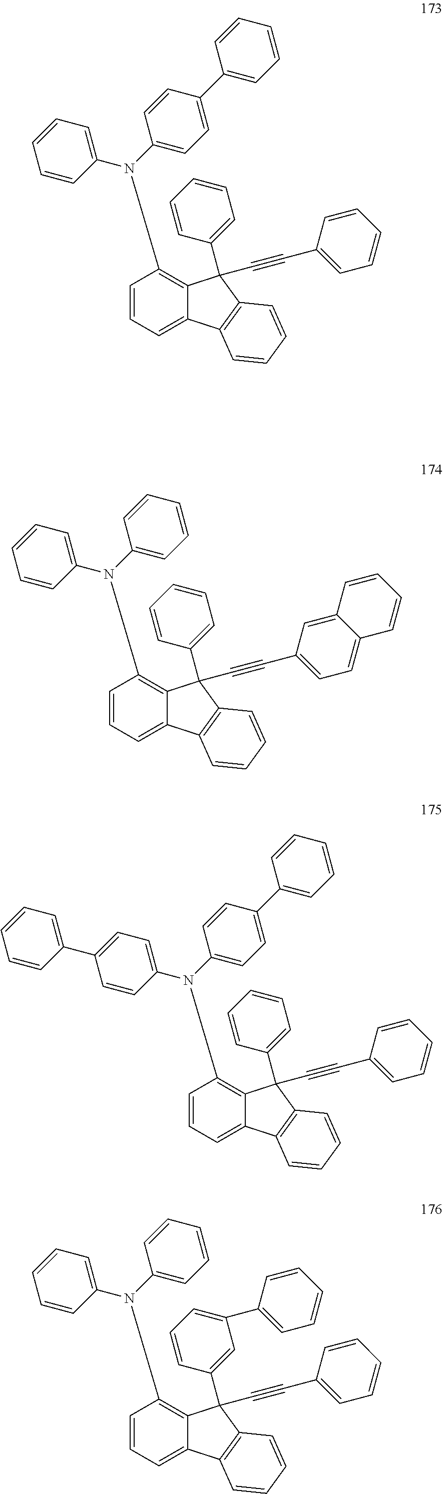 [SMILES: C(#CC1(c2ccccc2)c2ccccc2-c2cccc(N(c3ccccc3)c3ccc(-c4ccccc4)cc3)c21)c1ccccc1 ; C(#CC1(c2ccccc2)c2ccccc2-c2cccc(N(c3ccccc3)c3ccccc3)c21)c1ccc2ccccc2c1 ; C(#CC1(c2ccccc2)c2ccccc2-c2cccc(N(c3ccc(-c4ccccc4)cc3)c3ccc(-c4ccccc4)cc3)c21)c1ccccc1 ; C(#CC1(c2cccc(-c3ccccc3)c2)c2ccccc2-c2cccc(N(c3ccccc3)c3ccccc3)c21)c1ccccc1]

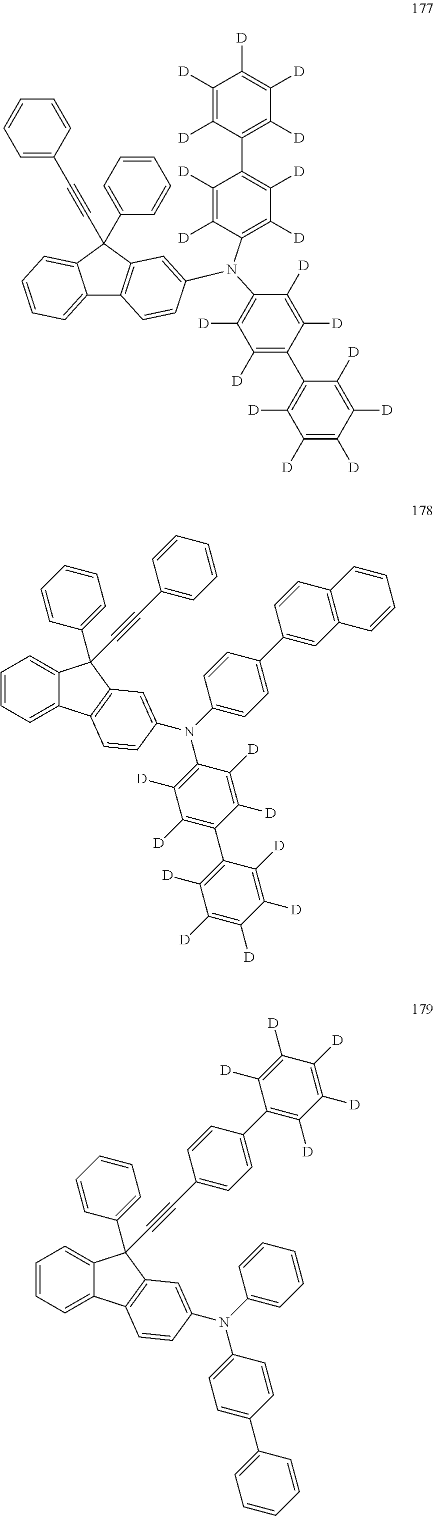 [SMILES: [2H]c1c([2H])c([2H])c(-c2c([2H])c([2H])c(N(c3ccc4c(c3)C(C#Cc3ccccc3)(c3ccccc3)c3ccccc3-4)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c(-c2c([2H])c([2H])c(N(c3ccc(-c4ccc5ccccc5c4)cc3)c3ccc4c(c3)C(C#Cc3ccccc3)(c3ccccc3)c3ccccc3-4)c([2H])c2[2H])c([2H])c1[2H] ; [2H]c1c([2H])c([2H])c(-c2ccc(C#CC3(c4ccccc4)c4ccccc4-c4ccc(N(c5ccccc5)c5ccc(-c6ccccc6)cc5)cc43)cc2)c([2H])c1[2H]]

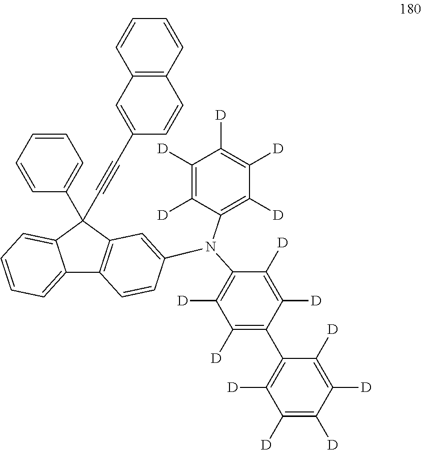 [SMILES: [2H]c1c([2H])c([2H])c(-c2c([2H])c([2H])c(N(c3ccc4c(c3)C(C#Cc3ccc5ccccc5c3)(c3ccccc3)c3ccccc3-4)c3c([2H])c([2H])c([2H])c([2H])c3[2H])c([2H])c2[2H])c([2H])c1[2H]]

In addition, by introducing various substituents to the structure of Chemical Formula 1, compounds having unique properties of the introduced substituents may be synthesized. For example, by introducing substituents normally used as a hole injection layer material, a hole transfer layer material, an electron blocking layer material, a light emitting auxiliary layer material, a light emitting layer material, an electron transfer layer material, a hole blocking layer material and an electron injection layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound represented by Chemical Formula 1 has a high glass transition temperature (Tg), and has excellent thermal stability. Such an increase in the thermal stability becomes an important factor that provides driving stability to a device.

In addition, one embodiment of the present disclosure provides an organic light emitting device comprising: a first electrode; a second electrode provided to face the first electrode; and one or more organic material layers provided between the first electrode and the second electrode, wherein one or more layers of the organic material layers include the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic material layer may include one or more types selected from the group consisting of an electron injection layer, an electron transfer layer, a hole blocking layer, a light emitting layer, a light emitting auxiliary layer, an electron blocking layer, a hole transfer layer and a hole injection layer, and the one or more types of layers selected from the group consisting of an electron injection layer, an electron transfer layer, a hole blocking layer, a light emitting layer, a light emitting auxiliary layer, an electron blocking layer, a hole transfer layer and a hole injection layer may include the heterocyclic compound represented by Chemical Formula 1. The light emitting auxiliary layer performs a role of increasing light emission efficiency by compensating an optical resonance distance resulting from a wavelength of light emitted from a light emitting layer, and the electron blocking layer may perform a role of preventing electron injection from an electron transfer region. In addition, the light emitting auxiliary layer is a layer positioned between a negative electrode and a light emitting layer, or between a positive electrode and a light emitting layer, and when the light emitting auxiliary layer is positioned between the negative electrode and the light emitting layer, the light emitting auxiliary layer may be used to facilitate hole injection and/or transfer, or block electron overflow, and when the light emitting auxiliary layer is positioned between the positive electrode and the light emitting layer, the light emitting auxiliary layer may be used to facilitate electron injection and/or transfer, or block hole overflow.

In another embodiment of the present disclosure, the organic material layer may include a hole transfer layer, and the hole transfer layer may include the heterocyclic compound represented by Chemical Formula 1.

In another embodiment of the present disclosure, the organic material layer may include an electron blocking layer, and the electron blocking layer may include the heterocyclic compound represented by Chemical Formula 1.

In another embodiment of the present disclosure, the organic material layer may include a light emitting auxiliary layer, and the light emitting auxiliary layer may include the heterocyclic compound represented by Chemical Formula 1.

In one embodiment of the present disclosure, the organic light emitting device may be a blue organic light emitting device, and the heterocyclic compound represented by Chemical Formula 1 may be used as a material of the blue organic light emitting device.

In one embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a material of the red organic light emitting device.

In one embodiment of the present disclosure, the organic light emitting device may be a blue organic light emitting device, and the heterocyclic compound represented by Chemical Formula 1 may be used as a light emitting layer material of the blue organic light emitting device.

In one embodiment of the present disclosure, the organic light emitting device may be a green organic light emitting device, and the heterocyclic compound represented by Chemical Formula 1 may be used as a light emitting layer material of the green organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

In the organic light emitting device according to one embodiment of the present disclosure, the organic material layer includes an electron injection layer or an electron transfer layer, and the electron injection layer or the electron transfer layer may include the heterocyclic compound.

In the organic light emitting device according to another embodiment, the organic material layer includes an electron blocking layer or a hole blocking layer, and the electron blocking layer or the hole blocking layer may include the heterocyclic compound.

In the organic light emitting device according to another embodiment, the organic material layer includes an electron transfer layer, a light emitting layer or a hole blocking layer, and the electron transfer layer, the light emitting layer or the hole blocking layer may include the heterocyclic compound.

In the organic light emitting device according to another embodiment, the organic material layer includes a hole transfer layer, an electron blocking layer or a light emitting auxiliary layer, and the hole transfer layer, the electron blocking layer or the light emitting auxiliary layer may include the heterocyclic compound.

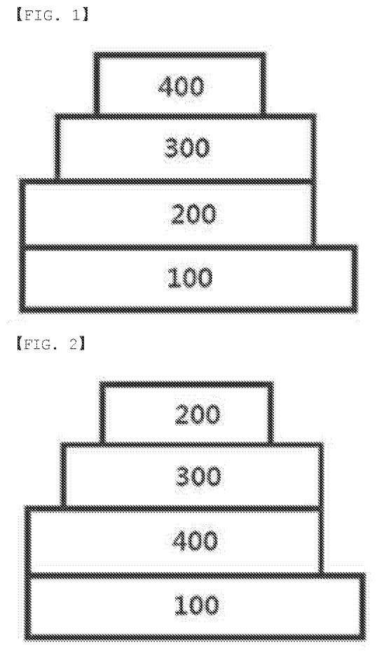

FIG. 1 to FIG. 3 illustrate a lamination order of electrodes and organic material layers of the organic light emitting device according to one embodiment of the present disclosure. However, the scope of the present application is not limited to these diagrams, and structures of organic light emitting devices known in the art may also be used in the present application.

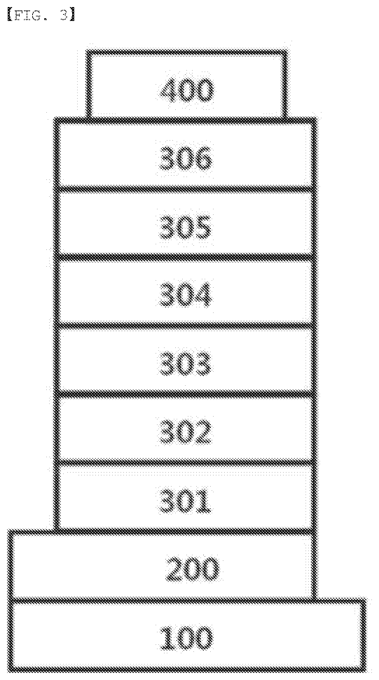

FIG. 1 illustrates the organic light emitting device in which a positive electrode (200), an organic material layer (300) and a negative electrode (400) are consecutively laminated on a substrate (100). However, the structure is not limited to such a structure, and as illustrated in FIG. 2, an organic light emitting device in which a negative electrode, an organic material layer and a positive electrode are consecutively laminated on a substrate may also be obtained.

FIG. 3 illustrates a case of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, the layers other than the light emitting layer may not be included, and other necessary functional layers may be further added. For example, a light emitting auxiliary layer may be added (not shown in FIG. 3).

In addition, one embodiment of the present disclosure provides a composition for an organic material layer of an organic light emitting device, the composition including the heterocyclic compound represented by Chemical Formula 1.

Specific descriptions on the heterocyclic compound represented by Chemical Formula 1 are the same as the descriptions provided above.

The composition for an organic material layer of an organic light emitting device may be used when forming an organic material of an organic light emitting device, and particularly, may be more preferably used when forming a hole transfer layer, an electron blocking layer or a light emitting auxiliary layer.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including an electron injection layer, an electron transfer layer, a hole blocking layer, a light emitting layer, a light emitting auxiliary layer, an electron blocking layer, an electron transfer layer, an electron injection layer and the like as the organic material layer. However, the structure of the organic light emitting device is not limited thereto, and may include a smaller number of organic material layers.

One embodiment of the present disclosure provides a method for manufacturing an organic light emitting device, the method comprising the steps of:
preparing a substrate; forming a first electrode on the substrate; forming one or more organic material layers on the first electrode; and forming a second electrode on the one or more organic material layers, wherein the forming of one or more organic material layers includes forming the one or more organic material layers using the composition for an organic material layer according to one embodiment of the present disclosure.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials having relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly (3-methylthiophene), poly[3,4-(ethylene-1,2-dioxy)thiophene] (PEDOT), polypyrrole and polyaniline, and the like, but are not limited thereto.

As the negative electrode material, materials having relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino) phenyl]benzene (m-MTDAPB) described in the literature [Advanced Material, 6, p. 677 (1994)], conductive polymers having solubility such as polyaniline/dodecylbenzene sulfonic acid or poly(3,4-ethylenedioxythiophene)/poly (4-styrene-sulfonate), polyaniline/camphor sulfonic acid or polyaniline/poly(4-styrenesulfonate), and the like, may be used.

As the hole transfer layer material, pyrazoline derivatives, arylamine-based derivatives, stilbene derivatives, triphenyldiamine derivatives and the like may be used, and low molecular or high molecular materials may also be used.

As the electron transfer layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light by binding holes and electrons injected from the positive electrode and the negative electrode, respectively, may be used alone, however, materials having a host material and a dopant material involving together in light emission may also be used.

When mixing hosts of the light emitting layer material, same series hosts may be mixed, or different series hosts may be mixed. For example, any two or more types of materials among n-type host materials or p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a similar principle used in the organic light emitting device.

Hereinafter, preferred examples are provided to illuminate the present disclosure, however, the following examples are provided to more readily understand the present disclosure, and the present disclosure is not limited thereto.

### PREPARATION EXAMPLE

### Preparation Example 1> Preparation of Compound 1

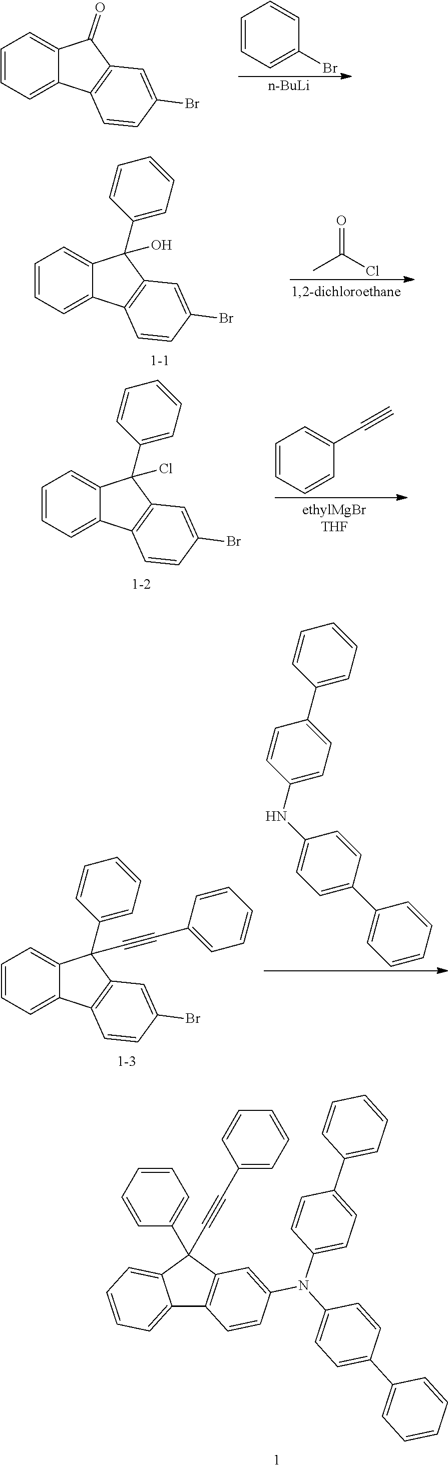

1) Preparation of Compound 1-1

In a one neck round bottom flask (one neck r.b.f), 2-bromo-9H-fluoren-9-one (50 g, 193 mmol) was dissolved in anhydrous tetrahydrofuran (THF) (500 ml), and after reducing the pressure to create a vacuum state, the flask was filled with nitrogen (N₂), and the bath temperature was lowered to −78° C. After that, N-BuLi (16 g, 250 mmol) was injected thereto, and after 1 hour, bromobenzene (30 g, 193 mmol) dissolved in anhydrous THF (300 ml (10 T)) was injected thereto, and the result was stirred under reflux for 4 hours at room temperature.

After the reaction was completed, an aqueous sodium thiosulfate solution was added dropwise thereto, and the result was extracted with methylene chloride (MC)/distilled water. The obtained organic layer was concentrated, and purified using an absorption column chromatography purification method to obtain Compound 1-1 (57 g, yield 88%).

### 2) Preparation of Compound 1-2

Into a one neck round bottom flask (one neck r.b.f), a mixture of Compound 1-1 (57 g, 169 mmol), 1,2-dichloroethane (500 ml) and acetyl chloride (16 g, 203 mmol) was injected, and the mixture refluxed for 24 hours. After that, distilled water was added thereto to terminate the reaction, and after adding methanol thereto, the result was purified by being passed through silica to obtain solid Compound 1-2 (45 g, yield 75%).

### 3) Preparation of Compound 1-3

Into a one neck round bottom flask (one neck r.b.f), ethynylbenzene (13 g, 132 mmol) and THF (200 ml) were added, and ethylmagnesium bromide (45 g, 378 mmol) was injected thereto. Compound 1-2 (45 g, 126 mmol) was added to THF, the mixture was added dropwise to the flask using a dropping funnel, and the result was stirred at 60° C. A large amount of distilled water was prepared in a plastic cup, and the reaction solution was added dropwise thereto. The result was extracted with MC/water, and the organic layer was concentrated and purified using a column chromatography purification method to obtain Compound 1-3 (43 g, yield 78%).

### 4) Preparation of Compound 1

Into a one neck round bottom flask (one neck r.b.f), Compound 1-3 (10 g, 24 mmol), di([1,1′-biphenyl]-4-yl)amine (8 g, 25 mmol), tris(dibenzylideneacetone)dipalladium (Pd₂ (dba)₃) (1 g, 1.2 mmol), dicyclohexyl (2′,4′,6′-triisopropyl-[1,1′-biphenyl]-2-yl)phosphine (Xphos) (1.1 g, 2.4 mmol) and NaOtBu (7 g, 72 mmol) were injected, and the mixture was stirred under reflux for 4 hours.

The reaction solution was filtered through celite, and purified using a column chromatography purification method to obtain Compound 1 (13 g, 20 mmol).

Target compounds of the following Table 1 were synthesized in the same manner as in Preparation Example 1 except that the following Compound A was used instead of bromobenzene, the following Compound B was used instead of ethynylbenzene, and the following Compound C was used instead of di([1,1′-biphenyl]-4-yl)amine.

TABLE 1

| Compound | A | B | C | Target Compound |
|---|---|---|---|---|
| 2 | 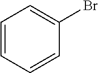 | 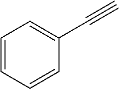 | 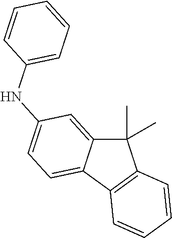 | 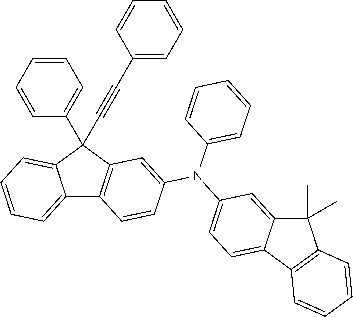 [SMILES: CC1(C)c2ccccc2-c2ccc(N(c3ccccc3)c3ccc4c(c3)C(C#Cc3ccccc3)(c3ccccc3)c3ccccc3-4)cc21] |
| 3 | 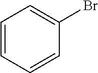 | 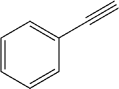 | 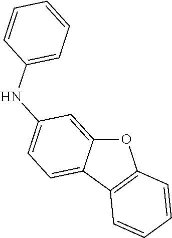 | 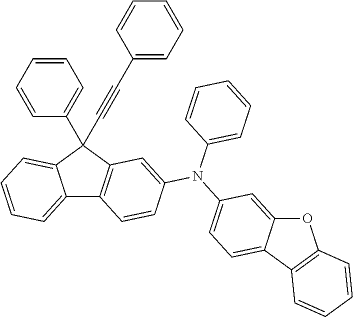 [SMILES: C(#CC1(c2ccccc2)c2ccccc2-c2ccc(N(c3ccccc3)c3ccc4c(c3)oc3ccccc34)cc21)c1ccccc1] |
| 4 | 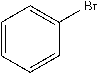 | 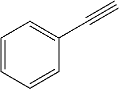 | 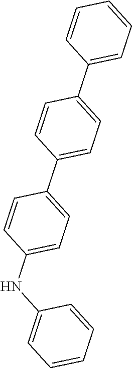 | 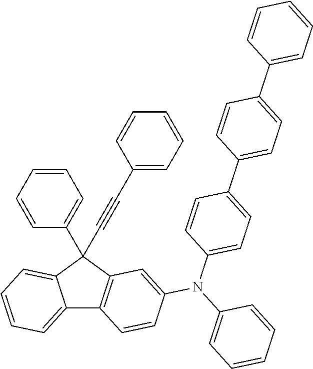 [SMILES: C(#CC1(c2ccccc2)c2ccccc2-c2ccc(N(c3ccccc3)c3ccc(-c4ccc(-c5ccccc5)cc4)cc3)cc21)c1ccccc1] |
| 5 | 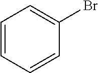 | 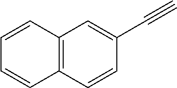 | 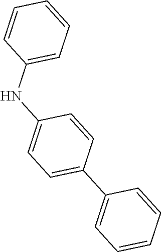 | 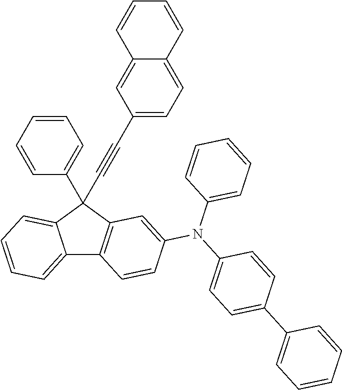 [SMILES: C(#CC1(c2ccccc2)c2ccccc2-c2ccc(N(c3ccccc3)c3ccc(-c4ccccc4)cc3)cc21)c1ccc2ccccc2c1] |
| 6 | 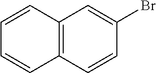 | 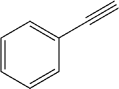 | 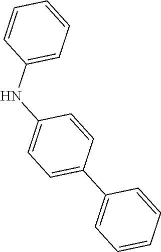 | 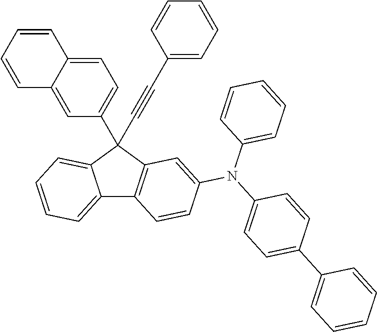 [SMILES: C(#CC1(c2ccc3ccccc3c2)c2ccccc2-c2ccc(N(c3ccccc3)c3ccc(-c4ccccc4)cc3)cc21)c1ccccc1] |
| 7 | 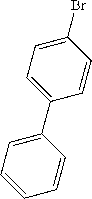 | 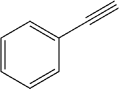 | 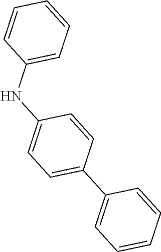 | 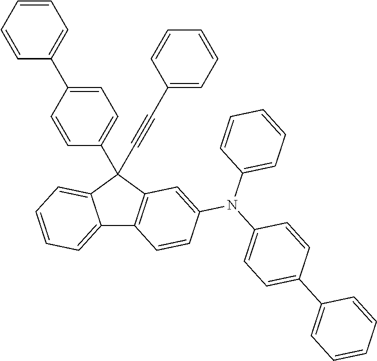 [SMILES: C(#CC1(c2ccc(-c3ccccc3)cc2)c2ccccc2-c2ccc(N(c3ccccc3)c3ccc(-c4ccccc4)cc3)cc21)c1ccccc1] |
| 8 | 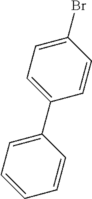 | 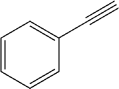 | 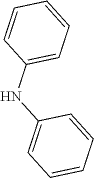 | 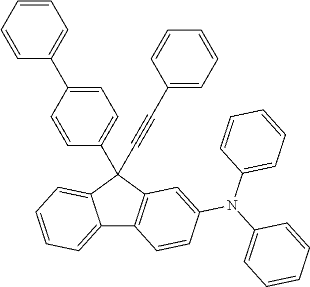 [SMILES: C(#CC1(c2ccc(-c3ccccc3)cc2)c2ccccc2-c2ccc(N(c3ccccc3)c3ccccc3)cc21)c1ccccc1] |
| 9 | 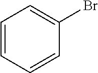 | 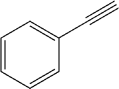 | 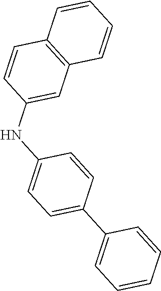 | 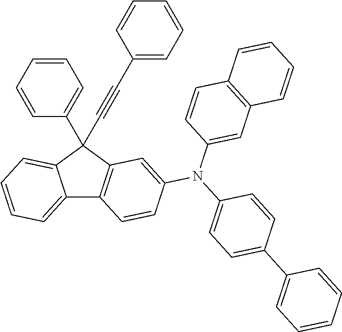 [SMILES: C(#CC1(c2ccccc2)c2ccccc2-c2ccc(N(c3ccc(-c4ccccc4)cc3)c3ccc4ccccc4c3)cc21)c1ccccc1] |
| 10 | 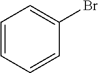 | 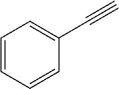 | 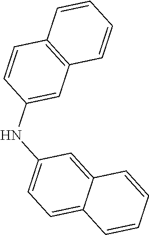 | 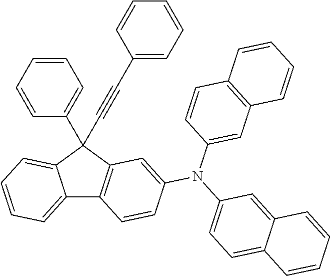 [SMILES: C(#CC1(c2ccccc2)c2ccccc2-c2ccc(N(c3ccc4ccccc4c3)c3ccc4ccccc4c3)cc21)c1ccccc1] |
| 11 | 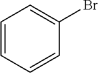 | 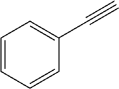 | 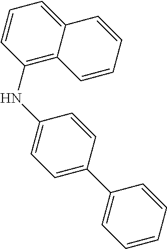 | 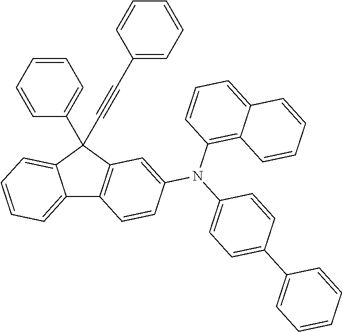 [SMILES: C(#CC1(c2ccccc2)c2ccccc2-c2ccc(N(c3ccc(-c4ccccc4)cc3)c3cccc4ccccc34)cc21)c1ccccc1] |
| 12 | 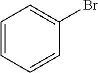 | 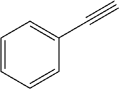 | 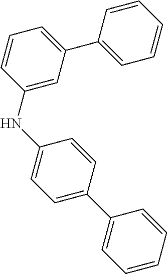 | 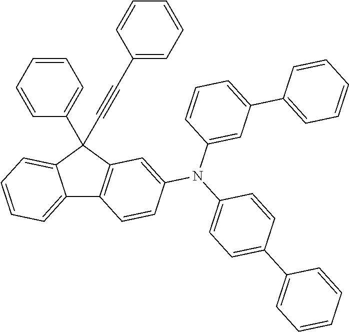 [SMILES: C(#CC1(c2ccccc2)c2ccccc2-c2ccc(N(c3ccc(-c4ccccc4)cc3)c3cccc(-c4ccccc4)c3)cc21)c1ccccc1] |
| 13 | 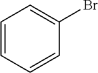 | 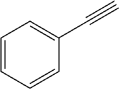 | 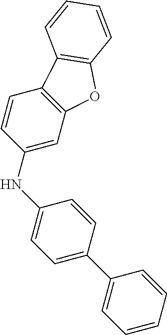 | 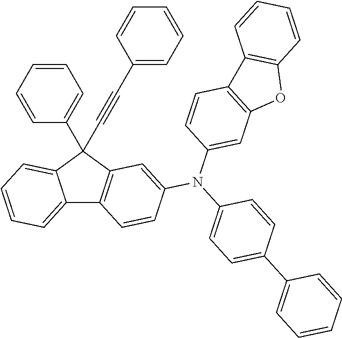 [SMILES: C(#CC1(c2ccccc2)c2ccccc2-c2ccc(N(c3ccc(-c4ccccc4)cc3)c3ccc4c(c3)oc3ccccc34)cc21)c1ccccc1] |
| 14 | 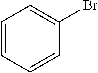 | 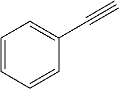 | 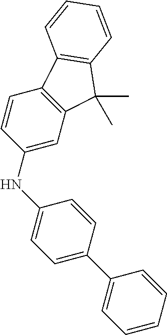 | 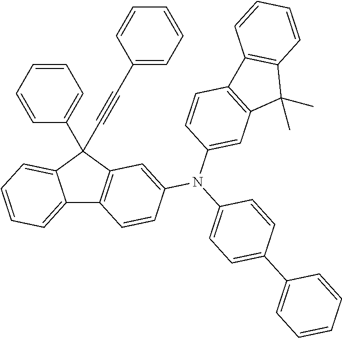 [SMILES: CC1(C)c2ccccc2-c2ccc(N(c3ccc(-c4ccccc4)cc3)c3ccc4c(c3)C(C#Cc3ccccc3)(c3ccccc3)c3ccccc3-4)cc21] |
| 15 | 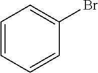 | 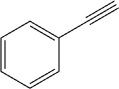 | 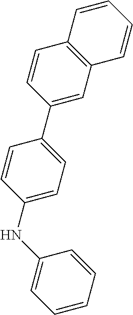 | 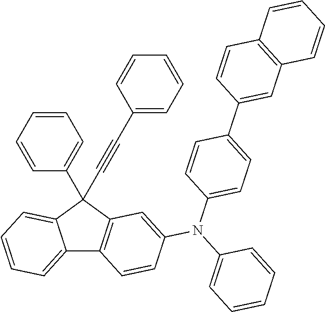 [SMILES: C(#CC1(c2ccccc2)c2ccccc2-c2ccc(N(c3ccccc3)c3ccc(-c4ccc5ccccc5c4)cc3)cc21)c1ccccc1] |
| 16 | 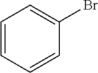 | 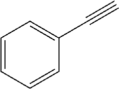 | 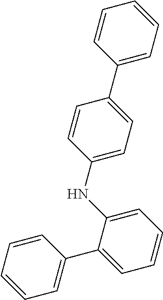 | 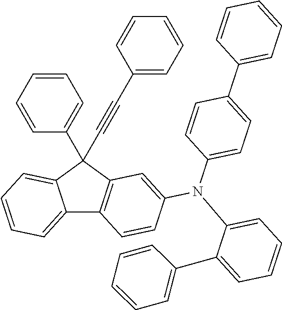 [SMILES: C(#CC1(c2ccccc2)c2ccccc2-c2ccc(N(c3ccc(-c4ccccc4)cc3)c3ccccc3-c3ccccc3)cc21)c1ccccc1] |
| 17 | 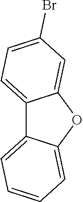 | 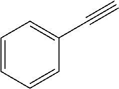 | 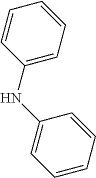 | 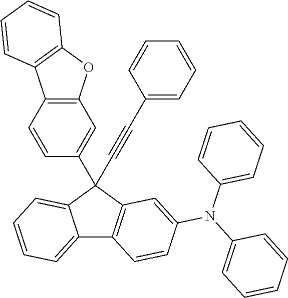 [SMILES: C(#CC1(c2ccc3c(c2)oc2ccccc23)c2ccccc2-c2ccc(N(c3ccccc3)c3ccccc3)cc21)c1ccccc1] |
| 18 | 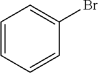 | 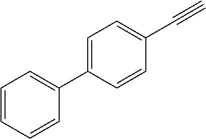 | 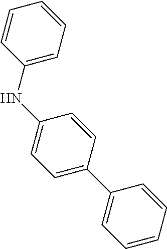 | 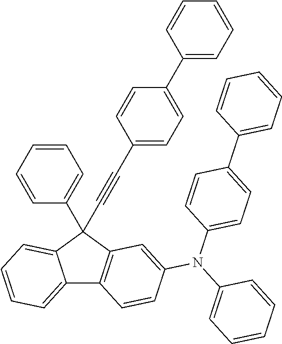 [SMILES: C(#CC1(c2ccccc2)c2ccccc2-c2ccc(N(c3ccccc3)c3ccc(-c4ccccc4)cc3)cc21)c1ccc(-c2ccccc2)cc1] |
| 19 | 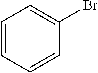 | 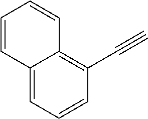 | 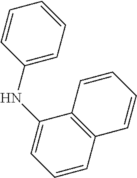 | 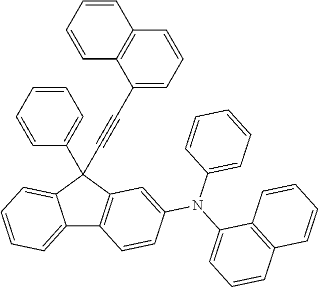 [SMILES: C(#CC1(c2ccccc2)c2ccccc2-c2ccc(N(c3ccccc3)c3cccc4ccccc34)cc21)c1cccc2ccccc12] |
| 20 | 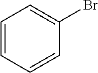 | 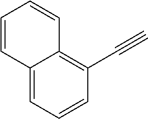 | 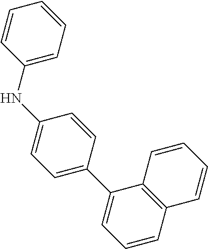 | 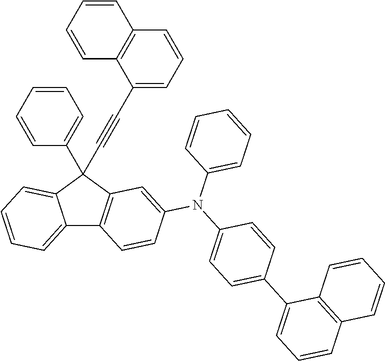 [SMILES: C(#CC1(c2ccccc2)c2ccccc2-c2ccc(N(c3ccccc3)c3ccc(-c4cccc5ccccc45)cc3)cc21)c1cccc2ccccc12] |
| 41 | 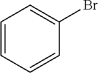 | 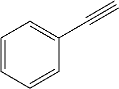 | 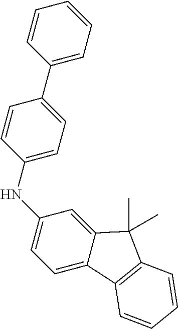 | 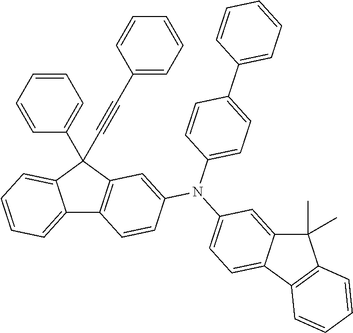 [SMILES: CC1(C)c2ccccc2-c2ccc(N(c3ccc(-c4ccccc4)cc3)c3ccc4c(c3)C(C#Cc3ccccc3)(c3ccccc3)c3ccccc3-4)cc21] |
| 42 | 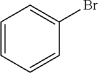 | 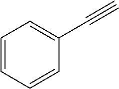 | 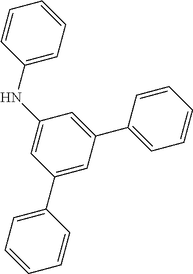 | 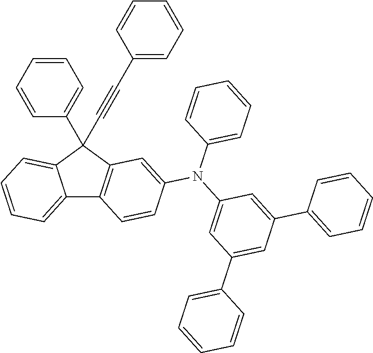 [SMILES: C(#CC1(c2ccccc2)c2ccccc2-c2ccc(N(c3ccccc3)c3cc(-c4ccccc4)cc(-c4ccccc4)c3)cc21)c1ccccc1] |
| 43 | 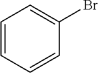 | 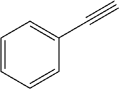 | 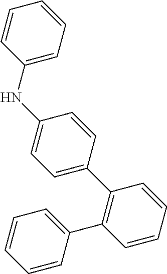 | 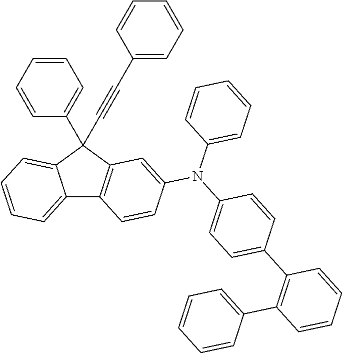 [SMILES: C(#CC1(c2ccccc2)c2ccccc2-c2ccc(N(c3ccccc3)c3ccc(-c4ccccc4-c4ccccc4)cc3)cc21)c1ccccc1] |
| 44 | 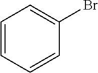 | 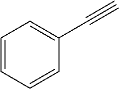 | 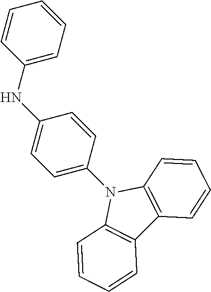 | 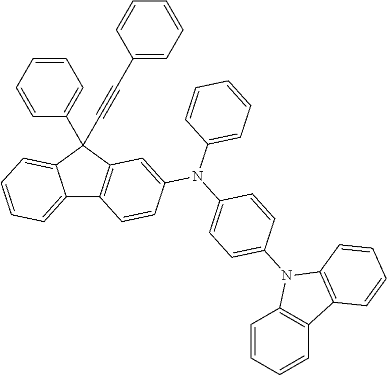 [SMILES: C(#CC1(c2ccccc2)c2ccccc2-c2ccc(N(c3ccccc3)c3ccc(-n4c5ccccc5c5ccccc54)cc3)cc21)c1ccccc1] |
| 45 | 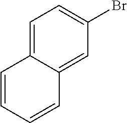 | 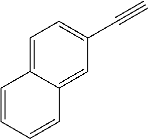 | 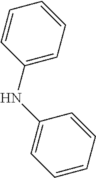 | 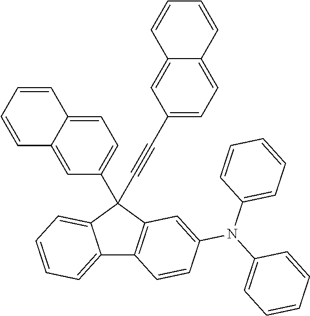 [SMILES: C(#CC1(c2ccc3ccccc3c2)c2ccccc2-c2ccc(N(c3ccccc3)c3ccccc3)cc21)c1ccc2ccccc2c1] |
| 46 | 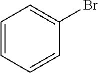 | 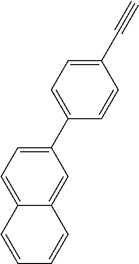 | 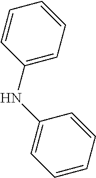 | 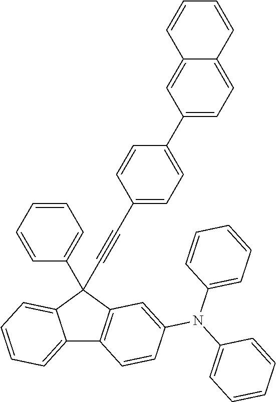 [SMILES: C(#CC1(c2ccccc2)c2ccccc2-c2ccc(N(c3ccccc3)c3ccccc3)cc21)c1ccc(-c2ccc3ccccc3c2)cc1] |
| 47 | 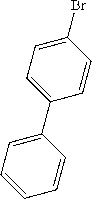 | 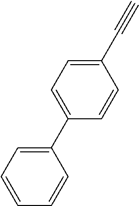 | 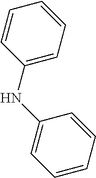 | 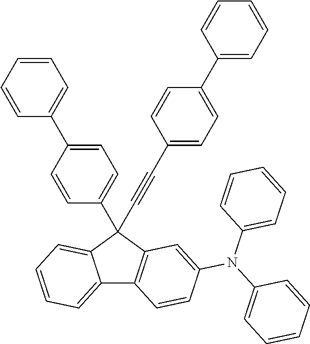 [SMILES: C(#CC1(c2ccc(-c3ccccc3)cc2)c2ccccc2-c2ccc(N(c3ccccc3)c3ccccc3)cc21)c1ccc(-c2ccccc2)cc1] |
| 48 | 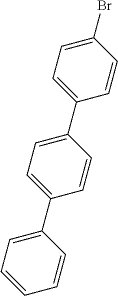 | 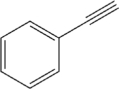 | 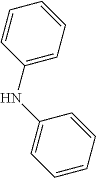 | 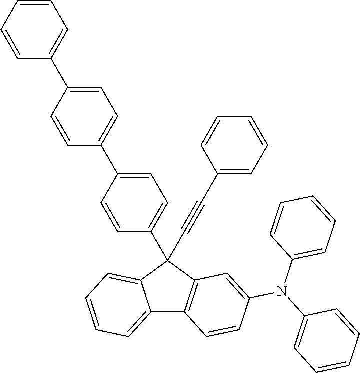 [SMILES: C(#CC1(c2ccc(-c3ccc(-c4ccccc4)cc3)cc2)c2ccccc2-c2ccc(N(c3ccccc3)c3ccccc3)cc21)c1ccccc1] |
| 49 | 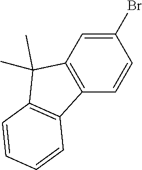 | 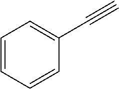 | 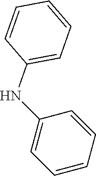 | 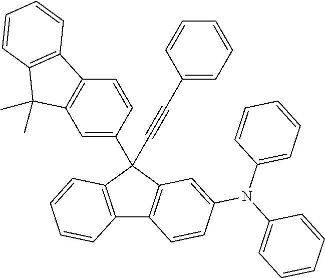 [SMILES: CC1(C)c2ccccc2-c2ccc(C3(C#Cc4ccccc4)c4ccccc4-c4ccc(N(c5ccccc5)c5ccccc5)cc43)cc21] |
| 50 | 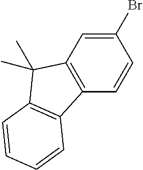 | 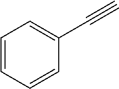 | 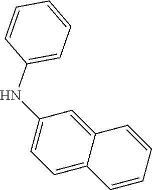 | 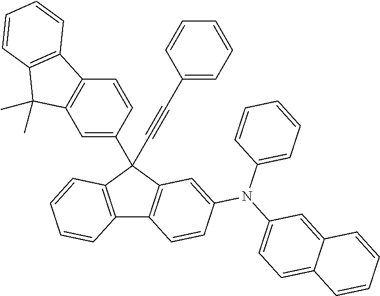 [SMILES: CC1(C)c2ccccc2-c2ccc(C3(C#Cc4ccccc4)c4ccccc4-c4ccc(N(c5ccccc5)c5ccc6ccccc6c5)cc43)cc21] |
| 51 | 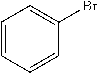 | 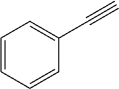 | 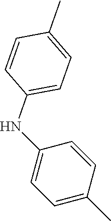 | 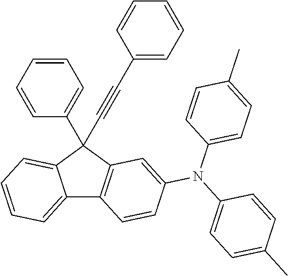 [SMILES: Cc1ccc(N(c2ccc(C)cc2)c2ccc3c(c2)C(C#Cc2ccccc2)(c2ccccc2)c2ccccc2-3)cc1] |
| 52 | 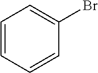 | 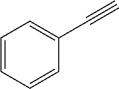 | 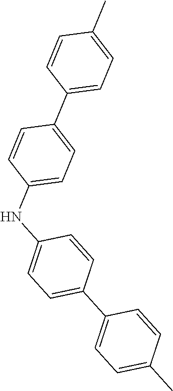 | 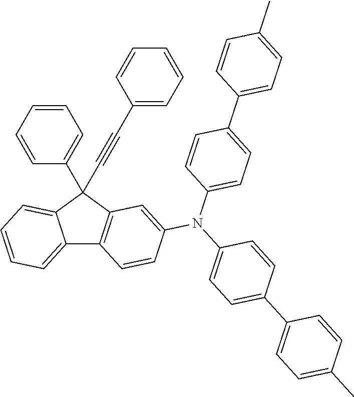 [SMILES: Cc1ccc(-c2ccc(N(c3ccc(-c4ccc(C)cc4)cc3)c3ccc4c(c3)C(C#Cc3ccccc3)(c3ccccc3)c3ccccc3-4)cc2)cc1] |
| 53 | 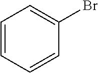 | 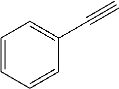 | 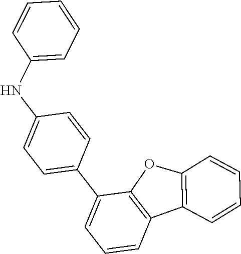 | 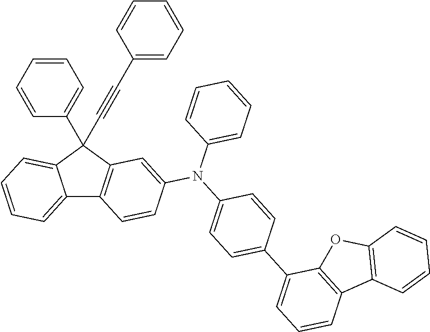 [SMILES: C(#CC1(c2ccccc2)c2ccccc2-c2ccc(N(c3ccccc3)c3ccc(-c4cccc5c4oc4ccccc45)cc3)cc21)c1ccccc1] |
| 54 | 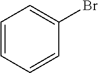 | 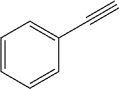 | 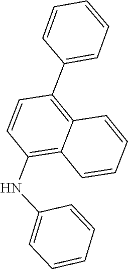 | 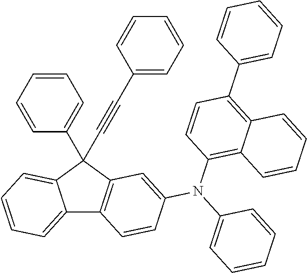 [SMILES: C(#CC1(c2ccccc2)c2ccccc2-c2ccc(N(c3ccccc3)c3ccc(-c4ccccc4)c4ccccc34)cc21)c1ccccc1] |
| 55 | 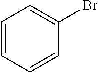 | 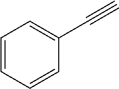 | 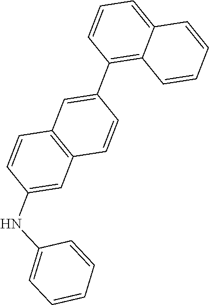 | 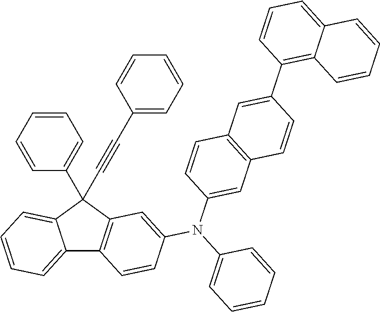 [SMILES: C(#CC1(c2ccccc2)c2ccccc2-c2ccc(N(c3ccccc3)c3ccc4cc(-c5cccc6ccccc56)ccc4c3)cc21)c1ccccc1] |
| 56 | 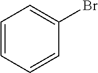 | 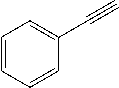 | 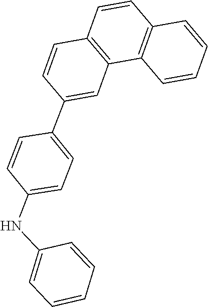 | 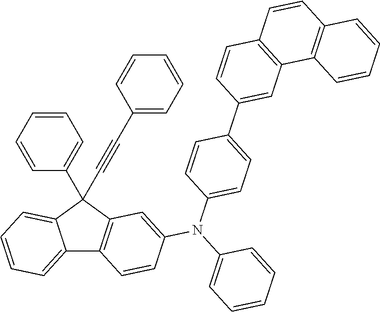 [SMILES: C(#CC1(c2ccccc2)c2ccccc2-c2ccc(N(c3ccccc3)c3ccc(-c4ccc5ccc6ccccc6c5c4)cc3)cc21)c1ccccc1] |
| 57 | 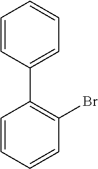 | 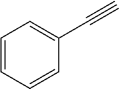 | 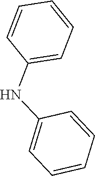 | 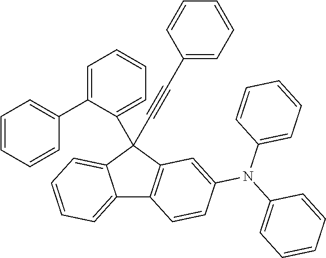 [SMILES: C(#CC1(c2ccccc2-c2ccccc2)c2ccccc2-c2ccc(N(c3ccccc3)c3ccccc3)cc21)c1ccccc1] |
| 58 | 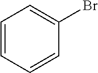 | 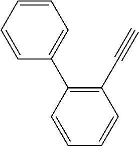 | 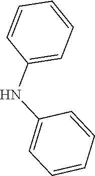 | 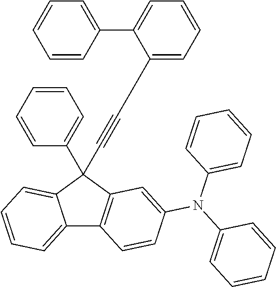 [SMILES: C(#CC1(c2ccccc2)c2ccccc2-c2ccc(N(c3ccccc3)c3ccccc3)cc21)Cc1ccccc1-c1ccccc1] |
| 59 | 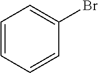 | 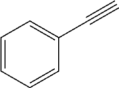 | 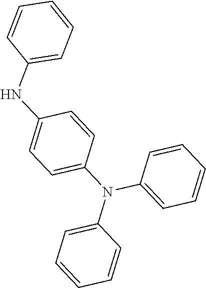 | 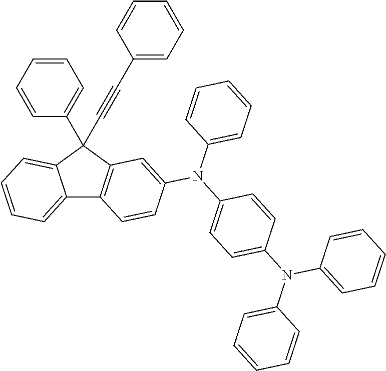 [SMILES: C(#CC1(c2ccccc2)c2ccccc2-c2ccc(N(c3ccccc3)c3ccc(N(c4ccccc4)c4ccccc4)cc3)cc21)c1ccccc1] |
| 60 | 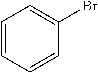 | 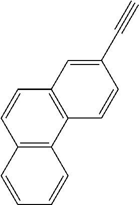 | 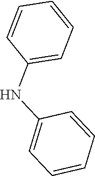 | 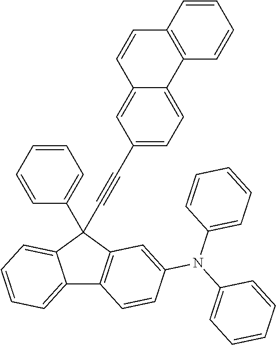 [SMILES: C(#CC1(c2ccccc2)c2ccccc2-c2ccc(N(c3ccccc3)c3ccccc3)cc21)c1ccc2c(ccc3ccccc32)c1] |
| 61 | 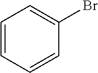 | 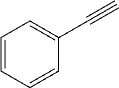 | 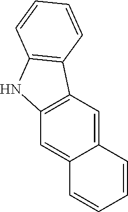 | 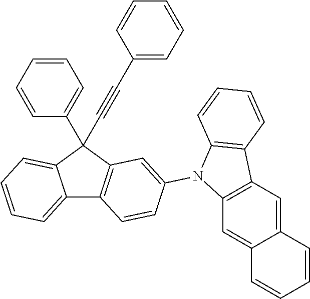 [SMILES: C(#CC1(c2ccccc2)c2ccccc2-c2ccc(-n3c4ccccc4c4cc5ccccc5cc43)cc21)c1ccccc1] |
| 62 | 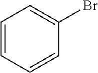 | 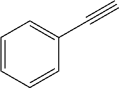 | 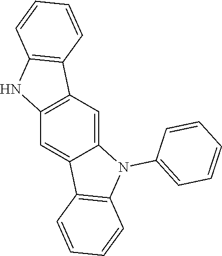 | 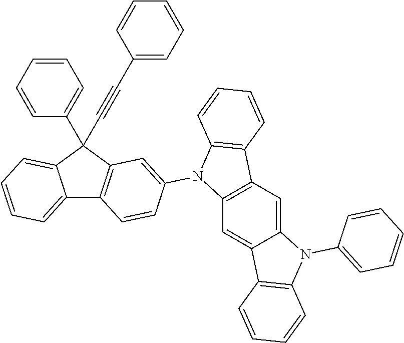 [SMILES: C(#CC1(c2ccccc2)c2ccccc2-c2ccc(-n3c4ccccc4c4cc5c(cc43)c3ccccc3n5-c3ccccc3)cc21)c1ccccc1] |
| 63 | 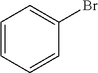 | 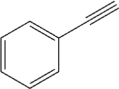 | 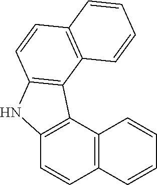 | 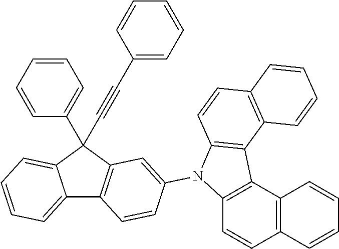 [SMILES: C(#CC1(c2ccccc2)c2ccccc2-c2ccc(-n3c4ccc5ccccc5c4c4c5ccccc5ccc43)cc21)c1ccccc1] |
| 64 | 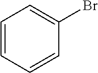 | 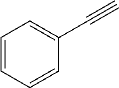 | 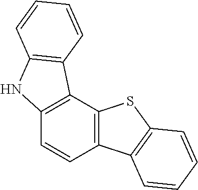 | 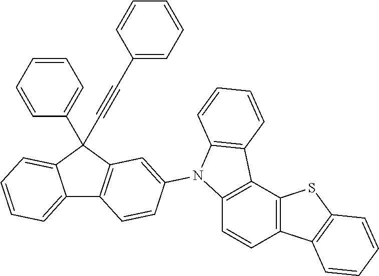 [SMILES: C(#CC1(c2ccccc2)c2ccccc2-c2ccc(-n3c4ccccc4c4c5sc6ccccc6c5ccc43)cc21)c1ccccc1] |
| 65 | 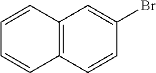 | 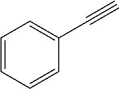 | 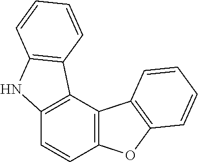 | 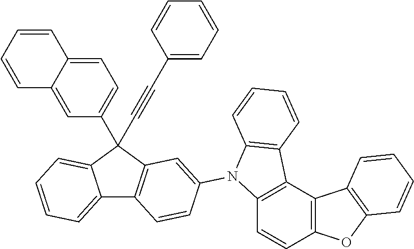 [SMILES: C(#CC1(c2ccc3ccccc3c2)c2ccccc2-c2ccc(-n3c4ccccc4c4c5c(ccc43)oc3ccccc35)cc21)c1ccccc1] |
| 66 | 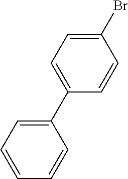 | 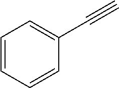 | 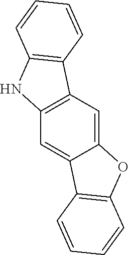 | 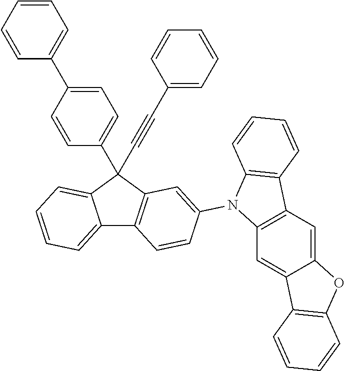 [SMILES: C(#CC1(c2ccc(-c3ccccc3)cc2)c2ccccc2-c2ccc(-n3c4ccccc4c4cc5oc6ccccc6c5cc43)cc21)c1ccccc1] |
| 67 | 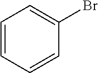 | 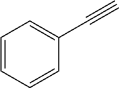 | 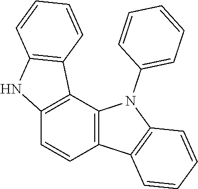 | 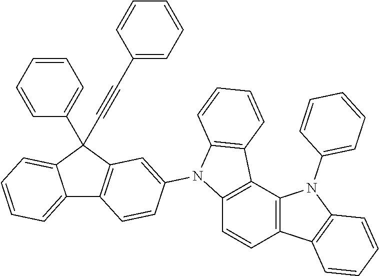 [SMILES: C(#CC1(c2ccccc2)c2ccccc2-c2ccc(-n3c4ccccc4c4c3ccc3c5ccccc5n(-c5ccccc5)c34)cc21)c1ccccc1] |
| 68 | 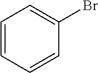 | 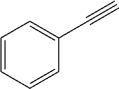 | 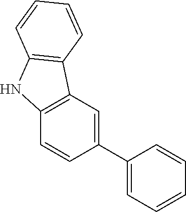 | 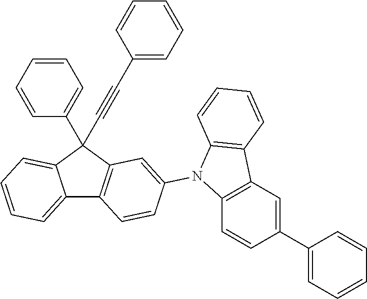 [SMILES: C(#CC1(c2ccccc2)c2ccccc2-c2ccc(-n3c4ccccc4c4cc(-c5ccccc5)ccc43)cc21)c1ccccc1] |
| 69 | 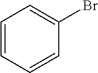 | 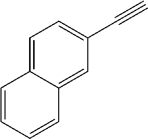 | 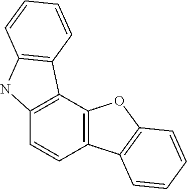 | 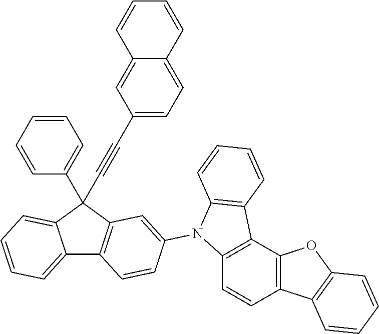 [SMILES: C(#CC1(c2ccccc2)c2ccccc2-c2ccc(-n3c4ccccc4c4c5oc6ccccc6c5ccc43)cc21)c1ccc2ccccc2c1] |
| 70 | 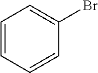 | 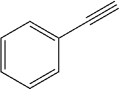 | 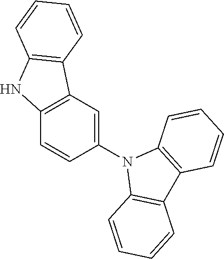 | 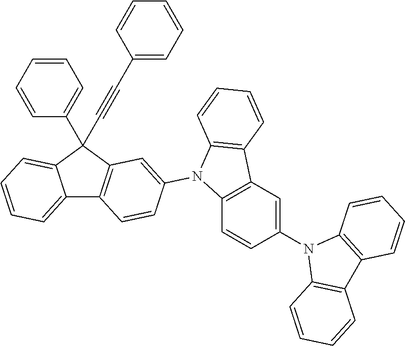 [SMILES: C(#CC1(c2ccccc2)c2ccccc2-c2ccc(-n3c4ccccc4c4cc(-n5c6ccccc6c6ccccc65)ccc43)cc21)c1ccccc1] |
| 71 | 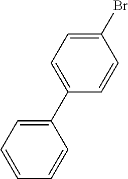 | 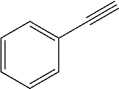 | 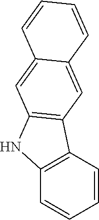 | 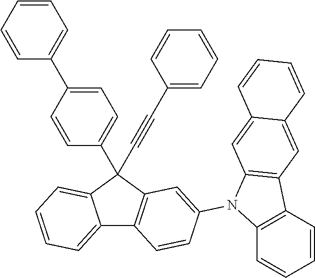 [SMILES: C(#CC1(c2ccc(-c3ccccc3)cc2)c2ccccc2-c2ccc(-n3c4ccccc4c4cc5ccccc5cc43)cc21)c1ccccc1] |
| 72 | 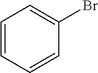 | 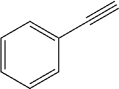 | 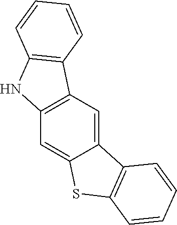 | 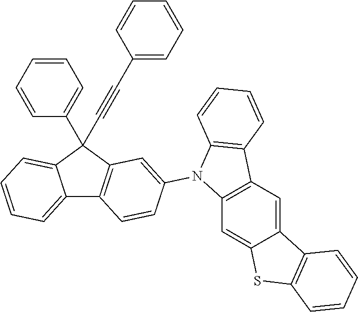 [SMILES: C(#CC1(c2ccccc2)c2ccccc2-c2ccc(-n3c4ccccc4c4cc5c(cc43)sc3ccccc35)cc21)c1ccccc1] |
| 73 | 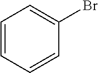 | 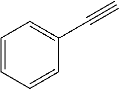 | 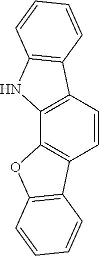 | 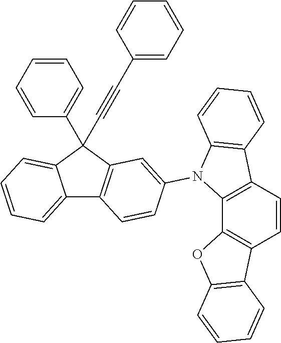 [SMILES: C(#CC1(c2ccccc2)c2ccccc2-c2ccc(-n3c4ccccc4c4ccc5c6ccccc6oc5c43)cc21)c1ccccc1] |
| 74 | 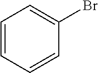 | 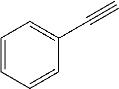 | 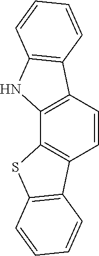 | 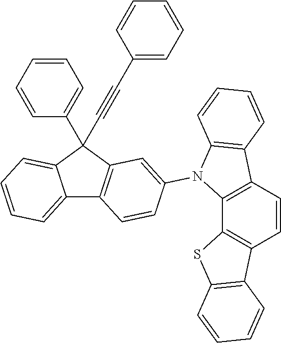 [SMILES: C(#CC1(c2ccccc2)c2ccccc2-c2ccc(-n3c4ccccc4c4ccc5c6ccccc6sc5c43)cc21)c1ccccc1] |
| 75 | 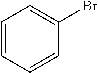 | 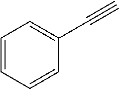 | 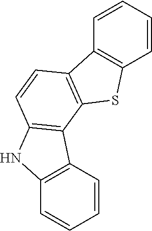 | 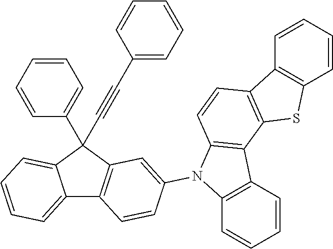 [SMILES: C(#CC1(c2ccccc2)c2ccccc2-c2ccc(-n3c4ccccc4c4c5sc6ccccc6c5ccc43)cc21)c1ccccc1] |
| 76 | 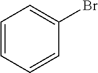 | 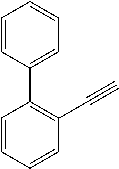 | 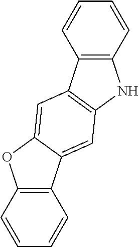 | 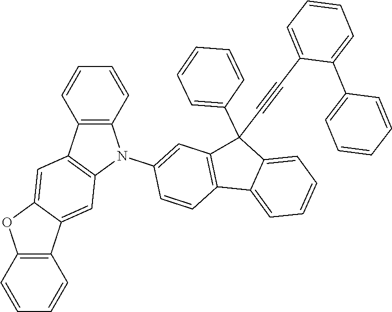 [SMILES: C(#CC1(c2ccccc2)c2ccccc2-c2ccc(-n3c4ccccc4c4cc5oc6ccccc6c5cc43)cc21)c1ccccc1-c1ccccc1] |
| 77 | 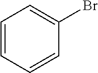 | 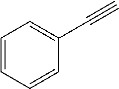 | 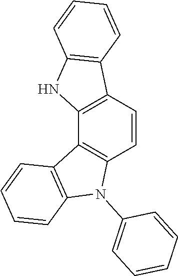 | 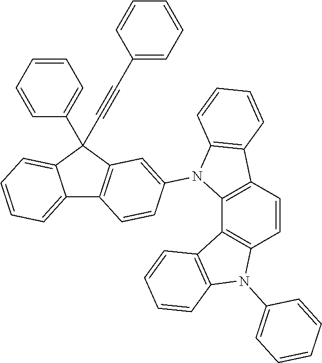 [SMILES: C(#CC1(c2ccccc2)c2ccccc2-c2ccc(-n3c4ccccc4c4ccc5c(c6ccccc6n5-c5ccccc5)c43)cc21)c1ccccc1] |
| 78 | 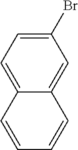 | 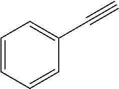 | 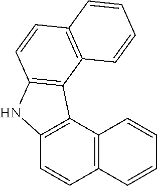 | 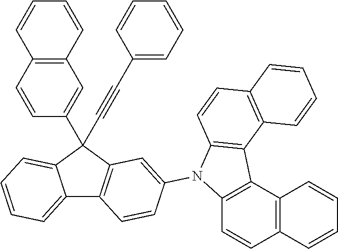 [SMILES: C(#CC1(c2ccc3ccccc3c2)c2ccccc2-c2ccc(-n3c4ccc5ccccc5c4c4c5ccccc5ccc43)cc21)c1ccccc1] |
| 79 | 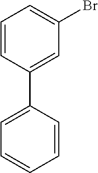 | 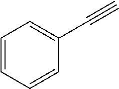 | 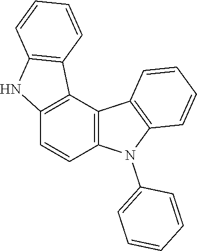 | 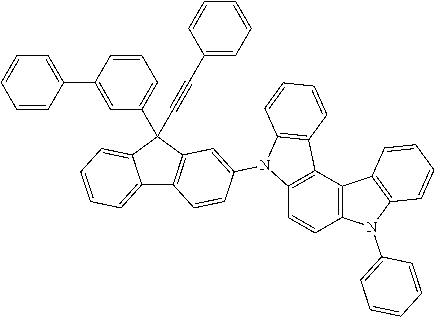 [SMILES: C(#CC1(c2cccc(-c3ccccc3)c2)c2ccccc2-c2ccc(-n3c4ccccc4c4c5c6ccccc6n(-c6ccccc6)c5ccc43)cc21)c1ccccc1] |
| 80 | 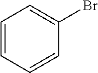 | 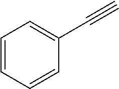 | 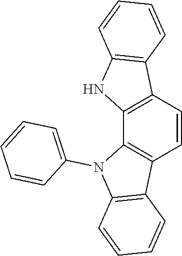 | 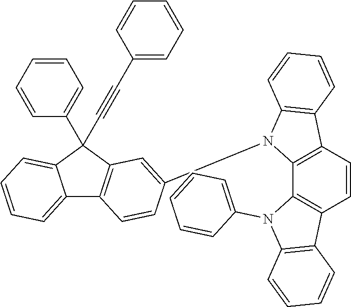 [SMILES: C(#CC1(c2ccccc2)c2ccccc2-c2ccc(-n3c4ccccc4c4ccc5c6ccccc6n(-c6ccccc6)c5c43)cc21)c1ccccc1] |
| 169 | 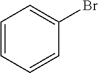 | 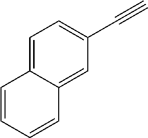 | 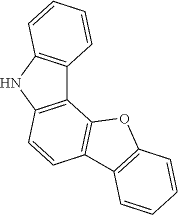 | 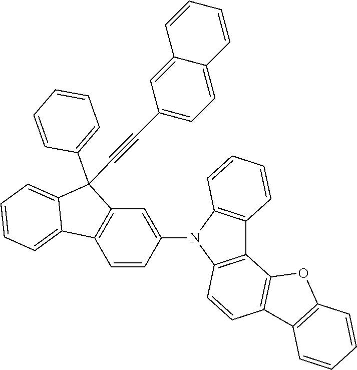 [SMILES: C(#CC1(c2ccccc2)c2ccccc2-c2ccc(-n3c4ccccc4c4c5oc6ccccc6c5ccc43)cc21)c1ccc2ccccc2c1] |
| 170 | 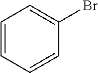 | 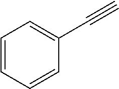 | 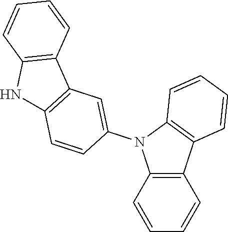 | 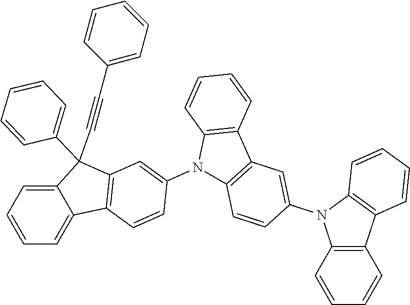 [SMILES: C(#CC1(c2ccccc2)c2ccccc2-c2ccc(-n3c4ccccc4c4cc(-n5c6ccccc6c6ccccc65)ccc43)cc21)c1ccccc1] |
| 177 | 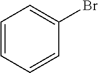 | 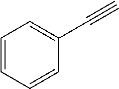 | 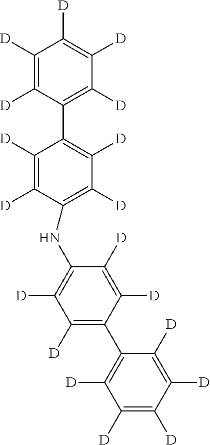 | 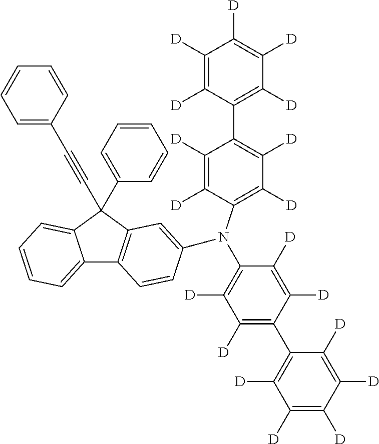 [SMILES: [2H]c1c([2H])c([2H])c(-c2c([2H])c([2H])c(N(c3ccc4c(c3)C(C#Cc3ccccc3)(c3ccccc3)c3ccccc3-4)c3c([2H])c([2H])c(-c4c([2H])c([2H])c([2H])c([2H])c4[2H])c([2H])c3[2H])c([2H])c2[2H])c([2H])c1[2H]] |
| 178 | 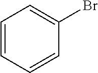 | 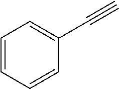 | 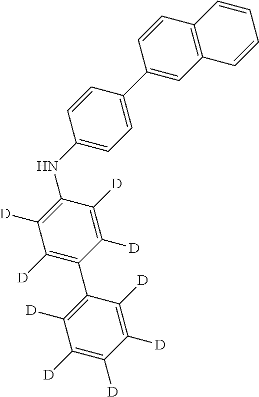 | 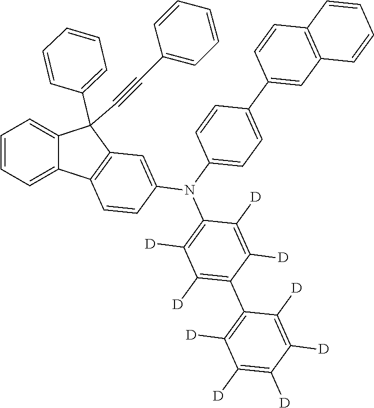 [SMILES: [2H]c1c([2H])c([2H])c(-c2c([2H])c([2H])c(N(c3ccc(-c4ccc5ccccc5c4)cc3)c3ccc4c(c3)C(C#Cc3ccccc3)(c3ccccc3)c3ccccc3-4)c([2H])c2[2H])c([2H])c1[2H]] |
| 179 | 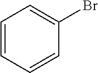 | 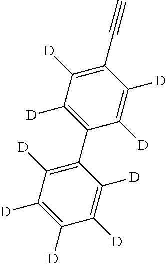 | 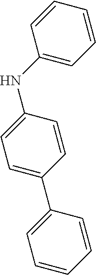 | 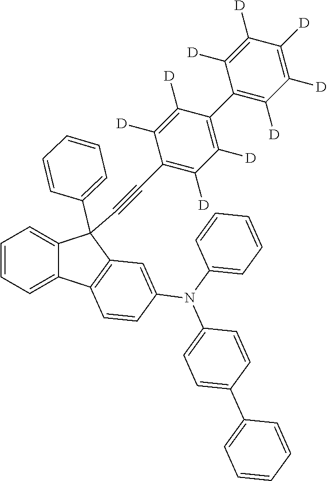 [SMILES: [2H]c1c([2H])c([2H])c(-c2c([2H])c([2H])c(C#CC3(c4ccccc4)c4ccccc4-c4ccc(N(c5ccccc5)c5ccc(-c6ccccc6)cc5)cc43)c([2H])c2[2H])c([2H])c1[2H]] |
| 180 | 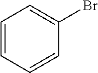 | 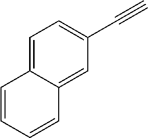 | 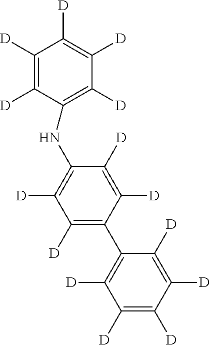 | 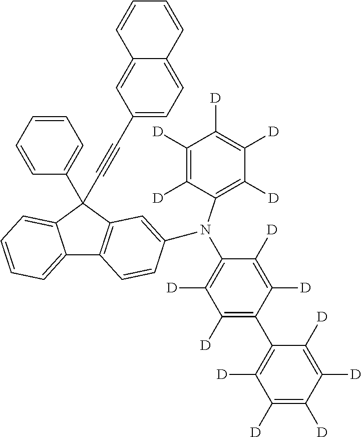 [SMILES: [2H]c1c([2H])c([2H])c(-c2c([2H])c([2H])c(N(c3ccc4c(c3)C(C#Cc3ccc5ccccc5c3)(c3ccccc3)c3ccccc3-4)c3c([2H])c([2H])c([2H])c([2H])c3[2H])c([2H])c2[2H])c([2H])c1[2H]] |

Target compounds of the following Table 2 were synthesized in the same manner as in Preparation Example 1 except that 3-bromo-9H-fluoren-9-one was used instead of 2-bromo-9H-fluoren-9-one, the following Compound A was used instead of bromobenzene, the following Compound B was used instead of ethynylbenzene, and the following Compound C was used instead of di([1,1′-biphenyl]-4-yl)amine.

TABLE 2

| Com- | | | | |
|---|---|---|---|---|
| pound | A | B | C | Target Compound |
| 81 | 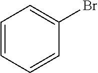 | 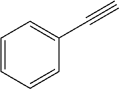 | 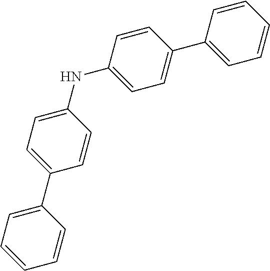 | 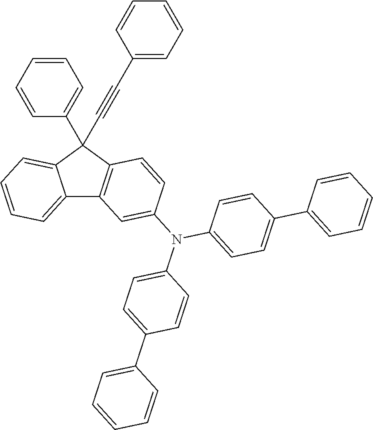 [SMILES: C(#CC1(c2ccccc2)c2ccccc2-c2cc(N(c3ccc(-c4ccccc4)cc3)c3ccc(-c4ccccc4)cc3)ccc21)c1ccccc1] |
| 82 | 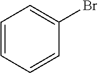 | 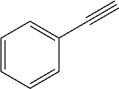 | 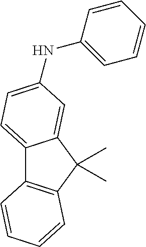 | 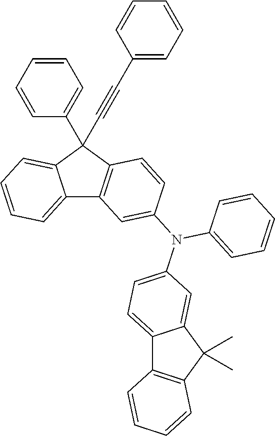 [SMILES: CC1(C)c2ccccc2-c2ccc(N(c3ccccc3)c3ccc4c(c3)-c3ccccc3C4(C#Cc3ccccc3)c3ccccc3)cc21] |
| 83 | 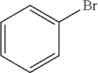 | 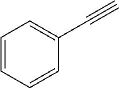 | 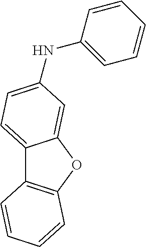 | 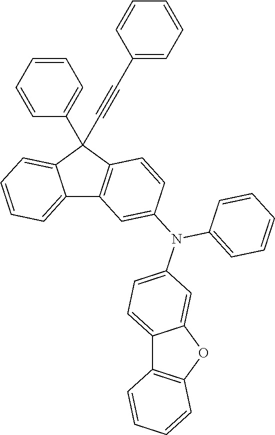 [SMILES: C(#CC1(c2ccccc2)c2ccccc2-c2cc(N(c3ccccc3)c3ccc4c(c3)oc3ccccc34)ccc21)c1ccccc1] |
| 84 | 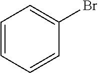 | 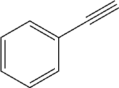 | 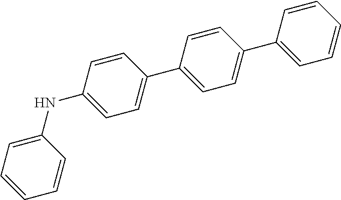 | 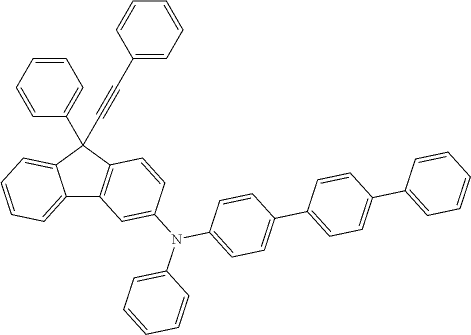 [SMILES: C(#CC1(c2ccccc2)c2ccccc2-c2cc(N(c3ccccc3)c3ccc(-c4ccc(-c5ccccc5)cc4)cc3)ccc21)c1ccccc1] |
| 85 | 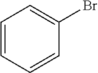 | 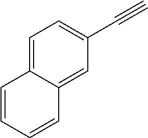 | 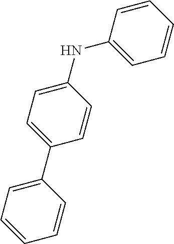 | 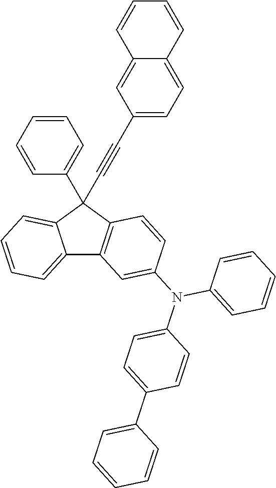 [SMILES: C(#CC1(c2ccccc2)c2ccccc2-c2cc(N(c3ccccc3)c3ccc(-c4ccccc4)cc3)ccc21)c1ccc2ccccc2c1] |
| 86 | 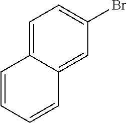 | 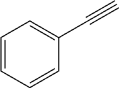 | 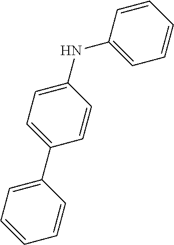 | 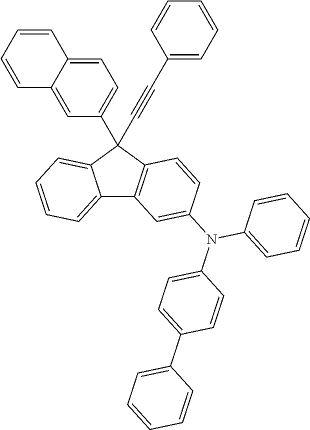 [SMILES: C(#CC1(c2ccc3ccccc3c2)c2ccccc2-c2cc(N(c3ccccc3)c3ccc(-c4ccccc4)cc3)ccc21)c1ccccc1] |
| 87 | 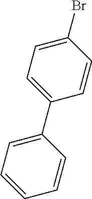 | 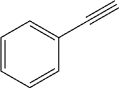 | 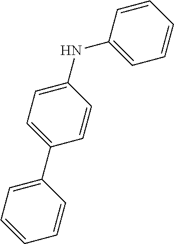 | 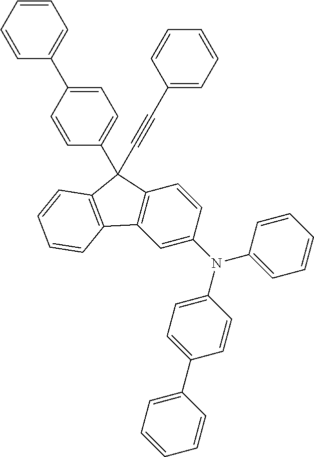 [SMILES: C(#CC1(c2ccc(-c3ccccc3)cc2)c2ccccc2-c2cc(N(c3ccccc3)c3ccc(-c4ccccc4)cc3)ccc21)c1ccccc1] |
| 88 | 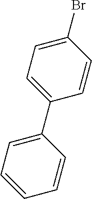 | 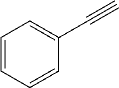 | 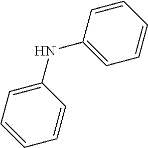 | 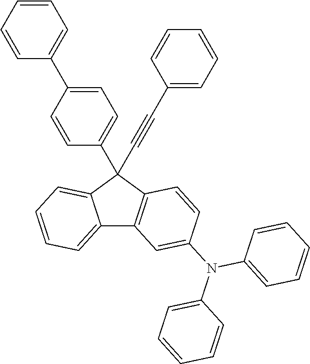 [SMILES: C(#CC1(c2ccc(-c3ccccc3)cc2)c2ccccc2-c2cc(N(c3ccccc3)c3ccccc3)ccc21)c1ccccc1] |
| 89 | 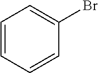 | 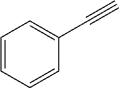 | 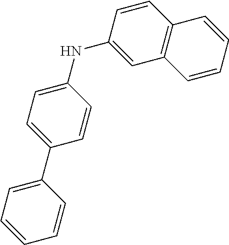 | 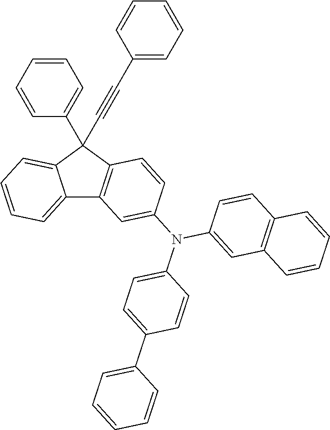 [SMILES: C(#CC1(c2ccccc2)c2ccccc2-c2cc(N(c3ccc(-c4ccccc4)cc3)c3ccc4ccccc4c3)ccc21)c1ccccc1] |
| 90 | 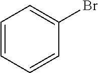 | 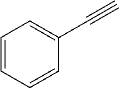 | 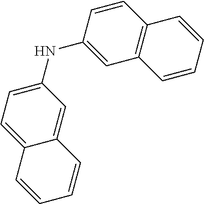 | 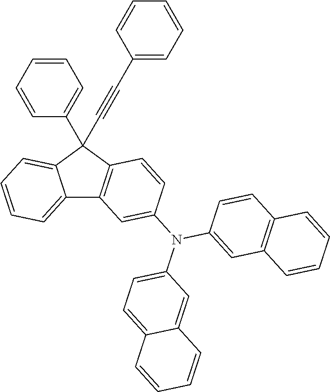 [SMILES: C(#CC1(c2ccccc2)c2ccccc2-c2cc(N(c3ccc4ccccc4c3)c3ccc4ccccc4c3)ccc21)c1ccccc1] |
| 91 | 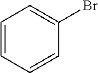 | 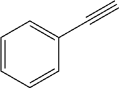 | 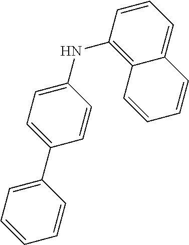 | 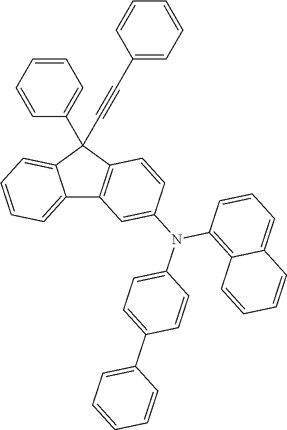 [SMILES: C(#CC1(c2ccccc2)c2ccccc2-c2cc(N(c3ccc(-c4ccccc4)cc3)c3cccc4ccccc34)ccc21)c1ccccc1] |
| 92 | 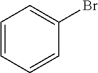 | 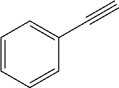 | 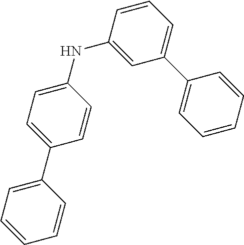 | 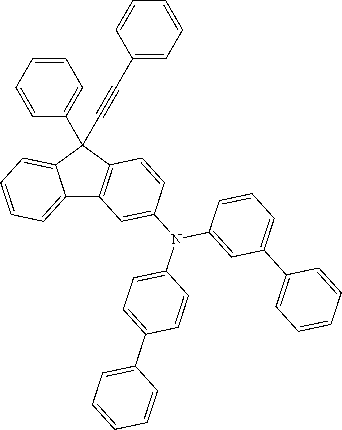 [SMILES: C(#CC1(c2ccccc2)c2ccccc2-c2cc(N(c3ccc(-c4ccccc4)cc3)c3cccc(-c4ccccc4)c3)ccc21)c1ccccc1] |
| 93 | 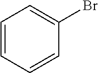 | 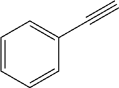 | 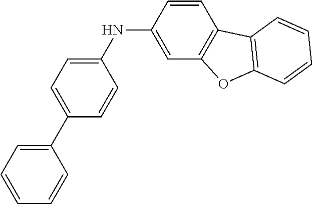 | 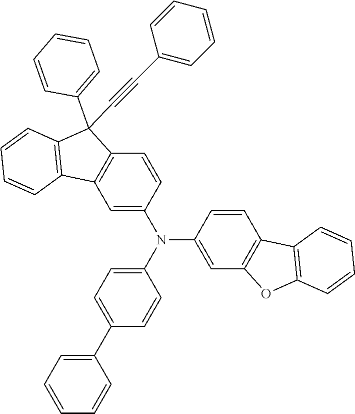 [SMILES: C(#CC1(c2ccccc2)c2ccccc2-c2cc(N(c3ccc(-c4ccccc4)cc3)c3ccc4c(c3)oc3ccccc34)ccc21)c1ccccc1] |
| 94 | 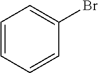 | 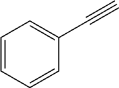 | 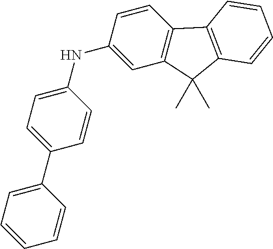 | 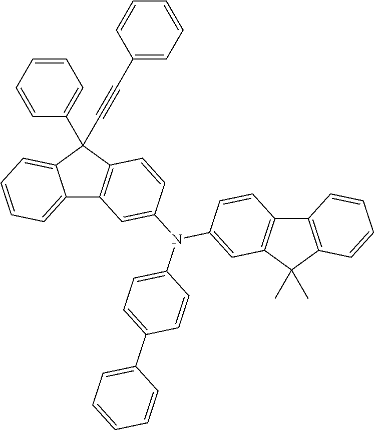 [SMILES: CC1(C)c2ccccc2-c2ccc(N(c3ccc(-c4ccccc4)cc3)c3ccc4c(c3)-c3ccccc3C4(C#Cc3ccccc3)c3ccccc3)cc21] |
| 95 | 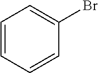 | 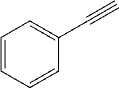 | 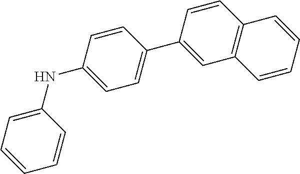 | 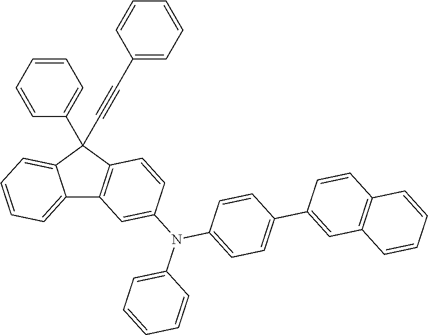 [SMILES: C(#CC1(c2ccccc2)c2ccccc2-c2cc(N(c3ccccc3)c3ccc(-c4ccc5ccccc5c4)cc3)ccc21)c1ccccc1] |
| 96 | 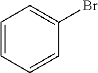 | 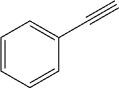 | 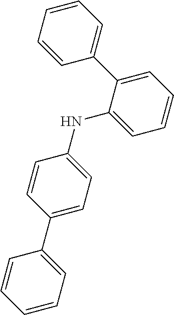 | 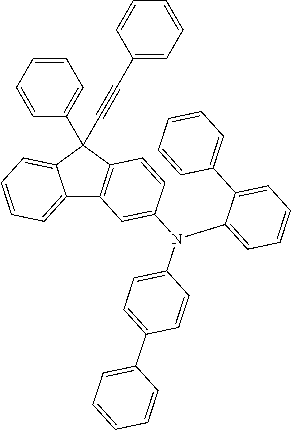 [SMILES: C(#CC1(c2ccccc2)c2ccccc2-c2cc(N(c3ccc(-c4ccccc4)cc3)c3ccccc3-c3ccccc3)ccc21)c1ccccc1] |
| 97 | 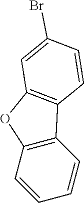 | 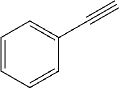 | 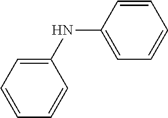 | 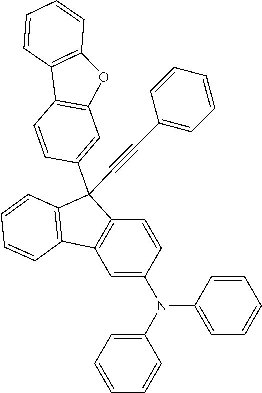 [SMILES: C(#CC1(c2ccc3c(c2)oc2ccccc23)c2ccccc2-c2cc(N(c3ccccc3)c3ccccc3)ccc21)c1ccccc1] |
| 98 | 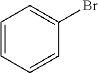 | 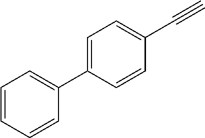 | 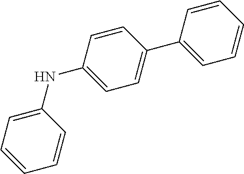 | 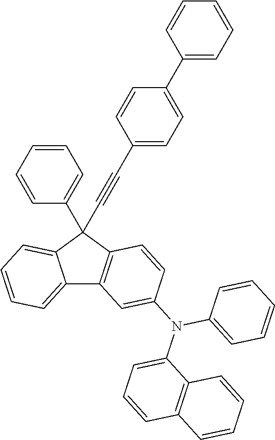 [SMILES: C(#CC1(c2ccccc2)c2ccccc2-c2cc(N(c3ccccc3)c3cccc4ccccc34)ccc21)c1ccc(-c2ccccc2)cc1] |
| 99 | 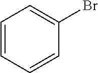 | 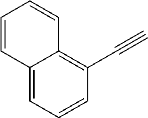 | 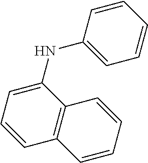 | 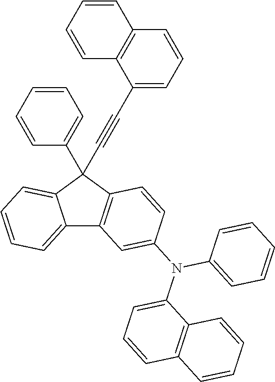 [SMILES: C(#CC1(c2ccccc2)c2ccccc2-c2cc(N(c3ccccc3)c3cccc4ccccc34)ccc21)c1cccc2ccccc12] |
| 100 | 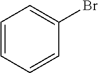 | 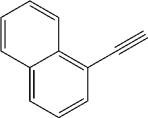 | 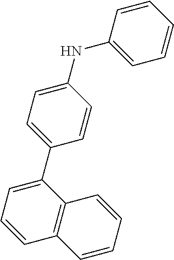 | 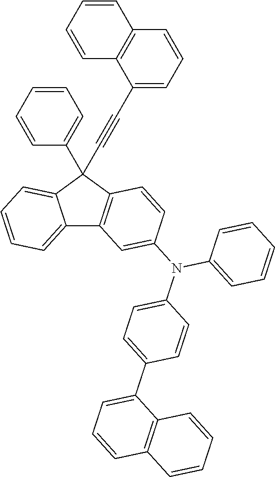 [SMILES: C(#CC1(c2ccccc2)c2ccccc2-c2cc(N(c3ccccc3)c3ccc(-c4cccc5ccccc45)cc3)ccc21)c1cccc2ccccc12] |
| 101 | 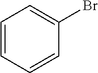 | 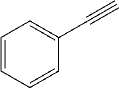 | 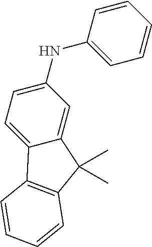 | 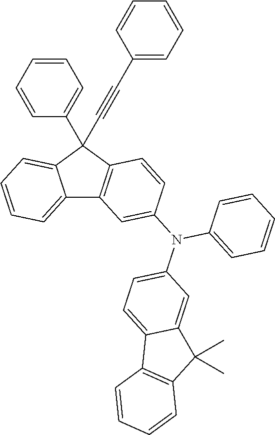 [SMILES: CC1(C)c2ccccc2-c2ccc(N(c3ccccc3)c3ccc4c(c3)-c3ccccc3C4(C#Cc3ccccc3)c3ccccc3)cc21] |
| 102 | 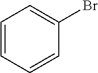 | 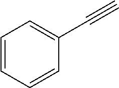 | 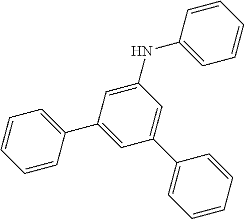 | 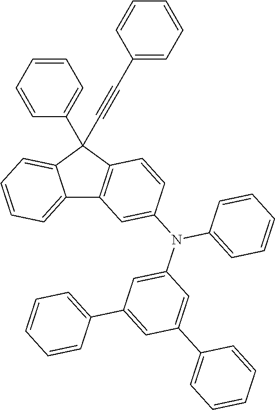 [SMILES: C(#CC1(c2ccccc2)c2ccccc2-c2cc(N(c3ccccc3)c3cc(-c4ccccc4)cc(-c4ccccc4)c3)ccc21)c1ccccc1] |
| 103 | 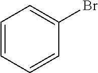 | 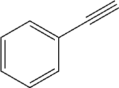 | 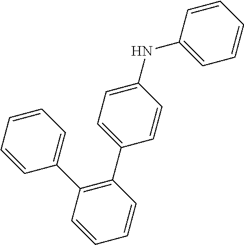 | 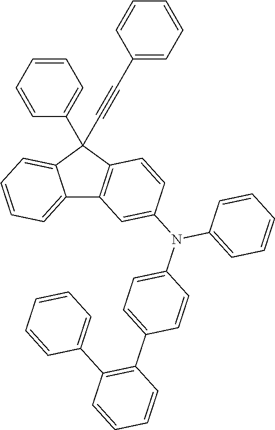 [SMILES: C(#CC1(c2ccccc2)c2ccccc2-c2cc(N(c3ccccc3)c3ccc(-c4ccccc4-c4ccccc4)cc3)ccc21)c1ccccc1] |
| 104 | 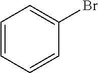 | 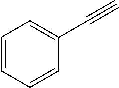 | 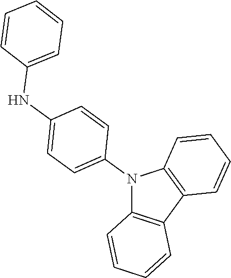 | 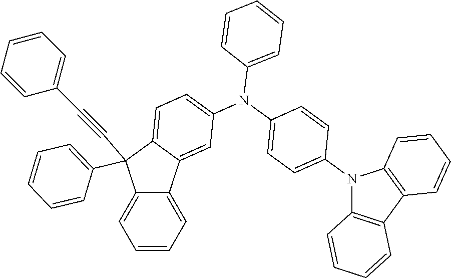 [SMILES: C(#CC1(c2ccccc2)c2ccccc2-c2cc(N(c3ccccc3)c3ccc(-n4c5ccccc5c5ccccc54)cc3)ccc21)c1ccccc1] |
| 105 | 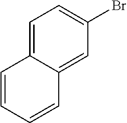 | 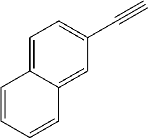 | 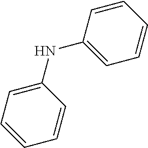 | 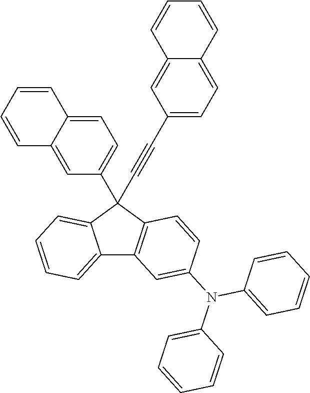 [SMILES: C(#CC1(c2ccc3ccccc3c2)c2ccccc2-c2cc(N(c3ccccc3)c3ccccc3)ccc21)c1ccc2ccccc2c1] |
| 106 | 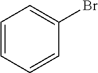 | 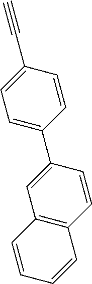 | 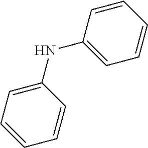 | 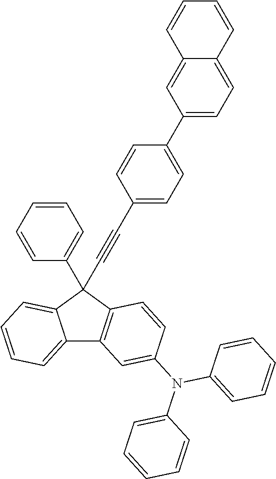 [SMILES: C(#CC1(c2ccccc2)c2ccccc2-c2cc(N(c3ccccc3)c3ccccc3)ccc21)c1ccc(-c2ccc3ccccc3c2)cc1] |
| 107 | 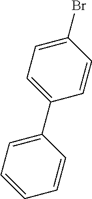 | 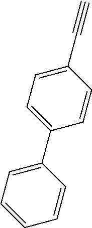 | 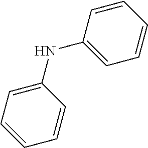 | 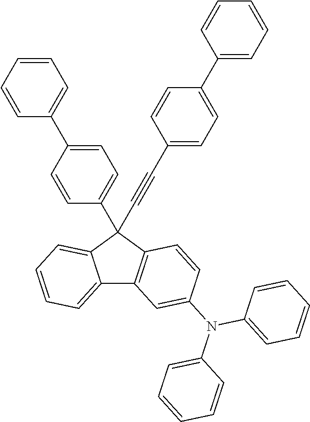 [SMILES: C(#CC1(c2ccc(-c3ccccc3)cc2)c2ccccc2-c2cc(N(c3ccccc3)c3ccccc3)ccc21)c1ccc(-c2ccccc2)cc1] |
| 108 | 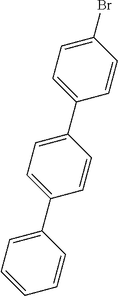 | 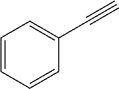 | 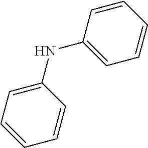 | 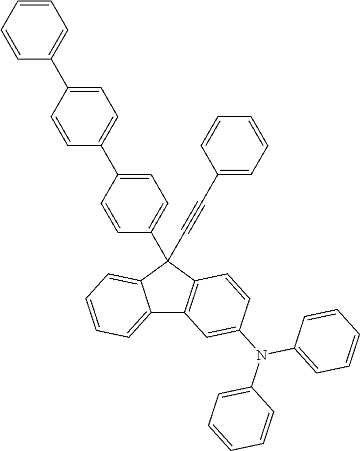 [SMILES: C(#CC1(c2ccc(-c3ccc(-c4ccccc4)cc3)cc2)c2ccccc2-c2cc(N(c3ccccc3)c3ccccc3)ccc21)c1ccccc1] |
| 109 | 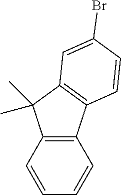 | 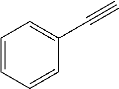 | 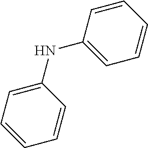 | 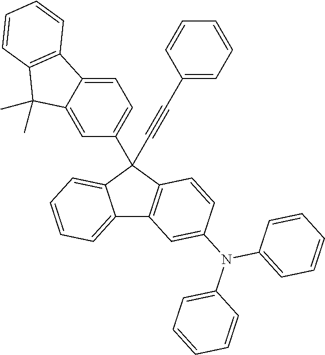 [SMILES: CC1(C)c2ccccc2-c2ccc(C3(C#Cc4ccccc4)c4ccccc4-c4cc(N(c5ccccc5)c5ccccc5)ccc43)cc21] |
| 110 | 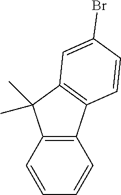 | 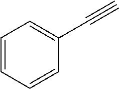 | 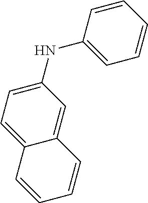 | 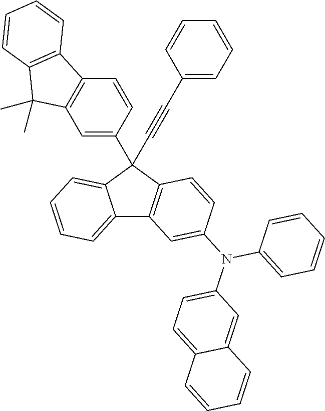 [SMILES: CC1(C)c2ccccc2-c2ccc(C3(C#Cc4ccccc4)c4ccccc4-c4cc(N(c5ccccc5)c5ccc6ccccc6c5)ccc43)cc21] |
| 111 | 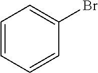 | 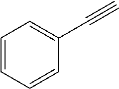 | 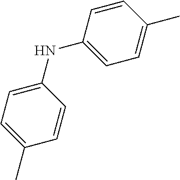 | 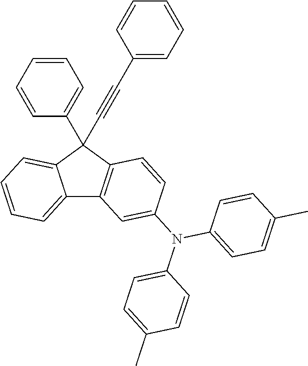 [SMILES: Cc1ccc(N(c2ccc(C)cc2)c2ccc3c(c2)-c2ccccc2C3(C#Cc2ccccc2)c2ccccc2)cc1] |
| 112 | 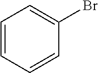 | 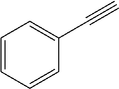 | 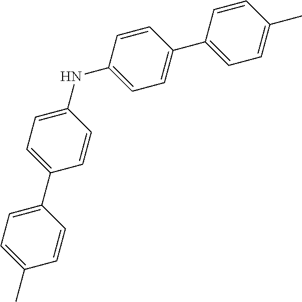 | 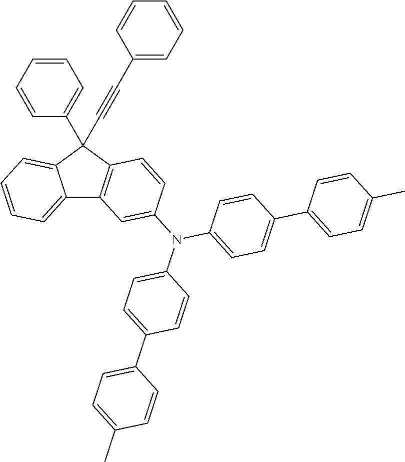 [SMILES: Cc1ccc(-c2ccc(N(c3ccc(-c4ccc(C)cc4)cc3)c3ccc4c(c3)-c3ccccc3C4(C#Cc3ccccc3)c3ccccc3)cc2)cc1] |
| 113 | 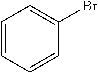 | 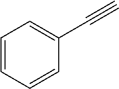 | 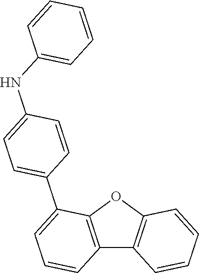 | 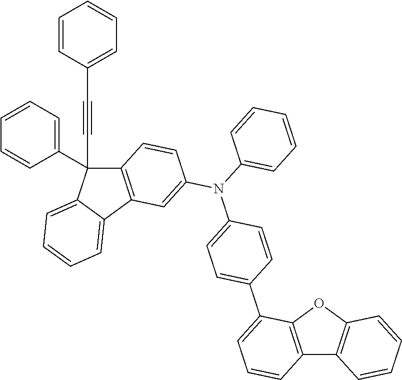 [SMILES: C(#CC1(c2ccccc2)c2ccccc2-c2cc(N(c3ccccc3)c3ccc(-c4cccc5c4oc4ccccc45)cc3)ccc21)c1ccccc1] |
| 114 | 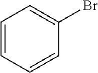 | 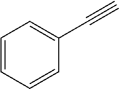 | 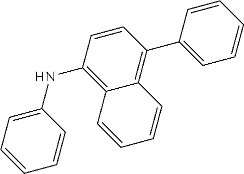 | 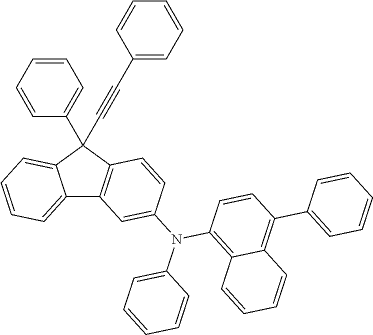 [SMILES: C(#CC1(c2ccccc2)c2ccccc2-c2cc(N(c3ccccc3)c3ccc(-c4ccccc4)c4ccccc34)ccc21)c1ccccc1] |
| 115 | 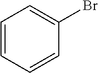 | 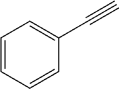 | 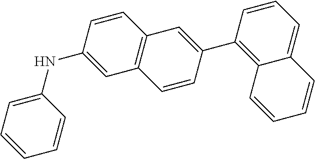 | 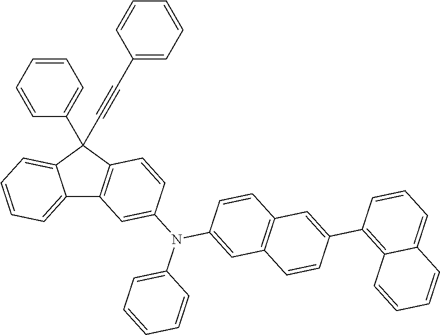 [SMILES: C(#CC1(c2ccccc2)c2ccccc2-c2cc(N(c3ccccc3)c3ccc4cc(-c5cccc6ccccc56)ccc4c3)ccc21)c1ccccc1] |
| 116 | 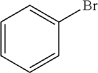 | 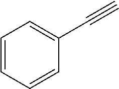 | 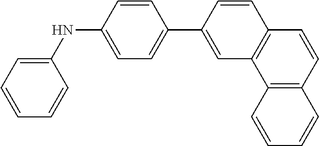 | 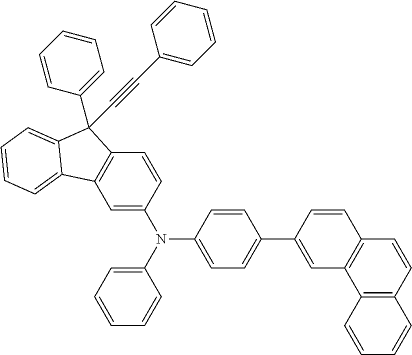 [SMILES: C(#CC1(c2ccccc2)c2ccccc2-c2cc(N(c3ccccc3)c3ccc(-c4ccc5ccc6ccccc6c5c4)cc3)ccc21)c1ccccc1] |
| 117 | 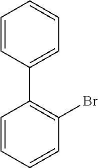 | 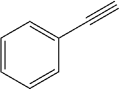 | 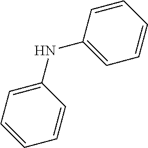 | 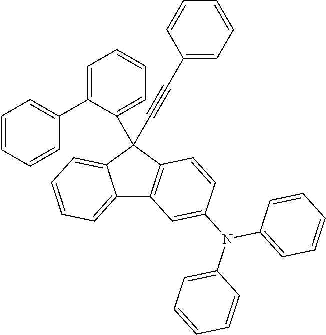 [SMILES: C(#CC1(c2ccccc2-c2ccccc2)c2ccccc2-c2cc(N(c3ccccc3)c3ccccc3)ccc21)c1ccccc1] |
| 118 | 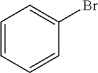 | 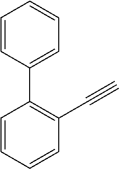 | 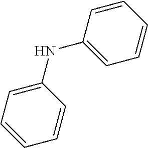 | 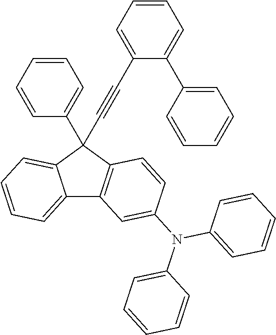 [SMILES: C(#CC1(c2ccccc2)c2ccccc2-c2cc(N(c3ccccc3)c3ccccc3)ccc21)c1ccccc1-c1ccccc1] |
| 119 | 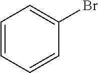 | 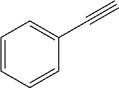 | 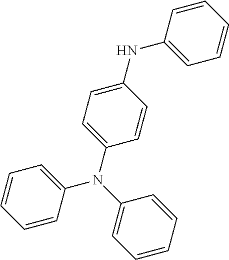 | 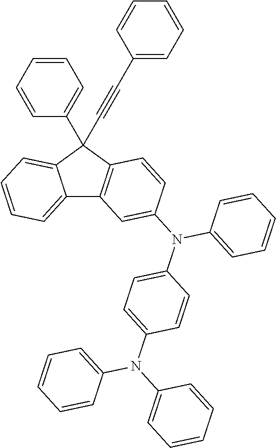 [SMILES: C(#CC1(c2ccccc2)c2ccccc2-c2cc(N(c3ccccc3)c3ccc(N(c4ccccc4)c4ccccc4)cc3)ccc21)c1ccccc1] |
| 120 | 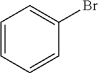 | 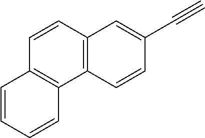 | 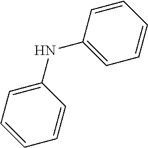 | 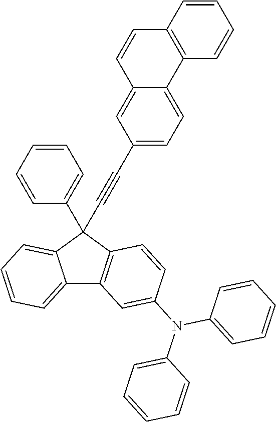 [SMILES: C(#CC1(c2ccccc2)c2ccccc2-c2cc(N(c3ccccc3)c3ccccc3)ccc21)c1ccc2c(ccc3ccccc32)c1] |
| 161 | 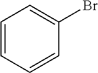 | 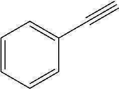 | 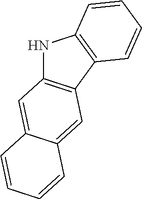 | 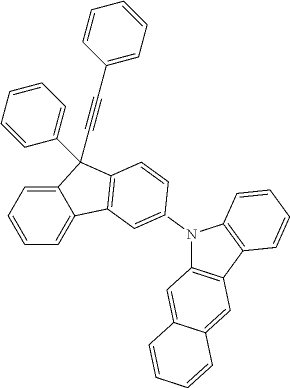 |
| 162 | 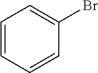 | 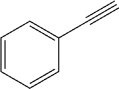 | 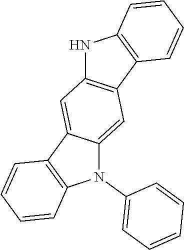 | 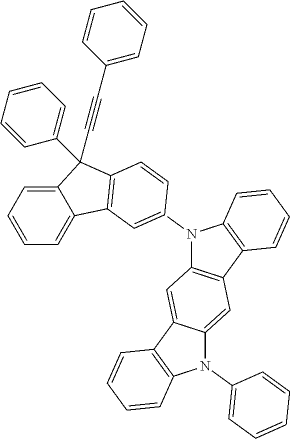 |
| 163 | 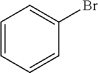 | 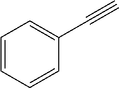 | 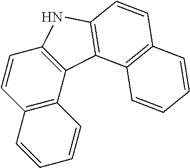 | 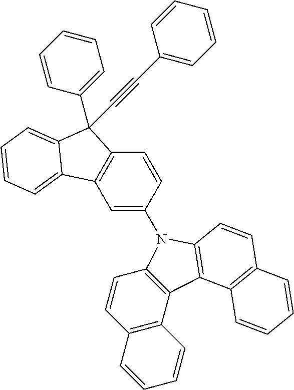 [SMILES: C(#CC1(c2ccccc2)c2ccccc2-c2cc(-n3c4ccc5ccccc5c4c4c5ccccc5ccc43)ccc21)c1ccccc1] |
| 164 | 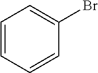 | 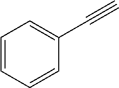 | 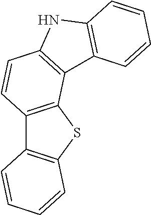 | 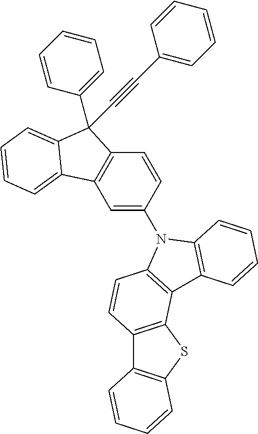 [SMILES: C(#CC1(c2ccccc2)c2ccccc2-c2cc(-n3c4ccccc4c4c5sc6ccccc6c5ccc43)ccc21)c1ccccc1] |

Target compounds of the following Table 3 were synthesized in the same manner as in Preparation Example 1 except that 4-bromo-9H-fluoren-9-one was used instead of 2-bromo-9H-fluoren-9-one, the following Compound A was used instead of bromobenzene, the following Compound B was used instead of ethynylbenzene, and the following Compound C was used instead of di(([1,1′-biphenyl]-4-yl)amine.

TABLE 3

| Compound | A | B | C |
|---|---|---|---|
| 121 | 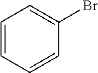 | 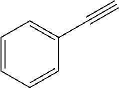 | 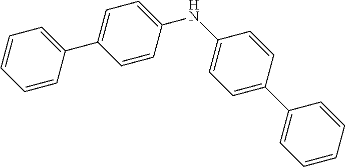 |
| 122 | 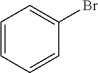 | 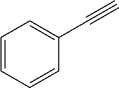 | 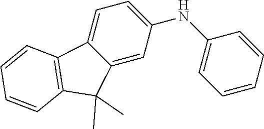 |
| 123 | 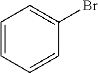 | 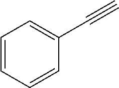 | 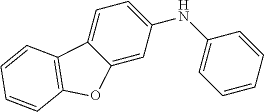 |
| 124 | 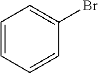 | 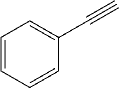 | 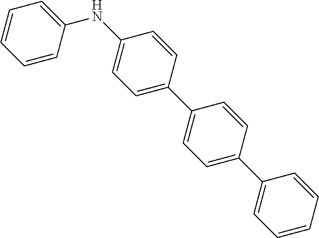 |
| 125 | 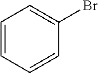 | 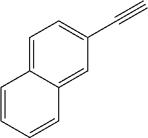 | 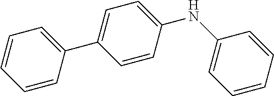 |
| 126 | 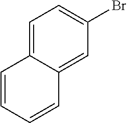 | 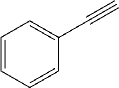 | 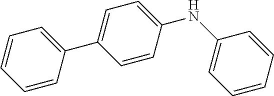 |
| 127 | 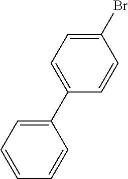 | 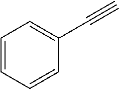 | 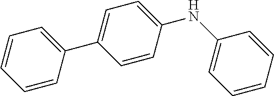 |
| 128 | 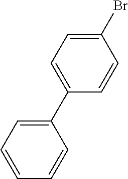 | 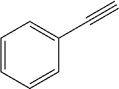 | 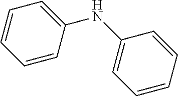 |
| 129 | 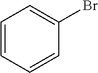 | 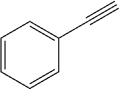 | 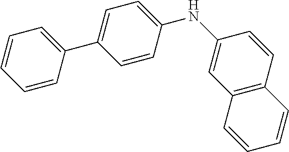 |
| 130 | 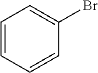 | 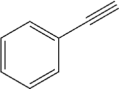 | 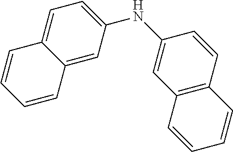 |
| 131 | 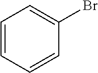 | 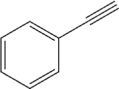 | 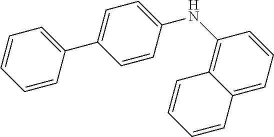 |
| 132 | 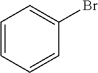 | 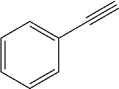 | 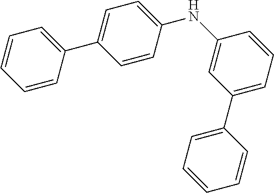 |
| 133 | 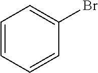 | 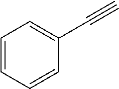 | 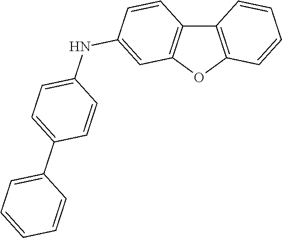 |
| 134 | 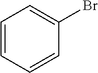 | 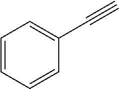 | 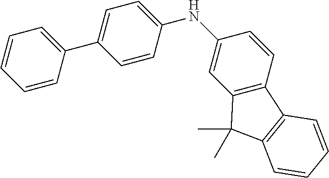 |
| 135 | 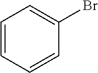 | 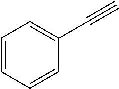 | 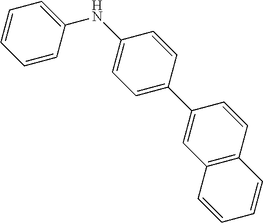 |
| 136 | 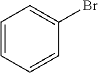 | 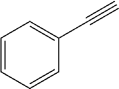 | 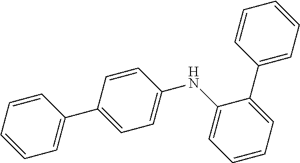 |
| 137 | 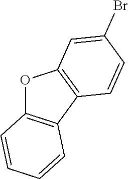 | 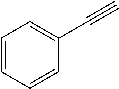 | 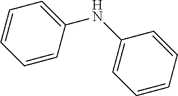 |
| 138 | 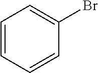 | 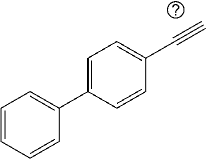 | 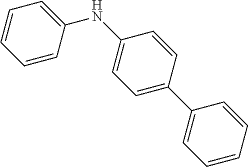 |
| 139 | 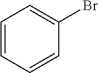 | 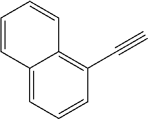 | 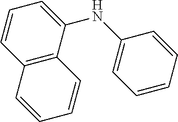 |
| 140 | 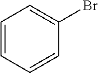 | 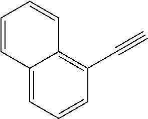 | 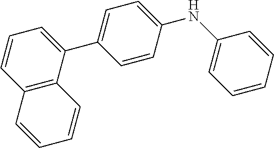 |
| 141 | 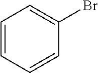 | 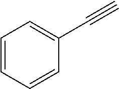 | 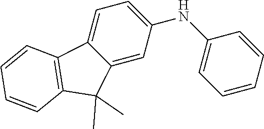 |
| 142 | 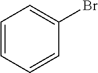 | 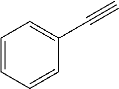 | 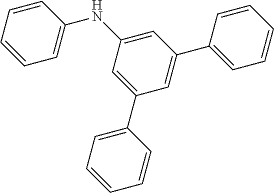 |
| 143 | 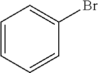 | 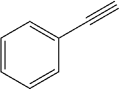 | 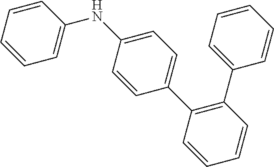 |
| 144 | 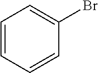 | 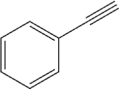 | 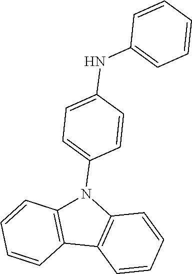 |
| 145 | 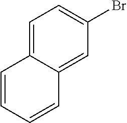 | 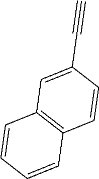 | 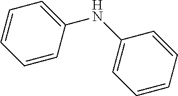 |
| 146 | 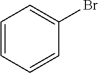 | 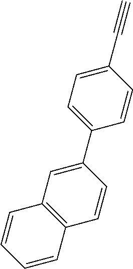 | 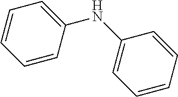 |
| 147 | 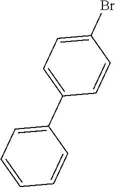 | 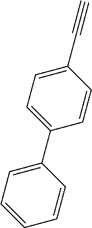 | 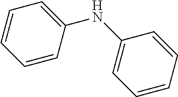 |
| 148 | 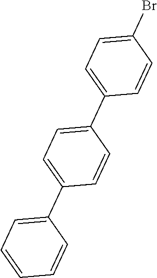 | 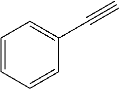 | 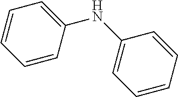 |
| 149 | 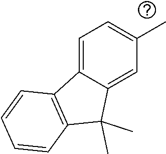 | 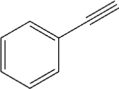 | 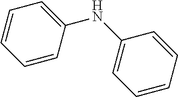 |
| 150 | 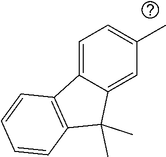 | 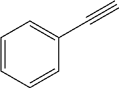 | 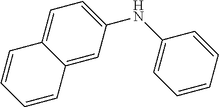 |
| 151 | 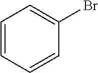 | 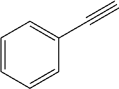 | 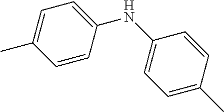 |
| 152 | 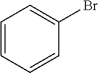 | 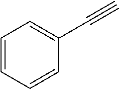 | 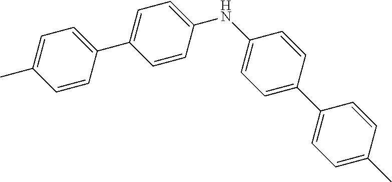 |
| 153 | 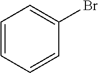 | 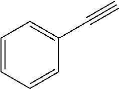 | 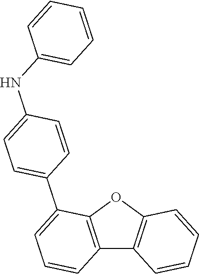 |
| 154 | 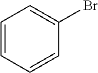 | 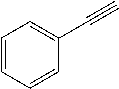 | 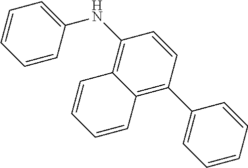 |
| 155 | 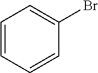 | 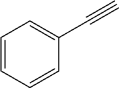 | 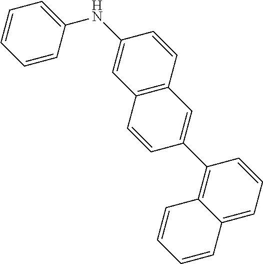 |
| 156 | 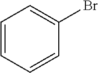 | 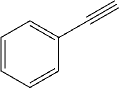 | 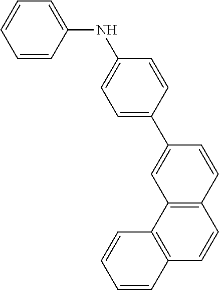 |
| 157 | 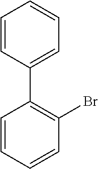 | 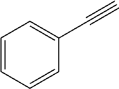 | 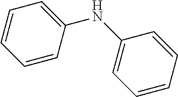 |
| 158 | 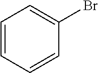 | 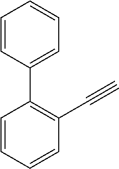 | 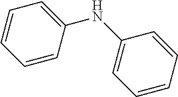 |
| 159 | 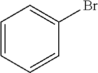 | 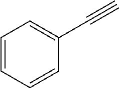 | 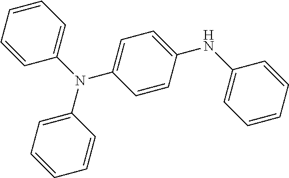 |
| 160 | 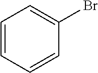 | 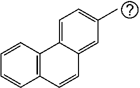 | 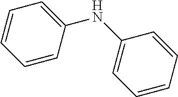 |
| 165 | 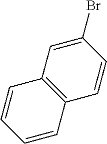 | 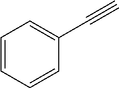 | 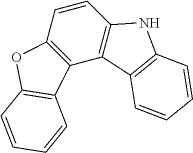 |
| 166 | 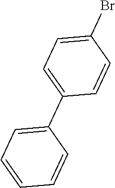 | 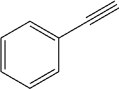 | 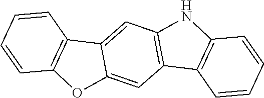 |
| 167 | 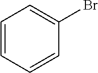 | 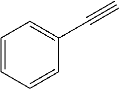 | 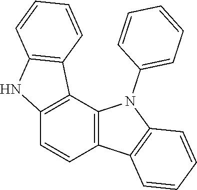 |
| 168 | 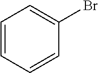 | 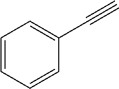 | 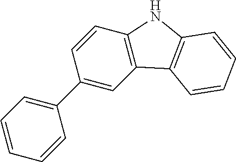 |

| Compound | Target Compound |
|---|---|
| 121 | 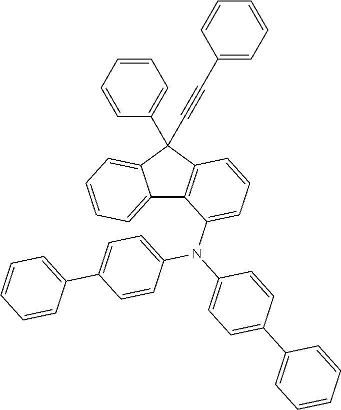 [SMILES: C(#CC1(c2ccccc2)c2ccccc2-c2c(N(c3ccc(-c4ccccc4)cc3)c3ccc(-c4ccccc4)cc3)cccc21)c1ccccc1] |
| 122 | 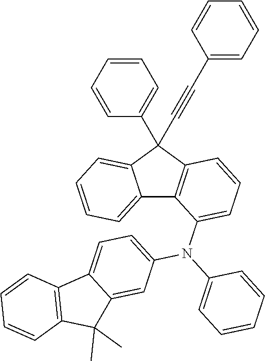 [SMILES: CC1(C)c2ccccc2-c2ccc(N(c3ccccc3)c3cccc4c3-c3ccccc3C4(C#Cc3ccccc3)c3ccccc3)cc21] |
| 123 | 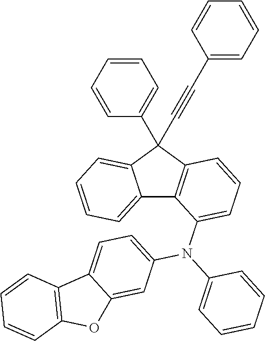 [SMILES: C(#CC1(c2ccccc2)c2ccccc2-c2c(N(c3ccccc3)c3ccc4c(c3)oc3ccccc34)cccc21)c1ccccc1] |
| 124 | 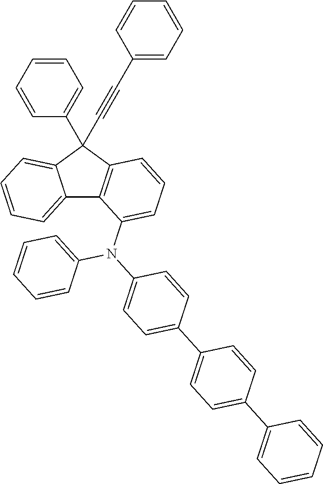 [SMILES: C(#CC1(c2ccccc2)c2ccccc2-c2c(N(c3ccccc3)c3ccc(-c4ccc(-c5ccccc5)cc4)cc3)cccc21)c1ccccc1] |
| 125 | 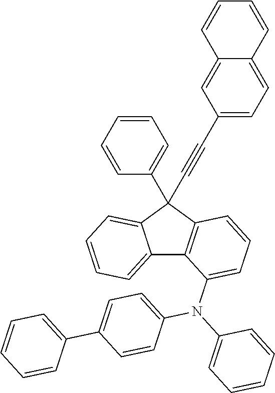 [SMILES: C(#CC1(c2ccccc2)c2ccccc2-c2c(N(c3ccccc3)c3ccc(-c4ccccc4)cc3)cccc21)c1ccc2ccccc2c1] |
| 126 | 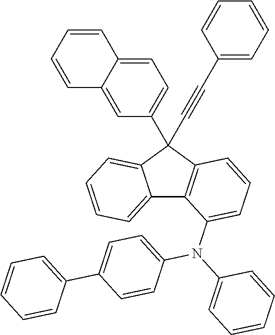 [SMILES: C(#CC1(c2ccc3ccccc3c2)c2ccccc2-c2c(N(c3ccccc3)c3ccc(-c4ccccc4)cc3)cccc21)c1ccccc1] |
| 127 | 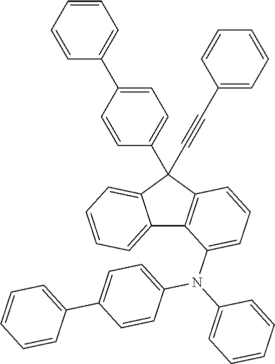 [SMILES: C(#CC1(c2ccc(-c3ccccc3)cc2)c2ccccc2-c2c(N(c3ccccc3)c3ccc(-c4ccccc4)cc3)cccc21)c1ccccc1] |
| 128 | 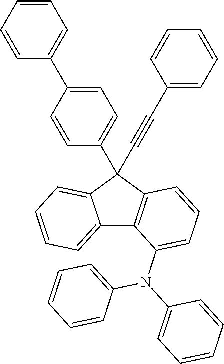 [SMILES: C(#CC1(c2ccc(-c3ccccc3)cc2)c2ccccc2-c2c(N(c3ccccc3)c3ccccc3)cccc21)c1ccccc1] |
| 129 | 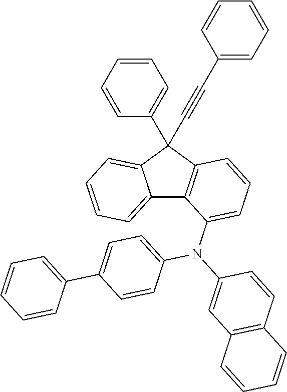 [SMILES: C(#CC1(c2ccccc2)c2ccccc2-c2c(N(c3ccc(-c4ccccc4)cc3)c3ccc4ccccc4c3)cccc21)c1ccccc1] |
| 130 | 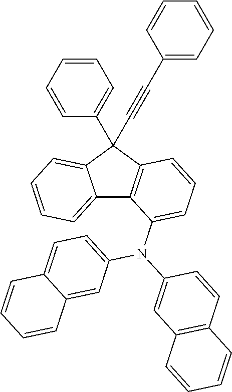 [SMILES: C(#CC1(c2ccccc2)c2ccccc2-c2c(N(c3ccc4ccccc4c3)c3ccc4ccccc4c3)cccc21)c1ccccc1] |
| 131 | 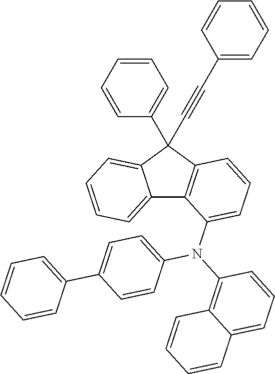 [SMILES: C(#CC1(c2ccccc2)c2ccccc2-c2c(N(c3ccc(-c4ccccc4)cc3)c3cccc4ccccc34)cccc21)c1ccccc1] |
| 132 | 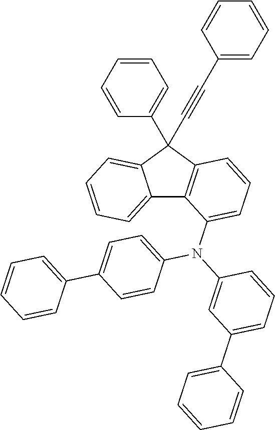 [SMILES: C(#CC1(c2ccccc2)c2ccccc2-c2c(N(c3ccc(-c4ccccc4)cc3)c3cccc(-c4ccccc4)c3)cccc21)c1ccccc1] |
| 133 | 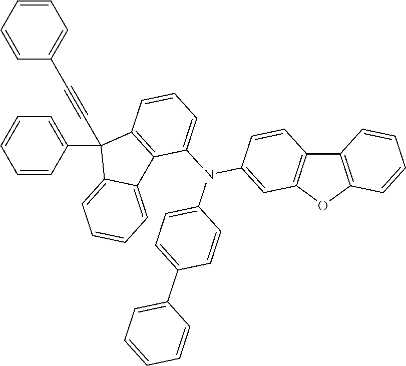 [SMILES: C(#CC1(c2ccccc2)c2ccccc2-c2c(N(c3ccc(-c4ccccc4)cc3)c3ccc4c(c3)oc3ccccc34)cccc21)c1ccccc1] |
| 134 | 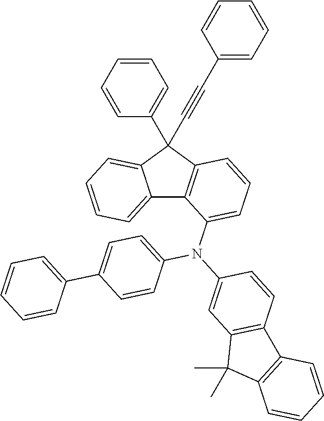 [SMILES: CC1(C)c2ccccc2-c2ccc(N(c3ccc(-c4ccccc4)cc3)c3cccc4c3-c3ccccc3C4(C#Cc3ccccc3)c3ccccc3)cc21] |
| 135 | 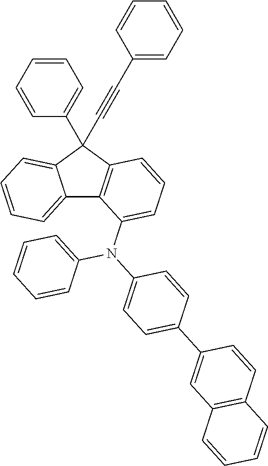 [SMILES: C(#CC1(c2ccccc2)c2ccccc2-c2c(N(c3ccccc3)c3ccc(-c4ccc5ccccc5c4)cc3)cccc21)c1ccccc1] |
| 136 | 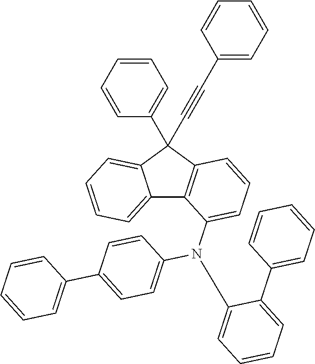 [SMILES: C(#CC1(c2ccccc2)c2ccccc2-c2c(N(c3ccc(-c4ccccc4)cc3)c3ccccc3-c3ccccc3)cccc21)c1ccccc1] |
| 137 | 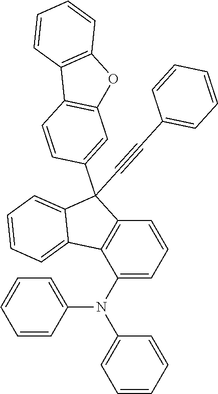 [SMILES: C(#CC1(c2ccc3c(c2)oc2ccccc23)c2ccccc2-c2c(N(c3ccccc3)c3ccccc3)cccc21)c1ccccc1] |
| 138 | 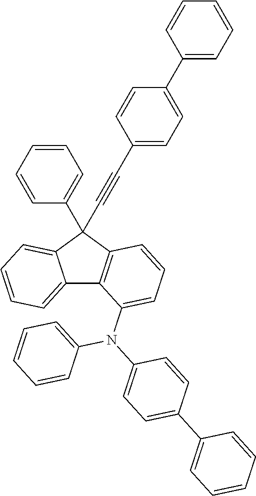 [SMILES: C(#CC1(c2ccccc2)c2ccccc2-c2c(N(c3ccccc3)c3ccc(-c4ccccc4)cc3)cccc21)c1ccc(-c2ccccc2)cc1] |
| 139 | 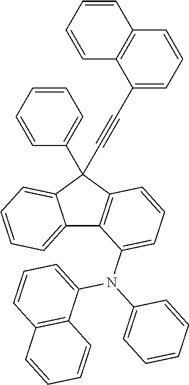 [SMILES: C(#CC1(c2ccccc2)c2ccccc2-c2c(N(c3ccccc3)c3cccc4ccccc34)cccc21)c1cccc2ccccc12] |
| 140 | 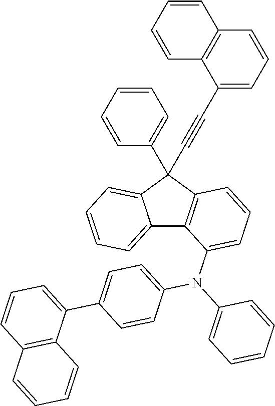 [SMILES: C(#CC1(c2ccccc2)c2ccccc2-c2c(N(c3ccccc3)c3ccc(-c4cccc5ccccc45)cc3)cccc21)c1cccc2ccccc12] |
| 141 | 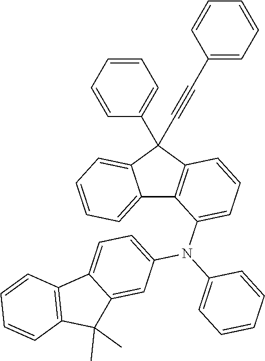 [SMILES: CC1(C)c2ccccc2-c2ccc(N(c3ccccc3)c3cccc4c3-c3ccccc3C4(C#Cc3ccccc3)c3ccccc3)cc21] |
| 142 | 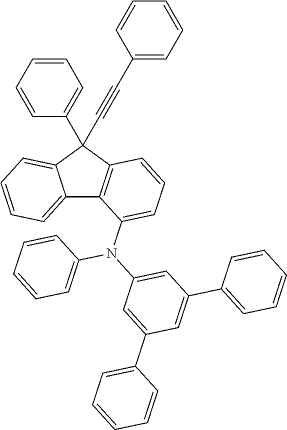 [SMILES: C(#CC1(c2ccccc2)c2ccccc2-c2c(N(c3ccccc3)c3cc(-c4ccccc4)cc(-c4ccccc4)c3)cccc21)c1ccccc1] |
| 143 | 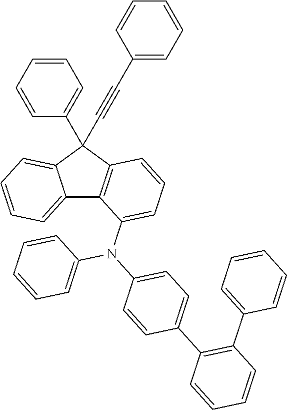 [SMILES: C(#CC1(c2ccccc2)c2ccccc2-c2c(N(c3ccccc3)c3ccc(-c4ccccc4-c4ccccc4)cc3)cccc21)c1ccccc1] |
| 144 | 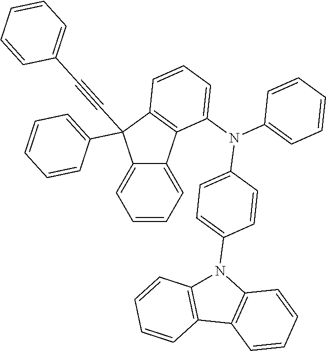 [SMILES: C(#CC1(c2ccccc2)c2ccccc2-c2c(N(c3ccccc3)c3ccc(-n4c5ccccc5c5ccccc54)cc3)cccc21)c1ccccc1] |
| 145 | 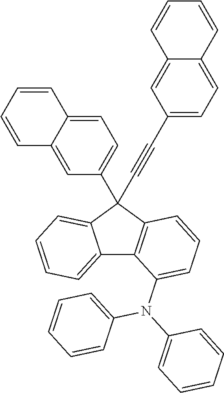 [SMILES: C(#CC1(c2ccc3ccccc3c2)c2ccccc2-c2c(N(c3ccccc3)c3ccccc3)cccc21)c1ccc2ccccc2c1] |
| 146 | 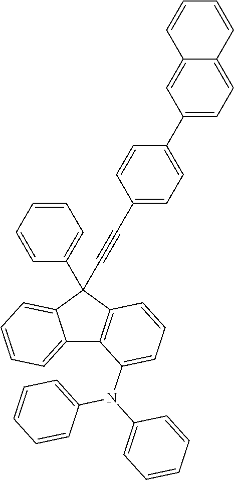 [SMILES: C(#CC1(c2ccccc2)c2ccccc2-c2c(N(c3ccccc3)c3ccccc3)cccc21)c1ccc(-c2ccc3ccccc3c2)cc1] |
| 147 | 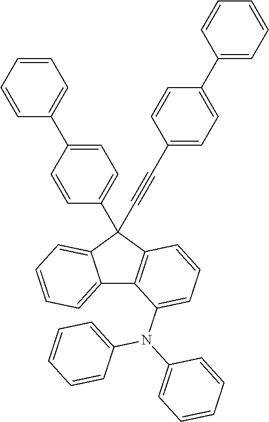 [SMILES: C(#CC1(c2ccc(-c3ccccc3)cc2)c2ccccc2-c2c(N(c3ccccc3)c3ccccc3)cccc21)c1ccc(-c2ccccc2)cc1] |
| 148 | 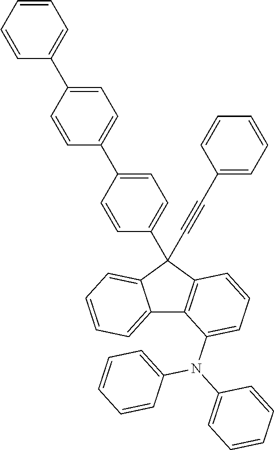 [SMILES: C(#CC1(c2ccc(-c3ccc(-c4ccccc4)cc3)cc2)c2ccccc2-c2c(N(c3ccccc3)c3ccccc3)cccc21)c1ccccc1] |
| 149 | 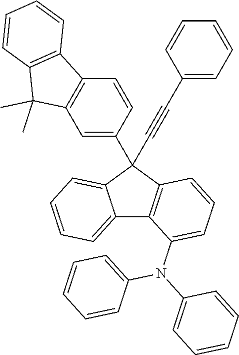 [SMILES: CC1(C)c2ccccc2-c2ccc(C3(C#Cc4ccccc4)c4ccccc4-c4c(N(c5ccccc5)c5ccccc5)cccc43)cc21] |
| 150 | 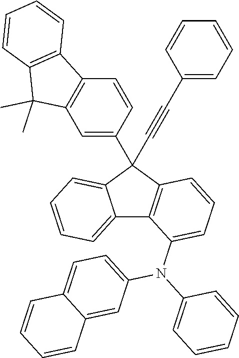 [SMILES: CC1(C)c2ccccc2-c2ccc(C3(C#Cc4ccccc4)c4ccccc4-c4c(N(c5ccccc5)c5ccc6ccccc6c5)cccc43)cc21] |
| 151 | 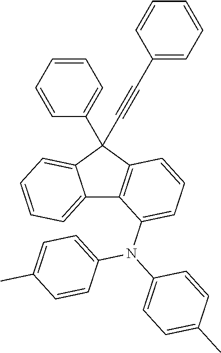 |
| 152 | 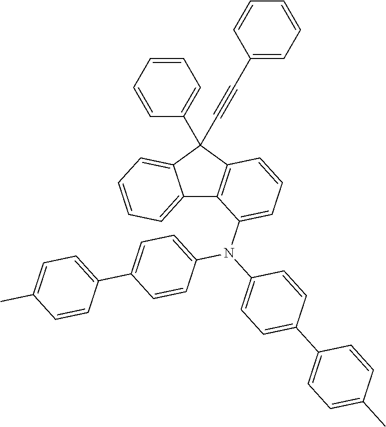 |
| 153 | 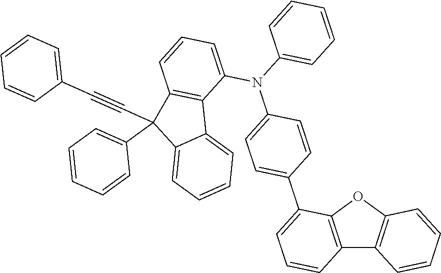 [SMILES: C(#CC1(c2ccccc2)c2ccccc2-c2c(N(c3ccccc3)c3ccc(-c4cccc5c4oc4ccccc45)cc3)cccc21)c1ccccc1] |
| 154 | 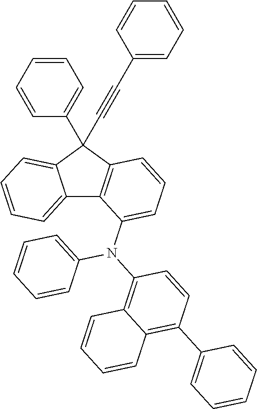 [SMILES: C(#CC1(c2ccccc2)c2ccccc2-c2c(N(c3ccccc3)c3ccc(-c4ccccc4)c4ccccc34)cccc21)c1ccccc1] |
| 155 | 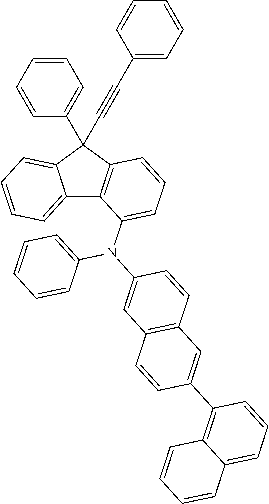 [SMILES: C(#CC1(c2ccccc2)c2ccccc2-c2c(N(c3ccccc3)c3ccc4cc(-c5cccc6ccccc56)ccc4c3)cccc21)c1ccccc1] |
| 156 | 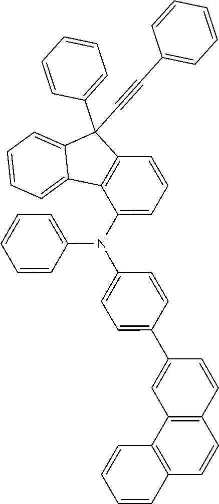 [SMILES: C(#CC1(c2ccccc2)c2ccccc2-c2c(N(c3ccccc3)c3ccc(-c4ccc5ccc6ccccc6c5c4)cc3)cccc21)c1ccccc1] |
| 157 | 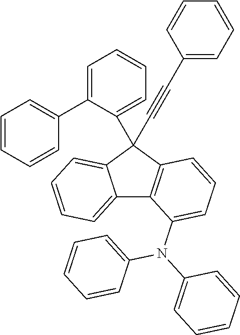 [SMILES: C(#CC1(c2ccccc2-c2ccccc2)c2ccccc2-c2c(N(c3ccccc3)c3ccccc3)cccc21)c1ccccc1] |
| 158 | 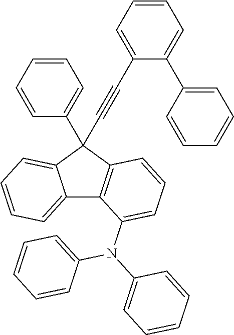 [SMILES: C(#CC1(c2ccccc2)c2ccccc2-c2c(N(c3ccccc3)c3ccccc3)cccc21)c1ccccc1-c1ccccc1] |
| 159 | 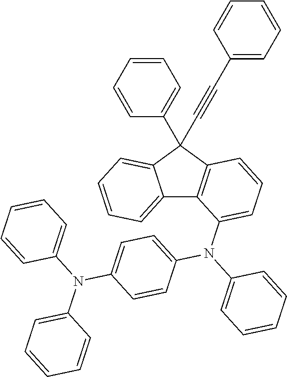 [SMILES: C(#CC1(c2ccccc2)c2ccccc2-c2c(N(c3ccccc3)c3ccc(N(c4ccccc4)c4ccccc4)cc3)cccc21)c1ccccc1] |
| 160 | 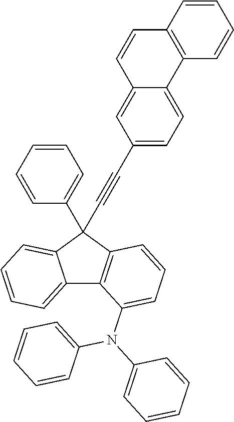 [SMILES: C(#CC1(c2ccccc2)c2ccccc2-c2c(N(c3ccccc3)c3ccccc3)cccc21)c1ccc2c(ccc3ccccc32)c1] |
| 165 | 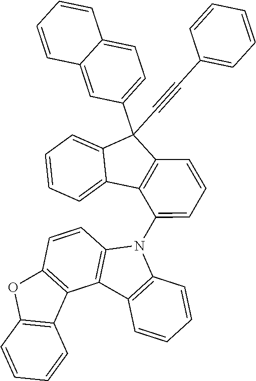 [SMILES: C(#CC1(c2ccc3ccccc3c2)c2ccccc2-c2c(-n3c4ccccc4c4c5c(ccc43)oc3ccccc35)cccc21)c1ccccc1] |
| 166 | 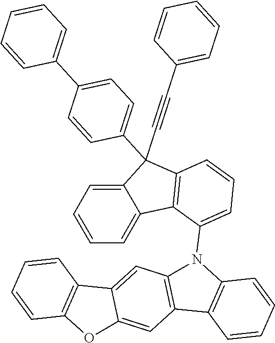 [SMILES: C(#CC1(c2ccc(-c3ccccc3)cc2)c2ccccc2-c2c(-n3c4ccccc4c4cc5oc6ccccc6c5cc43)cccc21)c1ccccc1] |
| 167 | 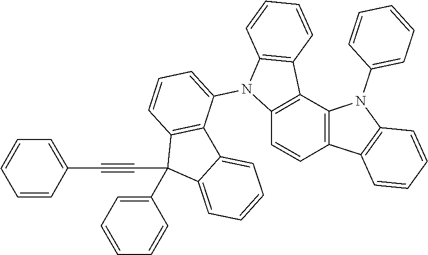 [SMILES: C(#CC1(c2ccccc2)c2ccccc2-c2c(-n3c4ccccc4c4c3ccc3c5ccccc5n(-c5ccccc5)c34)cccc21)c1ccccc1] |
| 168 | 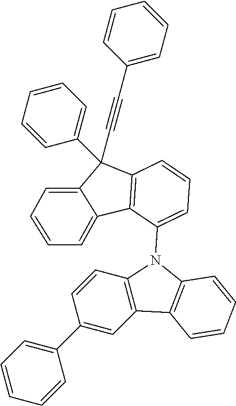 [SMILES: C(#CC1(c2ccccc2)c2ccccc2-c2c(-n3c4ccccc4c4cc(-c5ccccc5)ccc43)cccc21)c1ccccc1] |

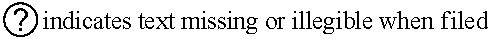

Target compounds of the following Table 4 were synthesized in the same manner as in Preparation Example 1 except that 1-bromo-9H-fluoren-9-one was used instead of 2-bromo-9H-fluoren-9-one, the following Compound A was used instead of bromobenzene, the following Compound B was used instead of ethynylbenzene, and the following Compound C was used instead of di([1,1′-biphenyl]-4-yl)amine.

TABLE 4

| Compound | A | B | C |
|---|---|---|---|
| 173 | 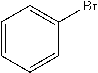 | 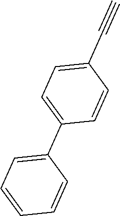 | 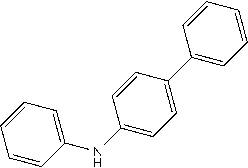 |
| 174 | 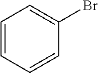 | 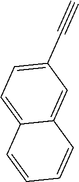 | 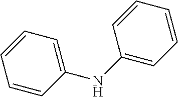 |
| 175 | 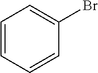 | 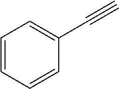 | 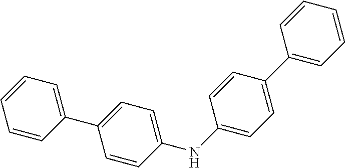 |
| 176 | 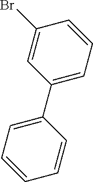 | 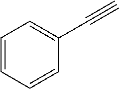 | 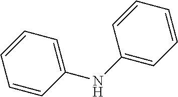 |

| Compound | Target Compound |
|---|---|
| 173 | 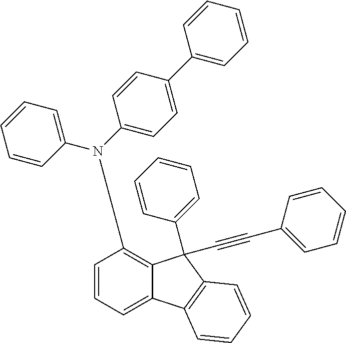 [SMILES: C(#CC1(c2ccccc2)c2ccccc2-c2cccc(N(c3ccccc3)c3ccc(-c4ccccc4)cc3)c21)c1ccccc1] |
| 174 | 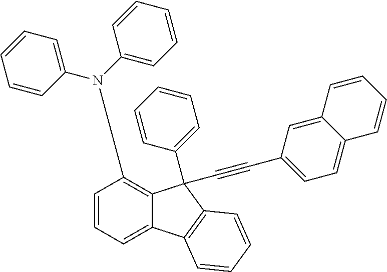 [SMILES: C(#CC1(c2ccccc2)c2ccccc2-c2cccc(N(c3ccccc3)c3ccccc3)c21)c1ccc2ccccc2c1] |
| 175 | 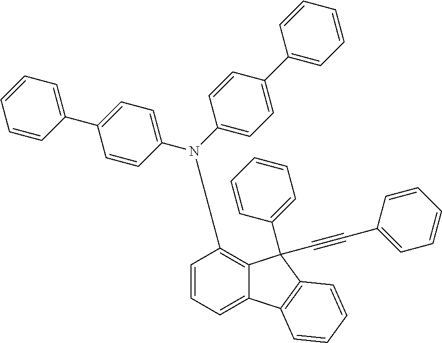 [SMILES: C(#CC1(c2ccccc2)c2ccccc2-c2cccc(N(c3ccc(-c4ccccc4)cc3)c3ccc(-c4ccccc4)cc3)c21)c1ccccc1] |
| 176 | 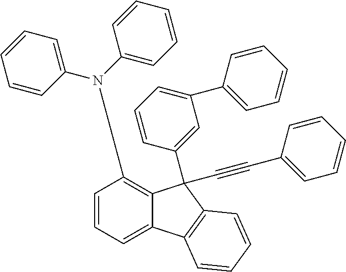 [SMILES: C(#CC1(c2cccc(-c3ccccc3)c2)c2ccccc2-c2cccc(N(c3ccccc3)c3ccccc3)c21)c1ccccc1] |

### <Preparation Example 2> Preparation of Compound 21

From Compound 1-1 obtained in Preparation Example 1, Compound 21 was obtained using the following synthesis method.

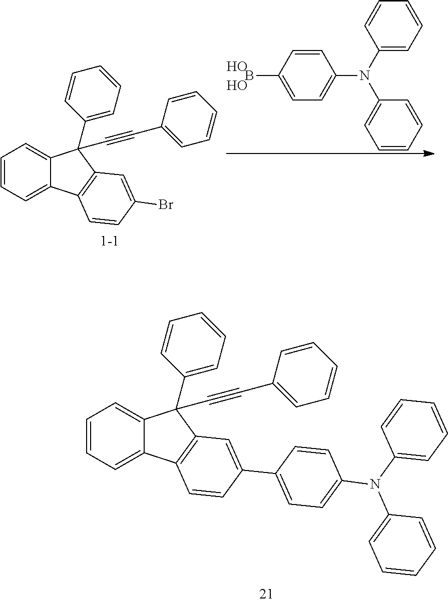

### Preparation of Compound 21

Into a one neck round bottom flask (one neck r.b.f), 2-bromo-9-phenyl-9-(phenylethynyl)-9H-fluorene (10 g, 14 mmol), (4-(diphenylamino)phenyl)boronic acid (6.6 g, 24 mmol), tetrakis(triphenylhosphine)palladium(0) (Pd(PPh₃)₄) (1.3 g, 1.2 mmol), K₂CO₃ (10 g, 72 mmol), 1,4-dioxane (100 ml) and distilled water (30 ml) were added dropwise, and the mixture was stirred under reflux for 4 hours.

The reaction solution was extracted, then adsorbed using silica, and purified using a column chromatography purification method to obtain Compound 21 (12 g, yield 86%).

Target compounds of the following Table 5 were synthesized in the same manner as in Preparation Example 2 except that the following Compound A was used instead of 2-bromo-9-phenyl-9-(phenylethynyl)-9H-fluorene, and the following Compound B was used instead of (4-(diphenylamino)phenyl)boronic acid.

TABLE 5

| Compound | A | B |
|---|---|---|
| 22 | 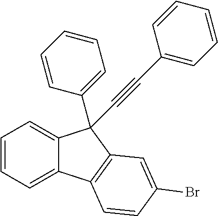 | 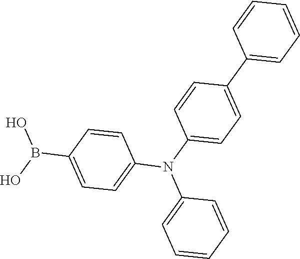 |
| 23 | 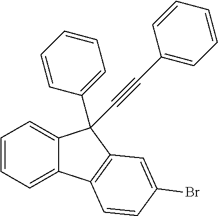 | 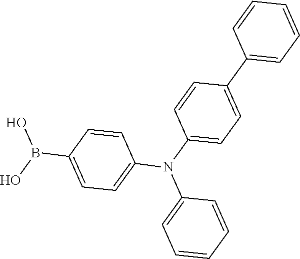 |
| 24 | 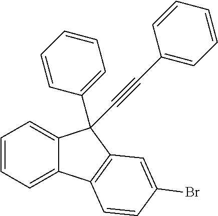 | 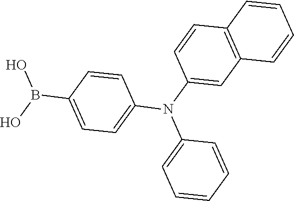 |
| 25 | 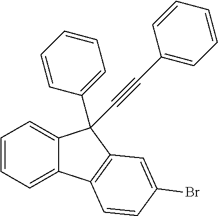 | 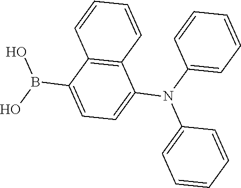 |
| 26 | 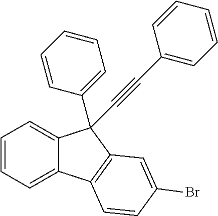 | 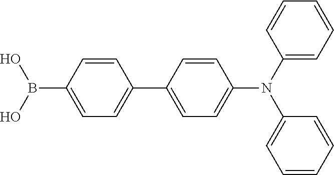 |
| 27 | 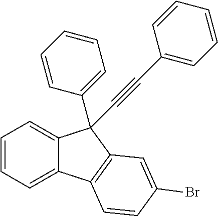 | 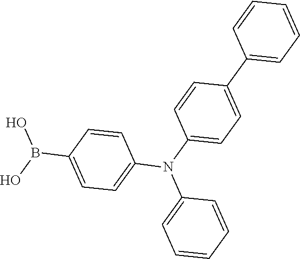 |
| 28 | 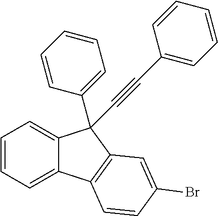 | 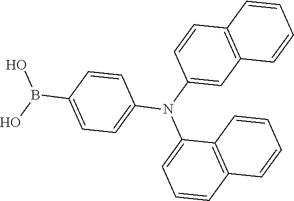 |
| 29 | 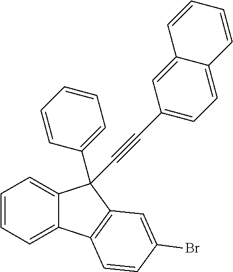 | 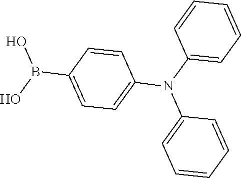 |
| 30 | 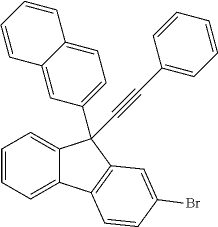 | 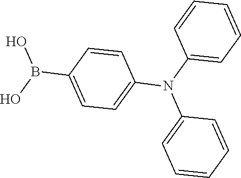 |
| 31 | 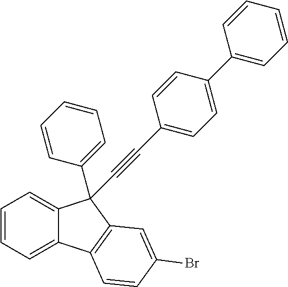 | 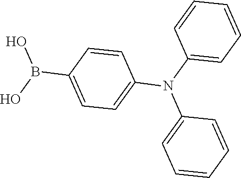 |
| 32 | 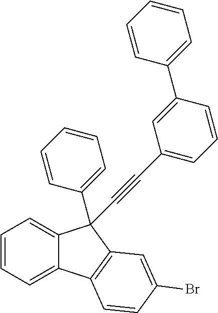 | 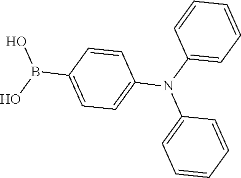 |
| 33 | 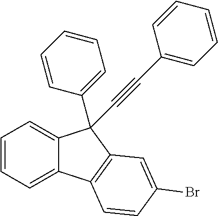 | 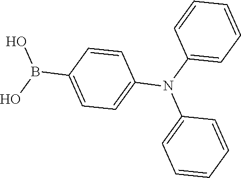 |
| 34 | 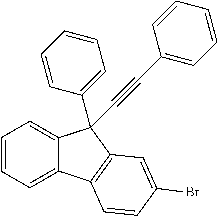 | 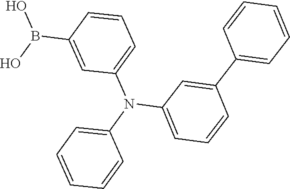 |
| 35 | 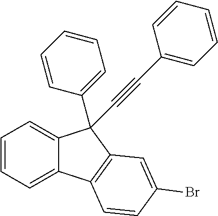 | 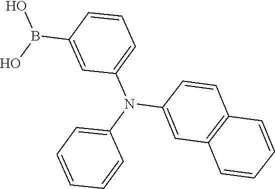 |
| 36 | 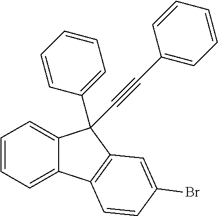 | 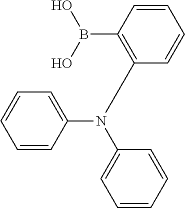 |
| 37 | 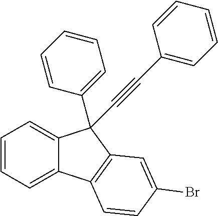 | 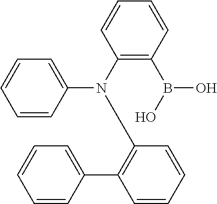 |
| 38 | 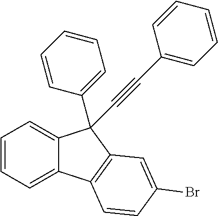 | 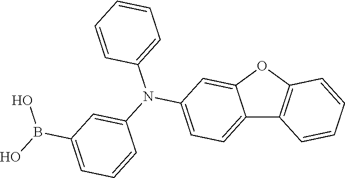 |
| 39 | 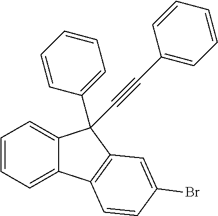 | 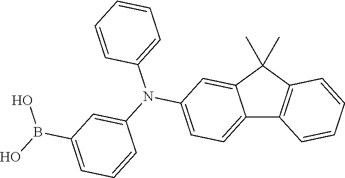 |
| 40 | 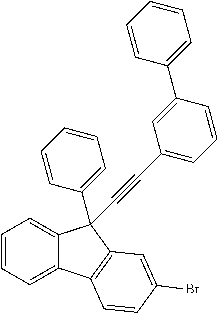 | 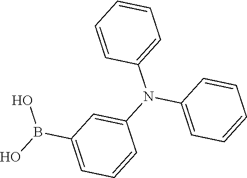 |

| Compound | Target Compound |
|---|---|
| 22 | 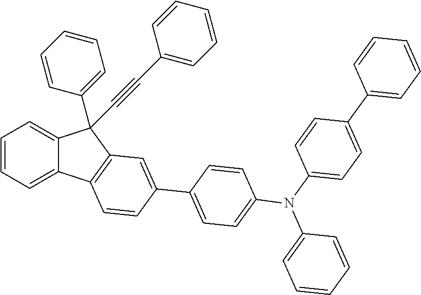 [SMILES: C(#CC1(c2ccccc2)c2ccccc2-c2ccc(-c3ccc(N(c4ccccc4)c4ccc(-c5ccccc5)cc4)cc3)cc21)c1ccccc1] |
| 23 | 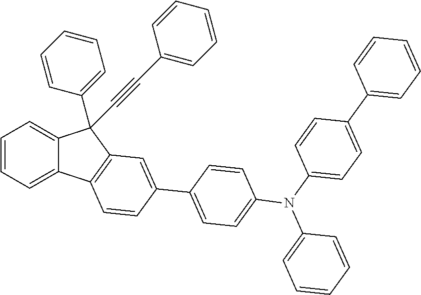 [SMILES: C(#CC1(c2ccccc2)c2ccccc2-c2ccc(-c3ccc(N(c4ccccc4)c4ccc(-c5ccccc5)cc4)cc3)cc21)c1ccccc1] |
| 24 | 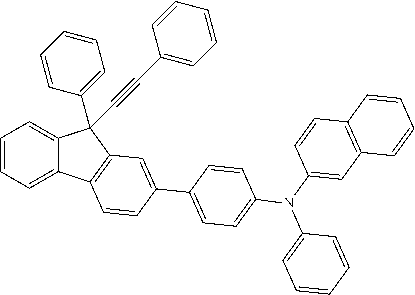 [SMILES: C(#CC1(c2ccccc2)c2ccccc2-c2ccc(-c3ccc(N(c4ccccc4)c4ccc5ccccc5c4)cc3)cc21)c1ccccc1] |
| 25 | 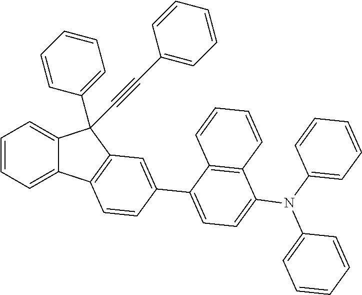 [SMILES: C(#CC1(c2ccccc2)c2ccccc2-c2ccc(-c3ccc(N(c4ccccc4)c4ccccc4)c4ccccc34)cc21)c1ccccc1] |
| 26 | 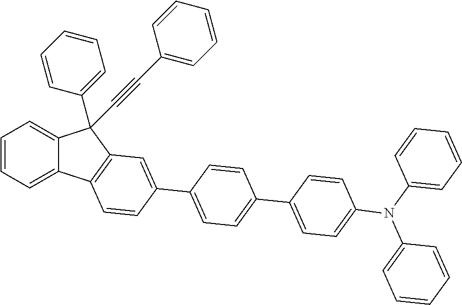 [SMILES: C(#CC1(c2ccccc2)c2ccccc2-c2ccc(-c3ccc(-c4ccc(N(c5ccccc5)c5ccccc5)cc4)cc3)cc21)c1ccccc1] |
| 27 | 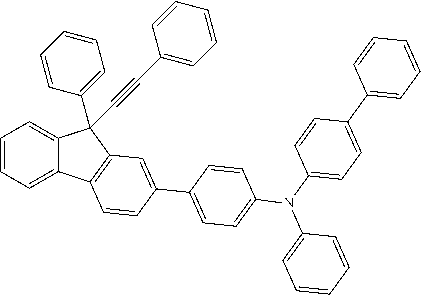 [SMILES: C(#CC1(c2ccccc2)c2ccccc2-c2ccc(-c3ccc(N(c4ccccc4)c4ccc(-c5ccccc5)cc4)cc3)cc21)c1ccccc1] |
| 28 | 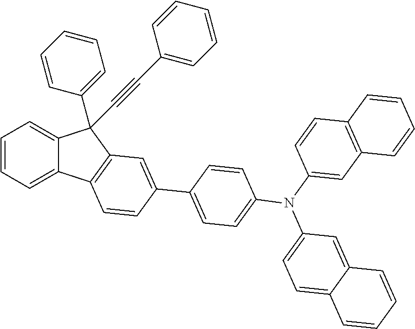 [SMILES: C(#CC1(c2ccccc2)c2ccccc2-c2ccc(-c3ccc(N(c4ccc5ccccc5c4)c4ccc5ccccc5c4)cc3)cc21)c1ccccc1] |
| 29 | 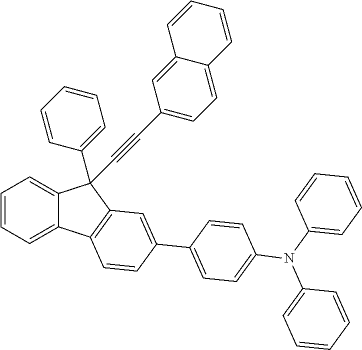 [SMILES: C(#CC1(c2ccccc2)c2ccccc2-c2ccc(-c3ccc(N(c4ccccc4)c4ccccc4)cc3)cc21)c1ccc2ccccc2c1] |
| 30 | 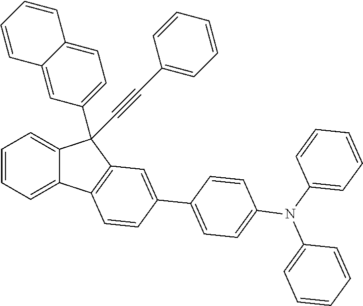 [SMILES: C(#CC1(c2ccc3ccccc3c2)c2ccccc2-c2ccc(-c3ccc(N(c4ccccc4)c4ccccc4)cc3)cc21)c1ccccc1] |
| 31 | 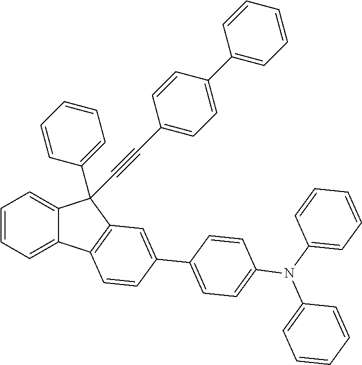 [SMILES: C(#CC1(c2ccccc2)c2ccccc2-c2ccc(-c3ccc(N(c4ccccc4)c4ccccc4)cc3)cc21)c1ccc(-c2ccccc2)cc1] |
| 32 | 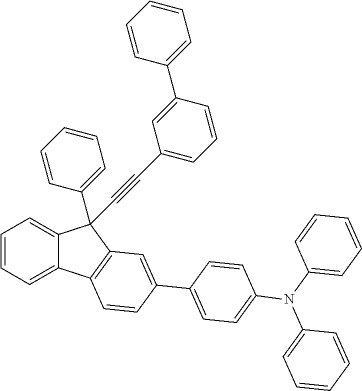 [SMILES: C(#CC1(c2ccccc2)c2ccccc2-c2ccc(-c3ccc(N(c4ccccc4)c4ccccc4)cc3)cc21)c1cccc(-c2ccccc2)c1] |
| 33 | 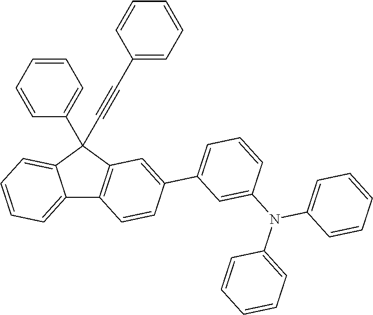 [SMILES: C(#CC1(c2ccccc2)c2ccccc2-c2ccc(-c3cccc(N(c4ccccc4)c4ccccc4)c3)cc21)c1ccccc1] |
| 34 | 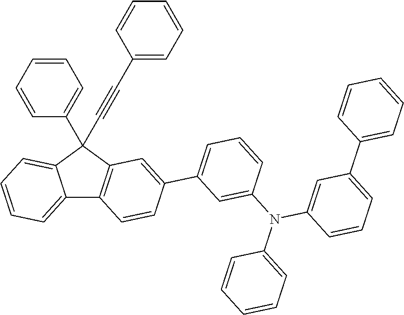 [SMILES: C(#CC1(c2ccccc2)c2ccccc2-c2ccc(-c3cccc(N(c4ccccc4)c4cccc(-c5ccccc5)c4)c3)cc21)c1ccccc1] |
| 35 | 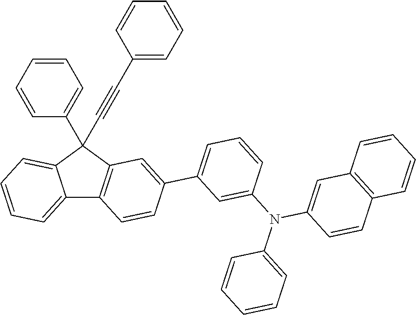 [SMILES: C(#CC1(c2ccccc2)c2ccccc2-c2ccc(-c3cccc(N(c4ccccc4)c4ccc5ccccc5c4)c3)cc21)c1ccccc1] |
| 36 | 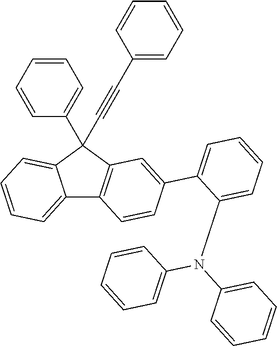 [SMILES: C(#CC1(c2ccccc2)c2ccccc2-c2ccc(-c3ccccc3N(c3ccccc3)c3ccccc3)cc21)c1ccccc1] |
| 37 | 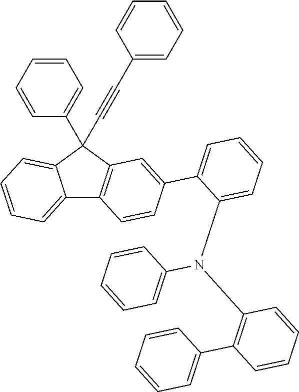 [SMILES: C(#CC1(c2ccccc2)c2ccccc2-c2ccc(-c3ccccc3N(c3ccccc3)c3ccccc3-c3ccccc3)cc21)c1ccccc1] |
| 38 | 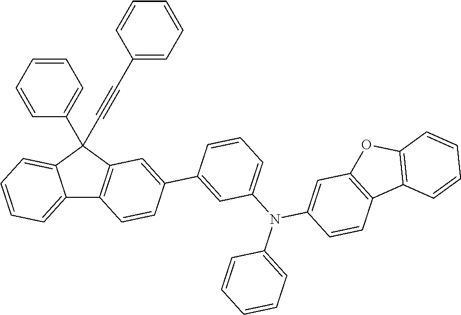 [SMILES: C(#CC1(c2ccccc2)c2ccccc2-c2ccc(-c3cccc(N(c4ccccc4)c4ccc5c(c4)oc4ccccc45)c3)cc21)c1ccccc1] |
| 39 | 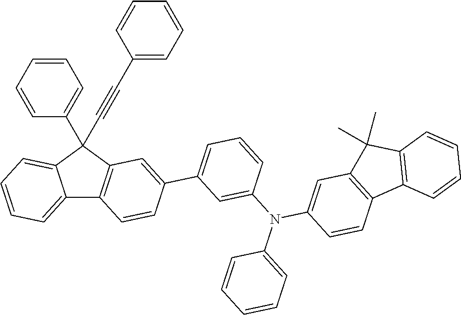 [SMILES: CC1(C)c2ccccc2-c2ccc(N(c3ccccc3)c3cccc(-c4ccc5c(c4)C(C#Cc4ccccc4)(c4ccccc4)c4ccccc4-5)c3)cc21] |
| 40 | 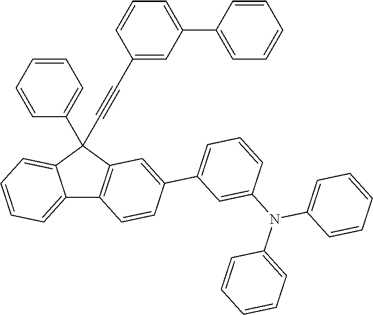 [SMILES: C(#CC1(c2ccccc2)c2ccccc2-c2ccc(-c3cccc(N(c4ccccc4)c4ccccc4)c3)cc21)c1cccc(-c2ccccc2)c1] |

Compounds other than the compounds described in Preparation Examples 1 and 2 and Table 1 to Table 5 were also prepared in the same manner as in the methods described in the preparation examples, and the synthesis results are shown in the following Table 6 and Table 7. The following Table 6 shows measurement values of FD-mass spectrometry (FD-MS: field desorption mass spectrometry), and the following Table 7 shows measurement values of ¹H NMR (CDCl₃, 300 MHz).

TABLE 6

| Compound | FD-MS |
|---|---|
| 1 | m/z = 661.83 |
| | (C51H35N = 661.28) |
| 3 | m/z = 599.72 |
| | (C45H29NO = 599.22) |
| 5 | m/z = 635.79 |
| | (C49H33N = 635.26) |
| 7 | m/z = 661.83 |
| | (C51H35N = 661.28) |
| 9 | m/z = 635.79 |
| | (C49H33N = 635.26) |
| 11 | m/z = 635.79 |
| | (C49H33N = 635.26) |
| 13 | m/z = 675.81 |
| | (C51H33NO = 675.26) |
| 15 | m/z = 635.79 |
| | (C49H33N = 635.26) |
| 17 | m/z = 599.72 |
| | (C45H29NO = 599.22) |
| 19 | m/z = 609.76 |
| | (C47H31N = 609.25) |
| 21 | m/z = 585.73 |
| | (C45H31N = 585.25) |
| 23 | m/z = 661.83 |
| | (C51H35N = 661.28) |
| 25 | m/z = 635.79 |
| | (C49H33N = 635.26) |
| 27 | m/z = 661.83 |
| | (C51H35N = 661.28) |
| 29 | m/z = 635.79 |
| | (C49H33N = 635.26) |
| 31 | m/z = 661.83 |
| | (C51H35N = 661.28) |
| 33 | m/z = 585.73 |
| | (C45H31N = 585.25) |
| 35 | m/z = 635.79 |
| | (C49H33N = 635.26) |
| 37 | m/z = 661.83 |
| | (C51H35N = 661.28) |
| 39 | m/z = 701.89 |
| | (C54H39N = 701.31) |
| 41 | m/z = 701.89 |
| | (C54H39N = 701.31) |
| 43 | m/z = 661.83 |
| | (C51H35N = 661.28) |
| 45 | m/z = 609.76 |
| | (C47H31N = 609.25) |
| 47 | m/z = 661.83 |
| | (C51H35N = 661.28) |
| 49 | m/z = 625.80 |
| | (C48H35N = 625.28) |
| 51 | m/z = 537.69 |
| | (C41H31N = 537.25) |
| 53 | m/z = 675.81 |
| | (C51H33NO = 675.26) |
| 55 | m/z = 685.85 |
| | (C53H35N = 685.28) |
| 57 | m/z = 585.73 |
| | (C45H31N = 585.25) |
| 59 | m/z = 676.84 |
| | (C51H36N2 = 676.29) |
| 61 | m/z = 557.68 |
| | (C43H27N = 557.21) |
| 63 | m/z = 607.74 |
| | (C47H29N = 607.23) |
| 65 | m/z = 647.76 |
| | (C49H29NO = 647.22) |
| 67 | m/z = 672.81 |
| | (C51H32N2 = 672.26) |
| 69 | m/z = 647.76 |
| | (C49H29NO = 647.22) |
| 71 | m/z = 633.78 |
| | (C49H31N = 633.25) |
| 73 | m/z = 597.70 |
| | (C45H27NO = 597.21) |
| 75 | m/z = 613.77 |
| | (C45H27NS = 613.19) |
| 77 | m/z = 672.81 |
| | (C51H32N2 = 672.26) |
| 79 | m/z = 748.91 |
| | (C57H36N2 = 748.29) |
| 81 | m/z = 661.83 |
| | (C51H35N = 661.28) |
| 83 | m/z = 599.72 |
| | (C45H29NO = 599.22) |
| 85 | m/z = 635.79 |
| | (C49H33N = 635.26) |
| 87 | m/z = 661.83 |
| | (C51H35N = 661.28) |
| 89 | m/z = 635.79 |
| | (C49H33N = 635.26) |
| 91 | m/z = 635.79 |
| | (C49H33N = 635.26) |
| 93 | m/z = 675.81 |
| | (C51H33NO = 675.26) |
| 95 | m/z = 635.79 |
| | (C49H33N = 635.26) |
| 97 | m/z = 599.72 |
| | (C45H29NO = 599.22) |
| 99 | m/z = 609.76 |
| | (C47H31N = 609.25) |
| 101 | m/z = 625.80 |
| | (C48H35N = 625.28) |
| 103 | m/z = 661.83 |
| | (C51H35N = 661.28) |
| 105 | m/z = 609.76 |
| | (C47H31N = 609.25) |
| 107 | m/z = 661.83 |
| | (C51H35N = 661.28) |
| 109 | m/z = 625.80 |
| | (C48H35N = 625.28) |
| 111 | m/z = 537.69 |
| | (C41H31N = 537.25) |
| 113 | m/z = 675.81 |
| | (C51H33NO = 675.26) |
| 115 | m/z = 685.85 |
| | (C53H35N = 685.28) |
| 117 | m/z = 585.73 |
| | (C45H31N = 585.25) |
| 119 | m/z = 676.84 |
| | (C51H36N2 = 676.29) |
| 121 | m/z = 661.83 |
| | (C51H35N = 661.28) |
| 123 | m/z = 599.72 |
| | (C45H29NO = 599.22) |
| 125 | m/z = 635.79 |
| | (C49H33N = 635.26) |
| 127 | m/z = 661.83 |
| | (C51H35N = 661.28) |
| 129 | m/z = 635.79 |
| | (C49H33N = 635.26) |
| 131 | m/z = 635.79 |
| | (C49H33N = 635.26) |
| 133 | m/z = 675.81 |
| | (C51H33NO = 675.26) |
| 135 | m/z = 635.79 |
| | (C49H33N = 635.26) |
| 137 | m/z = 599.72 |
| | (C45H29NO = 599.22) |
| 139 | m/z = 609.76 |
| | (C47H31N = 609.25) |
| 141 | m/z = 625.80 |
| | (C48H35N = 625.28) |
| 143 | m/z = 661.83 |
| | (C51H35N = 661.28) |
| 145 | m/z = 609.76 |
| | (C47H31N = 609.25) |
| 147 | m/z = 661.83 |
| | (C51H35N = 661.28) |
| 149 | m/z = 625.80 |
| | (C48H35N = 625.28) |
| 151 | m/z = 537.69 |
| | (C41H31N = 537.25) |
| 153 | m/z = 675.81 |
| | (C51H33NO = 675.26) |
| 155 | m/z = 685.85 |
| | (C53H35N = 685.28) |
| 157 | m/z = 585.73 |
| | (C45H31N = 585.25) |
| 159 | m/z = 676.84 |
| | (C51H36N2 = 676.29) |
| 161 | m/z = 557.68 |
| | (C43H27N = 557.21) |
| 163 | m/z = 607.74 |
| | (C47H29N = 607.23) |
| 165 | m/z = 647.76 |
| | (C49H29NO = 647.22) |
| 167 | m/z = 672.81 |
| | (C51H32N2 = 672.26) |
| 169 | m/z = 647.76 |
| | (C49H29NO = 647.22) |
| 171 | m/z = 633.78 |
| | (C49H31N = 633.25) |
| 173 | m/z = 585.73 |
| | (C45H31N = 585.25) |
| 175 | m/z = 661.83 |
| | (C51H35N = 661.28) |
| 177 | m/z = 679.94 |
| | (C51H17D18N = 679.39) |
| 179 | m/z = 666.86 |
| | (C51H30D5N = 666.31) |

TABLE 7

| NO | ¹H NMR (CDCl₃, 300 Mz) |
|---|---|
| 1 | 7.87 (d, 1H), 7.62~7.26 (m, 27H), 6.75~6.69(m, 5H), |
| | 6.58 (d, 1H) |
| 2 | 7.87 (m, 2H), 7.62~7.55 (m, 6H), 7.43~7.20 (m, 14H), |
| | 6.81~6.75 (m, 3H), 6.63~6.53 (m, 4H), 1.72 (s, 6H) |
| 4 | 7.87 (d, 1H), 7.62~7.20 (m, 27H), 6.81~6.58 (m, 7H) |
| 5 | 8.29 (s, 1H), 8.00 (m, 2H), 7.87 (d, 1H), 7.78 (d, 1H), |
| | 7.62~7.26 (m, 21H), 6.81~6.85 (m, 7H) |
| 8 | 7.87 (d, 1H), 7.62~7.20 (m, 22H), 6.81~6.75 (m, 3H), |
| | 6.63~6.58 (m, 5H) |
| 10 | 7.88~7.74 (m, 9H), 7.55~7.26 (m, 19H), 6.75 (s, 1H), |
| | 6.58 (d, 1H) |
| 13 | 7.89 (m, 1H), 7.66~7.23 (m, 26H), 6.75~6.69 (m, 3H), |
| | 6.58 (m, 1H), 6.33 (d, 1H) |
| 14 | 7.87 (d, 1H), 7.55~7.28 (m, 21H), 6.75~6.69 (m, 4H), |
| | 6.58 (d, 2H) |
| 15 | 8.00~7.87 (m, 4H), 7.42 (d, 1H), 7.62~7.55 (m, 9H), |
| | 7.43~7.20 (m, 12H), 6.81~6.58 (m, 7H) |
| 16 | 7.87 (d, 1H), 7.55~7.08 (m, 28H), 6.87 (m, 1H), |
| | 6.75~6.69 (m, 4H), 6.58 (d, 1H) |
| 17 | 7.89~7.86 (m, 2H), 7.66~7.55 (m, 4H), 7.38~7.20 (m, 14H), |
| | 6.81~6.75 (m, 3H), 6.63~6.58 (m, 5H) |
| 18 | 7.87 (d, 1H), 7.62~7.20 (m, 27H), 6.81~6.75 (m, 2H), |
| | 6.69~6.58 (m, 5H) |
| 21 | 7.93~7.87 (m, 2H), 7.77 (s, 1H), 7.63~7.54 (m, 6H), |
| | 7.43~7.20 (m, 14H), 6.81 (m, 2H), 6.69~6.63 (m, 6H) |
| 23 | 7.93~7.87 (m, 2H), 7.77 (s, 1H), 7.63~7.20 (m, 25H), |
| | 6.81 (m, 1H), 6.69~6.63 (m, 6H) |
| 26 | 7.93~7.87 (m, 2H), 7.77 (d, 1H), 7.63 (d, 1H), |
| | 7.55~7.54 (m, 5H), 7.42~7.20 (m, 18H), 6.81 (m, 2H), |
| | 6.69~6.63 (m, 6H) |
| 33 | 7.93~7.87 (m, 2H), 7.77 (d, 1H), 7.63~7.55 (m, 4H), |
| | 7.43~7.20 (m, 15H), 6.89~6.81 (m, 4H), 6.63~6.59 (m, 5H) |
| 38 | 7.93~7.87 (m, 3H), 7.77 (d, 1H), 7.66~7.55 (m, 6H), |
| | 7.43~7.20 (m, 16H), 6.89~6.81 (m, 3H), 6.63~6.59 (m, 3H), |
| | 6.33 (d, 1H) |
| 39 | 7.93~7.87 (d, 3H), 7.77 (s, 1H), 7.62~7.55 (m, 6H), |
| | 7.44~7.20 (m, 15H), 6.89~6.75 (m, 4H), 6.63~6.58 (m, 4H), |
| | 1.72 (s, 6H) |
| 41 | 7.87 (d, 1H), 7.62~7.26 (m, 25H), 6.75~6.69 (m, 4H), |
| | 6.58 (d, 2H), 1.72 (s, 6H) |
| 42 | 7.87 (d, 1H), 7.55~7.20 (m, 26H), 7.06 (s, 1H), |
| | 6.85~6.75 (m, 4H), 6.63~6.58 (m, 3H) |
| 44 | 8.55 (d, 1H), 8.12 (d, 1H), 7.94~7.87 (m, 2H), |
| | 7.63~7.20 (m, 23H), 6.81~6.75 (m, 2H), 6.63~6.58 (m, 5H) |
| 61 | 8.55 (d, 1H), 8.16 (m, 2H), 7.94~7.87 (m, 3H), 7.67 (m, 2H), |
| | 7.55 (m, 2H), 7.43~7.17 (m, 15H), 7.07 (m, 1H) |
| 62 | 8.55 (d, 1H), 8.12 (d, 1H), 7.94~7.87 (m, 3H), |
| | 7.63~7.17 (m, 25H), 7.07 (t, 1H) |
| 63 | 8.54 (d, 2H), 8.16 (m, 2H), 7.96~7.87(m, 4H), |
| | 7.67~7.55 (m, 8H), 7.43~7.17 (m, 12H) |
| 70 | 8.55 (m, 2H), 8.12 (d, 1H), 7.98~7.87 (m, 5H), |
| | 7.55~7.17 (m, 23H), 7.07 (m, 1H) |
| 71 | 8.55 (d, 1H), 8.16 (m, 2H), 7.94~7.87 (m, 3H), 7.67 (m, 2H), |
| | 7.55~7.17 (m, 24H), 7.07 (t, 1H) |
| 79 | 8.55 (m, 2H), 7.94~7.87 (m, 4H), 7.79 (d, 1H), |
| | 7.58~7.17 (m, 28H), 7.07 (m, 1H) |
| 81 | 7.87 (d, 1H), 7.55~7.23 (m, 28H), 7.04 (s, 1H), 6.69 (d, |
| | 4H), 6.48 (d, 1H) |
| 82 | 7.87 (m, 2H), 7.62~7.55 (m, 5H), 7.43~7.20 (m, 14H), |
| | 7.04 (s, 1H), 6.81~6.75 (m, 2H), 6.63~6.58 (m, 3H), |
| | 6.48 (m, 1H) |
| 83 | 7.89~7.87 (m, 2H), 7.66~7.64 (m, 2H), 7.55 (m, 3H), |
| | 7.43~7.20 (m, 16H), 7.04 (s, 1H), 6.81 (m, 1H), 6.63 (d, |
| | 2H), 6.48 (d, 1H), 6.33 (d, 1H), 1.72 (s, 6H) |
| 94 | 7.87 (m, 1H), 7.55~7.23 (m, 25H), 7.04 (s, 1H), |
| | 6.75~6.69 (m, 3H), 6.58 (d, 1H), 6.48 (d, 1H), 1.72 (s, 6H) |
| 98 | 7.87 (d, 1H), 7.63~7.20 (m, 25H), 7.04 (s, 1H), 6.81 (t, |
| | 1H), 6.69~6.63 (m, 4H), 6.48 (d, 1H) |
| 101 | 7.87 (d, 2H), 7.62~7.55 (m, 5H), 7.43~7.20 (m, 15H), |
| | 7.04 (s, 1H), 6.81~6.75 (m, 2H), 6.63~6.58 (m, 3H), |
| | 6.48 (d, 1H), 1.72 (s, 6H) |
| 107 | 7.87 (d, 1H), 7.63~7.20 (m, 26H), 7.04 (s, 1H), 6.81 (m, |
| | 2H), 6.63 (m, 4H), 6.48 (d, 1H) |
| 121 | 6.69 (m, 1H), 7.52~7.26 (m, 27H), 7.03 (t, 1H), 6.91 (d, |
| | 1H), 6.69 (d, 4H), 6.58 (d, 1H) |
| 123 | 7.89~7.87 (m, 2H), 7.66~7.64 (m, 2H), 7.55 (m, 3H), |
| | 7.43~7.20 (m, 15H), 7.03 (t, 1H), 6.91 (d, 1H), 6.81 (m, |
| | 2H), 6.63~6.58 (m, 3H), 6.33 (d, 1H) |
| 130 | 7.88~7.77 (m, 9H), 7.55~7.23 (m, 19H), 7.03 (t, 1H), |
| | 6.91 (d, 1H), 6.58 (d, 1H) |
| 141 | 7.87 (m, 2H), 7.87~7.55 (m, 5H), 7.43~7.20 (m, 14H), |
| | 7.03 (t, 1H), 6.91 (d, 1H), 6.81~6.75 (m, 2H), |
| | 6.63~6.58 (m, 4H), 1.72 (s, 6H) |
| 161 | 8.55 (d, 1H), 8.16 (d, 2H), 7.94~7.87 (m, 2H), |
| | 7.67~7.55 (m, 6H), 7.43~7.17 (m, 14H), 7.07 (d, 2H) |
| 175 | 7.87 (d, 1H), 7.54~7.23 (m, 28H), 7.13 (t, 1H), 6.69 (d, |
| | 4H), 6.48 (d, 1H) |
| 177 | 7.87 (d, 1H), 7.62~7.55 (m, 4H), 7.43~7.26 (m, 10H), |
| | 6.75 (s, 1H), 6.58 (d, 1H) |

### EXPERIMENTAL EXAMPLE

### Experimental Example 1

(1) Manufacture of Organic Light Emitting Device

A glass substrate on which ITO (indium tin oxide) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function increase and residual film removal, the substrate was transferred to a thermal deposition apparatus for organic deposition.

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate. To another cell in the vacuum deposition apparatus, the compound represented by Chemical Formula 1 or the following comparative compound described in the following Table 8 was introduced, and evaporated by applying a current to the cell to deposit a hole transfer layer on the hole injection layer to a thickness of 300 Å.

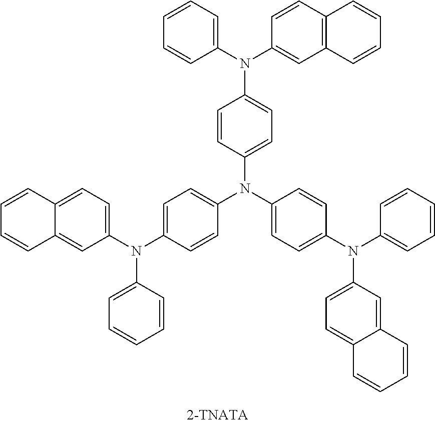 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

A light emitting layer was thermal vacuum deposited thereon as follows. As the light emitting layer, a compound of 9-[4-(4,6-diphenyl-1,3,5-triazin-2-yl)phenyl]-9′-phenyl-3,3′-bi-9H-carbazole was deposited to a thickness of 400 Å as a host, and a green phosphorescent dopant [Ir(ppy)₃] was doped and deposited by 7% with respect to the deposited thickness of the light emitting layer. After that, BCP (bathocuproine) was deposited to a thickness of 60 Å as a hole blocking layer, and Alq₃ was deposited to a thickness of 200 Å thereon as an electron transfer layer. Lastly, an electron injection layer was formed on the electron transfer layer by depositing lithium fluoride (LiF) to a thickness of 10 Å, and then a negative electrode was formed on the electron injection layer by depositing an aluminum (Al) negative electrode to a thickness of 1,220 Å, and as a result, an organic light emitting device was manufactured.

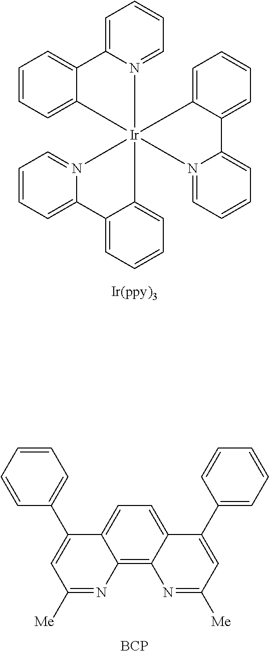

Meanwhile, all the organic compounds required to manufacture the organic light emitting device were vacuum sublimation purified under 10⁻¹ torr to 10⁻⁶ torr for each material to be used in the organic light emitting device manufacture.

Herein, the comparative compounds used as the hole transfer layer of the following comparative examples are as follows.

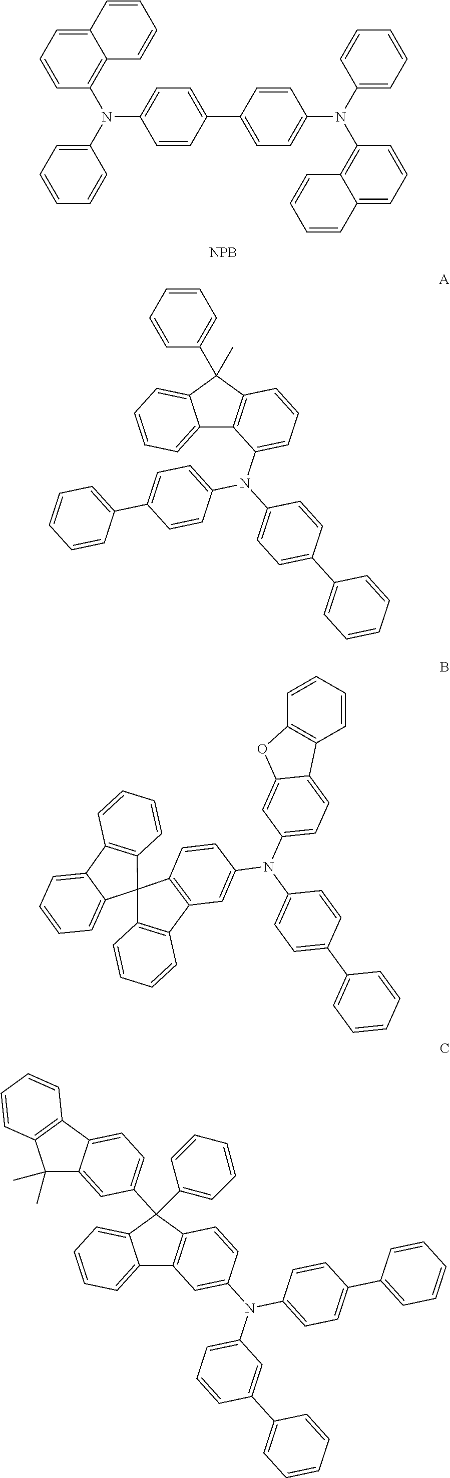 [SMILES: c1ccc(N(c2ccc(-c3ccc(N(c4ccccc4)c4cccc5ccccc45)cc3)cc2)c2cccc3ccccc23)cc1 ; "NPB" ; CC1(c2ccccc2)c2ccccc2-c2c(N(c3ccc(-c4ccccc4)cc3)c3ccc(-c4ccccc4)cc3)cccc21 ; c1ccc(-c2ccc(N(c3ccc4c(c3)-c3ccccc3C43c4ccccc4-c4ccccc43)c3ccc4c(c3)oc3ccccc34)cc2)cc1 ; CC1(C)c2ccccc2-c2ccc(C3(c4ccccc4)c4ccccc4-c4cc(N(c5ccc(-c6ccccc6)cc5)c5cccc(-c6ccccc6)c5)ccc43)cc21]

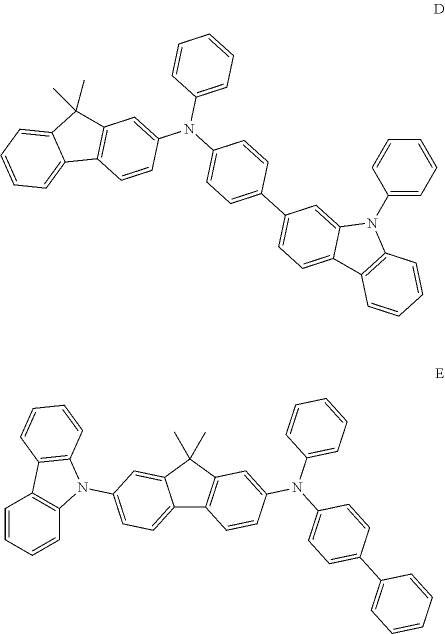 [SMILES: CC1(C)c2ccccc2-c2ccc(N(c3ccccc3)c3ccc(-c4ccc5c6ccccc6n(-c6ccccc6)c5c4)cc3)cc21 ; CC1(C)c2cc(N(c3ccccc3)c3ccc(-c4ccccc4)cc3)ccc2-c2ccc(-n3c4ccccc4c4ccccc43)cc21]

(2) Driving Voltage and Light Emission Efficiency of Organic Light Emitting Device

For each of the organic light emitting devices of Examples 1 to 14 and Comparative Examples 1 to 5 manufactured as above, electroluminescent (EL) properties were measured using M7000 manufactured by McScience Inc., and with the measurement results, a lifetime T₉₀ (unit: h, hour), time taken to become 90% with respect to initial luminance, was measured when standard luminance was 6,000 cd/m² through a lifetime measurement system (M6000) manufactured by McScience Inc.

Properties of the organic light emitting devices of the present disclosure obtained from the measurement results are as shown in the following Table 8.

TABLE 8

| | | Driving | Efficiency | Lifetime |
|---|---|---|---|---|
| | Compound | Voltage (V) | (cd/A) | (T₉₀) |
| Example 1 | Compound 1 | 3.51 | 140.5 | 210 |
| Example 2 | Compound 9 | 3.63 | 138.9 | 198 |
| Example 3 | Compound 14 | 3.70 | 142.6 | 215 |
| Example 4 | Compound 21 | 3.16 | 135.3 | 189 |
| Example 5 | Compound 22 | 3.28 | 136.7 | 179 |
| Example 6 | Compound 30 | 3.33 | 131.3 | 205 |
| Example 7 | Compound 41 | 3.56 | 132.5 | 193 |
| Example 8 | Compound 61 | 3.61 | 143.8 | 206 |
| Example 9 | Compound 62 | 3.29 | 145.9 | 215 |
| Example 10 | Compound 63 | 3.40 | 139.6 | 188 |
| Example 11 | Compound 81 | 3.36 | 142.1 | 196 |
| Example 12 | Compound 82 | 3.29 | 147.4 | 203 |
| Example 13 | Compound 86 | 3.18 | 142.6 | 201 |
| Example 14 | Compound 177 | 3.23 | 145.9 | 250 |
| Comparative | Comparative | 4.53 | 98.26 | 74 |
| Example 1 | Compound A | | | |
| Comparative | Comparative | 4.63 | 81.2 | 90 |
| Example 2 | Compound B | | | |
| Comparative | Comparative | 4.56 | 79.8 | 71 |
| Example 3 | Compound C | | | |
| Comparative | Comparative | 4.67 | 96.57 | 86 |
| Example 4 | Compound D | | | |
| Comparative | Comparative | 4.71 | 85.5 | 95 |
| Example 5 | Compound E | | | |

As seen from the results of Table 8, it was identified that the organic light emitting device using the hole transfer layer material including the heterocyclic compound according to the present disclosure had lower driving voltage, and significantly improved light emission efficiency and lifetime compared to the comparative example.

The heterocyclic compound represented by Chemical Formula 1 of the present disclosure is a compound in which an alkyne is introduced to the No. 9 position of the fluorene group, and makes changes in the LUMO density while having a similar energy level with existing compounds.

Specifically, the arylamine or carbazole moiety substituting the heterocyclic compound represented by Chemical Formula 1 of the present disclosure exhibits strong hole properties, and therefore, LUMO is distributed in the dimethyl fluorene group or the spirofluorene group connected thereto. The dimethyl fluorene group or the spirofluorene group has relative high density LUMO distributed therein, and therefore, the compound has a high dipole-moment value.

Hole properties refer to properties capable of forming holes by donating electrons when applying an electric field, and mean properties facilitating injection of holes formed in a positive electrode to a light emitting layer, migration of holes formed in a light emitting layer to a positive electrode and migration in a light emitting layer by having conductive properties along the HOMO level. Electron properties refer to properties capable of receiving electrons when applying an electric field, and mean properties facilitating injection of electrons formed in a negative electrode to a light emitting layer, migration of electrons formed in a light emitting layer to a negative electrode and migration in a light emitting layer by having conductive properties along the LUMO level.

In a general organic light emitting material, acceptors having strong electron properties are moieties occupying a large portion of the molecule, however, the alkyne of the present disclosure has a triple bond causing abundant electrons of the bond itself, which balances electron properties and hole properties without impairing properties of the whole molecule. The charge balance improves efficiency and lifetime of the device. In addition, as shown in Table 8, it was identified that, from the organic light emitting device of the example using the heterocyclic compound represented by Chemical Formula 1 of the present disclosure having a lower driving voltage compared to the comparative example, hole properties of the hole transfer layer of the organic light emitting device of the example were relatively improved compared to the comparative example, which facilitated hole injection.

### Experimental Example 2

(1) Manufacture of Organic Light Emitting Device

A glass substrate on which ITO (indium tin oxide) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function increase and residual film removal, the substrate was transferred to a thermal deposition apparatus for organic deposition.

Subsequently, the chamber was evacuated until the degree of vacuum therein reached 10⁻⁶ torr, and then 2-TNATA was evaporated by applying a current to the cell to deposit a hole injection layer on the ITO substrate to a thickness of 600 Å. To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer on the hole injection layer to a thickness of 300 Å. After that, the compound represented by Chemical Formula 1 or the comparative compound described in the following Table 9 was deposited to a thickness of 100 Å as a light emitting auxiliary layer.

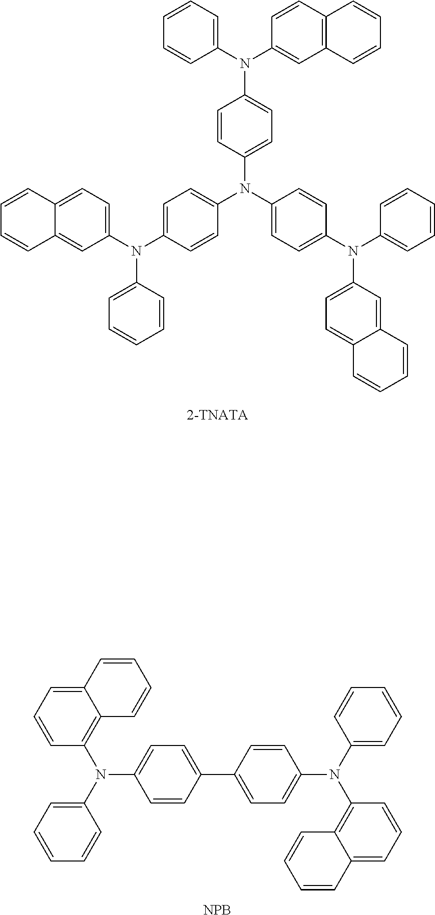 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. As the light emitting layer, a compound of 9-[4-(4,6-diphenyl-1,3,5-triazin-2-yl)phenyl]-9′-phenyl-3,3′-bi-9H-carbazole was deposited to a thickness of 400 Å as a host, and, as a green phosphorescent dopant, [Ir(ppy)₃] was doped and deposited by 7% with respect to the deposited thickness of the light emitting layer. After that, BCP (bathocuproine) was deposited to a thickness of 60 Å as a hole blocking layer, and Alq₃ was deposited to a thickness of 200 Å thereon as an electron transfer layer. Lastly, an electron injection layer was formed on the electron transfer layer by depositing lithium fluoride (LiE) to a thickness of 10 Å, and then a negative electrode was formed on the electron injection layer by depositing an aluminum (Al) negative electrode to a thickness of 1,200 Å, and as a result, an organic light emitting device was manufactured.

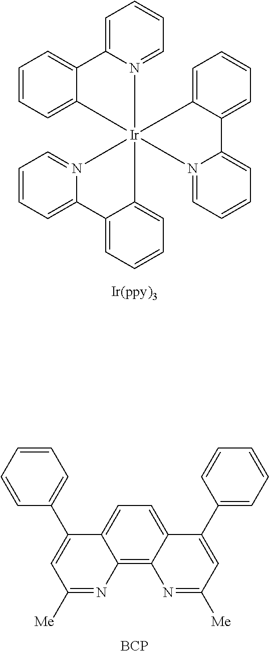

Meanwhile, all the organic compounds required to manufacture the organic light emitting device were vacuum sublimation purified under 10⁻⁸ torr to 10⁻⁶ torr for each material to be used in the organic light emitting device manufacture.

(2) Driving Voltage and Light Emission Efficiency of Organic Light Emitting Device

For each of the organic light emitting devices of Examples 15 to 28 and Comparative Examples 6 to 10 manufactured as above, electroluminescent (EL) properties were measured using M7000 manufactured by McScience Inc., and with the measurement results, a lifetime T₉₀ (unit: h, hour), time taken to become 90% with respect to initial luminance, was measured when standard luminance was 6,000 cd/m² through a lifetime measurement system (M6000) manufactured by McScience Inc.

Properties of the organic light emitting devices of the present disclosure obtained from the measurement results are as shown in the following Table 9.

TABLE 9

| | | Driving | Efficiency | Lifetime |
|---|---|---|---|---|
| | Compound | Voltage (V) | (cd/A) | (T₉₀) |
| Example 15 | Compound 1 | 4.10 | 134.6 | 170 |
| Example 16 | Compound 9 | 4.05 | 137.7 | 169 |
| Example 17 | Compound 14 | 3.98 | 141.9 | 159 |
| Example 18 | Compound 21 | 4.10 | 142.5 | 148 |
| Example 19 | Compound 22 | 3.77 | 138.5 | 162 |
| Example 20 | Compound 30 | 3.86 | 129.1 | 172 |
| Example 21 | Compound 41 | 3.91 | 140.5 | 173 |
| Example 22 | Compound 61 | 4.05 | 138.6 | 165 |
| Example 23 | Compound 62 | 4.13 | 125.7 | 149 |
| Example 24 | Compound 63 | 3.78 | 131.8 | 150 |
| Example 25 | Compound 81 | 3.98 | 138.3 | 156 |
| Example 26 | Compound 82 | 4.05 | 140.2 | 161 |
| Example 27 | Compound 86 | 4.12 | 145.6 | 158 |
| Example 28 | Compound 177 | 3.87 | 137.9 | 193 |
| Comparative | Comparative | 4.81 | 85.5 | 99 |
| Example 6 | Compound A | | | |
| Comparative | Comparative | 4.75 | 76.7 | 87 |
| Example 7 | Compound B | | | |
| Comparative | Comparative | 4.68 | 99.8 | 90 |
| Example 8 | Compound C | | | |
| Comparative | Comparative | 4.82 | 96.7 | 78 |
| Example 9 | Compound D | | | |
| Comparative | Comparative | 4.66 | 80.7 | 98 |
| Example 10 | Compound E | | | |

As seen from the results of Table 9, it was identified that the organic light emitting device using the light emitting auxiliary layer material including the heterocyclic compound according to the present disclosure had lower driving voltage, and significantly improved light emission efficiency and lifetime compared to the comparative example.

Herein, efficiency and lifetime are reduced in the OLED when electrons pass through the hole transfer layer and migrate to the positive electrode without binding in the light emitting layer. When a compound having a high LUMO level is used as the light emitting auxiliary layer in order to prevent such a phenomenon, electrons to migrate to the positive electrode after passing through the light emitting layer are blocked from migrating by an energy barrier of the light emitting auxiliary layer. Accordingly, holes and electrons very likely form excitons, which increases possibility of being emitted as light in the light emitting layer, and as a result, the organic light emitting device has excellency in all aspects of driving voltage, efficiency and lifetime when using the heterocyclic compound according to the present disclosure as the light emitting auxiliary layer.

Particularly, it was identified that using the heterocyclic compound according to the present disclosure as the light emitting auxiliary layer was capable of suppressing degradation of the hole transfer material caused by electrons invading the hole transfer layer, and, by the substituent having strengthened hole properties and the amine moiety bonding in the heterocyclic compound according to the present disclosure, planarity and glass transition temperature of the amine derivative increase, which enhanced thermal stability of the compound.

In addition, it was identified that, through adjusting the band gap and the triplet energy level (T₁ level) value, hole transfer ability was enhanced and molecular stability increased, and as a result, the organic light emitting device had lowered driving voltage and enhanced light efficiency, and the organic light emitting device had enhanced lifetime properties by enhanced thermal stability of the compound since.

Simple modifications and changes of the present disclosure all fall within the category of the present disclosure, and the specific scope of protection of the present disclosure will become clear by the appended claims.

### REFERENCE NUMERAL

100: Substrate
200: Positive electrode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Negative electrode

1. A heterocyclic compound represented by the following Chemical Formula 1: 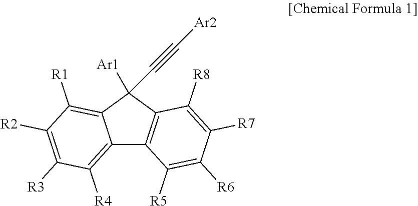
wherein, in Chemical Formula 1,
Ar1 and Ar2 are the same as or different from each other, and each independently a substituted or unsubstituted C6 to C60 aryl group; or a substituted or unsubstituted C2 to C60 heteroaryl group;
R1 to R8 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of R1 to R8 is a group represented by the following Chemical Formula 2, 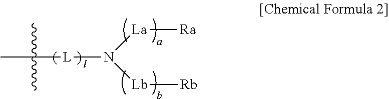
in Chemical Formula 2,
Ra and Rb are the same as or different from each other, and each independently a substituted or unsubstituted C6 to C60 aryl group; or a substituted or unsubstituted C2 to C60 heteroaryl group, or Ra and Rb bond to each other to form a substituted or unsubstituted C6 to C60 aromatic hydrocarbon ring or a substituted or unsubstituted C2 to C60 heteroring;
L, La and Lb are the same as or different from each other, and each independently a direct bond; a substituted or unsubstituted C6 to C60 arylene group; or a substituted or unsubstituted C2 to C60 heteroarylene group; and
l, a and b are the same as or different from each other, and each independently an integer of 0 to 5, and when l is 2 or greater, each L is the same as or different from each other, when a is 2 or greater, each La is the same as or different from each other, and when b is 2 or greater, each Lb is the same as or different from each other.

2. The heterocyclic compound of claim 1, wherein Chemical Formula 2 is represented by any one of the following Chemical Formula 2-1 and Chemical Formula 2-2: 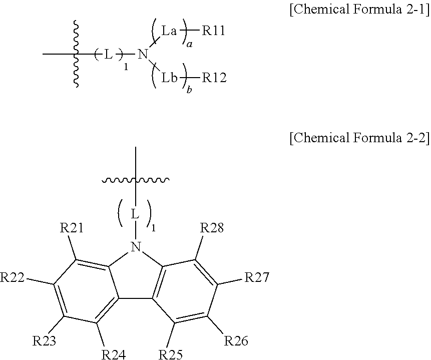
in Chemical Formula 2-1 and Chemical Formula 2-2,
R11 and R12 are the same as or different from each other, and each independently a substituted or unsubstituted C6 to C60 aryl group; or a substituted or unsubstituted C2 to C60 heteroaryl group;
R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
L, La, Lb, l, a and b have the same definitions as in Chemical Formula 2.

3. The heterocyclic compound of claim 2, wherein Chemical Formula 2-2 is represented by any one of the following Chemical Formula 2-2-a to Chemical Formula 2-2-d: 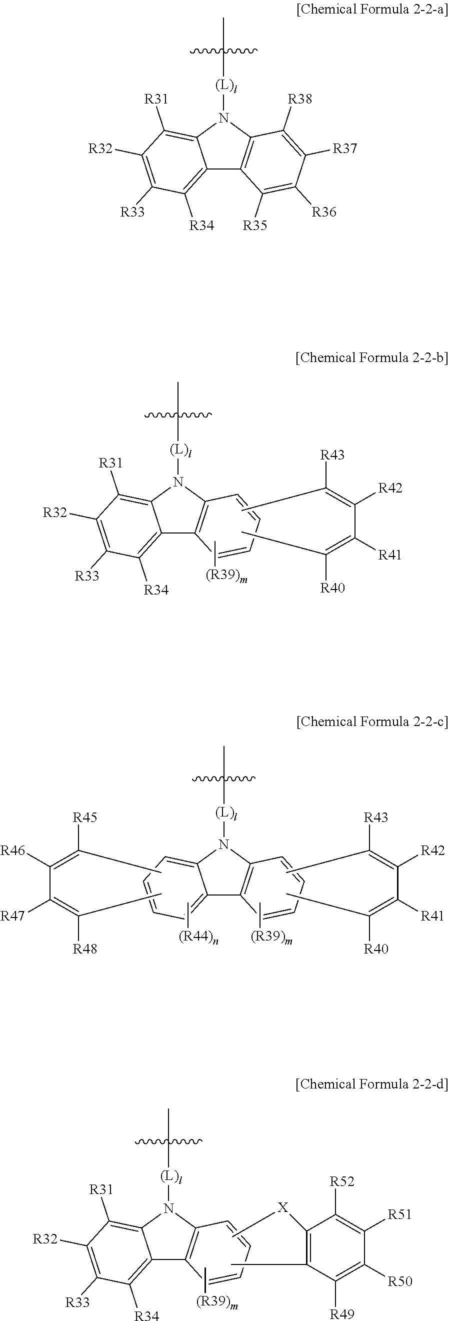
in Chemical Formula 2-2-a to Chemical Formula 2-2-d,
R31 to R52 are the same as or different from each other, and each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; or —NR201R202, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
X is O, S or NR53;
R53 is a substituted or unsubstituted C6 to C60 aryl group; or a substituted or unsubstituted C2 to C60 heteroaryl group;
m and n are the same as or different from each other, and each independently an integer of 0 to 2, and when m is 2, each R39 is the same as or different from each other, and when n is 2, each R44 is the same as or different from each other; and
L and 1 have the same definitions as in Chemical Formula 2.

4. The heterocyclic compound of claim 1, wherein Chemical Formula 1 is represented by any one of the following compounds:

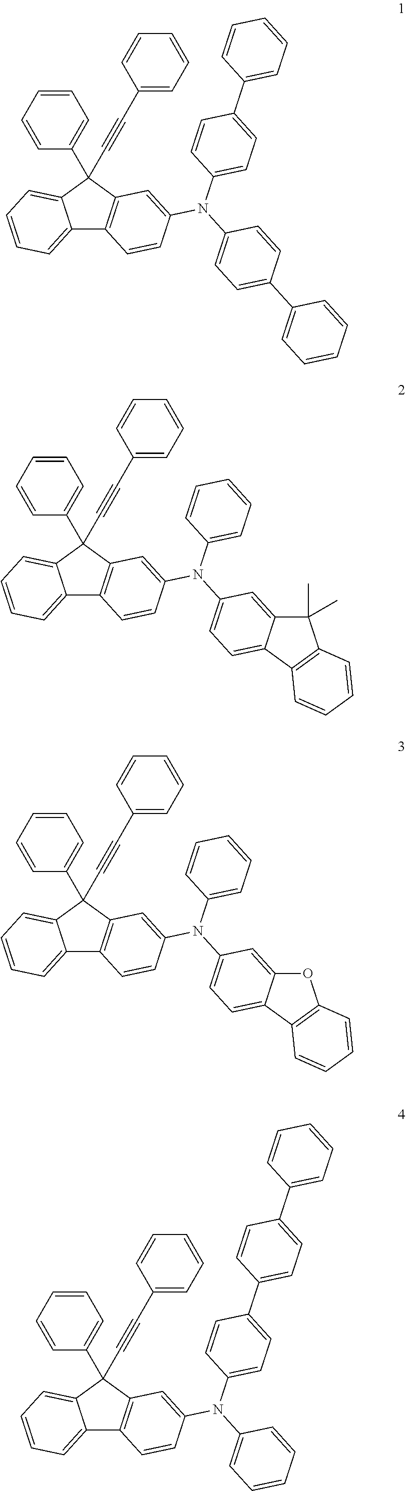 [SMILES: C(#CC1(c2ccccc2)c2ccccc2-c2ccc(N(c3ccc(-c4ccccc4)cc3)c3ccc(-c4ccccc4)cc3)cc21)c1ccccc1 ; CC1(C)c2ccccc2-c2ccc(N(c3ccccc3)c3ccc4c(c3)C(C#Cc3ccccc3)(c3ccccc3)c3ccccc3-4)cc21 ; C(#CC1(c2ccccc2)c2ccccc2-c2ccc(N(c3ccccc3)c3ccc4c(c3)oc3ccccc34)cc21)c1ccccc1 ; C(#CC1(c2ccccc2)c2ccccc2-c2ccc(N(c3ccccc3)c3ccc(-c4ccc(-c5ccccc5)cc4)cc3)cc21)c1ccccc1]

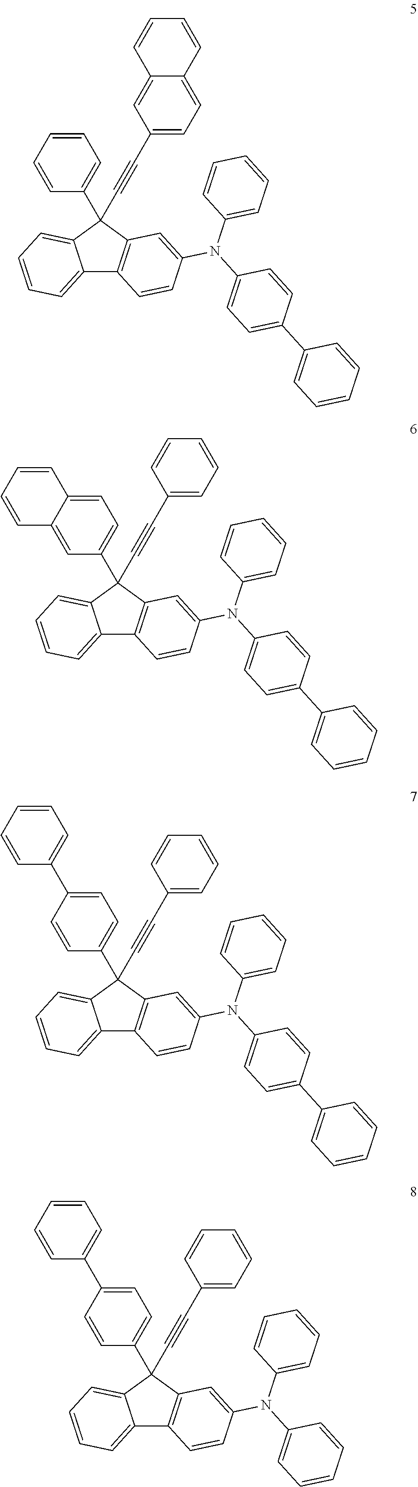 [SMILES: C(#CC1(c2ccccc2)c2ccccc2-c2ccc(N(c3ccccc3)c3ccc(-c4ccccc4)cc3)cc21)c1ccc2ccccc2c1 ; C(#CC1(c2ccc3ccccc3c2)c2ccccc2-c2ccc(N(c3ccccc3)c3ccc(-c4ccccc4)cc3)cc21)c1ccccc1 ; C(#CC1(c2ccc(-c3ccccc3)cc2)c2ccccc2-c2ccc(N(c3ccccc3)c3ccc(-c4ccccc4)cc3)cc21)c1ccccc1 ; C(#CC1(c2ccc(-c3ccccc3)cc2)c2ccccc2-c2ccc(N(c3ccccc3)c3ccccc3)cc21)c1ccccc1]

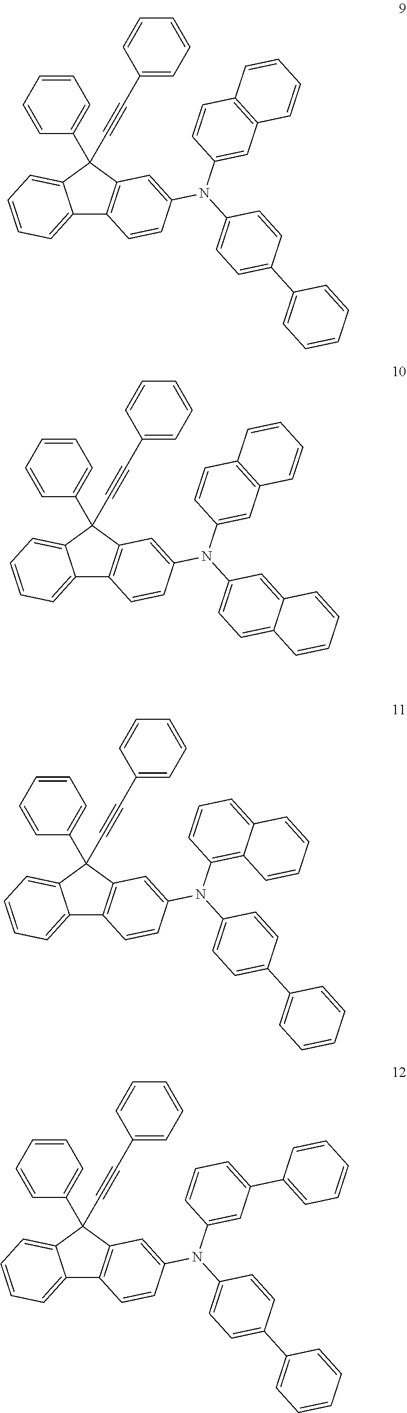 [SMILES: C(#CC1(c2ccccc2)c2ccccc2-c2ccc(N(c3ccc(-c4ccccc4)cc3)c3ccc4ccccc4c3)cc21)c1ccccc1 ; C(#CC1(c2ccccc2)c2ccccc2-c2ccc(N(c3ccc4ccccc4c3)c3ccc4ccccc4c3)cc21)c1ccccc1 ; C(#CC1(c2ccccc2)c2ccccc2-c2ccc(N(c3ccc(-c4ccccc4)cc3)c3cccc4ccccc34)cc21)c1ccccc1 ; C(#CC1(c2ccccc2)c2ccccc2-c2ccc(N(c3ccc(-c4ccccc4)cc3)c3cccc(-c4ccccc4)c3)cc21)c1ccccc1]

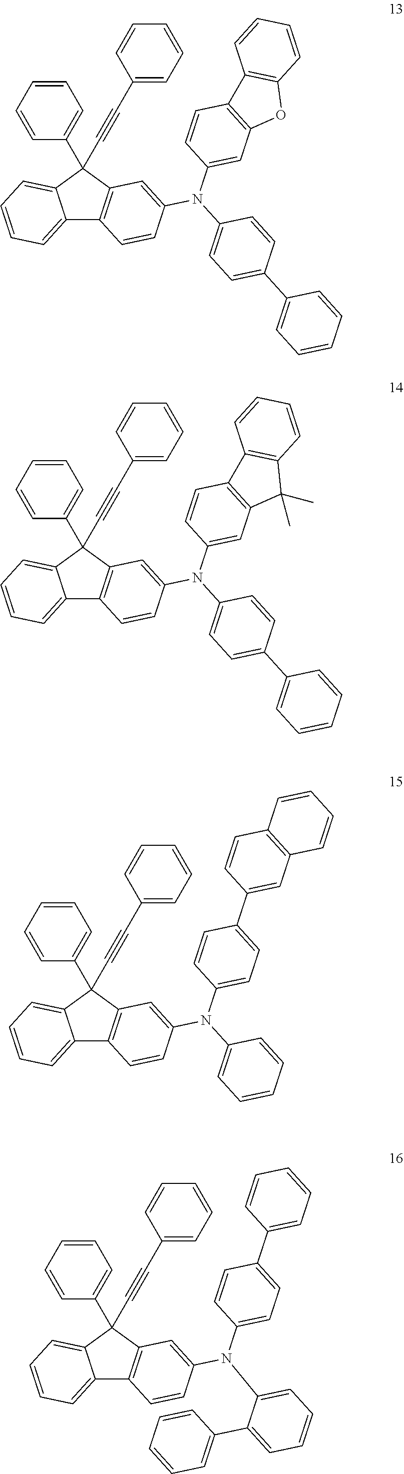 [SMILES: C(#CC1(c2ccccc2)c2ccccc2-c2ccc(N(c3ccc(-c4ccccc4)cc3)c3ccc4c(c3)oc3ccccc34)cc21)c1ccccc1 ; CC1(C)c2ccccc2-c2ccc(N(c3ccc(-c4ccccc4)cc3)c3ccc4c(c3)C(C#Cc3ccccc3)(c3ccccc3)c3ccccc3-4)cc21 ; C(#CC1(c2ccccc2)c2ccccc2-c2ccc(N(c3ccccc3)c3ccc(-c4ccc5ccccc5c4)cc3)cc21)c1ccccc1 ; C(#CC1(c2ccccc2)c2ccccc2-c2ccc(N(c3ccc(-c4ccccc4)cc3)c3ccccc3-c3ccccc3)cc21)c1ccccc1]

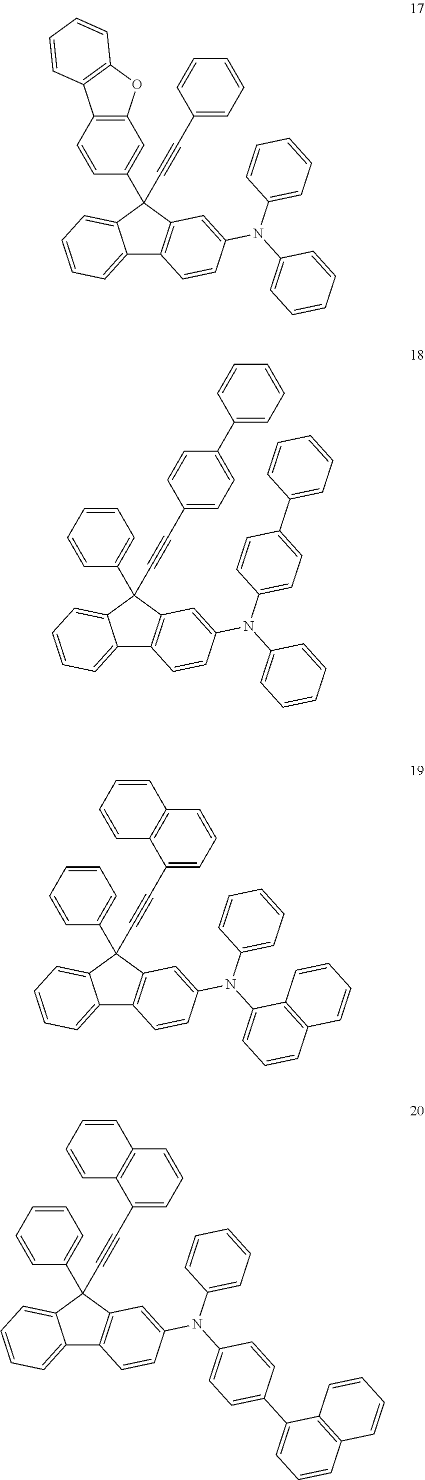 [SMILES: C(#CC1(c2ccc3c(c2)oc2ccccc23)c2ccccc2-c2ccc(N(c3ccccc3)c3ccccc3)cc21)c1ccccc1 ; C(#CC1(c2ccccc2)c2ccccc2-c2ccc(N(c3ccccc3)c3ccc(-c4ccccc4)cc3)cc21)c1ccc(-c2ccccc2)cc1 ; C(#CC1(c2ccccc2)c2ccccc2-c2ccc(N(c3ccccc3)c3cccc4ccccc34)cc21)c1cccc2ccccc12 ; C(#CC1(c2ccccc2)c2ccccc2-c2ccc(N(c3ccccc3)c3ccc(-c4cccc5ccccc45)cc3)cc21)c1cccc2ccccc12]

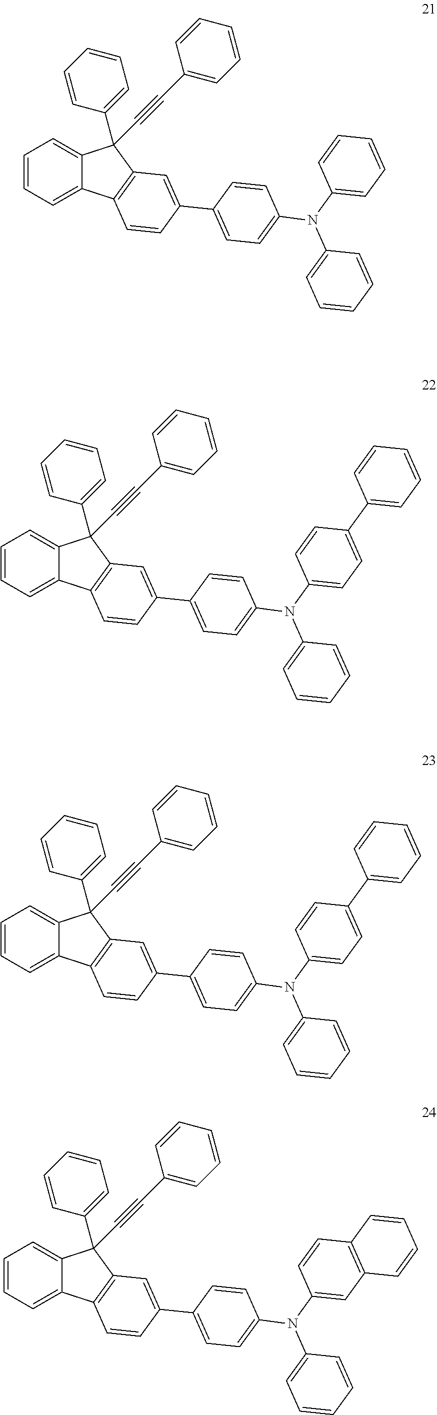 [SMILES: C(#CC1(c2ccccc2)c2ccccc2-c2ccc(-c3ccc(N(c4ccccc4)c4ccccc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccccc4)c4ccc5ccccc5c4)cc3)cc21)c1ccccc1]

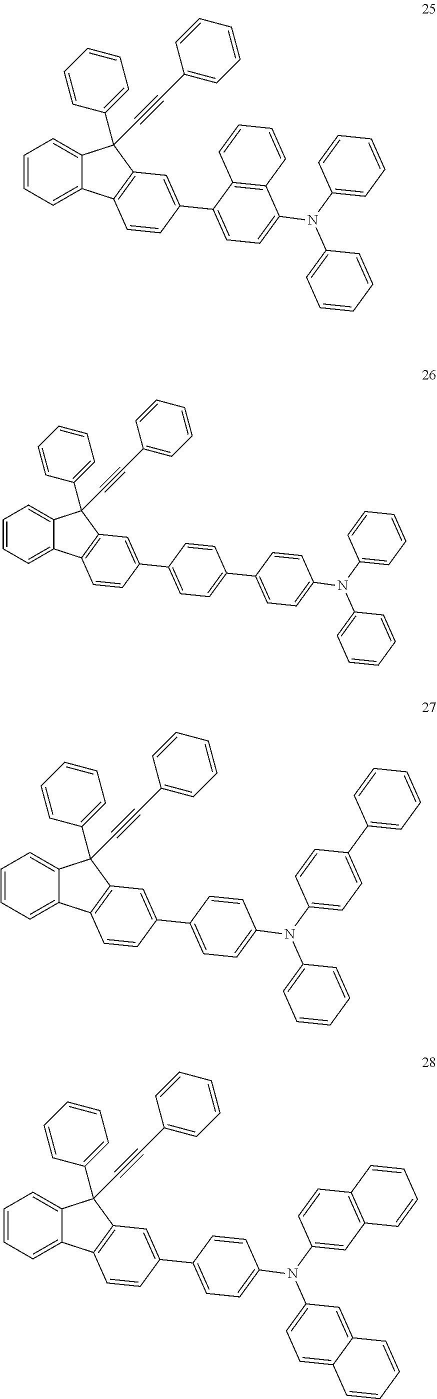 [SMILES: C(#CC1(c2ccccc2)c2ccccc2-c2ccc(-c3ccc(N(c4ccccc4)c4ccccc4)c4ccccc34)cc21)c1ccccc1 ; C(#CC1(c2ccccc2)c2ccccc2-c2ccc(-c3ccc(-c4ccc(N(c5ccccc5)c5ccccc5)cc4)cc3)cc21)c1ccccc1 ; C(#CC1(c2ccccc2)c2ccccc2-c2ccc(-c3ccc(N(c4ccccc4)c4ccc(-c5ccccc5)cc4)cc3)cc21)c1ccccc1 ; C(#CC1(c2ccccc2)c2ccccc2-c2ccc(-c3ccc(N(c4ccc5ccccc5c4)c4ccc5ccccc5c4)cc3)cc21)c1ccccc1]

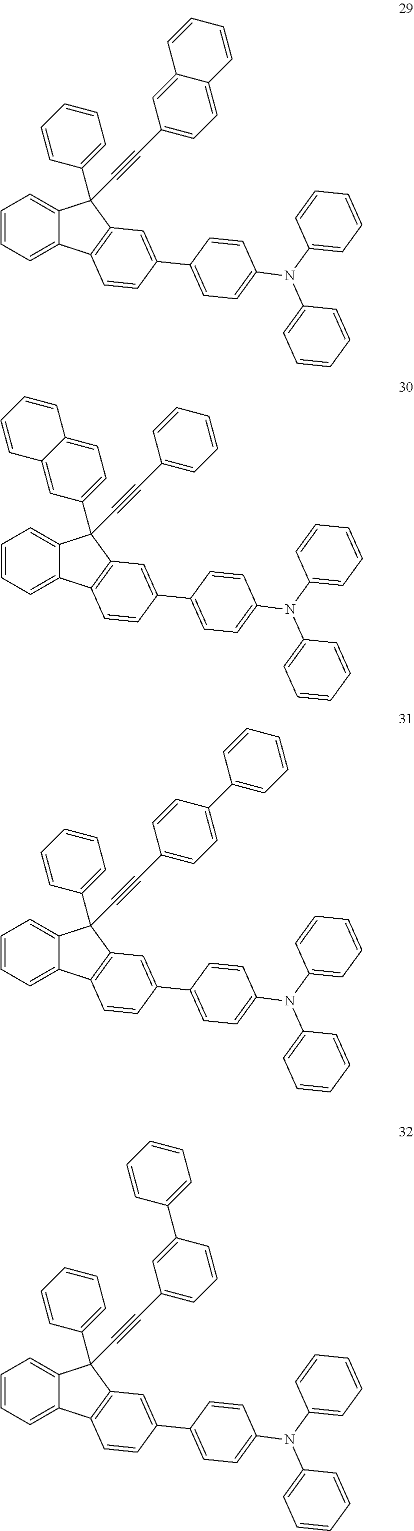

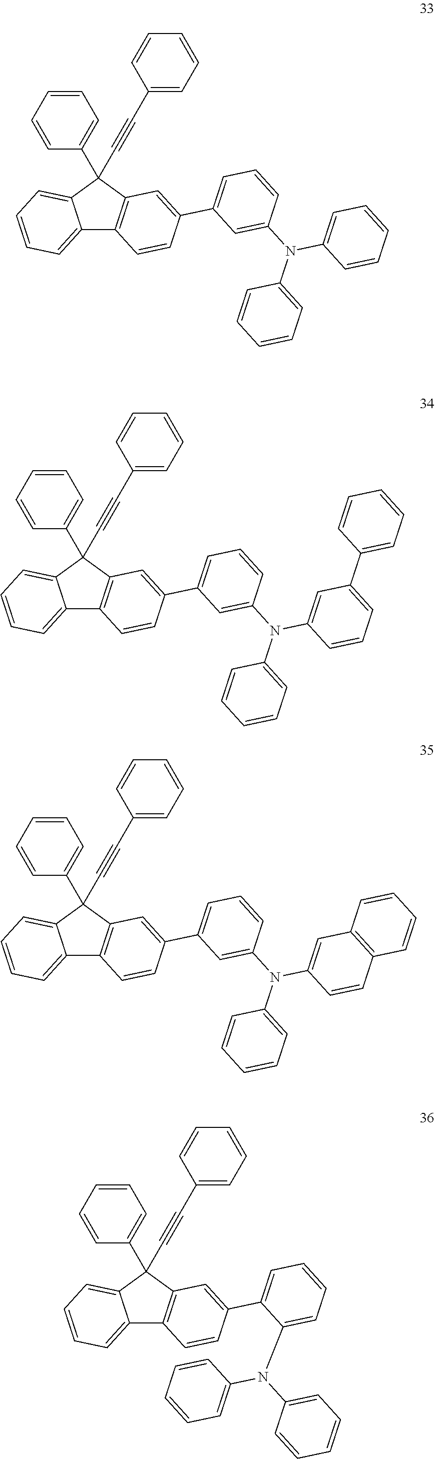 [SMILES: C(#CC1(c2ccccc2)c2ccccc2-c2ccc(-c3cccc(N(c4ccccc4)c4ccccc4)c3)cc21)c1ccccc1 ; C(#CC1(c2ccccc2)c2ccccc2-c2ccc(-c3cccc(N(c4ccccc4)c4cccc(-c5ccccc5)c4)c3)cc21)c1ccccc1 ; C(#CC1(c2ccccc2)c2ccccc2-c2ccc(-c3cccc(N(c4ccccc4)c4ccc5ccccc5c4)c3)cc21)c1ccccc1 ; C(#CC1(c2ccccc2)c2ccccc2-c2ccc(-c3ccccc3N(c3ccccc3)c3ccccc3)cc21)c1ccccc1]

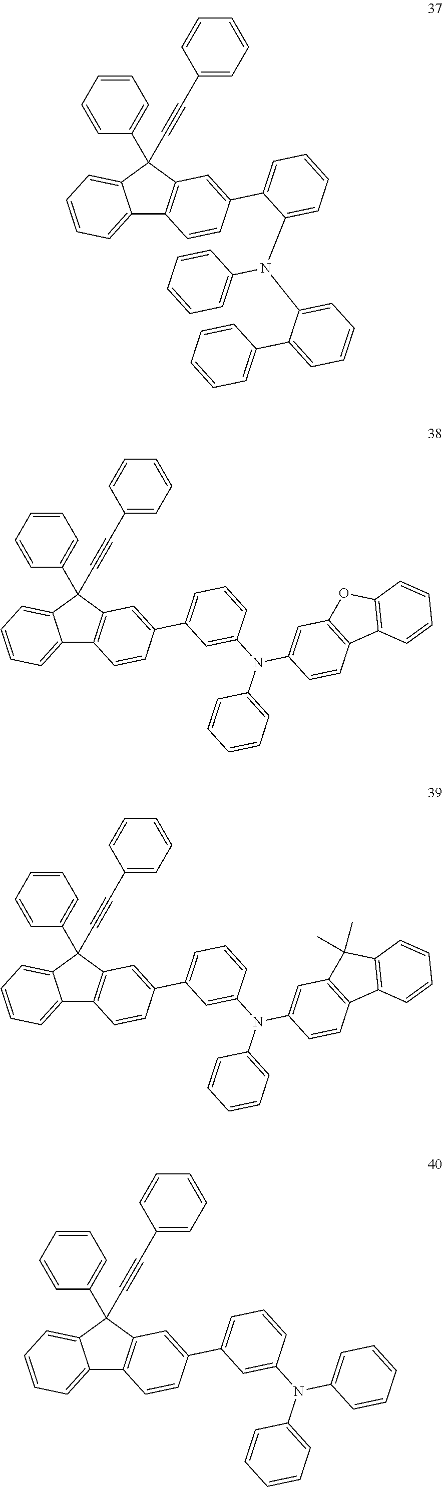 [SMILES: C(#CC1(c2ccccc2)c2ccccc2-c2ccc(-c3ccccc3N(c3ccccc3)c3ccccc3-c3ccccc3)cc21)c1ccccc1 ; C(#CC1(c2ccccc2)c2ccccc2-c2ccc(-c3cccc(N(c4ccccc4)c4ccc5c(c4)oc4ccccc45)c3)cc21)c1ccccc1 ; CC1(C)c2ccccc2-c2ccc(N(c3ccccc3)c3cccc(-c4ccc5c(c4)C(C#Cc4ccccc4)(c4ccccc4)c4ccccc4-5)c3)cc21 ; C(#CC1(c2ccccc2)c2ccccc2-c2ccc(-c3cccc(N(c4ccccc4)c4ccccc4)c3)cc21)c1ccccc1]

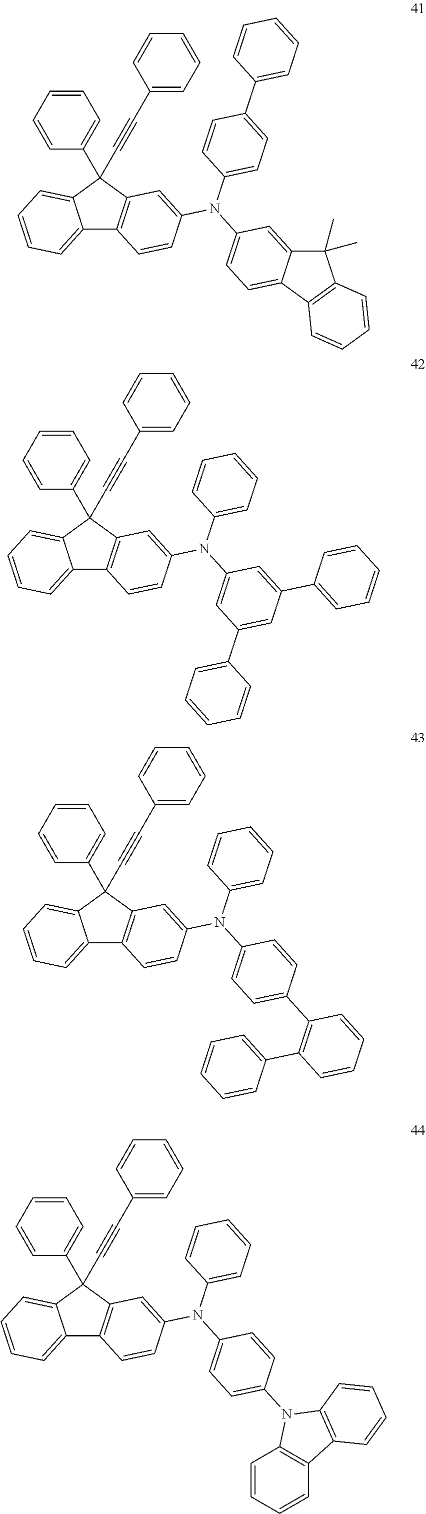

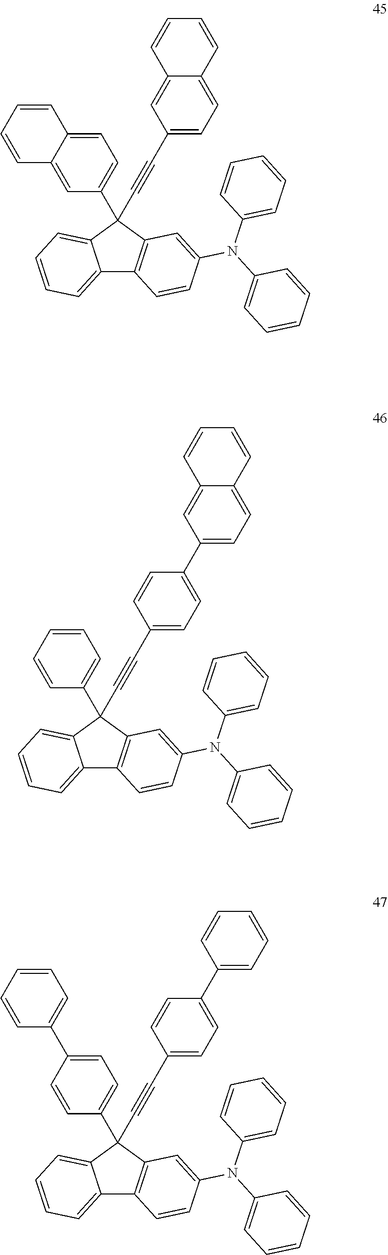 [SMILES: C(#CC1(c2ccc3ccccc3c2)c2ccccc2-c2ccc(N(c3ccccc3)c3ccccc3)cc21)c1ccc2ccccc2c1 ; C(#CC1(c2ccccc2)c2ccccc2-c2ccc(N(c3ccccc3)c3ccccc3)cc21)c1ccc(-c2ccc3ccccc3c2)cc1 ; C(#CC1(c2ccc(-c3ccccc3)cc2)c2ccccc2-c2ccc(N(c3ccccc3)c3ccccc3)cc21)c1ccc(-c2ccccc2)cc1]

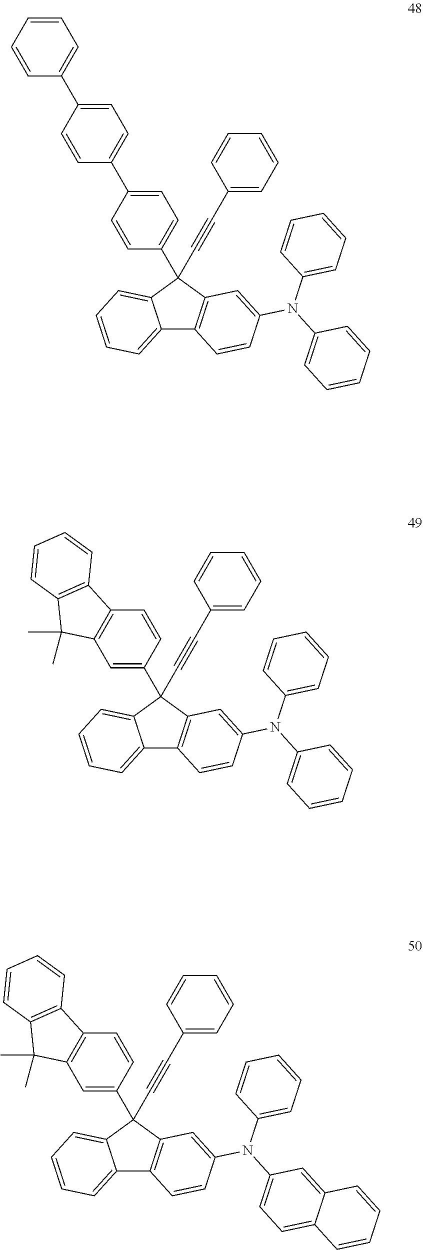 [SMILES: C(#CC1(c2ccc(-c3ccc(-c4ccccc4)cc3)cc2)c2ccccc2-c2ccc(N(c3ccccc3)c3ccccc3)cc21)c1ccccc1 ; CC1(C)c2ccccc2-c2ccc(C3(C#Cc4ccccc4)c4ccccc4-c4ccc(N(c5ccccc5)c5ccccc5)cc43)cc21 ; CC1(C)c2ccccc2-c2ccc(C3(C#Cc4ccccc4)c4ccccc4-c4ccc(N(c5ccccc5)c5ccc6ccccc6c5)cc43)cc21]

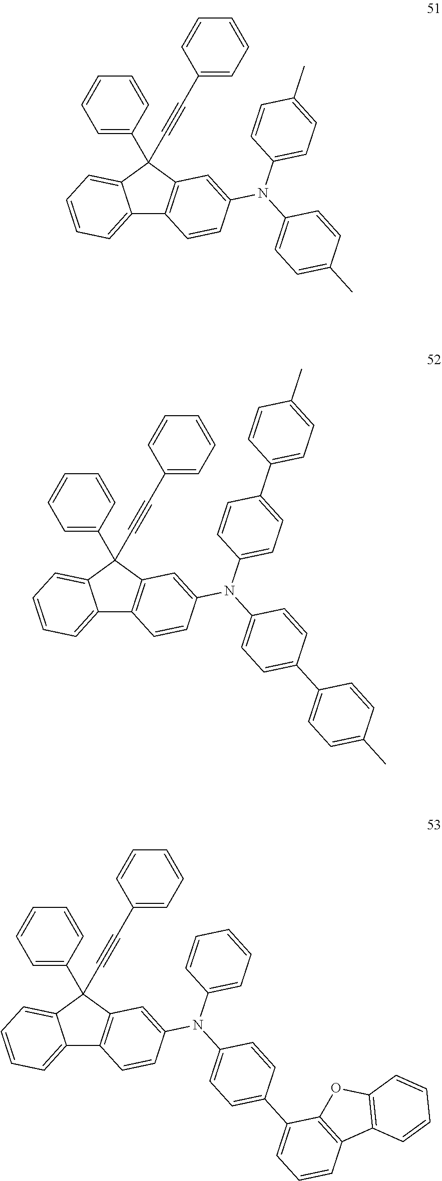 [SMILES: Cc1ccc(N(c2ccc(C)cc2)c2ccc3c(c2)C(C#Cc2ccccc2)(c2ccccc2)c2ccccc2-3)cc1 ; Cc1ccc(-c2ccc(N(c3ccc(-c4ccc(C)cc4)cc3)c3ccc4c(c3)C(C#Cc3ccccc3)(c3ccccc3)c3ccccc3-4)cc2)cc1 ; C(#CC1(c2ccccc2)c2ccccc2-c2ccc(N(c3ccccc3)c3ccc(-c4cccc5c4oc4ccccc45)cc3)cc21)c1ccccc1]

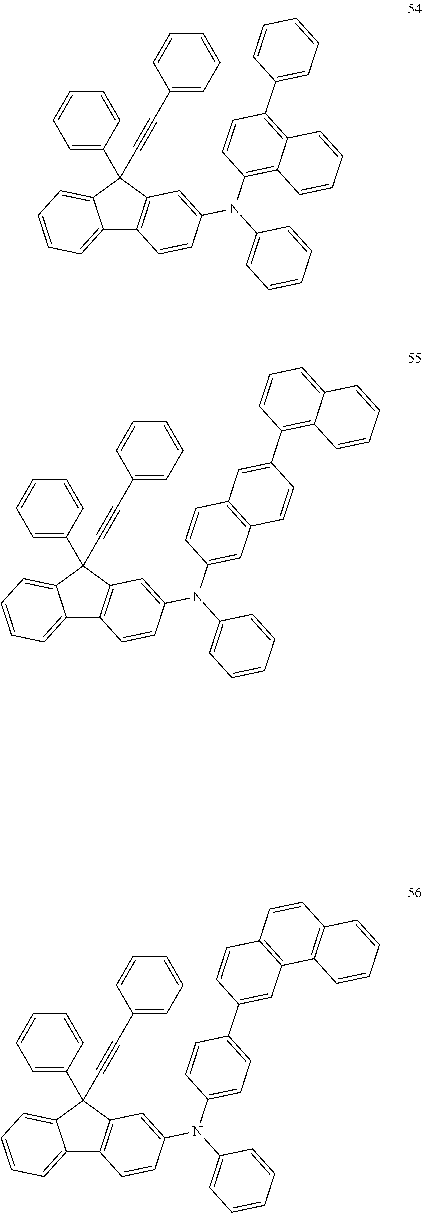 [SMILES: C(#CC1(c2ccccc2)c2ccccc2-c2ccc(N(c3ccccc3)c3ccc(-c4ccccc4)c4ccccc34)cc21)c1ccccc1 ; C(#CC1(c2ccccc2)c2ccccc2-c2ccc(N(c3ccccc3)c3ccc4cc(-c5cccc6ccccc56)ccc4c3)cc21)c1ccccc1 ; C(#CC1(c2ccccc2)c2ccccc2-c2ccc(N(c3ccccc3)c3ccc(-c4ccc5ccc6ccccc6c5c4)cc3)cc21)c1ccccc1]

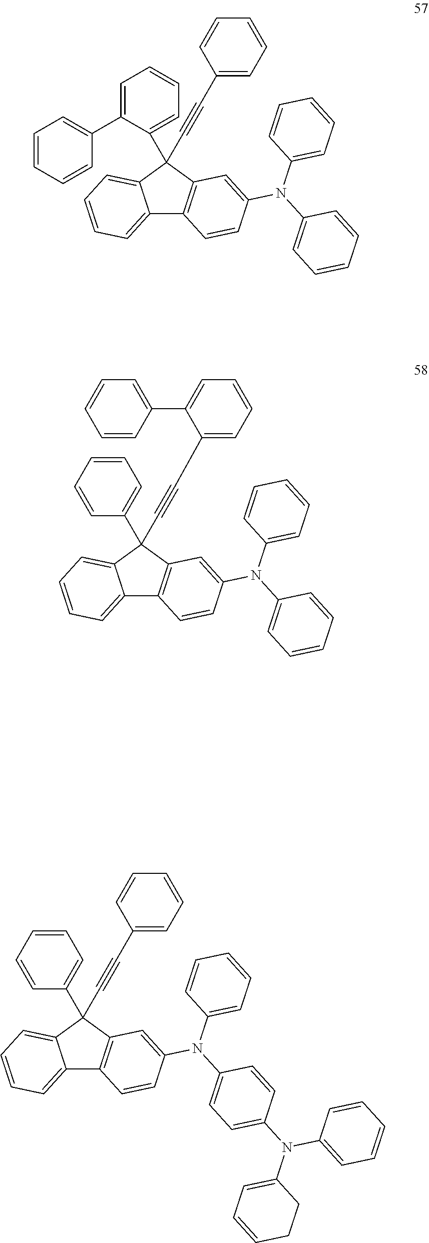 [SMILES: C(#CC1(c2ccccc2-c2ccccc2)c2ccccc2-c2ccc(N(c3ccccc3)c3ccccc3)cc21)c1ccccc1 ; C(#CC1(c2ccccc2)c2ccccc2-c2ccc(N(c3ccccc3)c3ccccc3)cc21)c1ccccc1-c1ccccc1 ; C(#CC1(c2ccccc2)c2ccccc2-c2ccc(N(c3ccccc3)c3ccc(N(C4=CC=CCC4)c4ccccc4)cc3)cc21)c1ccccc1]

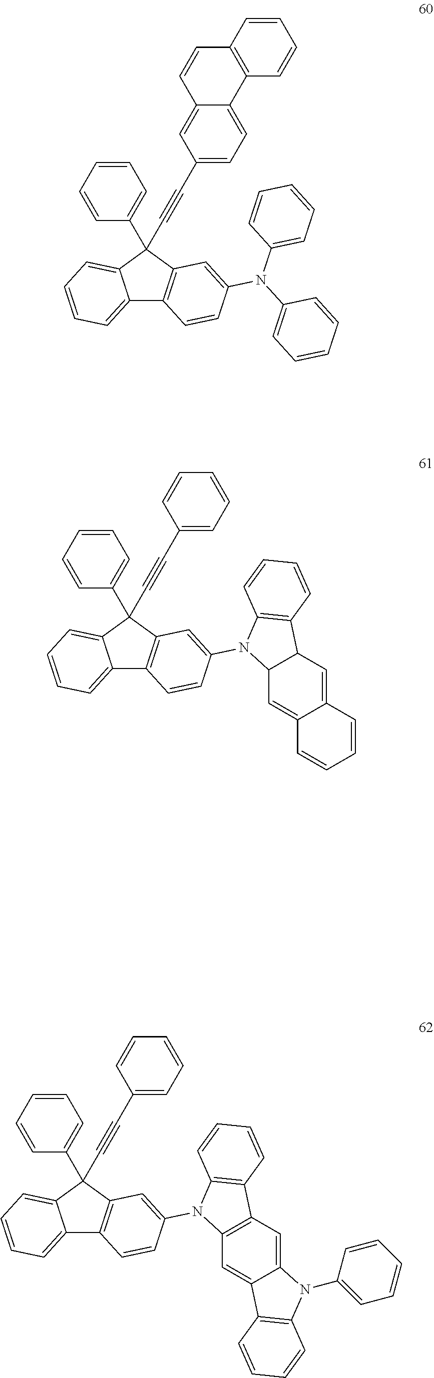 [SMILES: C(#CC1(c2ccccc2)c2ccccc2-c2ccc(N(c3ccccc3)c3ccccc3)cc21)c1ccc2c(ccc3ccccc32)c1 ; C(#CC1(c2ccccc2)c2ccccc2-c2ccc(N3c4ccccc4C4C=c5ccccc5=CC43)cc21)c1ccccc1 ; C(#CC1(c2ccccc2)c2ccccc2-c2ccc(-n3c4ccccc4c4cc5c(cc43)c3ccccc3n5-c3ccccc3)cc21)c1ccccc1]

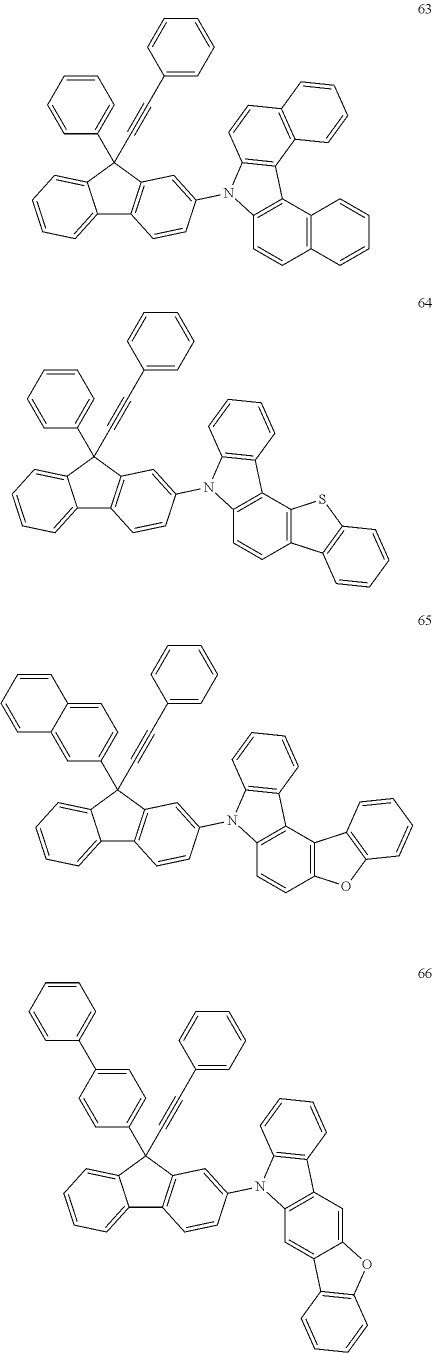 [SMILES: C(#CC1(c2ccccc2)c2ccccc2-c2ccc(-n3c4ccc5ccccc5c4c4c5ccccc5ccc43)cc21)c1ccccc1 ; C(#CC1(c2ccccc2)c2ccccc2-c2ccc(-n3c4ccccc4c4c5sc6ccccc6c5ccc43)cc21)c1ccccc1 ; C(#CC1(c2ccc3ccccc3c2)c2ccccc2-c2ccc(-n3c4ccccc4c4c5c(ccc43)oc3ccccc35)cc21)c1ccccc1 ; C(#CC1(c2ccc(-c3ccccc3)cc2)c2ccccc2-c2ccc(-n3c4ccccc4c4cc5oc6ccccc6c5cc43)cc21)c1ccccc1]

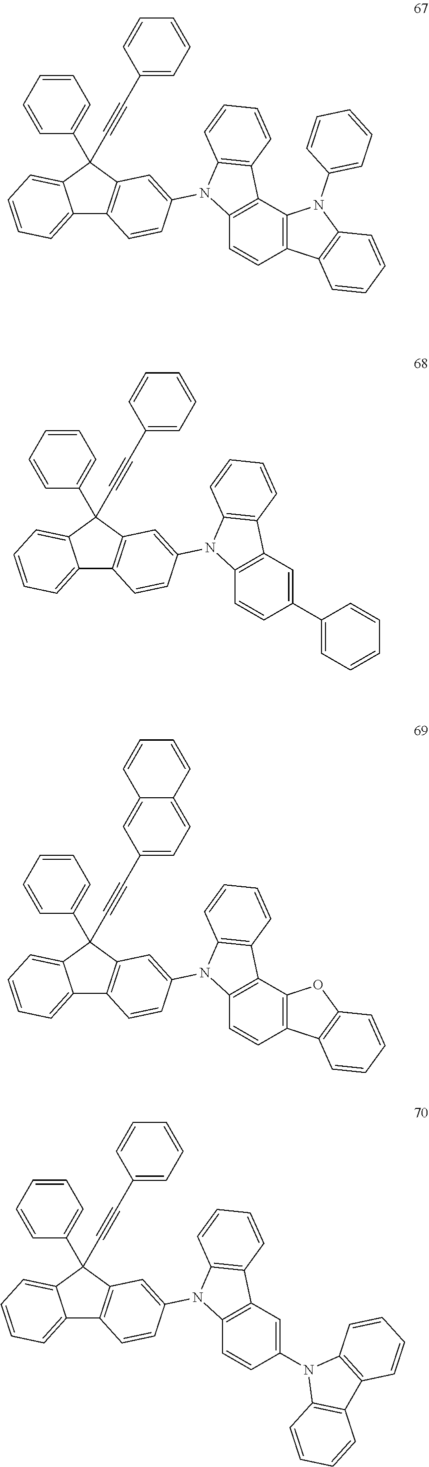 [SMILES: C(#CC1(c2ccccc2)c2ccccc2-c2ccc(-n3c4ccccc4c4c3ccc3c5ccccc5n(-c5ccccc5)c34)cc21)c1ccccc1 ; C(#CC1(c2ccccc2)c2ccccc2-c2ccc(-n3c4ccccc4c4cc(-c5ccccc5)ccc43)cc21)c1ccccc1 ; C(#CC1(c2ccccc2)c2ccccc2-c2ccc(-n3c4ccccc4c4c5oc6ccccc6c5ccc43)cc21)c1ccc2ccccc2c1 ; C(#CC1(c2ccccc2)c2ccccc2-c2ccc(-n3c4ccccc4c4cc(-n5c6ccccc6c6ccccc65)ccc43)cc21)c1ccccc1]

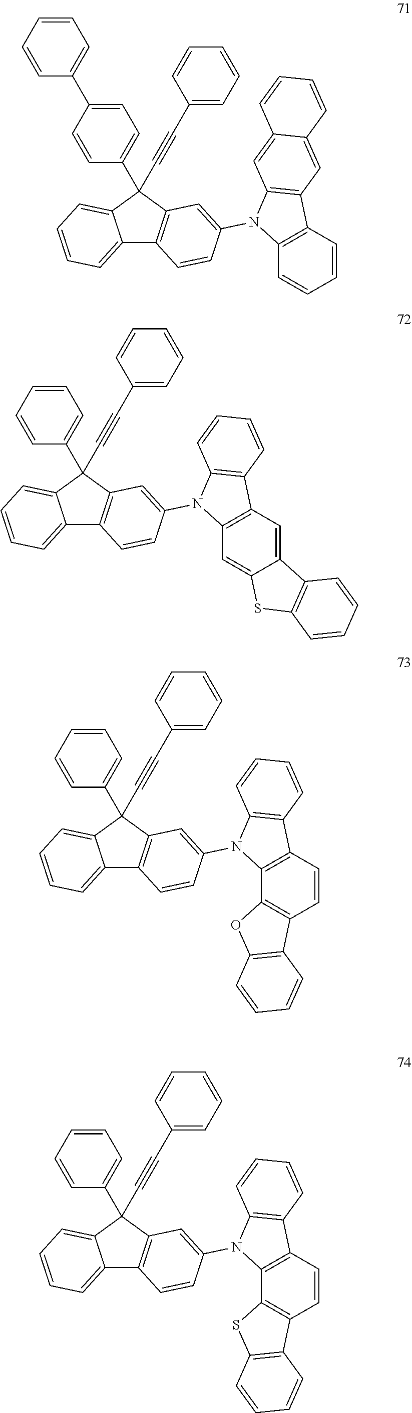 [SMILES: C(#CC1(c2ccc(-c3ccccc3)cc2)c2ccccc2-c2ccc(-n3c4ccccc4c4cc5ccccc5cc43)cc21)c1ccccc1 ; C(#CC1(c2ccccc2)c2ccccc2-c2ccc(-n3c4ccccc4c4cc5c(cc43)sc3ccccc35)cc21)c1ccccc1 ; C(#CC1(c2ccccc2)c2ccccc2-c2ccc(-n3c4ccccc4c4ccc5c6ccccc6oc5c43)cc21)c1ccccc1 ; C(#CC1(c2ccccc2)c2ccccc2-c2ccc(-n3c4ccccc4c4ccc5c6ccccc6sc5c43)cc21)c1ccccc1]

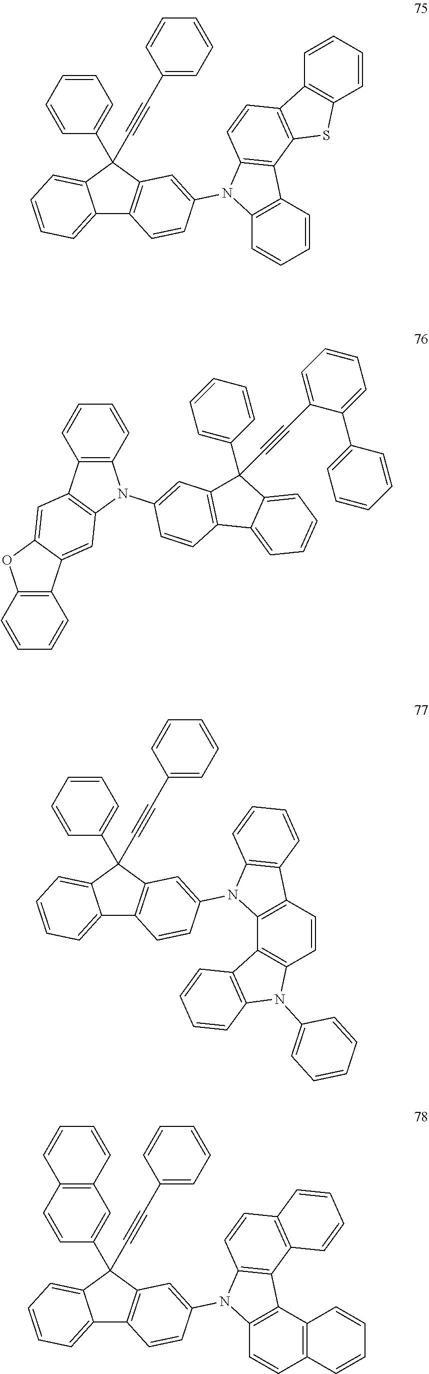 [SMILES: C(#CC1(c2ccccc2)c2ccccc2-c2ccc(-n3c4ccccc4c4c5sc6ccccc6c5ccc43)cc21)c1ccccc1 ; C(#CC1(c2ccccc2)c2ccccc2-c2ccc(-n3c4ccccc4c4cc5oc6ccccc6c5cc43)cc21)c1ccccc1-c1ccccc1 ; C(#CC1(c2ccccc2)c2ccccc2-c2ccc(-n3c4ccccc4c4ccc5c(c6ccccc6n5-c5ccccc5)c43)cc21)c1ccccc1 ; C(#CC1(c2ccc3ccccc3c2)c2ccccc2-c2ccc(-n3c4ccc5ccccc5c4c4c5ccccc5ccc43)cc21)c1ccccc1]

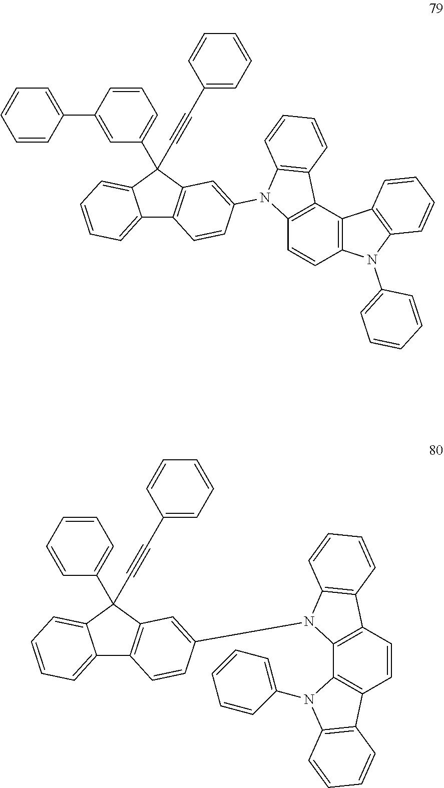 [SMILES: C(#CC1(c2cccc(-c3ccccc3)c2)c2ccccc2-c2ccc(-n3c4ccccc4c4c5c6ccccc6n(-c6ccccc6)c5ccc43)cc21)c1ccccc1 ; C(#CC1(c2ccccc2)c2ccccc2-c2ccc(-n3c4ccccc4c4ccc5c6ccccc6n(-c6ccccc6)c5c43)cc21)c1ccccc1]

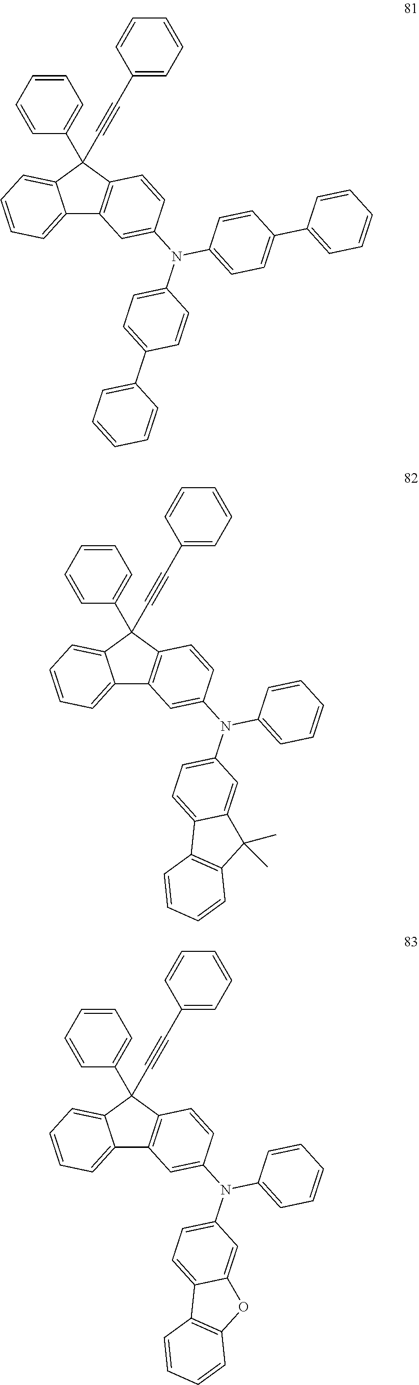

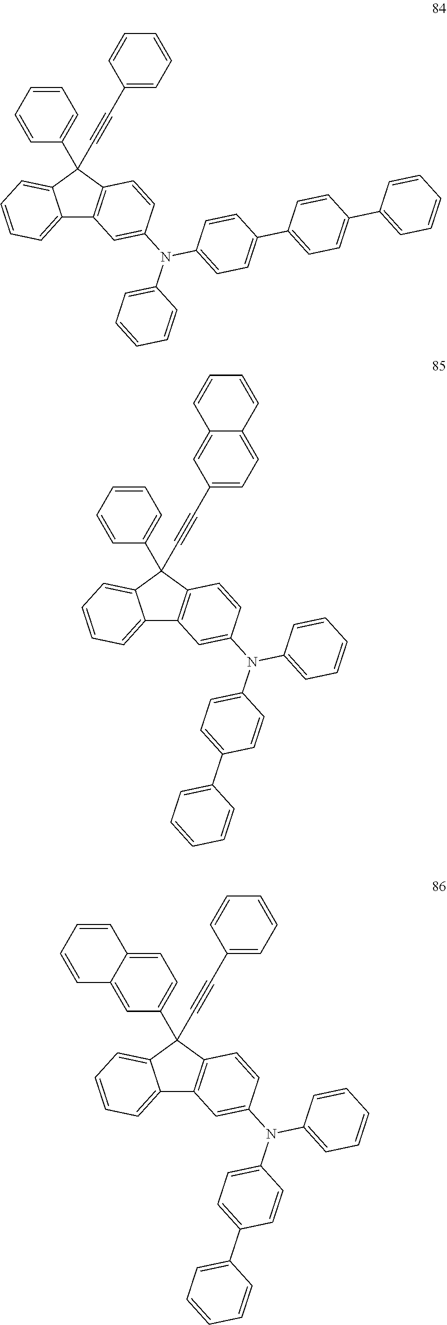 [SMILES: C(#CC1(c2ccccc2)c2ccccc2-c2cc(N(c3ccccc3)c3ccc(-c4ccc(-c5ccccc5)cc4)cc3)ccc21)c1ccccc1 ; C(#CC1(c2ccccc2)c2ccccc2-c2cc(N(c3ccccc3)c3ccc(-c4ccccc4)cc3)ccc21)c1ccc2ccccc2c1 ; C(#CC1(c2ccc3ccccc3c2)c2ccccc2-c2cc(N(c3ccccc3)c3ccc(-c4ccccc4)cc3)ccc21)c1ccccc1]

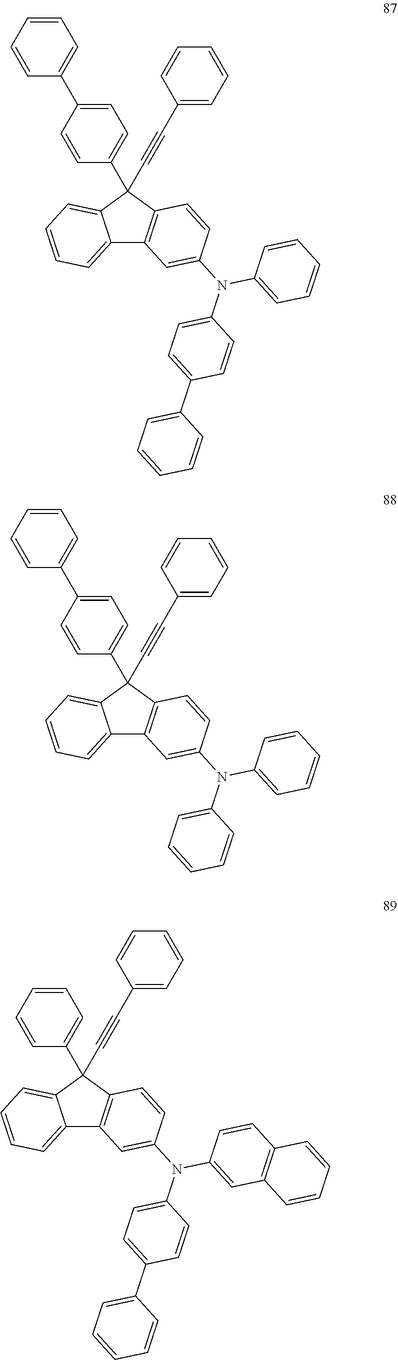 [SMILES: C(#CC1(c2ccc(-c3ccccc3)cc2)c2ccccc2-c2cc(N(c3ccccc3)c3ccc(-c4ccccc4)cc3)ccc21)c1ccccc1 ; C(#CC1(c2ccc(-c3ccccc3)cc2)c2ccccc2-c2cc(N(c3ccccc3)c3ccccc3)ccc21)c1ccccc1 ; C(#CC1(c2ccccc2)c2ccccc2-c2cc(N(c3ccc(-c4ccccc4)cc3)c3ccc4ccccc4c3)ccc21)c1ccccc1]

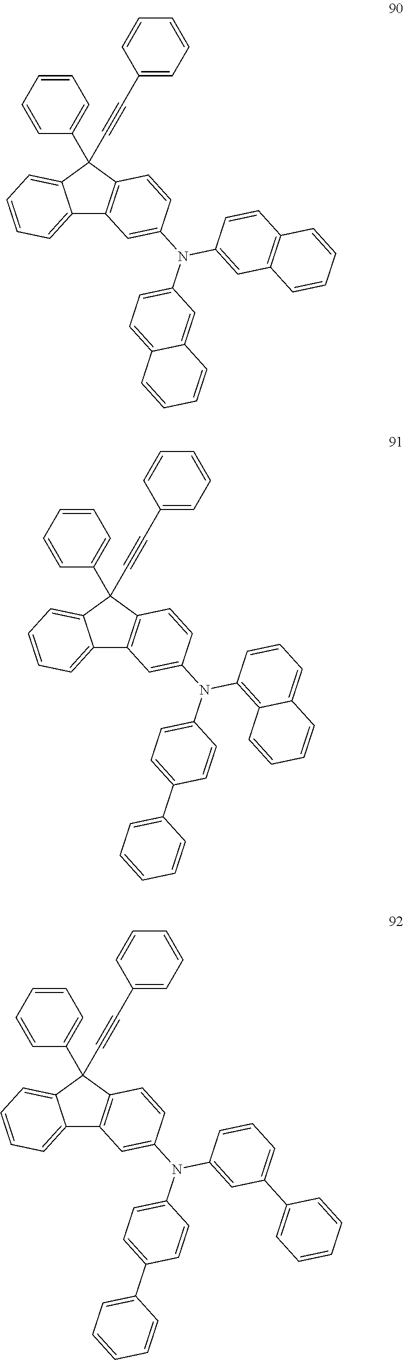

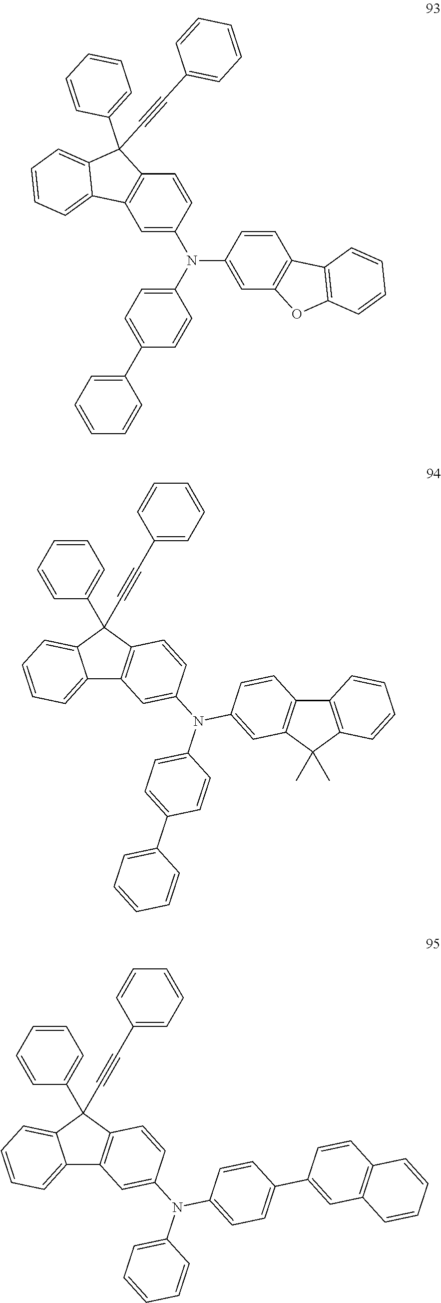 [SMILES: C(#CC1(c2ccccc2)c2ccccc2-c2cc(N(c3ccc(-c4ccccc4)cc3)c3ccc4c(c3)oc3ccccc34)ccc21)c1ccccc1 ; CC1(C)c2ccccc2-c2ccc(N(c3ccc(-c4ccccc4)cc3)c3ccc4c(c3)-c3ccccc3C4(C#Cc3ccccc3)c3ccccc3)cc21 ; C(#CC1(c2ccccc2)c2ccccc2-c2cc(N(c3ccccc3)c3ccc(-c4ccc5ccccc5c4)cc3)ccc21)c1ccccc1]

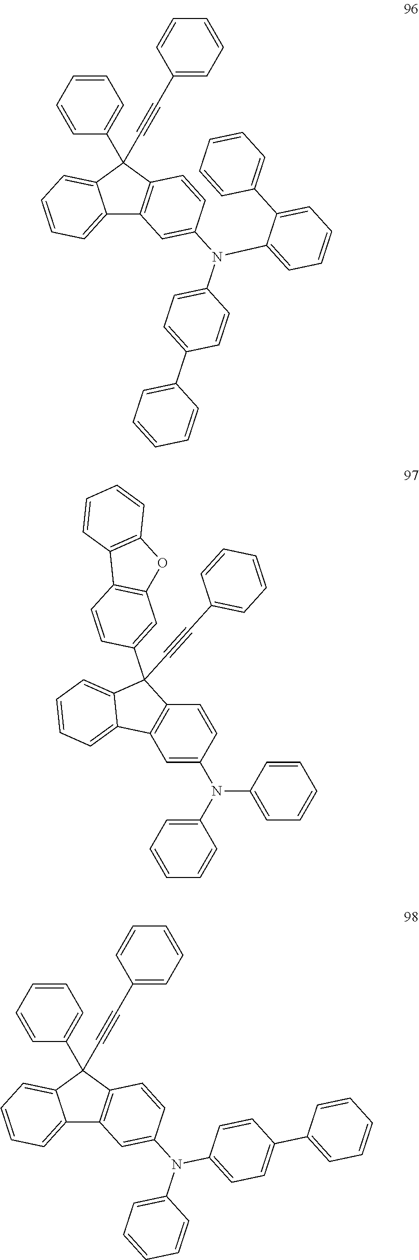 [SMILES: C(#CC1(c2ccccc2)c2ccccc2-c2cc(N(c3ccc(-c4ccccc4)cc3)c3ccccc3-c3ccccc3)ccc21)c1ccccc1 ; C(#CC1(c2ccc3c(c2)oc2ccccc23)c2ccccc2-c2cc(N(c3ccccc3)c3ccccc3)ccc21)c1ccccc1 ; C(#CC1(c2ccccc2)c2ccccc2-c2cc(N(c3ccccc3)c3ccc(-c4ccccc4)cc3)ccc21)c1ccccc1]

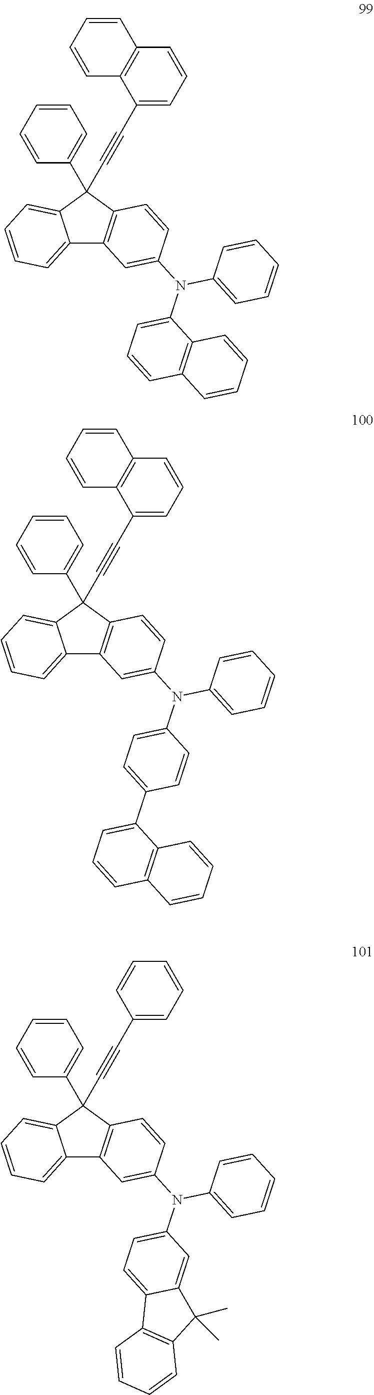 [SMILES: C(#CC1(c2ccccc2)c2ccccc2-c2cc(N(c3ccccc3)c3cccc4ccccc34)ccc21)c1cccc2ccccc12 ; C(#CC1(c2ccccc2)c2ccccc2-c2cc(N(c3ccccc3)c3ccc(-c4cccc5ccccc45)cc3)ccc21)c1cccc2ccccc12 ; CC1(C)c2ccccc2-c2ccc(N(c3ccccc3)c3ccc4c(c3)-c3ccccc3C4(C#Cc3ccccc3)c3ccccc3)cc21]

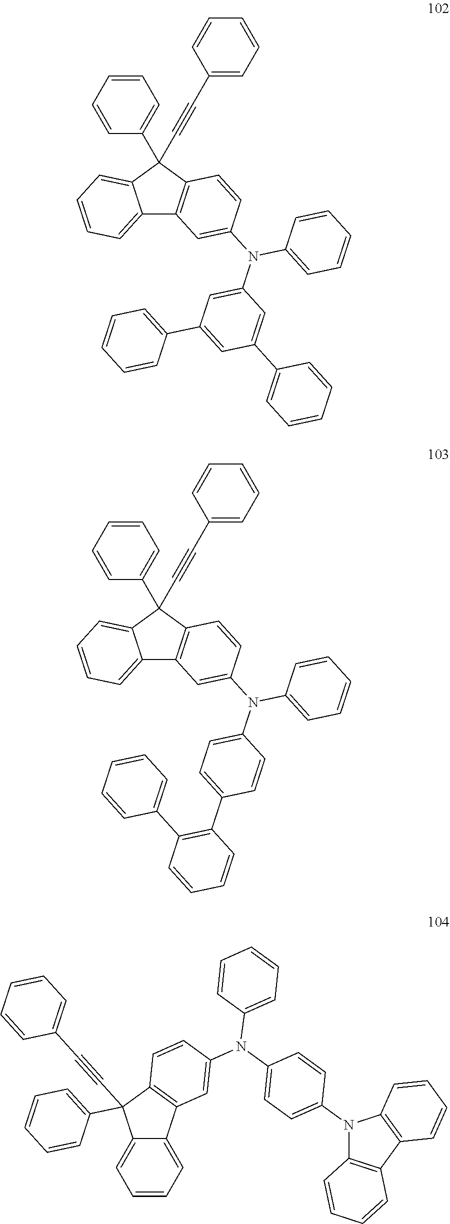 [SMILES: C(#CC1(c2ccccc2)c2ccccc2-c2cc(N(c3ccccc3)c3cc(-c4ccccc4)cc(-c4ccccc4)c3)ccc21)c1ccccc1 ; C(#CC1(c2ccccc2)c2ccccc2-c2cc(N(c3ccccc3)c3ccc(-c4ccccc4-c4ccccc4)cc3)ccc21)c1ccccc1 ; C(#CC1(c2ccccc2)c2ccccc2-c2cc(N(c3ccccc3)c3ccc(-n4c5ccccc5c5ccccc54)cc3)ccc21)c1ccccc1]

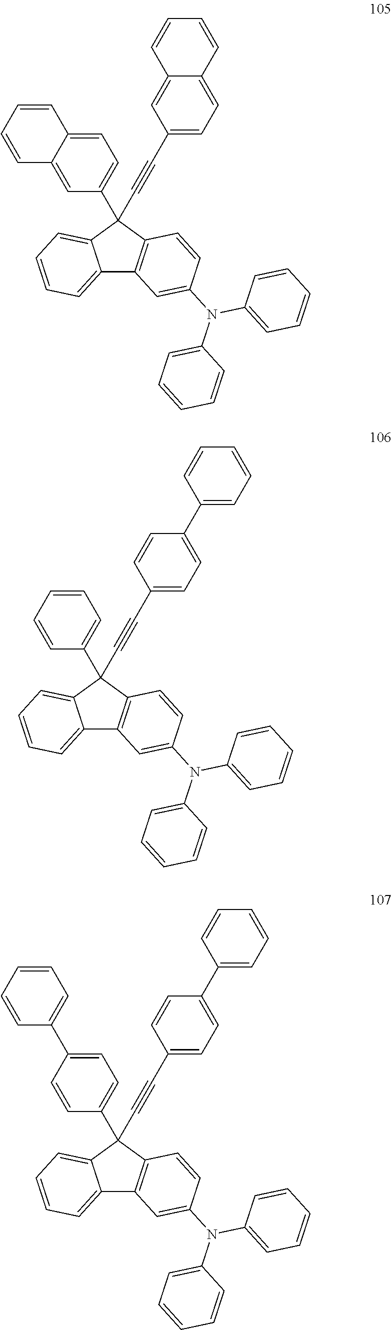

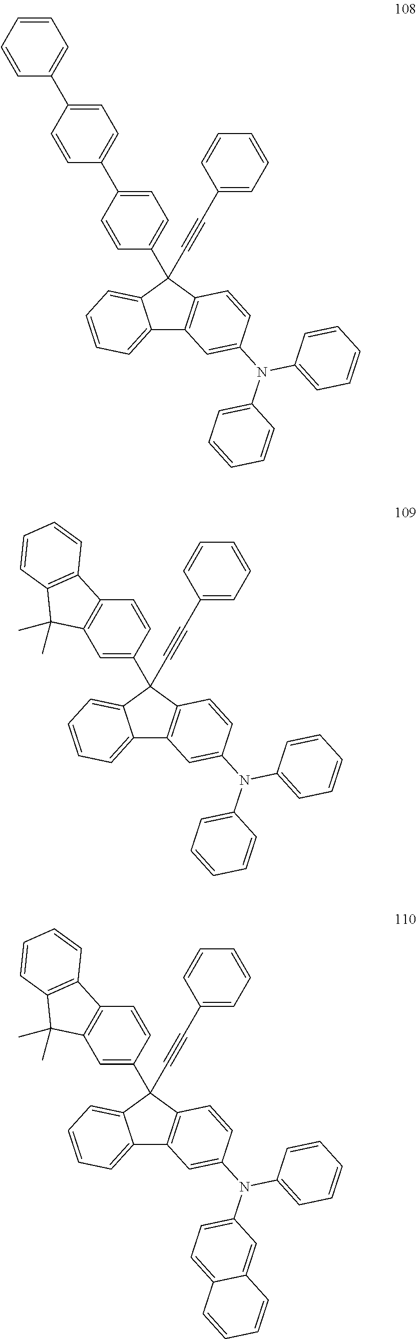 [SMILES: C(#CC1(c2ccc(-c3ccc(-c4ccccc4)cc3)cc2)c2ccccc2-c2cc(N(c3ccccc3)c3ccccc3)ccc21)c1ccccc1 ; CC1(C)c2ccccc2-c2ccc(C3(C#Cc4ccccc4)c4ccccc4-c4cc(N(c5ccccc5)c5ccccc5)ccc43)cc21 ; CC1(C)c2ccccc2-c2ccc(C3(C#Cc4ccccc4)c4ccccc4-c4cc(N(c5ccccc5)c5ccc6ccccc6c5)ccc43)cc21]

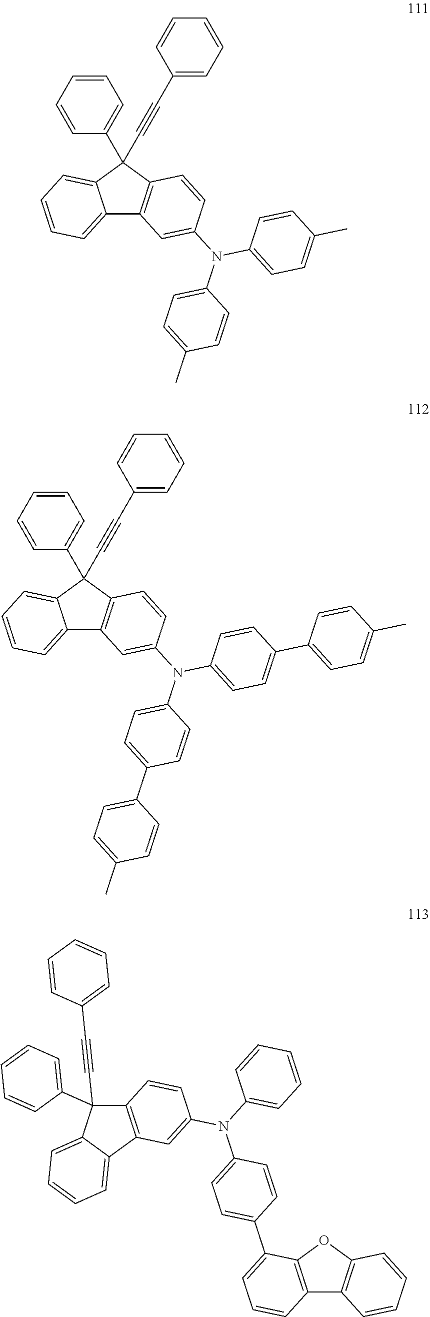 [SMILES: Cc1ccc(N(c2ccc(C)cc2)c2ccc3c(c2)-c2ccccc2C3(C#Cc2ccccc2)c2ccccc2)cc1 ; Cc1ccc(-c2ccc(N(c3ccc(-c4ccc(C)cc4)cc3)c3ccc4c(c3)-c3ccccc3C4(C#Cc3ccccc3)c3ccccc3)cc2)cc1 ; C(#CC1(c2ccccc2)c2ccccc2-c2cc(N(c3ccccc3)c3ccc(-c4cccc5c4oc4ccccc45)cc3)ccc21)c1ccccc1]

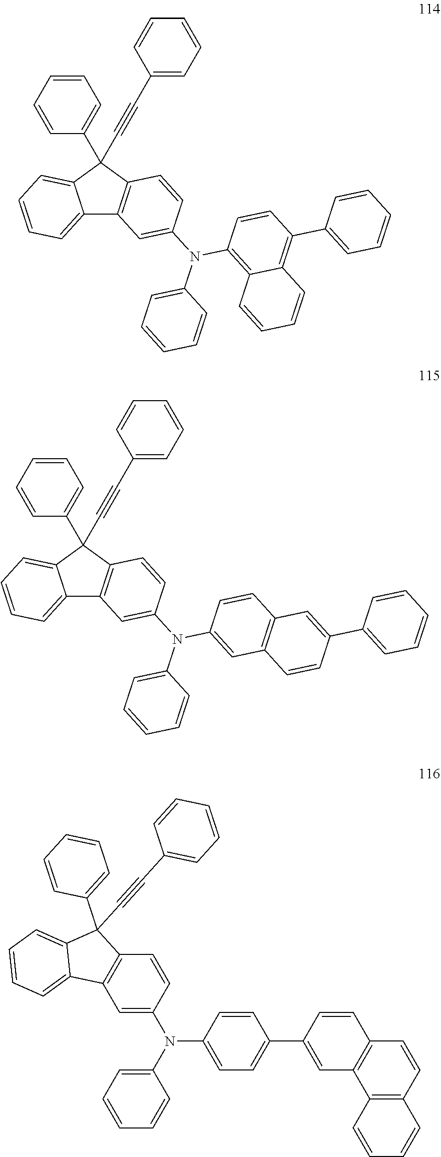 [SMILES: C(#CC1(c2ccccc2)c2ccccc2-c2cc(N(c3ccccc3)c3ccc(-c4ccccc4)c4ccccc34)ccc21)c1ccccc1 ; C(#CC1(c2ccccc2)c2ccccc2-c2cc(N(c3ccccc3)c3ccc4cc(-c5ccccc5)ccc4c3)ccc21)c1ccccc1 ; C(#CC1(c2ccccc2)c2ccccc2-c2cc(N(c3ccccc3)c3ccc(-c4ccc5ccc6ccccc6c5c4)cc3)ccc21)c1ccccc1]

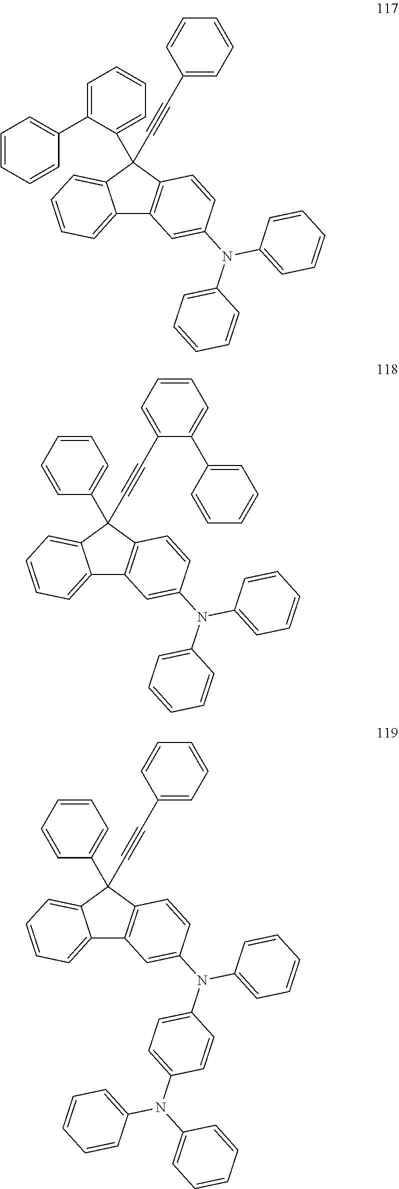

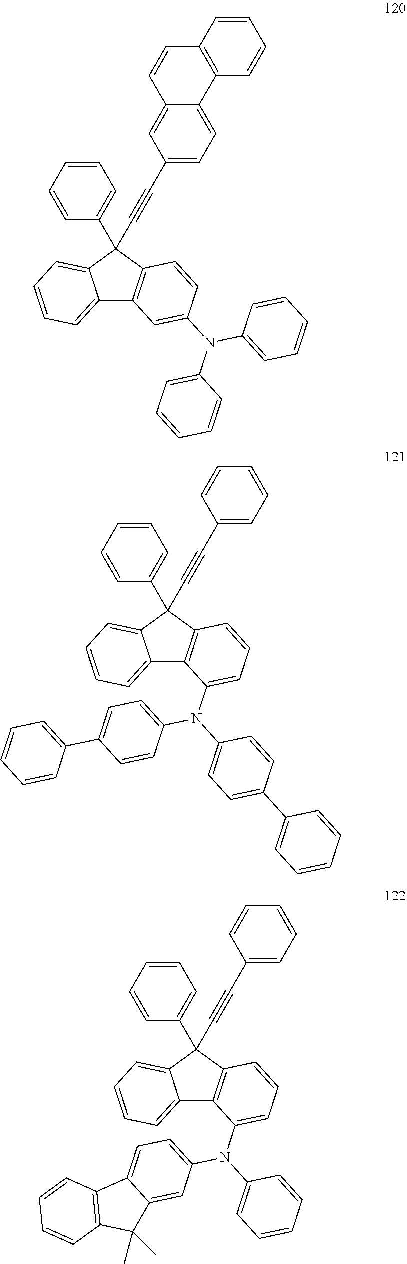

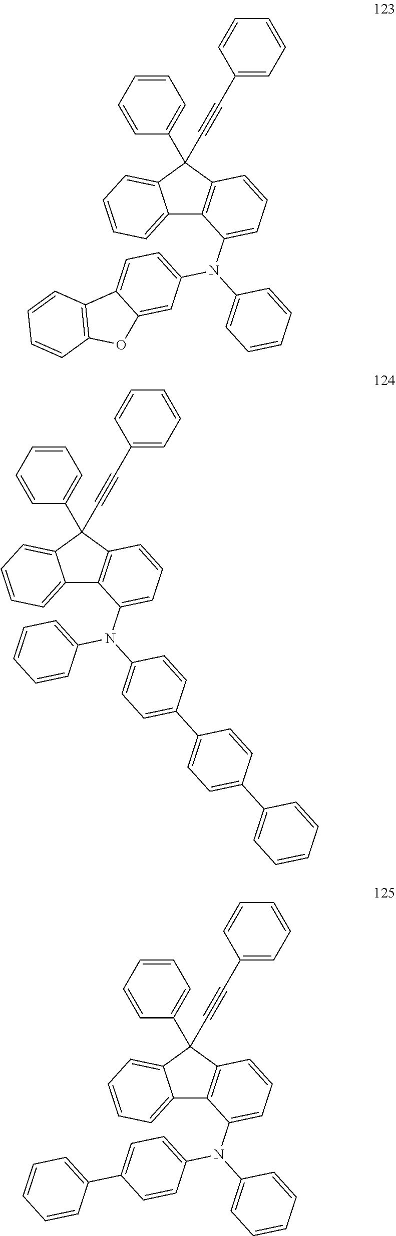

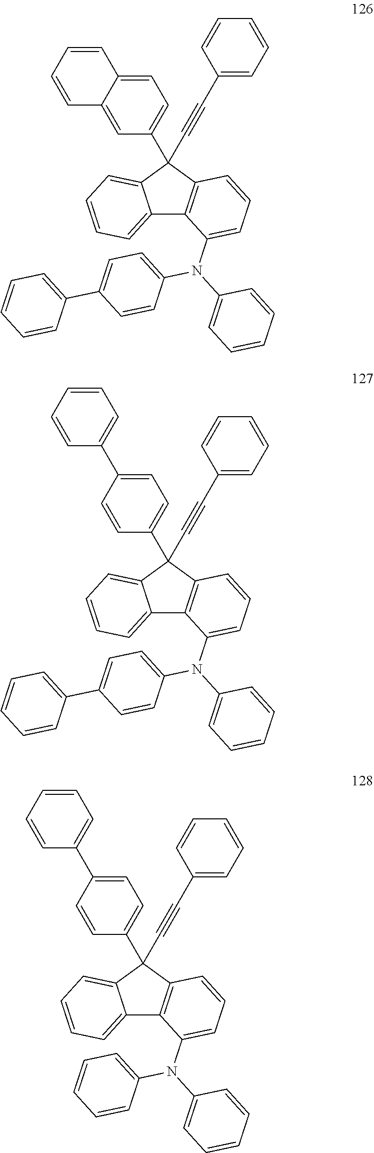 [SMILES: C(#CC1(c2ccc3ccccc3c2)c2ccccc2-c2c(N(c3ccccc3)c3ccc(-c4ccccc4)cc3)cccc21)c1ccccc1 ; C(#CC1(c2ccc(-c3ccccc3)cc2)c2ccccc2-c2c(N(c3ccccc3)c3ccc(-c4ccccc4)cc3)cccc21)c1ccccc1 ; C(#CC1(c2ccc(-c3ccccc3)cc2)c2ccccc2-c2c(N(c3ccccc3)c3ccccc3)cccc21)c1ccccc1]

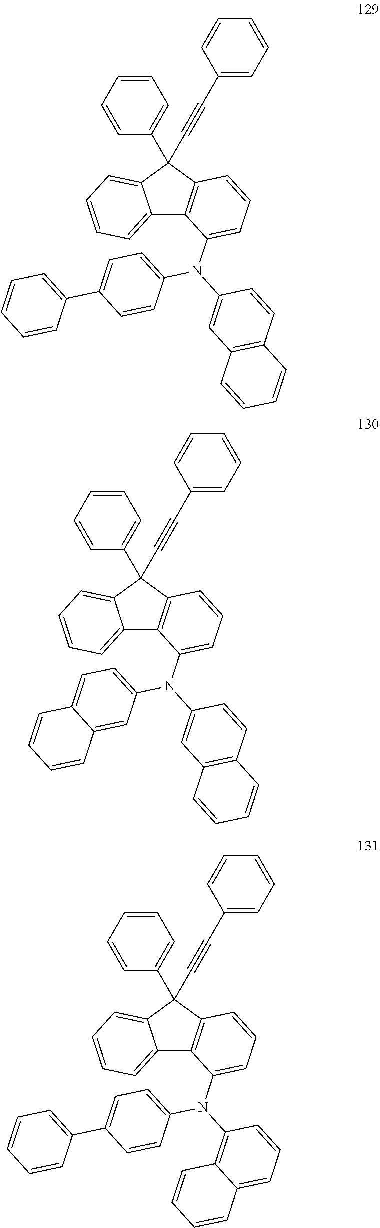

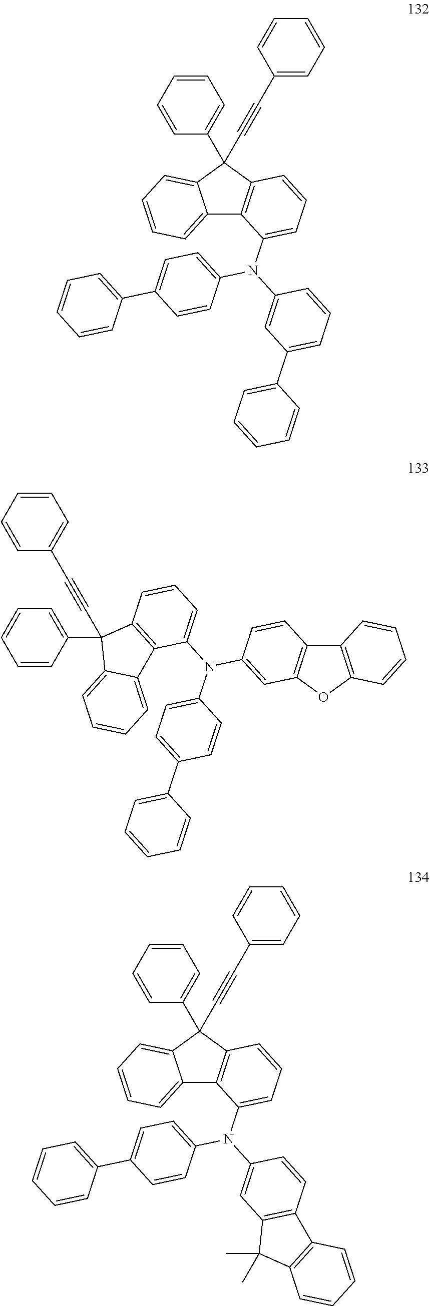 [SMILES: C(#CC1(c2ccccc2)c2ccccc2-c2c(N(c3ccc(-c4ccccc4)cc3)c3cccc(-c4ccccc4)c3)cccc21)c1ccccc1 ; C(#CC1(c2ccccc2)c2ccccc2-c2c(N(c3ccc(-c4ccccc4)cc3)c3ccc4c(c3)oc3ccccc34)cccc21)c1ccccc1 ; CC1(C)c2ccccc2-c2ccc(N(c3ccc(-c4ccccc4)cc3)c3cccc4c3-c3ccccc3C4(C#Cc3ccccc3)c3ccccc3)cc21]

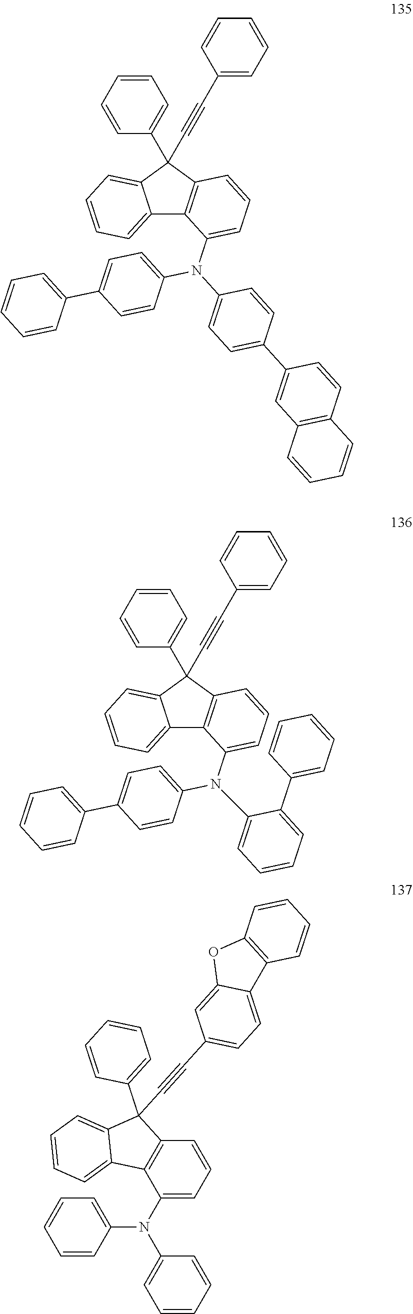

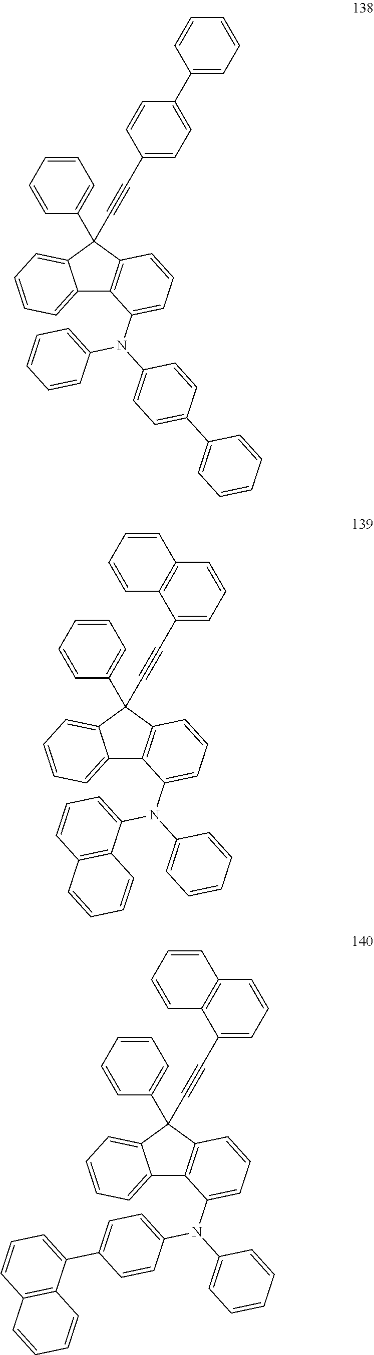 [SMILES: C(#CC1(c2ccccc2)c2ccccc2-c2c(N(c3ccccc3)c3ccc(-c4ccccc4)cc3)cccc21)c1ccc(-c2ccccc2)cc1 ; C(#CC1(c2ccccc2)c2ccccc2-c2c(N(c3ccccc3)c3cccc4ccccc34)cccc21)c1cccc2ccccc12 ; C(#CC1(c2ccccc2)c2ccccc2-c2c(N(c3ccccc3)c3ccc(-c4cccc5ccccc45)cc3)cccc21)c1cccc2ccccc12]

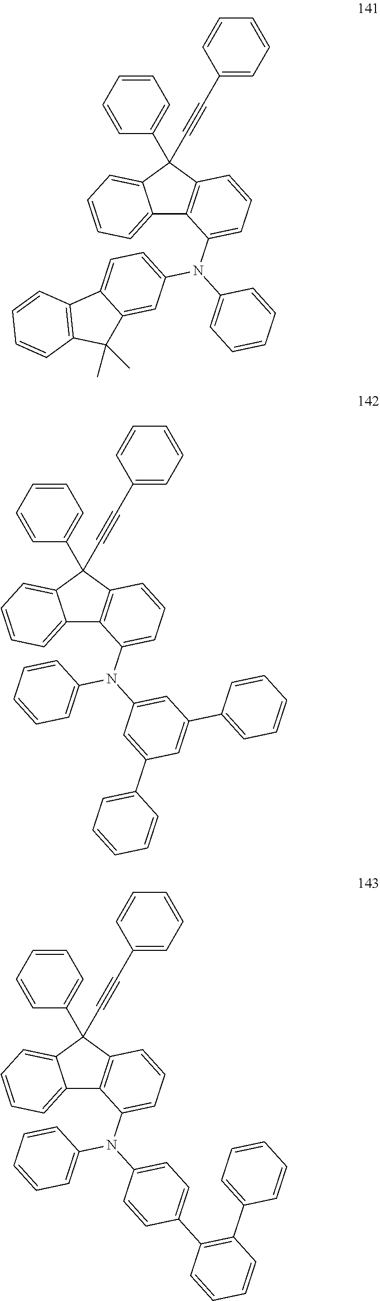

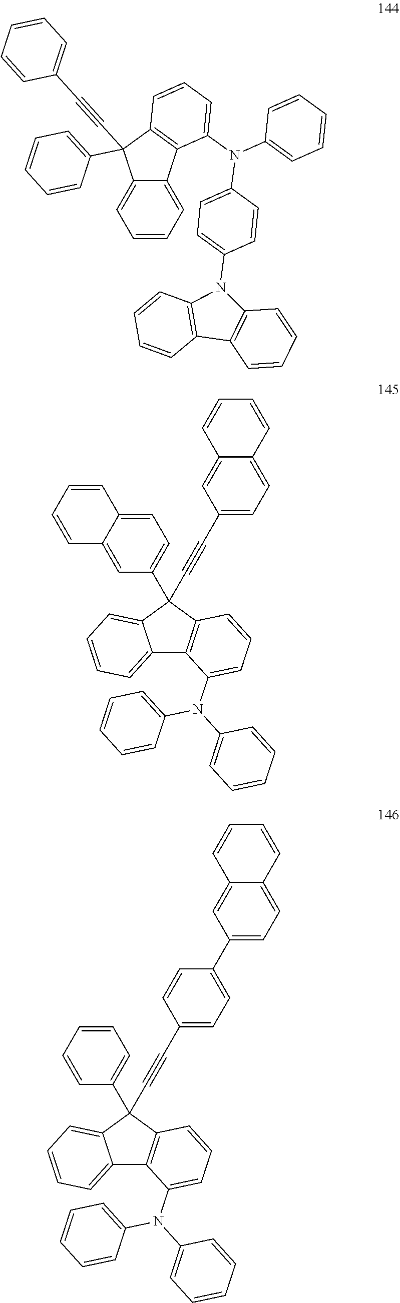

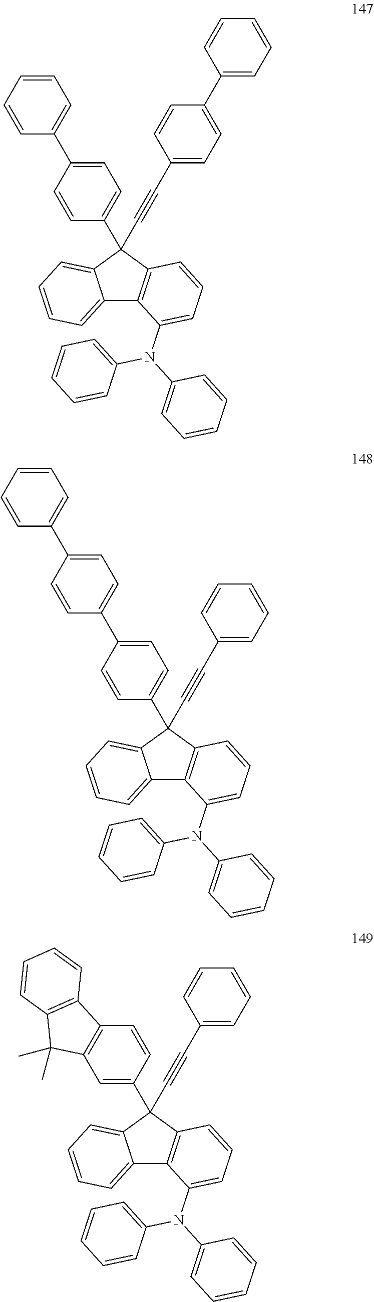 [SMILES: C(#CC1(c2ccc(-c3ccccc3)cc2)c2ccccc2-c2c(N(c3ccccc3)c3ccccc3)cccc21)c1ccc(-c2ccccc2)cc1 ; C(#CC1(c2ccc(-c3ccc(-c4ccccc4)cc3)cc2)c2ccccc2-c2c(N(c3ccccc3)c3ccccc3)cccc21)c1ccccc1 ; CC1(C)c2ccccc2-c2ccc(C3(C#Cc4ccccc4)c4ccccc4-c4c(N(c5ccccc5)c5ccccc5)cccc43)cc21]

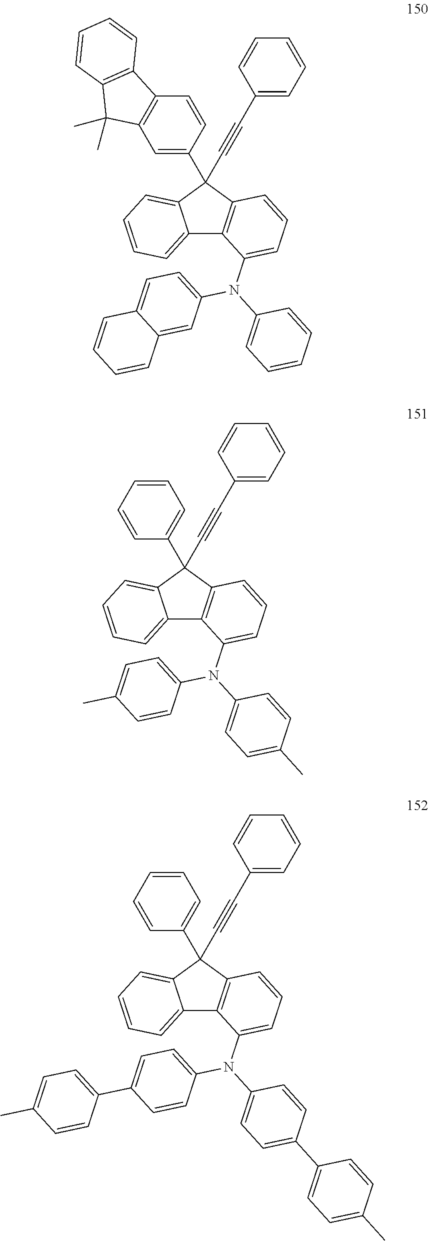

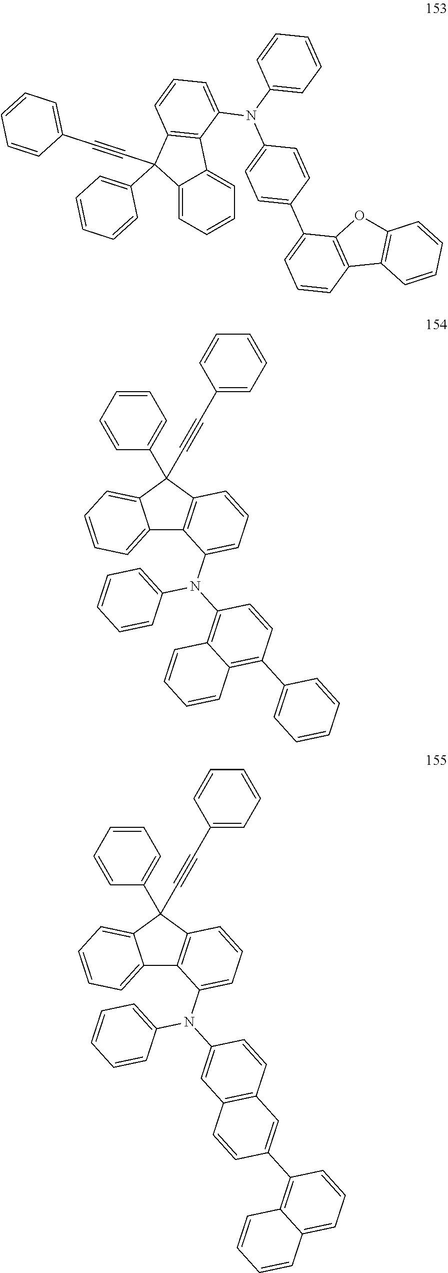 [SMILES: C(#CC1(c2ccccc2)c2ccccc2-c2c(N(c3ccccc3)c3ccc(-c4cccc5c4oc4ccccc45)cc3)cccc21)c1ccccc1 ; C(#CC1(c2ccccc2)c2ccccc2-c2c(N(c3ccccc3)c3ccc(-c4ccccc4)c4ccccc34)cccc21)c1ccccc1 ; C(#CC1(c2ccccc2)c2ccccc2-c2c(N(c3ccccc3)c3ccc4cc(-c5cccc6ccccc56)ccc4c3)cccc21)c1ccccc1]

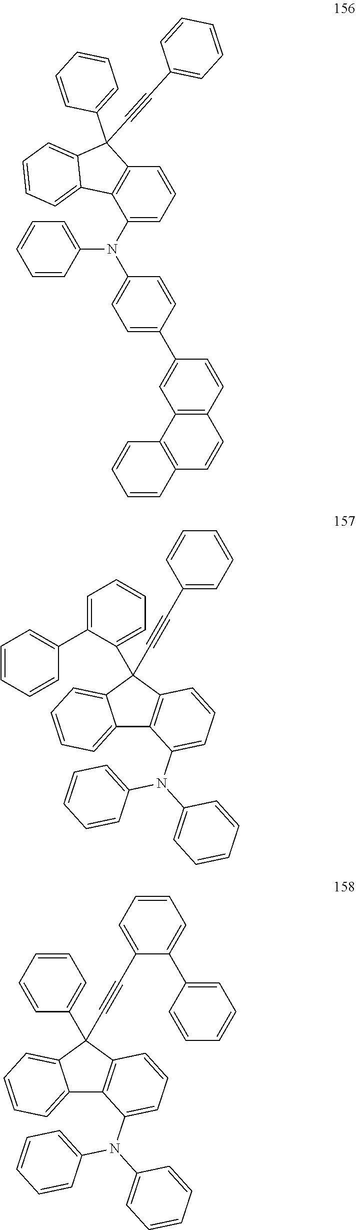

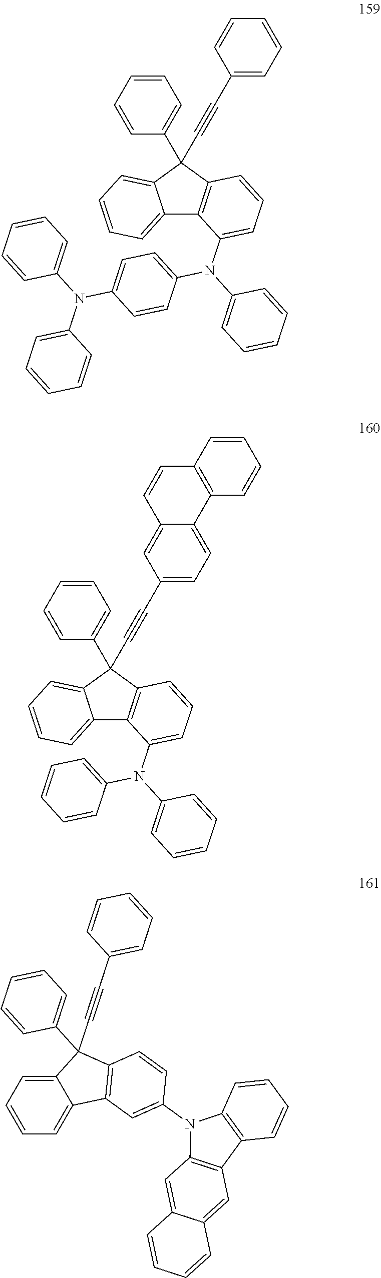 [SMILES: C(#CC1(c2ccccc2)c2ccccc2-c2c(N(c3ccccc3)c3ccc(N(c4ccccc4)c4ccccc4)cc3)cccc21)c1ccccc1 ; C(#CC1(c2ccccc2)c2ccccc2-c2c(N(c3ccccc3)c3ccccc3)cccc21)c1ccc2c(ccc3ccccc32)c1 ; C(#CC1(c2ccccc2)c2ccccc2-c2cc(-n3c4ccccc4c4cc5ccccc5cc43)ccc21)c1ccccc1]

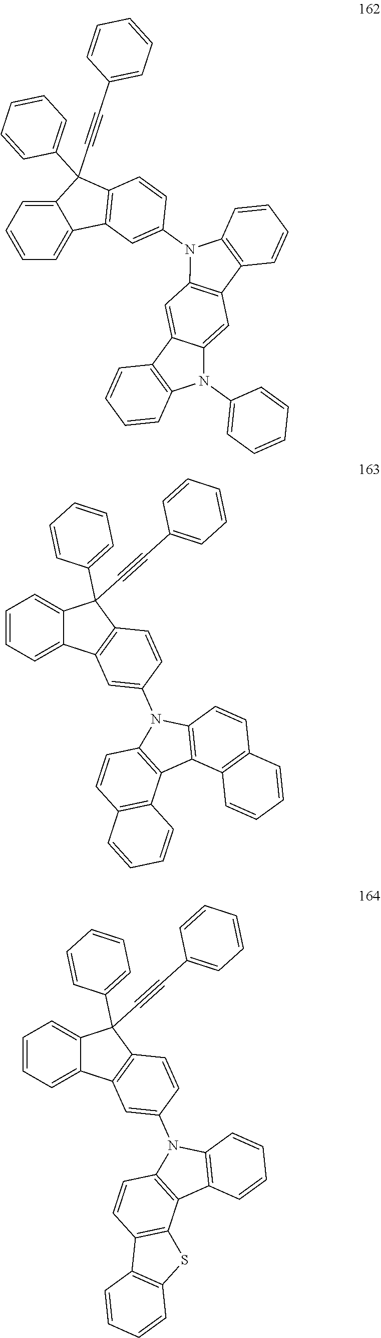 [SMILES: C(#CC1(c2ccccc2)c2ccccc2-c2cc(-n3c4ccccc4c4cc5c(cc43)c3ccccc3n5-c3ccccc3)ccc21)c1ccccc1 ; C(#CC1(c2ccccc2)c2ccccc2-c2cc(-n3c4ccc5ccccc5c4c4c5ccccc5ccc43)ccc21)c1ccccc1 ; C(#CC1(c2ccccc2)c2ccccc2-c2cc(-n3c4ccccc4c4c5sc6ccccc6c5ccc43)ccc21)c1ccccc1]

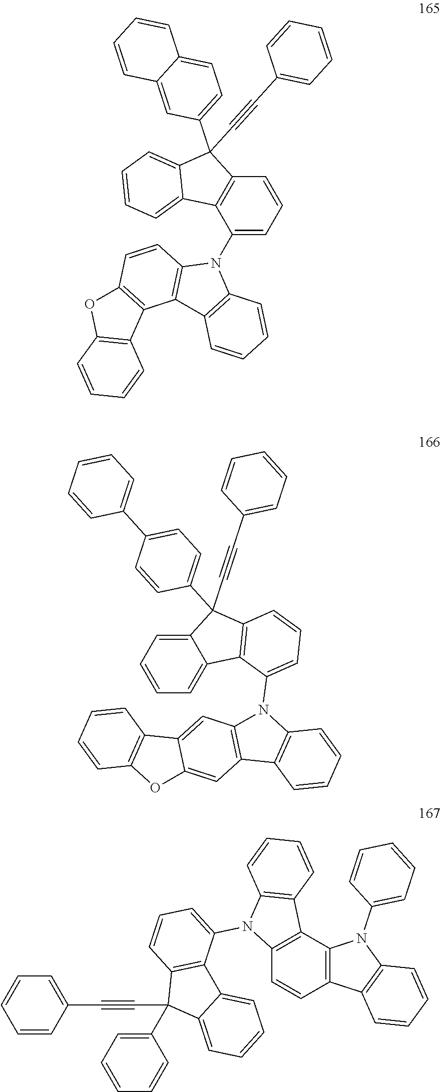 [SMILES: C(#CC1(c2ccc3ccccc3c2)c2ccccc2-c2c(-n3c4ccccc4c4c5c(ccc43)oc3ccccc35)cccc21)c1ccccc1 ; C(#CC1(c2ccc(-c3ccccc3)cc2)c2ccccc2-c2c(-n3c4ccccc4c4cc5oc6ccccc6c5cc43)cccc21)c1ccccc1 ; C(#CC1(c2ccccc2)c2ccccc2-c2c(-n3c4ccccc4c4c3ccc3c5ccccc5n(-c5ccccc5)c34)cccc21)c1ccccc1]

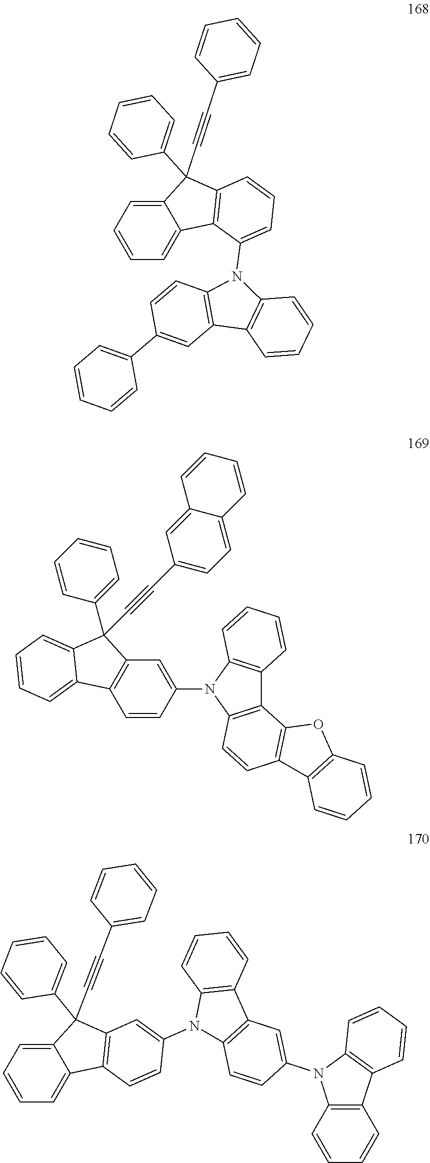 [SMILES: C(#CC1(c2ccccc2)c2ccccc2-c2c(-n3c4ccccc4c4cc(-c5ccccc5)ccc43)cccc21)c1ccccc1 ; C(#CC1(c2ccccc2)c2ccccc2-c2ccc(-n3c4ccccc4c4c5oc6ccccc6c5ccc43)cc21)c1ccc2ccccc2c1 ; C(#CC1(c2ccccc2)c2ccccc2-c2ccc(-n3c4ccccc4c4cc(-n5c6ccccc6c6ccccc65)ccc43)cc21)c1ccccc1]

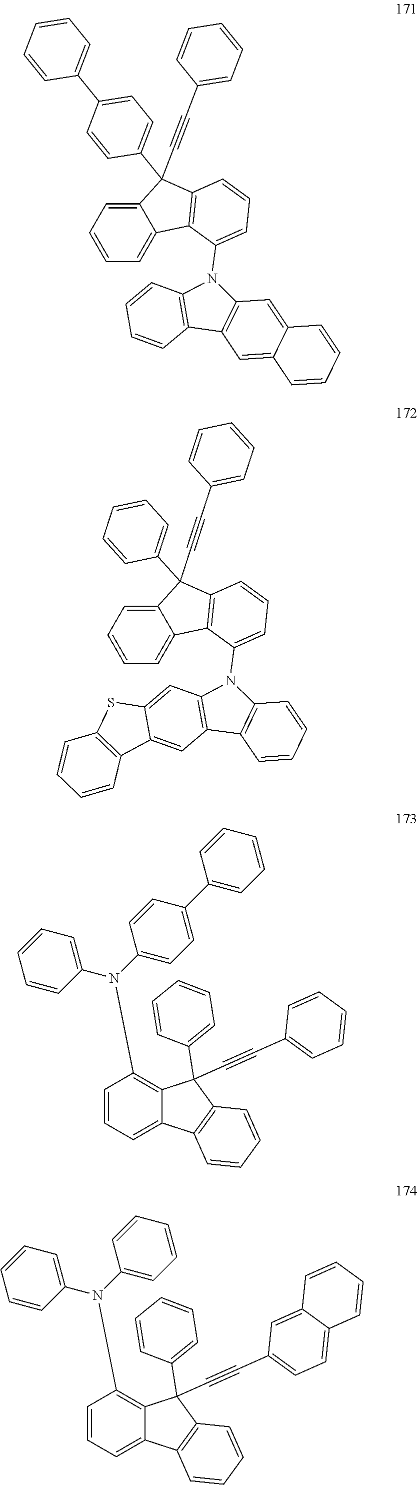 [SMILES: C(#CC1(c2ccc(-c3ccccc3)cc2)c2ccccc2-c2c(-n3c4ccccc4c4cc5ccccc5cc43)cccc21)c1ccccc1 ; C(#CC1(c2ccccc2)c2ccccc2-c2c(-n3c4ccccc4c4cc5c(cc43)sc3ccccc35)cccc21)c1ccccc1 ; C(#CC1(c2ccccc2)c2ccccc2-c2cccc(N(c3ccccc3)c3ccc(-c4ccccc4)cc3)c21)c1ccccc1 ; C(#CC1(c2ccccc2)c2ccccc2-c2cccc(N(c3ccccc3)c3ccccc3)c21)c1ccc2ccccc2c1]

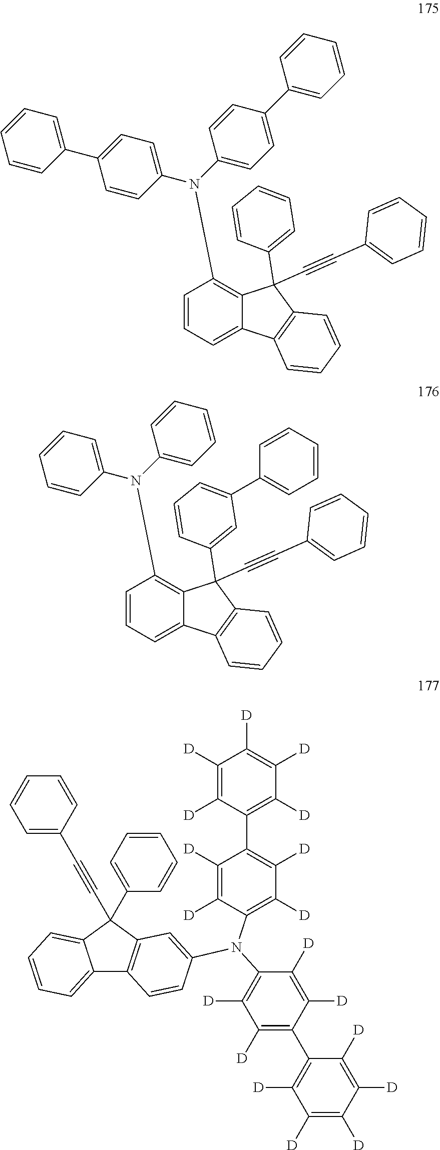 [SMILES: C(#CC1(c2ccccc2)c2ccccc2-c2cccc(N(c3ccc(-c4ccccc4)cc3)c3ccc(-c4ccccc4)cc3)c21)c1ccccc1 ; C(#CC1(c2cccc(-c3ccccc3)c2)c2ccccc2-c2cccc(N(c3ccccc3)c3ccccc3)c21)c1ccccc1 ; [2H]c1c([2H])c([2H])c(-c2c([2H])c([2H])c(N(c3ccc4c(c3)C(C#Cc3ccccc3)(c3ccccc3)c3ccccc3-4)c3c([2H])c([2H])c(-c4c([2H])c([2H])c([2H])c([2H])c4[2H])c([2H])c3[2H])c([2H])c2[2H])c([2H])c1[2H]]

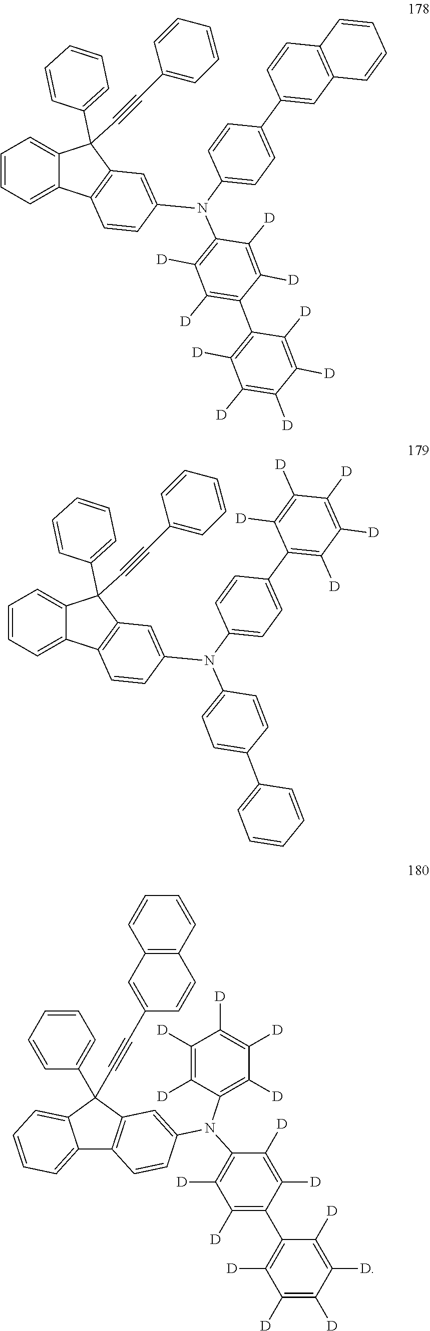

5. An organic light emitting device comprising:
a first electrode;
a second electrode provided to face the first electrode; and
one or more organic material layers provided between the first electrode and the second electrode,
wherein one or more layers of the organic material layers comprise the heterocyclic compound of claim 1.

6. The organic light emitting device of claim 5, wherein the organic material layer comprises a hole transfer layer, and the hole transfer layer comprises the heterocyclic compound.

7. The organic light emitting device of claim 5, wherein the organic material layer comprises an electron blocking layer, and the electron blocking layer comprises the heterocyclic compound.

8. The organic light emitting device of claim 5, wherein the organic material layer comprises a light emitting auxiliary layer, and the light emitting auxiliary layer comprises the heterocyclic compound.

9. The organic light emitting device of claim 5, wherein the organic material layer comprises an electron injection layer, a hole injection layer, an electron transfer layer or a hole blocking layer, and the electron transfer layer, the hole injection layer, the electron injection layer or the hole blocking layer comprises the heterocyclic compound.

10. The organic light emitting device of claim 5, further comprising one or more selected from the group consisting of a light emitting layer, a hole injection layer, a hole transfer layer, an electron injection layer, an electron transfer layer, an electron blocking layer and a hole blocking layer.
